(19) United States
(12) Patent Application Publication
(10) Pub. No.: US 2017/0045529 A1
(43) Pub. Date: Feb. 16, 2017

(54) METHODS OF MEASURING ANTIGEN-SPECIFIC T CELLS

(71) Applicant: ImmusanT, Inc., Cambridge, MA (US)

(72) Inventor: Robert P. ANDERSON, Shrewsbury, MA (US)

(73) Assignee: ImmusanT, Inc., Cambridge, MA (US)

(21) Appl. No.: 15/306,164

(22) PCT Filed: Apr. 24, 2015
(86) PCT No.: PCT/US2015/027497

§ 371 Date: Oct. 24, 2016

Related U.S. Application Data
(60) Provisional application No. 61/983,989, filed on Apr. 24, 2014.
(60) Provisional application No. 61/983,993, filed on Apr. 24, 2014.
(60) Provisional application No. 61/983,981, filed on Apr. 24, 2014.
(60) Provisional application No. 61/984,028, filed on Apr. 24, 2014.
(60) Provisional application No. 61/984,043, filed on Apr. 25, 2014.
(60) Provisional application No. 62/009,146, filed on Jun. 6, 2014.
(60) Provisional application No. 62/009,090, filed on Jun. 6, 2014.
(60) Provisional application No. 62/011,508, filed on Jun. 12, 2014.
(60) Provisional application No. 62/011,540, filed on Jun. 12, 2014.
(60) Provisional application No. 62/011,561, filed on Jun. 12, 2014.
(60) Provisional application No. 62/011,566, filed on Jun. 12, 2014.
(60) Provisional application No. 62/011,493, filed on Jun. 12, 2014.
(60) Provisional application No. 62/011,794, filed on Jun. 13, 2014.
(60) Provisional application No. 62/014,666, filed on Jun. 19, 2014.
(60) Provisional application No. 62/014,681, filed on Jun. 19, 2014.
(60) Provisional application No. 62/014,676, filed on Jun. 19, 2014.
(60) Provisional application No. 62/014,401, filed on Jun. 19, 2014.
(60) Provisional application No. 62/014,373, filed on Jun. 19, 2014.
(60) Provisional application No. 62/043,390, filed on Aug. 28, 2014.
(60) Provisional application No. 62/043,395, filed on Aug. 28, 2014.
(60) Provisional application No. 62/043,386, filed on Aug. 28, 2014.
(60) Provisional application No. 62/057,152, filed on Sep. 29, 2014.
(60) Provisional application No. 62/057,163, filed on Sep. 29, 2014.
(60) Provisional application No. 62/082,832, filed on Nov. 21, 2014.
(60) Provisional application No. 62/116,027, filed on Feb. 13, 2015.
(60) Provisional application No. 62/116,002, filed on Feb. 13, 2015.
(60) Provisional application No. 62/115,897, filed on Feb. 13, 2015.
(60) Provisional application No. 62/115,963, filed on Feb. 13, 2015.
(60) Provisional application No. 62/116,052, filed on Feb. 13, 2015.
(60) Provisional application No. 62/115,925, filed on Feb. 13, 2015.

(51) Int. Cl. G01N 33/68

(52) U.S. Cl. CPC G01N 33/6863; G01N 2333/522; G01N 2333/57; G01N 2333/55

(57) ABSTRACT

Provided herein are methods and kits for assaying antigen-specific T cell responses, such as rare autoantigen-specific T cell responses, by measuring a level of IP-10 in a sample from a subject having or suspected of having an autoimmune disease, an allergy, an infectious disease or condition, or an adverse immune condition caused by administration of an isolated, recombinant or synthetic protein or peptide. Also provided herein are methods and kits for assaying a T cell response to an antigen peptide, such as an islet autoantigen peptide, such as measuring a T cell response to at least one antigen peptide, such as an islet autoantigen peptide, in a sample from a subject, such as one having or suspected of having Type 1 Diabetes (TID), Celiac disease or both.

### RELATED APPLICATIONS

This application claims the benefit under 35 U.S.C. §119(e) of U.S. provisional application No. 61/983,981, filed Apr. 24, 2014, U.S. provisional application No. 62/011,561, filed Jun. 12, 2014, U.S. provisional application No. 62/014,676, filed Jun. 19, 2014, U.S. provisional application No. 62/057,152, filed Sep. 29, 2014, U.S. provisional application No. 62/115,925, filed Feb. 13, 2015, U.S. provisional application No. 61/984,028, filed Apr. 24, 2014, U.S. provisional application No. 61/984,043, filed Apr. 25, 2014, U.S. provisional application No. 62/011,566, filed Jun. 12, 2014, U.S. provisional application No. 62/014,681, filed Jun. 19, 2014, U.S. provisional application No. 62/057,163, filed Sep. 29, 2014, U.S. provisional application No. 62/115,897, filed Feb. 13, 2015, U.S. provisional application No. 61/983,989, filed Apr. 24, 2014, U.S. provisional application No. 62/014,666, filed Jun. 19, 2014, U.S. provisional application No. 62/009,146, filed Jun. 6, 2014, U.S. provisional application No. 62/043,386, filed Aug. 28, 2014, U.S. provisional application No. 62/115,963, filed Feb. 13, 2015, U.S. provisional application No. 61/983,993, filed Apr. 24, 2014, U.S. provisional application No. 62/011,508, filed Jun. 12, 2014, U.S. provisional application No. 62/116,052, filed Feb. 13, 2015, U.S. provisional application No. 62/043,395, filed Aug. 28, 2014, U.S. provisional application No. 62/082,832, filed Nov. 21, 2014, U.S. provisional application No. 62/009,090, filed Jun. 6, 2014, U.S. provisional application No. 62/014,373, filed Jun. 19, 2014, U.S. provisional application No. 62/043,390, filed Aug. 28, 2014, U.S. provisional application No. 62/116,002, filed Feb. 13, 2015, U.S. provisional application No. 62/011,493, filed Jun. 12, 2014, U.S. provisional application No. 62/011,794, filed Jun. 13, 2014, U.S. provisional application No. 62/014,401, filed Jun. 19, 2014, U.S. provisional application No. 62/116,027, filed Feb. 13, 2015, and U.S. provisional application No. 62/011,540, filed Jun. 12, 2014, the contents of each of which are incorporated by reference herein in their entirety.

### BACKGROUND

Identification of antigens is important for understanding disease pathology and for designing diagnostics and treatments. In some instances, T cells specific for such antigens may be rare or difficult to identify, meaning that the antigens themselves are also difficult to identify based on conventional T cell screening methods.

### SUMMARY

The disclosure relates, at least in part, to methods of assessing a T cell response to an antigen, e.g., an autoantigen. As described herein, methods for identifying antigen-specific T cells are desirable as such antigen-specific T cells may contribute to disease pathology and other phenotypes, e.g., autoimmune disease pathology or adverse reactions to therapeutics such as biologics. It is believed that such antigen-specific T cells can be detected using IP-10 as a biomarker. Accordingly, aspects of the disclosure relate to use of IP-10 as a marker for assessing antigen-specific T cell responses, e.g., rare antigen-specific T cell responses.

The disclosure also relates, at least in part, to methods of assessing a T cell response to an antigen peptide, such as an islet autoantigen peptide, e.g., either rare or common islet autoantigen-specific T cell responses. As described herein, a study is undertaken to assess whether a gluten challenge in patients with comorbid type 1 diabetes (T1D) and Celiac disease following a gluten free diet will mobilize both gluten-specific and islet autoantigen-specific T cells in blood. It is expected that such mobilization of gluten-specific and islet autoantigen-specific T cells will occur. Accordingly, other aspects of the disclosure relate to methods of assessing a T cell response to an antigen peptide, such as an islet autoantigen peptide, which may involve use of IP-10 or other biomarkers of a T cell response (e.g., IFN-γ and/or IL-2).

Aspects of the disclosure relate to a method of assaying an antigen-specific T cell response, the method comprising measuring a level of IP-10 in a sample comprising an antigen-specific T cell obtained from a subject. In some embodiments of any one of the methods provided, the antigen specific T cell response is a rare antigen-specific T cell response and wherein the antigen-specific T cell is a rare antigen-specific T cell.

In some embodiments of any one of the methods provided, the subject is a subject that has previously been administered IL-2 or an agent that stimulates IL-2 expression. In some embodiments of any one of the methods provided, the method further comprises administering IL-2 or an agent that stimulates IL-2 expression to the subject prior to the measuring.

In some embodiments of any one of the methods provided, the subject has or is suspected of having autoimmune disease, an allergy, an infectious disease or condition, or an adverse immune condition caused by administration of an isolated, recombinant or synthetic protein or peptide.

In some embodiments of any one of the methods provided, the subject has or is suspected of having the autoimmune disease and Celiac disease. In some embodiments of any one of the methods provided, the subject is a subject that has previously been administered a composition comprising a gluten peptide. In some embodiments of any one of the methods provided, the method further comprises administering a composition comprising a gluten peptide to the subject prior to the measuring. In some embodiments of any one of the methods provided, the composition is or has previously been administered to the subject more than once. In some embodiments of any one of the methods provided, the composition is or has previously been administered to the subject at least once a day for three days. In some embodiments of any one of the methods provided, the composition comprises at least one of a wheat gluten, a barley hordein, and a rye secalin. In some embodiments of any one of the methods provided, the composition comprises at least two of a wheat gluten, a barley hordein, and a rye secalin. In some embodiments of any one of the methods provided, the composition comprises a wheat gluten, a barley hordein, and a rye secalin. In some embodiments of any one of the methods provided, the administration of the composition is oral administration. In some embodiments of any one of the methods provided, the composition is a foodstuff. In some embodiments of any one of the methods provided, the sample is obtained from the subject six days after administration of the composition.

In some embodiments of any one of the methods provided, the sample comprises whole blood or peripheral blood mononuclear cells.

In some embodiments of any one of the methods provided, the measuring of the level of IP-10 in the sample comprises contacting the sample with an antigen peptide and measuring the level of IP-10 in the sample. In some embodiments of any one of the methods provided, the measuring of the level of IP-10 in the sample comprises contacting the sample with an antigen peptide, such as an autoantigen peptide, and measuring the level of IP-10 in the sample. In some embodiments of any one of the methods provided, the level of IP-10 is measured with an enzyme-linked immunosorbent assay (ELISA). In some embodiments of any one of the methods provided, the level of IP-10 is measured with a multiplex bead-based assay.

In some embodiments of any one of the methods provided, the method further comprises comparing the level of IP-10 with a control level of IP-10 to identify or aid in identifying the antigen peptide as being one that is recognized by the antigen-specific T cell. In some embodiments of any one of the methods provided, an elevated level of IP-10 compared to the control level indicates that the antigen peptide is recognized by the antigen-specific T cell and wherein a decreased or substantially the same level of IP-10 compared to the control level indicates that the antigen peptide is not recognized by the antigen-specific T cell. In some embodiments of any one of the methods provided, the subject has or is suspected of having an autoimmune disease and the antigen-specific T cell is a autoantigen-specific T cell. In some embodiments of any one of the methods provided, the method further comprises comparing the level of IP-10 with a control level of IP-10 to identify or aid in identifying the autoantigen peptide as being one that is recognized by the rare autoantigen-specific T cell. In some embodiments of any one of the methods provided, an elevated level of IP-10 compared to the control level indicates that the autoantigen peptide is recognized by the rare autoantigen-specific T cell and wherein a decreased or substantially the same level of IP-10 compared to the control level indicates that the autoantigen peptide is not recognized by the rare autoantigen-specific T cell. In some embodiments of any one of the methods provided, a level of IP-10 is elevated if the level of IP-10 is at least two-fold greater than a control level of IP-10. In some embodiments of any one of the methods provided, the control level of IP-10 is a level of IP-10 in a sample that has been contacted with a composition comprising phosphate buffered saline.

In some embodiments of any one of the methods provided, the method further comprises measuring a level of IFN-γ and/or IL-2 in the sample. In some embodiments of any one of the methods provided, the level of IFN-γ and/or IL-2 is compared to a control level of IFN-γ and/or IL-2. In some embodiments of any one of the methods provided, an elevated level of IFN-γ and/or IL-2 compared to the control level indicates that the autoantigen peptide is recognized by the rare autoantigen-specific T cell and wherein a decreased or substantially the same level of IFN-γ and/or IL-2 compared to the control level indicates that the autoantigen peptide is not recognized by the rare autoantigen-specific T cell. In some embodiments of any one of the methods provided, a level of IFN-γ and/or IL-2 is elevated if the level of IFN-γ and/or IL-2 is at least two-fold greater than a control level of IFN-γ and/or IL-2. In some embodiments of any one of the methods provided, the control level of IFN-γ and/or IL-2 is a level of IFN-γ and/or IL-2 in a sample that has been contacted with a composition comprising phosphate buffered saline.

Other aspects of the disclosure relate to a kit, comprising (a) a means for detecting a level of IP-10; and (b) at least one antigen peptide. In some embodiments of any one of the kits provided, the at least one antigen peptide is at least one autoantigen peptide. In some embodiments of any one of the kits provided, the at least one antigen peptide is at least one foreign antigen.

In some embodiments of any one of the kits provided, the means for detecting a level of IP-10 is an antibody that binds to IP-10. In some embodiments of any one of the kits provided, the kit further comprises IL-2 or an agent that stimulates IL-2 expression. In some embodiments of any one of the kits provided, the kit further comprises a composition comprising a gluten peptide. In some embodiments of any one of the kits provided, the composition comprises at least one of a wheat gluten, a barley hordein, and a rye secalin. In some embodiments of any one of the kits provided, the composition comprises at least two of a wheat gluten, a barley hordein, and a rye secalin. In some embodiments of any one of the kits provided, the composition comprises a wheat gluten, a barley hordein, and a rye secalin.

In some embodiments of any one of the kits provided, the kit comprises a container, such as a vial or tube, for whole blood. In some embodiments of any one of the kits provided, the at least antigen peptide is dried on the wall of the container for whole blood. In some embodiments of any one of the kits provided, the at least one antigen peptide is in a solution or lyophilized in a separate container. In some embodiments of any one of the kits provided, the kit further comprises an anticoagulant. In some embodiments of any one of the kits provided, the container for whole blood and/or other container are present in duplicate or triplicate. In some embodiments of any one of the kits provided, the kit further comprises a negative control container, such as a vial or tube. In some embodiments of any one of the kits provided, the kit further comprises a positive control container, such as a vial or tube.

In some embodiments of any one of the kits provided, the kit further comprises means for detecting a level of IFN-γ and/or IL-2. In some embodiments of any one of the kits provided, the means for detecting a level of IFN-γ is an antibody that binds to IFN-γ and/or the means for detecting a level of IL-2 is an antibody that binds to IL-2.

Other aspects of the disclosure relate to a method of assaying a T cell response to an islet autoantigen peptide, the method comprising:

(a) administering a composition comprising a gluten peptide to a first subject having or suspected of having Type 1 Diabetes (T1D) and Celiac disease; and

(b) measuring a first T cell response to at least one islet autoantigen peptide in a first sample obtained from the first subject after the administration of the composition. In some embodiments of any one of the methods provided, the at least one islet autoantigen peptide is selected from a proinsulin peptide, a 65-kDa isoform of glutamic acid decarboxylase (GAD 65) peptide, or an islet antigen-2 (IA-2) peptide. In some embodiments of any one of the methods provided, the at least one autoantigen peptide is a peptide comprising a sequence as put forth in Table 3. In some embodiments of any one of the methods provided, the first sample comprises whole blood or peripheral blood mononuclear cells.

In some embodiments of any one of the methods provided, the composition is administered to the first subject more than once. In some embodiments of any one of the methods provided, the composition is administered to the first subject at least once a day for three days. In some embodiments of any one of the methods provided, the composition comprises at least one of a wheat gluten, a barley hordein, and a rye secalin. In some embodiments of any one of the methods provided, the composition comprises at least two of a wheat gluten, a barley hordein, and a rye secalin. In some embodiments of any one of the methods provided, the composition comprises a wheat gluten, a barley hordein, and a rye secalin. In some embodiments of any one of the methods provided, the administration of the composition is oral administration. In some embodiments of any one of the methods provided, the composition is a foodstuff.

In some embodiments of any one of the methods provided, the measuring of the first T cell response in the first sample comprises contacting the first sample with the at least one antigen peptide, such as an islet autoantigen peptide, and measuring a level of at least one cytokine in the first sample. In some embodiments of any one of the methods provided, the at least one cytokine is IL-2 and/or IFN-γ and/or IP-10. In some embodiments of any one of the methods provided, the level of the at least one cytokine is measured with an enzyme-linked immunosorbent assay (ELISA). In some embodiments of any one of the methods provided, the level of the at least one cytokine is measured with a multiplex bead-based assay. In some embodiments of any one of the methods provided, the level of the at least one cytokine is measured with an enzyme-linked immunosorbent spot (ELISpot) assay.

In some embodiments of any one of the methods provided, the method further comprises comparing the first T cell response with a control T cell response to identify or aid in identifying the first subject as in need of further testing for T1D if the T cell response measured in the first sample is elevated compared to the control T cell response, or to identify or aid in identifying the first subject as not in need of further testing for T1D if the first T cell response is substantially the same or decreased compared to the control T cell response. In some embodiments of any one of the methods provided, the method further comprises performing further testing for T1D if the first subject is identified as in need of further testing for T1D. In some embodiments of any one of the methods provided, the further testing comprises a glycated hemoglobin test, a glucose tolerance test, a fasting blood sugar test, and/or an immunoassay for autoantibodies. In some embodiments of any one of the methods provided, autoantibodies comprises one or more of islet cell autoantibodies, insulin autoantibodies, 65-kDa isoform of glutamic acid decarboxylase (GAD65) autoantibodies, islet antigen-2 (IA-2) autoantibodies, and zinc transporter (ZnT8) autoantibodies.

In some embodiments of any one of the methods provided, the first sample is obtained from the first subject six days after administration of the composition.

In some embodiments of any one of the methods provided, the method further comprises:

(c) administering a placebo to a second subject having or suspected of having Type 1 Diabetes (T1D) and Celiac disease; and

(d) measuring a second T cell response to the at least one islet autoantigen peptide in a second sample obtained from the second subject after the administration of the placebo. In some embodiments of any one of the methods provided, the measuring of the first and second T cell response are performed together in one assay.

In some embodiments of any one of the methods provided, the composition is administered to the first subject more than once and the placebo is administered to the second subject more than once. In some embodiments of any one of the methods provided, the composition is administered to the first subject at least once a day for three days and the placebo is administered to the second subject at least once a day for three days. In some embodiments of any one of the methods provided, the administration of the composition and the placebo is oral administration. In some embodiments of any one of the methods provided, the composition and the placebo are foodstuffs.

In some embodiments of any one of the methods provided, the measuring of the first and second T cell response in the first and second sample comprises contacting the first and second samples with the at least one antigen peptide, such as an islet autoantigen peptide, and measuring a level of at least one cytokine in the first and second samples. In some embodiments of any one of the methods provided, the at least one cytokine is IL-2 and/or IFN-γ and/or IP-10. In some embodiments of any one of the methods provided, the level of the at least one cytokine is measured with an enzyme-linked immunosorbent assay (ELISA). In some embodiments of any one of the methods provided, the level of the at least one cytokine is measured with an enzyme-linked immunosorbent spot (ELISpot) assay. In some embodiments of any one of the methods provided, the level of the at least one cytokine is measured with a multiplex bead-based assay.

In some embodiments of any one of the methods provided, the method further comprises comparing the first T cell response with the second T cell response to identify or aid in identifying the first subject as in need of further testing for T1D if the T cell response measured in the first sample is elevated compared to the second T cell response, or to identify or aid in identifying the first subject as not in further testing for T1D if the first T cell response is substantially the same or decreased compared to the second T cell response. In some embodiments of any one of the methods provided, the method further comprises performing further testing for T1D if the first subject is identified as in need of further testing for T1D. In some embodiments of any one of the methods provided, the further testing comprises a glycated hemoglobin test, a glucose tolerance test, a fasting blood sugar test, and/or an immunoassay for autoantibodies. In some embodiments of any one of the methods provided, autoantibodies comprises one or more of islet cell autoantibodies, insulin autoantibodies, 65-kDa isoform of glutamic acid decarboxylase (GAD65) autoantibodies, islet antigen-2 (IA-2) autoantibodies, and zinc transporter (ZnT8) autoantibodies.

In some embodiments of any one of the methods provided, the second sample is obtained from the second subject six days after administration of the placebo.

In some embodiments of any one of the methods provided, the method further comprises performing another test on the first subject and/or second subject prior to or after the steps of the method, preferably, in some embodiments, performing a serology and/or genotyping assay. In some embodiments of any one of the methods provided, the performing a serology and/or genotyping assay occurs prior to all of the steps recited in the method. In some embodiments of any one of the methods provided, the performing a serology and/or genotyping assay occurs after all of the steps recited in the method.

In some embodiments of any one of the methods provided, the first subject and/or second subject is HLA-DQ2.5 positive.

Yet other aspects of the disclosure relate to a kit, comprising (a) a means for detecting a T cell response; and (b) at least one antigen peptide, such as an islet autoantigen peptide. In some embodiments of any one of the kits provided, the at least one islet autoantigen peptide is selected from a proinsulin peptide, a 65-kDa isoform of glutamic acid decarboxylase (GAD 65) peptide, or an islet antigen-2 (IA-2) peptide. In some embodiments of any one of the kits provided, the at least one autoantigen peptide is a peptide comprising a sequence as put forth in Table 3.

In some embodiments of any one of the kits provided, the means for detecting a T cell response is an antibody that binds to a cytokine. In some embodiments of any one of the kits provided, the antibody that binds to a cytokine is an antibody that binds to IL-2 and/or IFN-γ and/or IP-10.

In some embodiments of any one of the kits provided, the kit further comprises a composition comprising a gluten peptide. In some embodiments of any one of the kits provided, the kit further comprises a placebo.

In some embodiments of any one of the kits provided, the composition and the placebo are foodstuffs. In some embodiments of any one of the kits provided, the composition comprises at least one of a wheat gluten, a barley hordein, and a rye secalin. In some embodiments of any one of the kits provided, the composition comprises at least two of a wheat gluten, a barley hordein, and a rye secalin. In some embodiments of any one of the kits provided, the composition comprises a wheat gluten, a barley hordein, and a rye secalin.

In some embodiments of any one of the kits provided, the kit comprises a container, such as a vial or tube, for whole blood. In some embodiments of any one of the kits provided, the at least one antigen peptide, such as an islet autoantigen peptide, is dried on the wall of the container for whole blood. In some embodiments of any one of the kits provided, the at least one antigen peptide, such as an islet autoantigen peptide, is in a solution or lyophilized in a separate container.

In some embodiments of any one of the kits provided, the kit further comprises an anticoagulant. In some embodiments of any one of the kits provided, the container for whole blood and/or other container are present in duplicate or triplicate. In some embodiments of any one of the kits provided, the kit further comprises a negative control container, such as a vial or tube. In some embodiments of any one of the kits provided, the kit further comprises a positive control container, such as a vial or tube.

Other aspects of the disclosure relate to a method of screening for peptides that activate antigen-specific T cells, the method comprising providing a plurality of antigen peptides comprising sequences derived from an antigen; contacting a plurality of samples comprising antigen-specific T cells obtained from a subject with the plurality of antigen peptides; and measuring a level of IP-10 in each of the samples within the plurality of samples.

In some embodiments of any one of the methods provided, the plurality of antigen peptides is 10-10,000 peptides. In some embodiments of any one of the methods provided, each of the antigen peptides within the plurality of antigen peptides is 10 to 20 amino acids in length. In some embodiments of any one of the methods provided, the plurality of antigen peptides comprise one or more peptides comprising one or more deamidated variants of the sequences derived from the antigen. In some embodiments, the plurality of antigen peptides contacted with the plurality of samples is present in an amount of 0.4 micrograms/mL, 1 microgram/mL, 4 micrograms/mL, 5 micrograms/mL, 10 micrograms/mL, 20 micrograms/mL, 25 micrograms/mL, or 50 micrograms/mL.

In some embodiments of any one of the methods provided, the subject has or is suspected of having an autoimmune disease, an allergy, an infectious disease or condition, or an adverse immune condition caused by administration of an isolated, recombinant or synthetic protein or peptide. In some embodiments of any one of the methods provided, the antigen is an autoantigen or a foreign antigen.

In some embodiments of any one of the methods provided, the level of IP-10 is measured using an ELISA assay or a multiplex bead-based assay. In some embodiments of any one of the methods provided, the method further comprises measuring a level of IL-2 and/or IFN-γ in each of the samples within the plurality of samples. In some embodiments of any one of the methods provided, the level of IL-2 and/or IFN-γ is measured using an ELISA assay or a multiplex bead-based assay.

In some embodiments of any one of the methods provided, the method further comprises identifying a peptide within the plurality of antigen peptides as a peptide that activates antigen-specific T cells if the level of IP-10 is elevated compared to a control level of IP-10. In some embodiments of any one of the methods provided, the method further comprises identifying a peptide within the plurality of antigen peptides as a peptide that activates antigen-specific T cells if the level of IP-10 is at least two-fold greater than a control level of IP-10. In some embodiments of any one of the methods provided, the control level of IP-10 is a level of IP-10 in a sample that has been contacted with a composition comprising phosphate buffered saline.

In some embodiments of any one of the methods provided, the method further comprises identifying a peptide within the plurality of antigen peptides as a peptide that activates antigen-specific T cells if the level of IP-10 is elevated compared to a control level of IP-10 and the level of IL-2 and/or IFN-γ and is elevated compared to a control level of IL-2 and/or IFN-γ, respectively.

In some embodiments of any one of the methods provided, the method further comprises identifying a peptide within the plurality of antigen peptides as a peptide that activates antigen-specific T cells if the level of IP-10 is at least two-fold greater than a control level of IP-10 and the level of IL-2 and/or IFN-γ and is at least two-fold greater than a control level of IL-2 and/or IFN-γ, respectively. In some embodiments of any one of the methods provided, the control level of IP-10 and the control level of IL-2 and/or IFN-γ is a level of IP-10 and IL-2 and/or IFN-γ, respectively, in a sample that has been contacted with a composition comprising phosphate buffered saline.

In some embodiments of any one of the methods provided, the antigen-specific T cells are rare antigen-specific T cells.

In some embodiments of any one of the methods provided, the method further comprises recording the level(s), value(s), amount(s), or result(s) of a measuring, assessment, and/or identification step.

The details of one or more embodiments of the disclosure are set forth in the description below. Other features or advantages of the present disclosure will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a schematic of the study design in Example 1.

FIG. 2 is a graph showing the schedule of assessments in the study of Example 1.

### DETAILED DESCRIPTION

Abnormal T cell responses to antigens, such as autoantigens, contribute to diseases, such as autoimmune diseases (e.g., thyroid disease, type I diabetes, and multiple sclerosis, and other immune-mediated diseases such as Celiac Disease). T cell responses to foreign antigens can also contribute to disease pathology and conditions, such as adverse reactions to therapeutics. Identification of antigens, such as autoantigens and foreign antigens, is important for understanding disease pathology and critical for designing targeted diagnostics and treatments. In some instances, T cells specific for such antigens may be rare or difficult to identify, meaning that the antigens themselves are also difficult to identify based on conventional T cell screening methods. Without wishing to be bound by theory, it is believed that IFN-γ is a potent activator of innate immunity mediated by monocytes and neutrophils. Inducible protein-10 (IP-10) can be released in substantial amounts by a variety of human cells including monocytes and neutrophils upon exposure to IFN-γ (Luster, A. D. & Ravetch, J. V. Biochemical characterization of a gamma interferon-inducible cytokine (IP-10). The Journal of experimental medicine 166, 1084-1097 (1987); and Cassatella, M. A., et al. Regulated production of the interferon-gamma-inducible protein-10 (IP-10) chemokine by human neutrophils. European journal of immunology 27, 111-115 (1997)). Circulating antigen-specific memory CD4 T cells relevant to autoimmune disease including celiac disease may number no more 1-10 per milliliter of blood (Christophersen, A., et al. Tetramer-visualized gluten-specific CD4+ T cells in blood as a potential diagnostic marker for coeliac disease without oral gluten challenge. United European gastroenterology journal 2, 268-278 (2014). In contrast, the frequency of circulating moncytes and neutrophils capable of detecting and responding to IFN-γ is approximately a million-times higher than rare antigen-specific T cells. Assessing IP-10 levels in blood incubated with a potentially antigenic peptide of interest provides a desirable biomarker for assessing antigen-specific T cell responses, including rare antigen-specific T cell responses, as IP-10 provides a robust readout for rare T cells secreting IFN-γ.

In Celiac Disease, the lining mucosa of the upper gut becomes infiltrated by chronic inflammatory cells¹. In the small intestine where the damage is most marked, the surface finger-like projections (villi) become flattened causing the typical histological appearance of “villous atrophy and crypt hyperplasia”. The environmental factor causing celiac disease is a ubiquitous dietary protein, gluten, derived from wheat, barley and rye flour. Individuals are only susceptible to celiac disease if they possess the immune recognition genes HLA-DQ2 or HLA DQ8, that are collectively found in about half of most Indo-Europeans². An aggressive, pro-inflammatory immune response led by CD4 T cells targets partially digested gluten fragments and triggers tissue damage in organs exposed to gluten and as well as causing a variety of complications outside the gut—skin rash, hepatitis, osteoporosis, fatigue, migraines, infertility, developmental delay and reduced growth in children¹. Although gluten free diet has recently become popular, the prevalence of Celiac Disease has been about 1-2% for some time³. Medical awareness is improving, but still only about 10% of Americans truly affected by Celiac Disease have been formally diagnosed⁴. Diagnosis often requires small bowel biopsy and is supported by the presence of serum autoantibody specific for transglutaminase and/or deamidated gliadin peptides, and HLA-DQ2 or DQ8 genes⁵. In some cases, gluten exclusion followed by gluten challenge for a month or even up to two-years is needed to confirm whether Celiac Disease is present⁶. Currently, the only treatment for Celiac Disease is to avoid the causative antigen with a strict, life-long gluten-free diet¹. Other therapeutic approaches are being explored and include a therapeutic vaccine intended to restore immune tolerance to gluten (ImmusanT, Inc. Cambridge Mass.)⁷.

Celiac Disease stands out as an immune-mediated disease closely related to T1D; the most important susceptibility genes (HLA-DR3-DQ2, and HLA-DR4-DQ8) are the same⁸, both conditions are caused by an acquired cell-mediated immune response orchestrated by CD4 T cells, and the affected organs are adjacent (proximal small intestine and pancreas) meaning that immune cells migrating from the inflamed intestinal mucosa and pancreatic islets are likely to drain to the same local lymph nodes⁹. This suggests immune-mediated pathology in the proximal small intestine and pancreatic islets may be linked through paracrine effects of cytokines released by activated T cells in local draining lymph nodes. Indeed, Celiac Disease affects 10% of patients with T1D and both conditions have a similar age of onset^{10,11}. In clinical practice, patients with T1D are regularly tested for serum transglutaminase IgA ensuring prompt diagnosis, and consequently avoiding complications of Celiac Disease¹².

A case report of a 6-year old boy with recent onset T1D adopting gluten-free diet and entering extended disease remission without insulin therapy highlighted the importance of resolving the functional relationship between dietary gluten and islet autoimmunity¹³. Animal models have supported dietary gluten contributing to the development of autoimmune diabetes, and also to mild intestinal damage in NOD mice prone to islet autoimmunity¹⁴. Dietary gluten also contributes to reduction in the number of anti-inflammatory (regulatory) T cells in the intestinal lining of mice¹⁵. However, human studies addressing the link between gluten and T1D have been limited and inconclusive. Klemetti et al. reported isolating gluten-reactive T cells in seven of 29 patients with newly diagnosed T1D, significantly more than in non-diabetic controls (two of 37)¹⁶. But Hummel et al. were unable to demonstrate that delaying gluten introduction in the diet of infants reduced development of islet autoantibodies or T1D¹⁷.

As described herein, a study is undertaken to assess the mobilization of both gluten-specific and islet autoantigen-specific T cells in blood with a gluten challenge in patients with comorbid type 1 diabetes (T1D) and Celiac disease following a gluten free diet. Without wishing to be bound by theory, it is thought that the immune stimulation provided by gluten in patients with celiac disease who also have T1D might cause not only gluten-reactive T cells but also islet-autoantigen-specific T cells to appear in the peripheral blood. This “bystander” stimulation of islet-specific T cells can occur if T cells specific for gluten are activated and secrete T-cell growth factors such as interleukin-2 that stimulate proliferation of not just gluten-specific T cells but also islet autoantigen-specific T cells in the same local draining lymph nodes. This “bystander” stimulation can be measured, e.g., using IP-10 or other T cell response biomarkers (e.g., IFN-γ).

Accordingly, the disclosure provides methods and kits related to assaying a T cell response to an antigen, such as an islet autoantigen peptide.

It is also believed that a similar approach can be applied to other autoimmune diseases that may or may not co-exist with Celiac disease. Without wishing to be bound by theory, it is thought that a gluten challenge can be used to provoke bystander stimulation of rare autoantigen-specific T cells of other comorbid autoimmune diseases because of release of cytokines such IL-2 would stimulate all T cells not just those specific for gluten-derived epitopes. Other means of bystander stimulation of T cells could be substituted for the gluten challenge, e.g., an agent that drives IL-2 stimulation of T cells, such as IL-2 or an agent that stimulates IL-2 expression.

This approach may also be used in autoimmune diseases that are not co-morbid with Celiac disease such as an autoimmune disease described herein. In such cases, it may be advantageous to use an agent that drives IL-2 stimulation of T cells, such as IL-2 or an agent that stimulates IL-2 expression, as a gluten challenge may not result in IL-2 stimulation in subjects that do not have Celiac disease. This approach may also be used for any other disease or condition that might be associated with rare antigen-specific T cells, such as allergic and infectious diseases or conditions or diseases associated with administration or contact with foreign antigens, whether or not they might co-exist with Celiac disease.

Further, it is expected that IP-10 will be a robust readout for T cell responses, such as bystander stimulation of rare antigen-specific T cells, such as rare autoantigen-specific T cells. The IP-10 readout may also be used as a readout for rare antigen-specific T cell responses in the absence of stimulation of the T cells prior to assessment of the readout, such as in the absence of IL-2 stimulation.

Accordingly, the disclosure also provides methods and kits related to assaying a rare antigen-specific T cell response, e.g., a rare autoantigen-specific T cell response, using IP-10 as a biomarker. In some embodiments, of any one of the methods and kits assaying a rare antigen-specific T cell response can be done using IP-10 and/or IL-2 and/or IFN-γ as biomarker(s).

### Methods

One aspect of the disclosure relates to methods of assaying a T cell response to an antigen, such as an islet autoantigen peptide.

In some embodiments, the method comprises (a) administering a composition comprising a gluten peptide as described herein to a first subject having or suspected of having Type 1 Diabetes (T1D) and Celiac disease; and (b) measuring a first T cell response to at least one islet autoantigen peptide as described herein in a first sample obtained from the first subject after the administration of the composition. Assays for measuring a T cell response are described herein.

In some embodiments, the method further comprises comparing the first T cell response with a control T cell response to identify or aid in identifying the first subject as in need of further testing for T1D if the T cell response measured in the first sample is elevated compared to the control T cell response, or to identify or aid in identifying the first subject as not in need of further testing for T1D if the first T cell response is substantially the same or decreased compared to the control T cell response. In some embodiments, the T cell response measured in the first sample is elevated if it is at least two times higher than the control T cell response. In some embodiments, the control T cell response is a T cell response in a sample from the first subject obtained before the administration of the composition comprising the gluten peptide. Further testing for T1D is described herein.

In some embodiments of any one of the methods provided, the method further comprises (c) administering a placebo to a second subject having or suspected of having T1D and Celiac disease; and (d) measuring a second T cell response to the at least one islet autoantigen peptide in a second sample obtained from the second subject after the administration of the placebo. In some embodiments, the method further comprises comparing the first T cell response with the second T cell response to identify or aid in identifying the first subject as in need of further testing for T1D if the T cell response measured in the first sample is elevated compared to the second T cell response, or to identify or aid in identifying the first subject as not in need of further testing for T1D if the first T cell response is substantially the same or decreased compared to the second T cell response. In some embodiments, the T cell response measured in the first sample is elevated if it is at least two times higher than the second T cell response.

In some embodiments of any one of the methods provided, the method further comprises performing other testing on the first subject and/or second subject prior to or after the steps of the method, such as other testing for Celiac disease or T1D. Other testing is described herein.

Another aspect of the disclosure relates to assaying a rare antigen-specific T cell response, e.g., a rare autoantigen-specific T cell response. In some embodiments, the method comprises measuring a level of IP-10 in a sample comprising a rare antigen-specific T cell (e.g., a rare autoantigen-specific T cell) obtained from a subject as described herein, e.g., a subject having or suspected of having an autoimmune disease, an allergy, an infectious disease or condition, or an adverse immune condition caused by administration of an isolated, recombinant or synthetic protein or peptide, such as a therapeutic. In some embodiments of any one of the methods provided, the measuring of the level of IP-10 in the sample comprises contacting the sample with an antigen peptide (e.g., an autoantigen peptide) as described herein and measuring the level of IP-10 in the sample. Assays for measuring a level of IP-10 are described herein. In some embodiments of any one of the methods provided, the method further comprises comparing the level of IP-10 with a control level of IP-10 to identify or aid in identifying the antigen peptide as being one that is recognized by the rare antigen-specific T cell. In some embodiments of any one of the methods provided, an elevated level of IP-10 compared to the control level indicates that the antigen peptide (e.g., autoantigen peptide) is recognized by the rare antigen-specific T cell. In some embodiments, the level of IP-10 measured in the sample is elevated if it is at least two times higher than the control level of IP-10. In some embodiments of any one of the methods provided, a decreased or substantially the same level of IP-10 compared to the control level indicates that the antigen peptide (e.g., autoantigen peptide) is not recognized by the rare antigen-specific T cell. In some embodiments of any one of the methods provided, a level of IP-10 is elevated if the level of IP-10 is at least two-fold greater than a control level of IP-10. In some embodiments of any one of the methods provided, the control level of IP-10 is a level of IP-10 in a sample that has been contacted with a composition comprising phosphate buffered saline. In some embodiments of any one of the methods provided, the control level of IP-10 is a level of IP-10 in a sample from the subject, e.g., obtained before the administration to the subject of an agent that stimulates IL-2 expression or a composition comprising a gluten peptide as described herein.

In some embodiments of any one of the methods provided, the method further comprises measuring a level of IFN-γ and/or IL-2 in the sample. In some embodiments of any one of the methods provided, the level of IFN-γ and/or IL-2 is compared to a control level of IFN-γ and/or IL-2, respectively. In some embodiments of any one of the methods provided, an elevated level of IFN-γ and/or IL-2 compared to the control level indicates that the autoantigen peptide is recognized by the rare autoantigen-specific T cell and wherein a decreased or substantially the same level of IFN-γ and/or IL-2 compared to the control level indicates that the autoantigen peptide is not recognized by the rare autoantigen-specific T cell. In some embodiments of any one of the methods provided, a level of IFN-γ and/or IL-2 is elevated if the level of IFN-γ and/or IL-2 is at least two-fold greater than a control level of IFN-γ and/or IL-2, respectively. In some embodiments of any one of the methods provided, the control level of IFN-γ and/or IL-2 is a level of IFN-γ and/or IL-2, respectively, in a sample that has been contacted with a composition comprising phosphate buffered saline. In some embodiments of any one of the methods provided, the subject is a subject that has previously been administered an agent that stimulates IL-2 expression. In some embodiments of any one of the methods provided, the method further comprises administering an agent that stimulates IL-2 expression to the subject prior to the measuring. In some embodiments of any one of the methods provided, the subject has or is suspected of having an autoimmune disease. In some embodiments of any one of the methods provided, the subject has or is suspected of having the autoimmune disease and Celiac disease and the subject has previously been administered any of the compositions comprising a gluten peptide as described herein. In some embodiments of any one of the methods provided, the subject has or is suspected of having the autoimmune disease and Celiac disease and the method further comprises administering any one of the compositions comprising a gluten peptide to the subject prior to the measuring.

Another aspect of the disclosure relates to screening for rare antigen-specific T cell responses, e.g., a rare autoantigen-specific T cell response. In some embodiments, the method comprises measuring a level of IP-10 in a plurality of samples comprising a plurality of rare antigen-specific T cells (e.g., rare autoantigen-specific T cells) obtained from a subject as described herein (e.g., having or suspected of having an autoimmune disease, an allergy, an infectious disease or condition, or an adverse immune condition caused by administration of an isolated, recombinant or synthetic protein or peptide, such as a therapeutic) wherein the measuring comprises contacting the plurality of samples with an plurality of antigen peptides as described herein and measuring the level of IP-10 in each sample in the plurality of samples. Assays for measuring a level of IP-10 are described herein. In some embodiments of any one of the methods provided, the method further comprises comparing the level of IP-10 in each sample in the plurality of samples with a control level of IP-10 to identify or aid in identifying each of the antigen peptides in the plurality of antigen peptides as being one that is recognized by one or more of the plurality of rare antigen-specific T cells. In some embodiments of any one of the methods provided, an elevated level of IP-10 compared to the control level indicates that one or more of the antigen peptides in the plurality of antigen peptides is recognized by one or more of the plurality of rare antigen-specific T cells. In some embodiments of any one of the methods provided, the level of IP-10 measured in the sample is elevated if it is at least two times higher than the control level of IP-10. In some embodiments of any one of the methods provided, a decreased or substantially the same level of IP-10 compared to the control level indicates that one or more of the antigen peptides in the plurality of antigen peptides is not recognized by one or more of the plurality of rare antigen-specific T cells. In some embodiments of any one of the methods provided, the control level of IP-10 is a level of IP-10 in a sample from the subject, e.g., obtained before the administration to the subject of an agent that stimulates IL-2 expression or any one of the compositions comprising a gluten peptide as described herein. In some embodiments of any one of the methods provided, a level of IP-10 is elevated if the level of IP-10 is at least two-fold greater than a control level of IP-10. In some embodiments of any one of the methods provided, the control level of IP-10 is a level of IP-10 in a sample that has been contacted with a composition comprising phosphate buffered saline.

In some embodiments of any one of the methods provided, the method further comprises measuring a level of IFN-γ and/or IL-2 in the sample. In some embodiments of any one of the methods provided, the level of IFN-γ and/or IL-2 is compared to a control level of IFN-γ and/or IL-2, respectively. In some embodiments of any one of the methods provided, an elevated level of IFN-γ and/or IL-2 compared to the control level indicates that the autoantigen peptide is recognized by the rare autoantigen-specific T cell and wherein a decreased or substantially the same level of IFN-γ and/or IL-2 compared to the control level indicates that the autoantigen peptide is not recognized by the rare autoantigen-specific T cell. In some embodiments of any one of the methods provided, a level of IFN-γ and/or IL-2 is elevated if the level of IFN-γ and/or IL-2 is at least two-fold greater than a control level of IFN-γ and/or IL-2, respectively. In some embodiments of any one of the methods provided, the control level of IFN-γ and/or IL-2 is a level of IFN-γ and/or IL-2, respectively in a sample that has been contacted with a composition comprising phosphate buffered saline.

### Islet Autoantigen Peptides

Aspects of the disclosure relate to islet autoantigen peptides. As used herein the term “islet autoantigen peptide” includes any peptide comprising a sequence derived from, or encompassed within, one or more of islet autoantigens. Exemplary islet autoantigens include, but are not limited to, proinsulin, 65-kDa isoform of glutamic acid decarboxylase (GAD 65), 67-kDa isoform of glutamic acid decarboxylase (GAD 67) and islet antigen-2 (IA-2).

Accordingly, in some embodiments, the islet autoantigen peptide is selected from or part of a library of peptides (e.g., a library of peptides that are 10, 11, 12, 13, 14, 15, 16, 17, 18, 19 or 20 amino acids or more in length) that is designed to encompass all unique 11mer sequences in human proteins that are commonly recognized by autoantibodies circulating in patients with Type-1 diabetes, such as proinsulin, GAD 65 and IA-2 (see, e.g., Beissbarth T, Tye-Din J A, Smyth G K, Speed T P, Anderson R P. A systematic approach for comprehensive T-cell epitope discovery using peptide libraries. Bioinformatics. 2005; 21(s1):i29-i37). Such peptides derived from native protein sequences or sequences selectively deamidated by human transglutaminase-2 may provide epitopes recognized by pathogenic CD4 or CD8 T-cell epitopes that mediate islet-specific autoimmunity (see, e.g., Vader L W, et al. Specificity of tissue transglutaminase explains cereal toxicity in celiac disease. J Exp Med. 2002; 195(5):643-9). The library may be a plurality of peptides (e.g., at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 5000, at least 10000, at least 50000, at least 100000, or more peptides; or e.g., 10-100000, 10-10000, 10-1000, 10-100, 50-100000, 50-10000, 50-1000, 150-100, 100-100000, 100-10000, 100-1000, 500-100000, 500-10000, or 500-1000 peptides).

An exemplary method for designing a library utilizing a computer algorithm follows. Protein antigen sequence(s) are identified using Genbank or another sequence database. All possible 17-20mers derived from the sequence(s) are identified. All possible 11 or 12mers are derived in the same manner. 17-20mers that cover the most 11 or 12mers are selected, the 11 or 12mers that are now covered by the selected 17-20mers are marked. If not all of the 11 or 12mers are marked, a second round of selection of 17-20mers that cover the most non-marked 11 or 12mers is performed in the same manner. This is iterated until all 11 or 12mers are covered by 20mers. Using such an exemplary method can reduce the number of peptides to screen by, e.g., about 5- to 10-fold. In some embodiments, the library can be further modified or supplemented by deamidating one or more glutamate residues of peptide(s) in the library. The one or more glutamate residues of peptide(s) in the library may be generated by tissue transglutaminase (tTG) deamidation activity upon one or more glutamine residues of the peptide(s). This deamidation of glutamine to glutamate may cause the generation of peptides that can bind to HLA-DQ2 or -DQ8 molecules with high affinity. This reaction may occur in vitro by contacting the peptide with tTG outside of the subject or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, in some embodiments, a deamidation motif defined for transglutaminase-2 (QX₁PX₃, or QX₁X₂[F,Y,W,I,L,V], where X₁ and X₃ are not proline) may be used to identify glutamine residues for deamidation within peptide(s) in the library.

The library of islet autoantigen peptides may be derived from known protein sequences, e.g., sourced from Genbank or other sequence databases. Exemplary amino acid sequences from Genbank for generation of an islet autoantigen peptide library are shown below:

Insulin/Proinsulin/Preproinsulin
>CAA23828.1
(SEQ ID NO: 1)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AFK93533.1
(SEQ ID NO: 2)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>NP_001172026.1
(SEQ ID NO: 3)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>NP_001172027.1
(SEQ ID NO: 4)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>NP_000198.1
(SEQ ID NO: 5)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AAA59172.1
(SEQ ID NO: 6)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>CAA49913.1
(SEQ ID NO: 7)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>P01308.1
(SEQ ID NO: 8)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>0601246A
(SEQ ID NO: 9)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AAW83741.1
(SEQ ID NO: 10)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AAA59172.1
(SEQ ID NO: 11)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AAN39451.1
(SEQ ID NO: 12)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AAA59173.1
(SEQ ID NO: 13)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AAH05255.1
(SEQ ID NO: 14)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AAP35454.1
(SEQ ID NO: 15)
MALWMRLLPLLALLALWGPDPAAAFVNQHLCGSHLVEALYLVCGERGFFYTPKTRREA
EDLQVGQVELGGGPGAGSLQPLALEGSLQKRGIVEQCCTSICSLYQLENYCN
>AAA59179 .1
(SEQ ID NO: 16)
MALWMRLLPLLALLALWGPDPAAAFVNQHLCGSHLVEALYLVCGERGFFYTPKTRREA
EVGQVELGGGPGAGSLQPLALEGSLQKRGIVEQCCTSICSLYQLENYCN
>AEG19452 .1
(SEQ ID NO: 17)
WGPDPAAAFVNQHLCGSHLVEALYLVCGERGFFYTPKTRREAEDLQVGQVELGGGPGA
GSLQPLALEGSLQKRGIVEQCCTSICSLYQLENYCN
>CAA08766.1
(SEQ ID NO: 18)
MALWMRLLPLLALLALWGPDPAAAFVNQHLCGSHLVEALYLVCGERGFFYTPKTRREA
E
>ABI63346.1
(SEQ ID NO: 19)
MALWMRLLPLLALLALWGPDPAAAFVNQHLCGSHLVEALYLVCGERGFFYTPKTRREA
EDLQGSLQPLALEGSLQKRGIVEQCCTSICSLYQLENYCN
>AAP35454.1E
(SEQ ID NO: 20)
MALWMRLLPLLALLALWGPDPAAAFVNQHLCGSHLVEALYLVCGERGFFYTPKTRREA
EDLQVGEVELGGGPGAGSLQPLALEGSLQKRGIVEQCCTSICSLYQLENYCN
>AAA59179.1E
(SEQ ID NO: 21)
MALWMRLLPLLALLALWGPDPAAAFVNQHLCGSHLVEALYLVCGERGFFYTPKTRREA
EVGEVELGGGPGAGSLQPLALEGSLQKRGIVEQCCTSICSLYQLENYCN
>AEG19452.1E
(SEQ ID NO: 22)
WGPDPAAAFVNQHLCGSHLVEALYLVCGERGFFYTPKTRREAEDLQVGEVELGGGPGA
GSLQPLALEGSLQKRGIVEQCCTSICSLYQLENYCN
>CAA08766.1E
(SEQ ID NO: 23)
MALWMRLLPLLALLALWGPDPAAAFVNQHLCGSHLVEALYLVCGERGFFYTPKTRREA
E
>ABI63346.1E
(SEQ ID NO: 24)
MALWMRLLPLLALLALWGPDPAAAFVNQHLCGSHLVEALYLVCGERGFFYTPKTRREA
EDLEGSLQPLALEGSLQKRGIVEQCCTSICSLYQLENYCN
Glutamic decarboxylase 65 (GAD65) and 67 (GAD67)
>NP_001127838.1
(SEQ ID NO: 25)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>NP_001127838.1E
(SEQ ID NO: 26)
MASPGSGFWSFGSEDGSGDSENPGTARAWCQVAQKFTGGIGNKLCALLYGDAEKPAESG
GSQPPRAAARKAACACDEKPCSCSKVDVNYAFLHATDLLPACDGERPTLAFLQDVMNIL
LEYVVKSFDRSTKVIDFHYPNELLQEYNWELADQPQNLEEILMHCETTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKEKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>NP_000809.1
(SEQ ID NO: 27)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>Q05329.1
(SEQ ID NO: 28)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAA58491 .1
(SEQ ID NO: 29)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>CAC09233.1
(SEQ ID NO: 30)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAA62367.1
(SEQ ID NO: 31)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>IEAW86103.1
(SEQ ID NO: 32)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>EAW86104.1
(SEQ ID NO: 33)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDGLMQNCNQMHASYLFQQDKHYDLSYDTGDKALQCGRHVDVFKLWLMWRAK
GTTGFEAHVDKCLELAEYLYNIIKNREGYEMVFDGKPQHTNVCFWYIPPSLRTLEDNEER
MSRLSKVAPVIKARMMEYGTTMVSYQPLGDKVNFFRMVISNPAATHQDIDFLIEEIERLG
QDL
>EAW86102.1
(SEQ ID NO: 34)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAI26330.1
(SEQ ID NO: 35)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAI26328.1
(SEQ ID NO: 36)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAP88040.1
(SEQ ID NO: 37)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>CAH73659.1
(SEQ ID NO: 38)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>Q5VZ30
(SEQ ID NO: 39)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>CAB62572.1
(SEQ ID NO: 40)
LKYAIKTGHPRYFNQLSTGLDMVGLAADWLTSTANTNMFTYEIAPVFVLLEYVTLKKMR
EIIGWPGGSGDGIFSPGGAISNMYAMMIARFKMFPEVKEKGMAALPRLIAFTSEHSHFSLK
KGAAALGIGTDSVILIKCDERGKMIPSDLERRILEAKQKGFVPFLVSATAGTTVYGAFDPL
LAVADICKKYKIVVMHVDAAWGGGLLMSRKHKWKLSGVERANSVTWNPHKMMGVPLQ
CSALLVREEGLMQNCNQMHASYLFQQDKHYDLSYDTGDKALQCGRHVDVFKLWLMW
RAKGTTGFEAHVDKCLELAEYLYNIIKNREGYEMVFDGKPQHTNVCFWYIPPSLRTLEDN
EERMSRLSKVAPVIKARMMEYGTTMVSYQPLGDKVNFFRMVISNPAATHQDIDFLIEEIE
RLGQDL
>CAB62572.1E
(SEQ ID NO: 41)
LKYAIKTGHPRYFNQLSTGLDMVGLAADWLTSTANTNMFTYEIAPVFVLLEYVTLKKMR
EIIGWPGGSGDGIFSPGGAISNMYAMMIARFKMFPEVKEKGMAALPRLIAFTSEHSHFSLK
KGAAALGIGTDSVILIKCDERGKMIPSDLERRILEAKEKGFVPFLVSATAGTTVYGAFDPL
LAVADICKKYKIWMHVDAAWGGGLLMSRKHKWKLSGVERANSVTWNPHKMMGVPLQ
CSALLVREEGLMQNCNQMHASYLFQQDKHYDLSYDTGDKALQCGRHVDVFKLWLMW
RAKGTTGFEAHVDKCLELAEYLYNIIKNREGYEMVFDGKPQHTNVCFWYIPPSLRTLEDN
EERMSRLSKVAPVIKARMMEYGTTMVSYQPLGDKVNFFRMVISNPAATHQDIDFLIEEIE
RLGQDL
>CAA49554.1
(SEQ ID NO: 42)
MSPIHHHHHHLVPRGSEASNSGFWSFGSEDGSGDSENPGTARAWCQVAQKFTGGIGNKL
CALLYGDAEKPAESGGSQPPRAAARKAACACDQKPCSCSKVDVNYAFLHATDLLPACD
GERPTLAFLQDVMNILLQYVVKSFDRSTKVIDFHYPNELLQEYNWELADQPQNLEEILMH
CQTTLKYAIKTGHPRYFNQLSTGLDMVGLAADWLTSTANTNMFTYEIAPVFVLLEYVTL
KKMREIIGWPGGSGDGIFSPGGAISNMYAMMIARFKMFPEVKEKGMAALPRLIAFTSEHS
HFSLKKGAAALGIGTDSVILIKCDERGKMIPSDLERRILEAKQKGFVPFLVSATAGTTVYG
AFDPLLAVADICKKYKIWMHVDAAWGGGLLMSRKHKWKLSGVERANSVTWNPHKMM
GVPLQCSALLVREEGLMQNCNQMHASYLFQQDKHYDLSYDTGDKALQCGRHVDVFKL
WLMWRAKGTTGFEAHVDKCLELAEYLYNIIKNREGYEMVFDGKPQHTNVCFWYIPPSLR
TLEDNEERMSRLSKVAPVIKARMMEYGTTMVSYQPLGDKVNFFRMVISNPAATHQDIDF
LIEEIERLGQDL
>CAA49554.1E
(SEQ ID NO: 43)
MSPIHHHHHHLVPRGSEASNSGFWSFGSEDGSGDSENPGTARAWCQVAQKFTGGIGNKL
CALLYGDAEKPAESGGSQPPRAAARKAACACDEKPCSCSKVDVNYAFLHATDLLPACDG
ERPTLAFLQDVMNILLEYVVKSFDRSTKVIDFHYPNELLQEYNWELADQPQNLEEILMHC
ETTLKYAIKTGHPRYFNQLSTGLDMVGLAADWLTSTANTNMFTYEIAPVFVLLEYVTLK
KMREIIGWPGGSGDGIFSPGGAISNMYAMMIARFKMFPEVKEKGMAALPRLIAFTSEHSH
FSLKKGAAALGIGTDSVILIKCDERGKMIPSDLERRILEAKEKGFVPFLVSATAGTTVYGAF
DPLLAVADICKKYKIWMHVDAAWGGGLLMSRKHKWKLSGVERANSVTWNPHKMMGV
PLQCSALLVREEGLMQNCNQMHASYLFQEDKHYDLSYDTGDKALQCGRHVDVFKLWL
MWRAKGTTGFEAHVDKCLELAEYLYNIIKNREGYEMVFDGKPQHTNVCFWYIPPSLRTL
EDNEERMSRLSKVAPVIKARMMEYGTTMVSYQPLGDKVNFFRMVISNPAATHQDIDF6LIE
EIERLGQDL
>AAB28987.1
(SEQ ID NO: 44)
ISNMYAMMIARFKMFPEVKEKGMAALPRLIAFTSEHSHFSLKKGAAALGIGTDSVILIKCD
ERGKMIPSDLERRILEAKQKGFVPFLVSATAGTTVYGAFDPLLAVADICKKYKIWMHVDA
AWGGGLLMSRKHKWKLSGVERANSVTWNPHKMMGVPLQCSALLVREEGLMQNCNQM
HASYLFQQDKHYDLSYDTGDKALQCGRHVDVFKLWLMWRAKGTTGFEAHVDKCLELA
EYLYNIIKNREGYEMVFDGKPQHTNVCFWYIPPSLRTLEDNEERMSRLSKVAPVIKARMM
EYGTTMVSYQPLGDKVNFFRMVISNPAATHQDIDFLIEEIERLGQDL
>AAB28987.1E
(SEQ ID NO: 45)
ISNMYAMMIARFKMFPEVKEKGMAALPRLIAFTSEHSHFSLKKGAAALGIGTDSVILIKCD
ERGKMIPSDLERRILEAKEKGFVPFLVSATAGTTVYGAFDPLLAVADICKKYKIWMHVDA
AWGGGLLMSRKHKWKLSGVERANSVTWNPHKMMGVPLQCSALLVREEGLMQNCNQM
HASYLFQEDKHYDLSYDTGDKALQCGRHVDVFKLWLMWRAKGTTGFEAHVDKCLELA
EYLYNIIKNREGYEMVFDGKPQHTNVCFWYIPPSLRTLEDNEERMSRLSKVAPVIKARMM
EYGTTMVSYQPLGDKVNFFRMVISNPAATHQDIDFLIEEIERLGQDL
>EAW86101.1
(SEQ ID NO: 46)
MASPGSGFWSFGSEDGSGDSENPGTARAWCQVAQKFTGGIGNKLCDLLPACDGERPTLA
FLQDVMNILLQYVVKSFDR
>EAW86101.1E
(SEQ ID NO: 47)
MASPGSGFWSFGSEDGSGDSENPGTARAWCQVAQKFTGGIGNKLCDLLPACDGERPTLA
FLQDVMNILLEYVVKSFDR
>CAH73660.1
(SEQ ID NO: 48)
MNILLQYVVKSFDRSTKVIDFHYPNELLQEYNWELADQPQNLEEILMHCQTTLKYAIKTG
HPRYFNQLSTGLDMVGLAADWLTSTANTNMFTYEIAPVFVLLEYVTLKKMREIIGWPGG
SGDGIFSPGT
>CAH73660.1E
(SEQ ID NO: 49)
MNILLEYVVKSFDRSTKVIDFHYPNELLQEYNWELADQPQNLEEILMHCETTLKYAIKTG
HPRYFNQLSTGLDMVGLAADWLTSTANTNMFTYEIAPVFVLLEYVTLKKMREIIGWPGG
SGDGIFSPGT
>CAH73658.1
(SEQ ID NO: 50)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGIVSS
KIIKLFFRLQ
>CAH73658.1E
(SEQ ID NO: 51)
MASPGSGFWSFGSEDGSGDSENPGTARAWCQVAQKFTGGIGNKLCALLYGDAEKPAESG
GSQPPRAAARKAACACDEKPCSCSKVDVNYAFLHATDLLPACDGERPTLAFLQDVMNIL
LEYVVKSFDRSTKVIDFHYPNELLQEYNWELADQPQNLEEILMHCETTLKYAIKTGIVSSK
IIKLFFRLQ
Insulinoma antigen-2 or Tyrosine phosphatase like autoantigen or
(IA-2; ICA512, PTPRN)
>Q16849.1
(SEQ ID NO: 52)
MRRPRRPGGLGGSGGLRLLLCLLLLSSRPGGCSAVSAHGCLFDRRLCSHLEVCIQDGLFG
QCQVGVGQARPLLQVTSPVLQRLQGVLRQLMSQGLSWHDDLTQYVISQEMERIPRLRPP
EPRPRDRSGLAPKRPGPAGELLLQDIPTGSAPAAQHRLPQPPVGKGGAGASSSLSPLQAEL
LPPLLEHLLLPPQPPHPSLSYEPALLQPYLFHQFGSRDGSRVSEGSPGMVSVGPLPKAEAPA
LFSRTASKGIFGDHPGHSYGDLPGPSPAQLFQDSGLLYLAQELPAPSRARVPRLPEQGSSSR
AEDSPEGYEKEGLGDRGEKPASPAVQPDAALQRLAAVLAGYGVELRQLTPEQLSTLLTLL
QLLPKGAGRNPGGVVNVGADIKKTMEGPVEGRDTAELPARTSPMPGHPTASPTSSEVQQ
VPSPVSSEPPKAARPPVTPVLLEKKSPLGQSQPTVAGQPSARPAAEEYGYIVTDQKPLSLA
AGVKLLEILAEHVHMSSGSFINISVVGPALTFRIRHNEQNLSLADVTQQAGLVKSELEAQT
GLQILQTGVGQREEAAAVLPQTAHSTSPMRSVLLTLVALAGVAGLLVALAVALCVRQHA
RQQDKERLAALGPEGAHGDTTFEYQDLCRQHMATKSLFNRAEGPPEPSRVSSVSSQFSDA
AQASPSSHSSTPSWCEEPAQANMDISTGHMILAYMEDHLRNRDRLAKEWQALCAYQAEP
NTCATAQGEGNIKKNRHPDFLPYDHARIKLKVESSPSRSDYINASPIIEHDPRMPAYIATQG
PLSHTIADFWQMVWESGCTVIVMLTPLVEDGVKQCDRYWPDEGASLYHVYEVNLVSEHI
WCEDFLVRSFYLKNVQTQETRTLTQFHFLSWPAEGTPASTRPLLDFRRKVNKCYRGRSCP
IIVHCSDGAGRTGTYILIDMVLNRMAKGVKEIDIAATLEHVRDQRPGLVRSKDQFEFALTA
VAEEVNAILKALPQ
>Q16849.1E
(SEQ ID NO: 53)
MRRPRRPGGLGGSGGLRLLLCLLLLSSRPGGCSAVSAHGCLFDRRLCSHLEVCIEDGLFGE
CEVGVGQARPLLQVTSPVLQRLEGVLRQLMSQGLSWHDDLTEYVISQEMERIPRLRPPEP
RPRDRSGLAPKRPGPAGELLLQDIPTGSAPAAEHRLPQPPVGKGGAGASSSLSPLQAELLP
PLLEHLLLPPQPPHPSLSYEPALLQPYLFHQFGSRDGSRVSEGSPGMVSVGPLPKAEAPALF
SRTASKGIFGDHPGHSYGDLPGPSPAQLFQDSGLLYLAQELPAPSRARVPRLPEQGSSSRA
EDSPEGYEKEGLGDRGEKPASPAVQPDAALQRLAAVLAGYGVELRQLTPEQLSTLLTLLQ
LLPKGAGRNPGGVVNVGADIKKTMEGPVEGRDTAELPARTSPMPGHPTASPTSSEVQEVP
SPVSSEPPKAARPPVTPVLLEKKSPLGQSQPTVAGQPSARPAAEEYGYIVTDEKPLSLAAG
VKLLEILAEHVHMSSGSFINISVVGPALTFRIRHNEQNLSLADVTQEAGLVKSELEAETGL
QILQTGVGQREEAAAVLPQTAHSTSPMRSVLLTLVALAGVAGLLVALAVALCVRQHARQ
QDKERLAALGPEGAHGDTTFEYQDLCRQHMATKSLFNRAEGPPEPSRVSSVSSQFSDAAQ
ASPSSHSSTPSWCEEPAQANMDISTGHMILAYMEDHLRNRDRLAKEWQALCAYQAEPNT
CATAQGEGNIKKNRHPDFLPYDHARIKLKVESSPSRSDYINASPIIEHDPRMPAYIATEGPL
SHTIADFWEMVWESGCTVIVMLTPLVEDGVKQCDRYWPDEGASLYHVYEVNLVSEHIW
CEDFLVRSFYLKNVQTQETRTLTEFHFLSWPAEGTPASTRPLLDFRRKVNKCYRGRSCPII
VHCSDGAGRTGTYILIDMVLNRMAKGVKEIDIAATLEHVRDERPGLVRSKDEFEFALTAV
AEEVNAILKALPQ
>NP_001186692.1
(SEQ ID NO: 54)
MRRPRRPGGLGGSGGLRLLLCLLLLSSRPGGCSAVSAHGCLFDRRLCSHLEVCIQDGLFG
QCQVGVGQARPLLQVTSPVLQRLQGVLRQLMSQGLSWHDDLTQYVISQEMERIPRLRPP
EPRPRDRSGLAPKRPGPAGELLLQDIPTGSAPAAQHRLPQPPVGKGGAGASSSLSPLQAEL
LPPLLEHLLLPPQPPHPSLSYEPALLQPYLFHQFGSRDGSRVSEGSPGMVSVGPLPKAEAPA
LFSRTASKGIFGDHPGHSYGDLPGPSPAQLFQDSGLLYLAQELPAPSRARVPRLPEQGSSSR
AEDSPEGYEKEGLGDRGEKPASPAVQPDAALQRLAAVLAGYGVELRQLTPEQLSTLLTLL
QLLPKGAGRNPGGVVNVGADIKKTMEGPVEGRDTAELPARTSPMPGHPTASPTSSEVQQ
VPSPVSSEPPKAARPPVTPVLLEKKSPLGQSQPTVAGQPSARPAAEEYGYIVTDQNVVGPA
LTFRIRHNEQNLSLADVTQQAGLVKSELEAQTGLQILQTGVGQREEAAAVLPQTAHSTSP
MRSVLLTLVALAGVAGLLVALAVALCVRQHARQQDKERLAALGPEGAHGDTTFEYQDL
CRQHMATKSLFNRAEGPPEPSRVSSVSSQFSDAAQASPSSHSSTPSWCEEPAQANMDISTG
HMILAYMEDHLRNRDRLAKEWQALCAYQAEPNTCATAQGEGNIKKNRHPDFLPYDHAR
IKLKVESSPSRSDYINASPIIEHDPRMPAYIATQGPLSHTIADFWQMVWESGCTVIVMLTPL
VEDGVKQCDRYWPDEGASLYHVYEVNLVSEHIWCEDFLVRSFYLKNVQTQETRTLTQFH
FLSWPAEGTPASTRPLLDFRRKVNKCYRGRSCPIIVHCSDGAGRTGTYILIDMVLNRMAK
GVKEIDIAATLEHVRDQRPGLVRSKDQFEFALTAVAEEVNAILKALPQ
>NP_001186693.1
(SEQ ID NO: 55)
MSQGLSWHDDLTQYVISQEMERIPRLRPPEPRPRDRSGLAPKRPGPAGELLLQDIPTGSAP
AAQHRLPQPPVGKGGAGASSSLSPLQAELLPPLLEHLLLPPQPPHPSLSYEPALLQPYLFHQ
FGSRDGSRVSEGSPGMVSVGPLPKAEAPALFSRTASKGIFGDHPGHSYGDLPGPSPAQLFQ
DSGLLYLAQELPAPSRARVPRLPEQGSSSRAEDSPEGYEKEGLGDRGEKPASPAVQPDAA
LQRLAAVLAGYGVELRQLTPEQLSTLLTLLQLLPKGAGRNPGGVVNVGADIKKTMEGPV
EGRDTAELPARTSPMPGHPTASPTSSEVQQVPSPVSSEPPKAARPPVTPVLLEKKSPLGQSQ
PTVAGQPSARPAAEEYGYIVTDQKPLSLAAGVKLLEILAEHVHMSSGSFINISVVGPALTF
RIRHNEQNLSLADVTQQAGLVKSELEAQTGLQILQTGVGQREEAAAVLPQTAHSTSPMRS
VLLTLVALAGVAGLLVALAVALCVRQHARQQDKERLAALGPEGAHGDTTFEYQDLCRQ
HMATKSLFNRAEGPPEPSRVSSVSSQFSDAAQASPSSHSSTPSWCEEPAQANMDISTGHMI
LAYMEDHLRNRDRLAKEWQALCAYQAEPNTCATAQGEGNIKKNRHPDFLPYDHARIKL
KVESSPSRSDYINASPIIEHDPRMPAYIATQGPLSHTIADFWQMVWESGCTVIVMLTPLVE
DGVKQCDRYWPDEGASLYHVYEVNLVSEHIWCEDFLVRSFYLKNVQTQETRTLTQFHFL
SWPAEGTPASTRPLLDFRRKVNKCYRGRSCPIIVHCSDGAGRTGTYILIDMVLNRMAKGV
KEIDIAATLEHVRDQRPGLVRSKDQFEFALTAVAEEVNAILKALPQ

In some embodiments, the islet autoantigen peptide is a peptide (e.g., at least one peptide) comprising or consisting of an amino acid sequence provided in Table 3.

TABLE 3
Islet autoantigen peptide library

| | | SEQ ID |
|---|---|---|
| Islet Autoantigen | Sequence | NO: |
| IA-2 | H-VSSEPPKAARPPVTPVL-OH | 56 |
| IA-2 | H-HSSTPSWCEEPAQANMD-OH | 57 |
| IA-2 | H-KSLFNRAEGPPEPSRVS-OH | 58 |
| IA-2 | H-ALCVRQHARQQDKERLA-OH | 59 |
| IA-2 | H-TGHMILAYMEDHLRNRD-OH | 60 |
| IA-2 | H-LLPPLLEHLLLPPQPPH-OH | 61 |
| IA-2 | H-SPLQAELLPPLLEHLLL-OH | 62 |
| IA-2 | H-EHVRDQRPGLVRSKDQF-OH | 63 |
| IA-2 | H-ASPTSSEVQQVPSPVSS-OH | 64 |
| IA-2 | H-PGHPTASPTSSEVQQVP-OH | 65 |
| IA-2 | H-GVKLLEILAEHVHMSSG-OH | 66 |
| IA-2 | H-EGYEKEGLGDRGEKPAS-OH | 67 |
| IA-2 | H-TEFHFLSWPAEGTPAST-OH | 68 |
| IA-2 | H-LKNVQTQETRTLTQFHF-OH | 69 |
| IA-2 | H-AAEEYGYIVTDEKPLSL-OH | 70 |
| IA-2 | H-GRDTAELPARTSPMPGH-OH | 71 |
| IA-2 | H-QLLPKGAGRNPGGVVNV-OH | 72 |
| IA-2 | H-DFLVRSFYLKNVQTQET-OH | 73 |
| IA-2 | H-SSTPSWCEEPAQANMDI-OH | 74 |
| IA-2 | H-LAAVLAGYGVELRQLTP-OH | 75 |
| IA-2 | H-PIIEHDPRMPAYIATEG-OH | 76 |
| IA-2 | H-LLTLVALAGVAGLLVAL-OH | 77 |
| IA-2 | H-PASTRPLLDFRRKVNKC-OH | 78 |
| IA-2 | H-GYGVELRQLTPEQLSTL-OH | 79 |
| IA-2 | H-RQHARQQDKERLAALGP-OH | 80 |
| IA-2 | H-GCSAVSAHGCLFDRRLC-OH | 81 |
| IA-2 | H-QQDKERLAALGPEGAHG-OH | 82 |
| IA-2 | H-PGGLGGSGGLRLLLCLL-OH | 83 |
| IA-2 | H-AQGEGNIKKNRHPDFLP-OH | 84 |
| IA-2 | H-PGHSYGDLPGPSPAQLF-OH | 85 |
| IA-2 | H-YHVYEVNLVSEHIWCED-OH | 86 |
| IA-2 | H-LSWHDDLTQYVISQEME-OH | 87 |
| IA-2 | H-PVLLEKKSPLGQSQPTV-OH | 88 |
| IA-2 | H-GDHPGHSYGDLPGPSPA-OH | 89 |
| IA-2 | H-YWPDEGASLYHVYEVNL-OH | 90 |
| IA-2 | H-QGPLSHTIADFWQMVWE-OH | 91 |
| IA-2 | H-LVKSELEAQTGLQILQT-OH | 92 |
| IA-2 | H-GVKEIDIAATLEHVRDE-OH | 93 |
| IA-2 | H-VSEGSPGMVSVGPLPKA-OH | 94 |
| IA-2 | H-GRNPGGVVNVGADIKKT-OH | 95 |
| IA-2 | H-GLQILQTGVGQREEAAA-OH | 96 |
| IA-2 | H-HDDLTEYVISQEMERIP-OH | 97 |
| IA-2 | H-ALLQPYLFHQFGSRDGS-OH | 98 |
| IA-2 | H-YQAEPNTCATAQGEGNI-OH | 99 |
| IA-2 | H-LPKAEAPALFSRTASKG-OH | 100 |
| IA-2 | H-PKAARPPVTPVLLEKKS-OH | 101 |
| IA-2 | H-RPRDRSGLAPKRPGPAG-OH | 102 |
| IA-2 | H-ALTAVAEEVNAILKALP-OH | 103 |
| IA-2 | H-DFWQMVWESGCTVIVML-OH | 104 |
| IA-2 | H-DRLAKEWQALCAYQAEP-OH | 105 |
| IA-2 | H-RGRSCPIIVHCSDGAGR-OH | 106 |
| IA-2 | H-ARIKLKVESSPSRSDYI-OH | 107 |
| IA-2 | H-VSSVSSQFSDAAQASPS-OH | 108 |
| IA-2 | H-QFHFLSWPAEGTPASTR-OH | 109 |
| IA-2 | H-SDYINASPIIEHDPRMP-OH | 110 |
| IA-2 | H-PMPGHPTASPTSSEVQQ-OH | 111 |
| IA-2 | H-YILIDMVLNRMAKGVKE-OH | 112 |
| IA-2 | H-EQNLSLADVTQEAGLVK-OH | 113 |
| IA-2 | H-KGAGRNPGGVVNVGADI-OH | 114 |
| IA-2 | H-SGGLRLLLCLLLLSSRP-OH | 115 |
| IA-2 | H-AEEYGYIVTDQNVVGPA-OH | 116 |
| IA-2 | H-QAEPNTCATAQGEGNIK-OH | 117 |
| IA-2 | H-HGDTTFEYQDLCRQHMA-OH | 118 |
| IA-2 | H-TQYVISQEMERIPRLRP-OH | 119 |
| IA-2 | H-DIAATLEHVRDQRPGLV-OH | 120 |
| IA-2 | H-RIPRLRPPEPRPRDRSG-OH | 121 |
| IA-2 | H-KAEAPALFSRTASKGIF-OH | 122 |
| IA-2 | H-QEMERIPRLRPPEPRPR-OH | 123 |
| IA-2 | H-LCSHLEVCIQDGLFGQC-OH | 124 |
| IA-2 | H-WCEEPAQANMDISTGHM-OH | 125 |
| IA-2 | H-DLPGPSPAQLFQDSGLL-OH | 126 |
| IA-2 | H-REEAAAVLPQTAHSTSP-OH | 127 |
| IA-2 | H-GSFINISVVGPALTFRI-OH | 128 |
| IA-2 | H-PAQANMDISTGHMILAY-OH | 129 |
| IA-2 | H-PPQPPHPSLSYEPALLQ-OH | 130 |
| IA-2 | H-GLFGQCQVGVGQARPLL-OH | 131 |
| IA-2 | H-WQALCAYQAEPNTCATA-OH | 132 |
| IA-2 | H-AQHRLPQPPVGKGGAGA-OH | 133 |
| IA-2 | H-PPVTPVLLEKKSPLGQS-OH | 134 |
| IA-2 | H-SHTIADFWEMVWESGCT-OH | 135 |
| IA-2 | H-QDIPTGSAPAAEHRLPQ-OH | 136 |
| IA-2 | H-QLMSQGLSWHDDLTQYV-OH | 137 |
| IA-2 | H-GPALTFRIRHNEQNLSL-OH | 138 |
| IA-2 | H-EYGYIVTDQNVVGPALT-OH | 139 |
| IA-2 | H-RSKDEFEFALTAVAEEV-OH | 140 |
| IA-2 | H-SPMRSVLLTLVALAGVA-OH | 141 |
| IA-2 | H-TQQAGLVKSELEAQTGL-OH | 142 |
| IA-2 | H-LFDRRLCSHLEVCIQDG-OH | 143 |
| IA-2 | H-QSQPTVAGQPSARPAAE-OH | 144 |
| IA-2 | H-NEQNLSLADVTQQAGLV-OH | 145 |
| IA-2 | H-PAYIATQGPLSHTIADF-OH | 146 |
| IA-2 | H-IADFWEMVWESGCTVIV-OH | 147 |
| IA-2 | H-PSLSYEPALLQPYLFHQ-OH | 148 |
| IA-2 | H-EGNIKKNRHPDFLPYDH-OH | 149 |
| IA-2 | H-SHLEVCIEDGLFGECEV-OH | 150 |
| IA-2 | H-HCSDGAGRTGTYILIDM-OH | 151 |
| IA-2 | H-KNRHPDFLPYDHARIKL-OH | 152 |
| IA-2 | H-VLQRLQGVLRQLMSQGL-OH | 153 |
| IA-2 | H-QEVPSPVSSEPPKAARP-OH | 154 |
| IA-2 | H-AEGPPEPSRVSSVSSQF-OH | 155 |
| IA-2 | H-HPTASPTSSEVQEVPSP-OH | 156 |
| IA-2 | H-VQPDAALQRLAAVLAGY-OH | 157 |
| IA-2 | H-QTGLQILQTGVGQREEA-OH | 158 |
| IA-2 | H-CIEDGLFGECEVGVGQA-OH | 159 |
| IA-2 | H-ASSSLSPLQAELLPPLL-OH | 160 |
| IA-2 | H-ILAYMEDHLRNRDRLAK-OH | 161 |
| IA-2 | H-TPLVEDGVKQCDRYWPD-OH | 162 |
| IA-2 | H-IVMLTPLVEDGVKQCDR-OH | 163 |
| IA-2 | H-PQTAHSTSPMRSVLLTL-OH | 164 |
| IA-2 | H-TQETRTLTQFHFLSWPA-OH | 165 |
| IA-2 | H-QTGVGQREEAAAVLPQT-OH | 166 |
| IA-2 | H-ETRTLTQFHFLSWPAEG-OH | 167 |
| IA-2 | H-DLTEYVISQEMERIPRL-OH | 168 |
| IA-2 | H-LGPEGAHGDTTFEYQDL-OH | 169 |
| IA-2 | H-QFSDAAQASPSSHSSTP-OH | 170 |
| IA-2 | H-VESSPSRSDYINASPII-OH | 171 |
| IA-2 | H-LKNVQTQETRTLTEFHF-OH | 172 |
| IA-2 | H-RKVNKCYRGRSCPIIVH-OH | 173 |
| IA-2 | H-RPLLQVTSPVLQRLEGV-OH | 174 |
| IA-2 | H-FGSRDGSRVSEGSPGMV-OH | 175 |
| IA-2 | H-QRLAAVLAGYGVELRQL-OH | 176 |
| IA-2 | H-VCIQDGLFGQCQVGVGQ-OH | 177 |
| IA-2 | H-QDLCRQHMATKSLFNRA-OH | 178 |
| IA-2 | H-ADVTQEAGLVKSELEAE-OH | 179 |
| IA-2 | H-ANMDISTGHMILAYMED-OH | 180 |
| IA-2 | H-EDGVKQCDRYWPDEGAS-OH | 181 |
| IA-2 | H-QASPSSHSSTPSWCEEP-OH | 182 |
| IA-2 | H-SSEPPKAARPPVTPVLL-OH | 183 |
| IA-2 | H-LSSRPGGCSAVSAHGCL-OH | 184 |
| IA-2 | H-SSPSRSDYINASPIIEH-OH | 185 |
| IA-2 | H-KLLEILAEHVHMSSGSF-OH | 186 |
| IA-2 | H-PTSSEVQEVPSPVSSEP-OH | 187 |
| IA-2 | H-QRPGLVRSKDQFEFALT-OH | 188 |
| IA-2 | H-SAPAAEHRLPQPPVGKG-OH | 189 |
| IA-2 | H-EDHLRNRDRLAKEWQAL-OH | 190 |
| IA-2 | H-GLAPKRPGPAGELLLQD-OH | 191 |
| IA-2 | H-EHDPRMPAYIATQGPLS-OH | 192 |
| IA-2 | H-PRMPAYIATEGPLSHTI-OH | 193 |
| IA-2 | H-PPLLEHLLLPPQPPHPS-OH | 194 |
| IA-2 | H-VTDQKPLSLAAGVKLLE-OH | 195 |
| IA-2 | H-DEGASLYHVYEVNLVSE-OH | 196 |
| IA-2 | H-CATAQGEGNIKKNRHPD-OH | 197 |
| IA-2 | H-ALFSRTASKGIFGDHPG-OH | 198 |
| IA-2 | H-ELPAPSRARVPRLPEQG-OH | 199 |
| IA-2 | H-LVALAVALCVRQHARQQ-OH | 200 |
| IA-2 | H-LEHVRDERPGLVRSKDE-OH | 201 |
| IA-2 | H-VNLVSEHIWCEDFLVRS-OH | 202 |
| IA-2 | H-IAATLEHVRDERPGLVR-OH | 203 |
| IA-2 | H-GLFGECEVGVGQARPLL-OH | 204 |
| IA-2 | H-PYLFHQFGSRDGSRVSE-OH | 205 |
| IA-2 | H-AGASSSLSPLQAELLPP-OH | 206 |
| IA-2 | H-VRSKDQFEFALTAVAEE-OH | 207 |
| IA-2 | H-NRDRLAKEWQALCAYQA-OH | 208 |
| IA-2 | H-HLLLPPQPPHPSLSYEP-OH | 209 |
| IA-2 | H-GSPGMVSVGPLPKAEAP-OH | 210 |
| IA-2 | H-SFYLKNVQTQETRTLTQ-OH | 211 |
| IA-2 | H-KERLAALGPEGAHGDTT-OH | 212 |
| IA-2 | H-TLLTLLQLLPKGAGRNP-OH | 213 |
| IA-2 | H-AEEYGYIVTDQKPLSLA-OH | 214 |
| IA-2 | H-VALAGVAGLLVALAVAL-OH | 215 |
| IA-2 | H-GRTGTYILIDMVLNRMA-OH | 216 |
| IA-2 | H-KTMEGPVEGRDTAELPA-OH | 217 |
| IA-2 | H-HMSSGSFINISVVGPAL-OH | 218 |
| IA-2 | H-TSPVLQRLEGVLRQLMS-OH | 219 |
| IA-2 | H-EGLGDRGEKPASPAVQP-OH | 220 |
| IA-2 | H-LLQDIPTGSAPAAQHRL-OH | 221 |
| IA-2 | H-DVTQQAGLVKSELEAQT-OH | 222 |
| IA-2 | H-LNRMAKGVKEIDIAATL-OH | 223 |
| IA-2 | H-SELEAETGLQILQTGVG-OH | 224 |
| IA-2 | H-LAEHVHMSSGSFINISV-OH | 225 |
| IA-2 | H-IKKTMEGPVEGRDTAEL-OH | 226 |
| IA-2 | H-GDRGEKPASPAVQPDAA-OH | 227 |
| IA-2 | H-QETRTLTEFHFLSWPAE-OH | 228 |
| IA-2 | H-GPSPAQLFQDSGLLYLA-OH | 229 |
| IA-2 | H-NVGADIKKTMEGPVEGR-OH | 230 |
| IA-2 | H-SLADVTQQAGLVKSELE-OH | 231 |
| IA-2 | H-ERPGLVRSKDEFEFALT-OH | 232 |
| IA-2 | H-LTAVAEEVNAILKALPQ-OH | 233 |
| IA-2 | H-QPPVGKGGAGASSSLSP-OH | 234 |
| IA-2 | H-AHSTSPMRSVLLTLVAL-OH | 235 |
| IA-2 | H-HIWCEDFLVRSFYLKNV-OH | 236 |
| IA-2 | H-FEYQDLCRQHMATKSLF-OH | 237 |
| IA-2 | H-LPYDHARIKLKVESSPS-OH | 238 |
| IA-2 | H-EEYGYIVTDEKPLSLAA-OH | 239 |
| IA-2 | H-AGLVKSELEAETGLQIL-OH | 240 |
| IA-2 | H-AGVAGLLVALAVALCVR-OH | 241 |
| IA-2 | H-LFQDSGLLYLAQELPAP-OH | 242 |
| IA-2 | H-EKPLSLAAGVKLLEILA-OH | 243 |
| IA-2 | H-EQGSSSRAEDSPEGYEK-OH | 244 |
| IA-2 | H-PVEGRDTAELPARTSPM-OH | 245 |
| IA-2 | H-GVVNVGADIKKTMEGPV-OH | 246 |
| IA-2 | H-MVSVGPLPKAEAPALFS-OH | 247 |
| IA-2 | H-RQLTPEQLSTLLTLLQL-OH | 248 |
| IA-2 | H-LEAQTGLQILQTGVGQR-OH | 249 |
| IA-2 | H-PGPAGELLLQDIPTGSA-OH | 250 |
| IA-2 | H-PSRARVPRLPEQGSSSR-OH | 251 |
| IA-2 | H-GPAGELLLQDIPTGSAP-OH | 252 |
| IA-2 | H-PLLDFRRKVNKCYRGRS-OH | 253 |
| IA-2 | H-SQGLSWHDDLTEYVISQ-OH | 254 |
| IA-2 | H-DLTQYVISQEMERIPRL-OH | 255 |
| IA-2 | H-SARPAAEEYGYIVTDQK-OH | 256 |
| IA-2 | H-LQVTSPVLQRLQGVLRQ-OH | 257 |
| IA-2 | H-KQCDRYWPDEGASLYHV-OH | 258 |
| IA-2 | H-KKSPLGQSQPTVAGQPS-OH | 259 |
| IA-2 | H-QPTVAGQPSARPAAEEY-OH | 260 |
| IA-2 | H-SRVSEGSPGMVSVGPLP-OH | 261 |
| IA-2 | H-INASPIIEHDPRMPAYI-OH | 262 |
| IA-2 | H-VSAHGCLFDRRLCSHLE-OH | 263 |
| IA-2 | H-VGKGGAGASSSLSPLQA-OH | 264 |
| IA-2 | H-HMATKSLFNRAEGPPEP-OH | 265 |
| IA-2 | H-RRLCSHLEVCIEDGLFG-OH | 266 |
| IA-2 | H-RLEGVLRQLMSQGLSWH-OH | 267 |
| IA-2 | H-LEVCIQDGLFGQCQVGV-OH | 268 |
| IA-2 | H-CPIIVHCSDGAGRTGTY-OH | 269 |
| IA-2 | H-QGVLRQLMSQGLSWHDD-OH | 270 |
| IA-2 | H-PDAALQRLAAVLAGYGV-OH | 271 |
| IA-2 | H-FQDSGLLYLAQELPAPS-OH | 272 |
| IA-2 | H-ATEGPLSHTIADFWEMV-OH | 273 |
| IA-2 | H-ARPLLQVTSPVLQRLQG-OH | 274 |
| IA-2 | H-CTVIVMLTPLVEDGVKQ-OH | 275 |
| IA-2 | H-AAAVLPQTAHSTSPMRS-OH | 276 |
| IA-2 | H-RIRHNEQNLSLADVTQE-OH | 277 |
| IA-2 | H-TDQNVVGPALTFRIRHN-OH | 278 |
| IA-2 | H-PLSLAAGVKLLEILAEH-OH | 279 |
| IA-2 | H-LRPPEPRPRDRSGLAPK-OH | 280 |
| IA-2 | H-SLSYEPALLQPYLFHQF-OH | 281 |
| IA-2 | H-TLEHVRDERPGLVRSKD-OH | 282 |
| IA-2 | H-SSEVQQVPSPVSSEPPK-OH | 283 |
| IA-2 | H-WESGCTVIVMLTPLVED-OH | 284 |
| IA-2 | H-GQPSARPAAEEYGYIVT-OH | 285 |
| IA-2 | H-PASPAVQPDAALQRLAA-OH | 286 |
| IA-2 | H-RPRRPGGLGGSGGLRLL-OH | 287 |
| IA-2 | H-TGSAPAAQHRLPQPPVG-OH | 288 |
| IA-2 | H-SHTIADFWQMVWESGCT-OH | 289 |
| IA-2 | H-LYLAQELPAPSRARVPR-OH | 290 |
| IA-2 | H-YGYIVTDQKPLSLAAGV-OH | 291 |
| IA-2 | H-ISVVGPALTFRIRHNEQ-OH | 292 |
| IA-2 | H-QLSTLLTLLQLLPKGAG-OH | 293 |
| IA-2 | H-SLYHVYEVNLVSEHIWC-OH | 294 |
| IA-2 | H-PEPSRVSSVSSQFSDAA-OH | 295 |
| IA-2 | H-ASKGIFGDHPGHSYGDL-OH | 296 |
| IA-2 | H-DMVLNRMAKGVKEIDIA-OH | 297 |
| IA-2 | H-LLCLLLLSSRPGGCSAV-OH | 298 |
| IA-2 | H-QVGVGQARPLLQVTSPV-OH | 299 |
| IA-2 | H-YIATEGPLSHTIADFWE-OH | 300 |
| IA-2 | H-EFALTAVAEEVNAILKA-OH | 301 |
| IA-2 | H-VNKCYRGRSCPIIVHCS-OH | 302 |
| IA-2 | H-RAEDSPEGYEKEGLGDR-OH | 303 |
| IA-2 | H-MRRPRRPGGLGGSGGLR-OH | 304 |
| IA-2 | H-ECEVGVGQARPLLQVTS-OH | 305 |
| IA-2 | H-PRLPEQGSSSRAEDSPE-OH | 306 |
| IA-2 | H-GYIVTDEKPLSLAAGVK-OH | 307 |
| IA-2 | H-HPDFLPYDHARIKLKVE-OH | 308 |
| IA-2 | H-WPAEGTPASTRPLLDFR-OH | 309 |
| IA-2 | H-LPARTSPMPGHPTASPT-OH | 310 |
| GAD65/67 | H-PIHHHHHHLVPRGSEAS-OH | 311 |
| GAD65/67 | H-VILIKCDERGKMIPSDL-OH | 312 |
| GAD65/67 | H-GDKVNFFRMVISNPAAT-OH | 313 |
| GAD65/67 | H-MIPSDLERRILEAKEKG-OH | 314 |
| GAD65/67 | H-MASPGSGFWSFGSEDGS-OH | 315 |
| GAD65/67 | H-EMVFDGKPQHTNVCFWY-OH | 316 |
| GAD65/67 | H-NQMHASYLFQQDKHYDL-OH | 317 |
| GAD65/67 | H-TANTNMFTYEIAPVFVL-OH | 318 |
| GAD65/67 | H-PQNLEEILMHCQTTLKY-OH | 319 |
| GAD65/67 | H-GFWSFGSEDGSGDSENP-OH | 320 |
| GAD65/67 | H-EYVTLKKMREIIGWPGG-OH | 321 |
| GAD65/67 | H-YDTGDKALQCGRHVDVF-OH | 322 |
| GAD65/67 | H-LCDLLPACDGERPTLAF-OH | 323 |
| GAD65/67 | H-FTGGIGNKLCDLLPACD-OH | 324 |
| GAD65/67 | H-LHATDLLPACDGERPTL-OH | 325 |
| GAD65/67 | H-RLSKVAPVIKARMMEYG-OH | 326 |
| GAD65/67 | H-QNCNQMHASYLFQEDKH-OH | 327 |
| GAD65/67 | H-AFDPLLAVADICKKYKI-OH | 328 |
| GAD65/67 | H-EAKQKGFVPFLVSATAG-OH | 329 |
| GAD65/67 | H-CACDEKPCSCSKVDVNY-OH | 330 |
| GAD65/67 | H-FDRSTKVIDFHYPNELL-OH | 331 |
| GAD65/67 | H-VIDFHYPNELLQEYNWE-OH | 332 |
| GAD65/67 | H-KTGIVSSKIIKLFFRLQ-OH | 333 |
| GAD65/67 | H-PACDGERPTLAFLQDVM-OH | 334 |
| GAD65/67 | H-SDLERRILEAKQKGFVP-OH | 335 |
| GAD65/67 | H-IGTDSVILIKCDERGKM-OH | 336 |
| GAD65/67 | H-VISNPAATHQDIDFLIE-OH | 337 |
| GAD65/67 | H-AIKTGHPRYFNQLSTGL-OH | 338 |
| GAD65/67 | H-VAQKFTGGIGNKLCALL-OH | 339 |
| GAD65/67 | H-HVDAAWGGGLLMSRKHK-OH | 340 |
| GAD65/67 | H-MQNCNQMHASYLFQQDK-OH | 341 |
| GAD65/67 | H-YLFQQDKHYDLSYDTGD-OH | 342 |
| GAD65/67 | H-DFHYPNELLQEYNWELA-OH | 343 |
| GAD65/67 | H-ELLQEYNWELADQPQNL-OH | 344 |
| GAD65/67 | H-AAARKAACACDEKPCSC-OH | 345 |
| GAD65/67 | H-EKGMAALPRLIAFTSEH-OH | 346 |
| GAD65/67 | H-YKIWMHVDGLMQNCNQM-OH | 347 |
| GAD65/67 | H-VDVNYAFLHATDLLPAC-OH | 348 |
| GAD65/67 | H-PRYFNQLSTGLDMVGLA-OH | 349 |
| GAD65/67 | H-QDIDFLIEEIERLGQDL-OH | 350 |
| GAD65/67 | H-ENPGTARAWCQVAQKFT-OH | 351 |
| GAD65/67 | H-PQNLEEILMHCETTLKY-OH | 352 |
| GAD65/67 | H-RPTLAFLQDVMNILLQY-OH | 353 |
| GAD65/67 | H-AESGGSQPPRAAARKAA-OH | 354 |
| GAD65/67 | H-RTLEDNEERMSRLSKVA-OH | 355 |
| GAD65/67 | H-VPFLVSATAGTTVYGAF-OH | 356 |
| GAD65/67 | H-DLSYDTGDKALQCGRHV-OH | 357 |
| GAD65/67 | H-FNQLSTGLDMVGLAADW-OH | 358 |
| GAD65/67 | H-YVVKSFDRSTKVIDFHY-OH | 359 |
| GAD65/67 | H-NMFTYEIAPVFVLLEYV-OH | 360 |
| GAD65/67 | H-GFEAHVDKCLELAEYLY-OH | 361 |
| GAD65/67 | H-SEDGSGDSENPGTARAW-OH | 362 |
| GAD65/67 | H-RAAARKAACACDQKPCS-OH | 363 |
| GAD65/67 | H-CFWYIPPSLRTLEDNEE-OH | 364 |
| GAD65/67 | H-TGLDMVGLAADWLTSTA-OH | 365 |
| GAD65/67 | H-IKARMMEYGTTMVSYQP-OH | 366 |
| GAD65/67 | H-THQDIDFLIEEIERLGQ-OH | 367 |
| GAD65/67 | H-NMYAMMIARFKMFPEVK-OH | 368 |
| GAD65/67 | H-MNILLEYVVKSFDRSTK-OH | 369 |
| GAD65/67 | H-TARAWCQVAQKFTGGIG-OH | 370 |
| GAD65/67 | H-QDVMNILLQYVVKSFDR-OH | 371 |
| GAD65/67 | H-ASPGSGFWSFGSEDGSG-OH | 372 |
| GAD65/67 | H-HFSLKKGAAALGIGTDS-OH | 373 |
| GAD65/67 | H-YQPLGDKVNFFRMVISN-OH | 374 |
| GAD65/67 | H-ILLQYVVKSFDRSTKVI-OH | 375 |
| GAD65/67 | H-AACACDQKPCSCSKVDV-OH | 376 |
| GAD65/67 | H-AATHQDIDFLIEEPEAN-OH | 377 |
| GAD65/67 | H-HHLVPRGSEASNSGFWS-OH | 378 |
| GAD65/67 | H-KLSGVERANSVTWNPHK-OH | 379 |
| GAD65/67 | H-CSCSKVDVNYAFLHATD-OH | 380 |
| GAD65/67 | H-DPLLAVADICKKYKIWM-OH | 381 |
| GAD65/67 | H-AKEKGFVPFLVSATAGT-OH | 382 |
| GAD65/67 | H-KGTTGFEAHVDKCLELA-OH | 383 |
| GAD65/67 | H-GIFSPGGAISNMYAMMI-OH | 384 |
| GAD65/67 | H-HASYLFQEDKHYDLSYD-OH | 385 |
| GAD65/67 | H-GTTMVSYQPLGDKVNFF-OH | 386 |
| GAD65/67 | H-RRILEAKQKGFVPFLVS-OH | 387 |
| GAD65/67 | H-LADQPQNLEEILMHCET-OH | 388 |
| GAD65/67 | H-LQDVMNILLQYVVKSFD-OH | 389 |
| GAD65/67 | H-IARFKMFPEVKEKGMAA-OH | 390 |
| GAD65/67 | H-QCSALLVREEGLMQNCN-OH | 391 |
| GAD65/67 | H-FSLKKGAAALGIGTDSV-OH | 392 |
| GAD65/67 | H-LCALLYGDAEKPAESGG-OH | 393 |
| GAD65/67 | H-ELAEYLYNIIKNREGYE-OH | 394 |
| GAD65/67 | H-CQVAQKFTGGIGNKLCD-OH | 395 |
| GAD65/67 | H-QEDKHYDLSYDTGDKAL-OH | 396 |
| GAD65/67 | H-GIGNKLCALLYGDAEKP-OH | 397 |
| GAD65/67 | H-NYAFLHATDLLPACDGE-OH | 398 |
| GAD65/67 | H-SEASNSGFWSFGSEDGS-OH | 399 |
| GAD65/67 | H-GDAEKPAESGGSQPPRA-OH | 400 |
| GAD65/67 | H-AAALGIGTDSVILIKCD-OH | 401 |
| GAD65/67 | H-WNPHKMMGVPLQCSALL-OH | 402 |
| GAD65/67 | H-MMEYGTTMVSYQPLGDK-OH | 403 |
| GAD65/67 | H-IGNKLCDLLPACDGERP-OH | 404 |
| GAD65/67 | H-KYKIWMHVDAAWGGGLL-OH | 405 |
| GAD65/67 | H-CDQKPCSCSKVDVNYAF-OH | 406 |
| GAD65/67 | H-ERRILEAKEKGFVPFLV-OH | 407 |
| GAD65/67 | H-DGSGDSENPGTARAWCQ-OH | 408 |
| GAD65/67 | H-ETTLKYAIKTGHPRYFN-OH | 409 |
| GAD65/67 | H-RANSVTWNPHKMMGVPL-OH | 410 |
| GAD65/67 | H-LEYVVKSFDRSTKVIDF-OH | 411 |
| GAD65/67 | H-PGGSGDGIFSPGGAISN-OH | 412 |
| GAD65/67 | H-AALPRLIAFTSEHSHFS-OH | 413 |
| GAD65/67 | H-KHKWKLSGVERANSVTW-OH | 414 |
| GAD65/67 | H-FTSEHSHFSLKKGAAAL-OH | 415 |
| GAD65/67 | H-ADWLTSTANTNMFTYEI-OH | 416 |
| GAD65/67 | H-REGYEMVFDGKPQHTNV-OH | 417 |
| GAD65/67 | H-GTTVYGAFDPLLAVADI-OH | 418 |
| GAD65/67 | H-ADICKKYKIWMHVDGLM-OH | 419 |
| GAD65/67 | H-ICKKYKIWMHVDAAWGG-OH | 420 |
| GAD65/67 | H-NWELADQPQNLEEILMH-OH | 421 |
| GAD65/67 | H-EERMSRLSKVAPVIKAR-OH | 422 |
| GAD65/67 | H-ILMHCQTTLKYAIKTGH-OH | 423 |
| GAD65/67 | H-TYEIAPVFVLLEYVTLK-OH | 424 |
| GAD65/67 | H-GAISNMYAMMIARFKMF-OH | 425 |
| GAD65/67 | H-YIPPSLRTLEDNEERMS-OH | 426 |
| GAD65/67 | H-LMHCETTLKYAIKTGIV-OH | 427 |
| GAD65/67 | H-MSPIHHHHHHLVPRGSE-OH | 428 |
| GAD65/67 | H-GDKALQCGRHVDVFKLW-OH | 429 |
| GAD65/67 | H-GLAADWLTSTANTNMFT-OH | 430 |
| GAD65/67 | H-GLLMSRKHKWKLSGVER-OH | 431 |
| GAD65/67 | H-DVFKLWLMWRAKGTTGF-OH | 432 |
| GAD65/67 | H-LMWRAKGTTGFEAHVDK-OH | 433 |
| GAD65/67 | H-APVIKARMMEYGTTMVS-OH | 434 |
| GAD65/67 | H-MGVPLQCSALLVREEGL-OH | 435 |
| GAD65/67 | H-LKYAIKTGIVSSKIIKL-OH | 436 |
| GAD65/67 | H-IWMHVDGLMQNCNQMHA-OH | 437 |
| GAD65/67 | H-QPPRAAARKAACACDEK-OH | 438 |
| GAD65/67 | H-QCGRHVDVFKLWLMWRA-OH | 439 |
| GAD65/67 | H-FPEVKEKGMAALPRLIA-OH | 440 |
| GAD65/67 | H-TGGIGNKLCALLYGDAE-OH | 441 |
| GAD65/67 | H-RMVISNPAATHQDIDFL-OH | 442 |
| GAD65/67 | H-GKPQHTNVCFWYIPPSL-OH | 443 |
| GAD65/67 | H-IIGWPGGSGDGIFSPGT-OH | 444 |
| GAD65/67 | H-WGGGLLMSRKHKWKLSG-OH | 445 |
| GAD65/67 | H-VNFFRMVISNPAATHQD-OH | 446 |
| GAD65/67 | H-VSATAGTTVYGAFDPLL-OH | 447 |
| GAD65/67 | H-DERGKMIPSDLERRILE-OH | 448 |
| GAD65/67 | H-NVCFWYIPPSLRTLEDN-OH | 449 |
| GAD65/67 | H-PPRAAARKAACACDQKP-OH | 450 |
| GAD65/67 | H-GSEASNSGFWSFGSEDG-OH | 451 |
| GAD65/67 | H-ACACDEKPCSCSKVDVN-OH | 452 |
| GAD65/67 | H-AFLQDVMNILLEYVVKS-OH | 453 |
| GAD65/67 | H-VFVLLEYVTLKKMREII-OH | 454 |
| GAD65/67 | H-NLEEILMHCETTLKYAI-OH | 455 |
| GAD65/67 | H-LPRLIAFTSEHSHFSLK-OH | 456 |
| GAD65/67 | H-YNIIKNREGYEMVFDGK-OH | 457 |
| GAD65/67 | H-QTTLKYAIKTGIVSSKI-OH | 458 |
| GAD65/67 | H-KMREIIGWPGGSGDGIF-OH | 459 |
| GAD65/67 | H-VDKCLELAEYLYNIIKN-OH | 460 |
| GAD65/67 | H-VREEGLMQNCNQMHASY-OH | 461 |
| Insulin/Proinsulin | FYTPKTRREAEDLQGSL | 462 |
| Insulin/Proinsulin | EDLQVGEVELGGGPGAG | 463 |
| Insulin/Proinsulin | RREAEDLEGSLQPLALE | 464 |
| Insulin/Proinsulin | CGSHLVEALYLVCGERG | 465 |
| Insulin/Proinsulin | REAEDLQVGQVELGGGP | 466 |
| Insulin/Proinsulin | ALLALWGPDPAAAFVNQ | 467 |
| Insulin/Proinsulin | YTPKTRREAEVGQVELG | 468 |
| Insulin/Proinsulin | GPGAGSLQPLALEGSLQ | 469 |
| Insulin/Proinsulin | QCCTSICSLYQLENYCN | 470 |
| Insulin/Proinsulin | VEALYLVCGERGFFYTP | 471 |
| Insulin/Proinsulin | NQHLCGSHLVEALYLVC | 472 |
| Insulin/Proinsulin | TPKTRREAEDLEGSLQP | 473 |
| Insulin/Proinsulin | SLQKRGIVEQCCTSICS | 474 |
| Insulin/Proinsulin | PKTRREAEVGEVELGGG | 475 |
| Insulin/Proinsulin | LQGSLQPLALEGSLQKR | 476 |
| Insulin/Proinsulin | YTPKTRREAEDLQVGQV | 477 |
| Insulin/Proinsulin | CGERGFFYTPKTRREAE | 478 |
| Insulin/Proinsulin | LYLVCGERGFFYTPKTR | 479 |
| Insulin/Proinsulin | EDLQVGQVELGGGPGAG | 480 |
| Insulin/Proinsulin | PKTRREAEVGQVELGGG | 481 |
| Insulin/Proinsulin | PAAAFVNQHLCGSHLVE | 482 |
| Insulin/Proinsulin | DLEGSLQPLALEGSLQK | 483 |
| Insulin/Proinsulin | VELGGGPGAGSLQPLAL | 484 |
| Insulin/Proinsulin | PLLALLALWGPDPAAAF | 485 |
| Insulin/Proinsulin | WGPDPAAAFVNQHLCGS | 486 |
| Insulin/Proinsulin | EAEVGQVELGGGPGAGS | 487 |
| Insulin/Proinsulin | MALWMRLLPLLALLALW | 488 |
| Insulin/Proinsulin | MRLLPLLALLALWGPDP | 489 |
| Insulin/Proinsulin | AEVGEVELGGGPGAGSL | 490 |
| Insulin/Proinsulin | RGFFYTPKTRREAEVGE | 491 |
| Insulin/Proinsulin | RREAEDLQGSLQPLALE | 492 |
| Insulin/Proinsulin | IVEQCCTSICSLYQLEN | 493 |
| Insulin/Proinsulin | PLALEGSLQKRGIVEQC | 494 |
| Insulin/Proinsulin | TRREAEDLQVGEVELGG | 495 |

The length of the peptide may vary. In some embodiments of any one of the compositions, methods or kits provided, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments of any one of the compositions, methods or kits provided, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments of any one of the compositions, methods or kits provided, peptides are, e.g., 4-1000, 4-500, 4-100, 4-50, 4-40, 4-30, or 4-20 amino acids in length. In some embodiments of any one of the compositions, methods or kits provided, peptides are 4-20, 5-20, 6-20, 7-20, 8-20, 9-20, 10-20, 11-20, 12-20, 13-20, 14-20, or 15-20 amino acids in length. In some embodiments of any one of the compositions, methods or kits provided, peptides are e.g., 5-30, 10-30, 15-30 or 20-30 amino acids in length. In some embodiments of any one of the compositions, methods or kits provided, peptides are 4-50, 5-50, 6-50, 7-50, 8-50, 9-50, 10-50, 11-50, 12-50, 13-50, 14-50, or 15-50 amino acids in length. In some embodiments of any one of the compositions, methods or kits provided, peptides are 8-30 amino acids in length.

Modifications to a peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, pyrolation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc.

The phrases “protecting group” and “blocking group” as used herein, refers to modifications to the peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- and/or C-terminal. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell.

A peptide may also be chemically modified at the level of amino acid side chains, of amino acid chirality, and/or of the peptide backbone.

In some embodiments, a composition comprising at least one or one or more islet autoantigen peptide(s) is contemplated. In some embodiments of any one of the methods provided, the methods described herein comprise contacting the composition or the peptide with a sample from a subject (e.g., a sample comprising T cells).

### Rare Antigen-Specific T Cells and Antigen Peptides

Aspects of the disclosure relate to rare antigen-specific T cells (e.g., rare autoantigen-specific T cells) and methods and kits for detection of such T cells, e.g., using antigen peptides such as autoantigen peptides. A rare antigen-specific T cell (e.g., a rare autoantigen-specific T cell) is a T cell that recognizes an antigen (e.g., a peptide or protein expressed by a cell or tissue of the subject or a foreign antigen (e.g., a peptide or protein contacted with or administered to the subject)) and is present in less than 0.01% of the T cell population in a subject. Exemplary rare antigen-specific T cells include those specific for the immune-dominant gluten-derived epitopes DQ2.5-glia-α1 or DQ2.5-glia-α2 as described by Christophersen, A., et al. (Christophersen, A., et al. Tetramer-visualized gluten-specific CD4+ T cells in blood as a potential diagnostic marker for coeliac disease without oral gluten challenge. United European gastroenterology journal 2, 268-278 (2014)). Such an exemplary rare T-cell population may not be detected by IFNγ release assays (e.g., ELISpot, intracellular cytokine release measured by flow cytometry or whole blood cytokine release) but may be detectable by flow cytometry using cell labeling with MHC-peptide multimers combined with cell enrichment techniques as described by Christophersen, A., et al. 2014. However, MHC-peptide multimer staining of antigen-specific T cells requires a priori knowledge of the relevant epitope, or is applied by incubating preselected MHC variants with peptides of interest. The latter methodology is unsatisfactory for unbiased comprehensive epitope screening because a particular HLA heterodimer must be pre-selected, and HLA-peptide complexes may not form with sufficient speed or stability to reliably detect epitope-specific T cells (Yang et al. CD4+ T cells recognize diverse epitopes within GAD65: implications for repertoire development and diabetes monitoring. Immunology. 2013 March; 138(3):269-79).

In some embodiments of any one of the methods provided, a sample comprising a (i.e., at least one) rare antigen-specific T cell (e.g., a rare autoantigen-specific T cell) is provided and a level of IP-10 is measured to detect a rare antigen-specific T cell response (e.g., a rare autoantigen-specific T cell response). In some embodiments of any one of the methods provided, a sample comprising a rare antigen-specific T cell (e.g., a rare autoantigen-specific T cell) is contacted with at least one antigen peptide (e.g., at least one autoantigen peptide) and the level of IP-10 is measured subsequent to the contacting. The at least one antigen peptide (e.g., at least one autoantigen peptide) may be, e.g., a peptide derived from a protein or peptide sequence suspected of containing T cell epitopes, such that the antigen peptide may be a candidate antigen, e.g., in a library of candidate antigens. In some embodiments of any one of the methods provided, a antigen peptide is selected from or part of a library of peptides (e.g., a library of candidate peptides that are 10, 11, 12, 13, 14, 15, 16, 17, 18, 19 or 20 amino acids or more in length) that is designed to encompass all unique 8-, 9-, 10-, 11-, or 12mer sequences in a protein or peptide suspected of containing T cell epitopes. The library may be plurality of peptides (e.g., at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 5000, at least 10000, at least 50000, at least 100000, or more; or e.g., 10-100000, 10-10000, 10-1000, 10-100, 50-100000, 50-10000, 50-1000, 150-100, 100-100000, 100-10000, 100-1000, 500-100000, 500-10000, or 500-1000 peptides), e.g., a plurality of peptides derived from sequence(s) of a protein or proteins suspected of containing T cell epitopes, such that the plurality of antigen peptides may be a plurality of candidate antigen peptides. Candidate proteins or peptides suitable for designing antigen peptides (e.g., autoantigen peptides) for mapping potential T cell epitopes can be identified on the basis of known targets for disease-specific or condition-specific antibody responses, for example in Type-1 diabetes proinsulin, GAD65, and IA2. Equivalents of whole protein with a pool of overlapping peptides spanning the primary sequence of the corresponding protein is established in T cell cytokine release assays (see, e.g., Kern, F., et al. Eur J Immunol 2000; 30, 1676-1682. & Maecker, H. T., et al. J Immun Methods 2001; 255, 27-40). Furthermore, comprehensive peptide libraries can be readily designed by one of skill in the art using the primary sequence of proteins or peptides retrieved from databases such as NCBI Genbank (see, e.g., Beissbarth T, Tye-Din J A, Smyth G K, Speed T P, Anderson R P. A systematic approach for comprehensive T-cell epitope discovery using peptide libraries. Bioinformatics. 2005; 21(s1):i29-i37.)

An exemplary method for designing a library utilizing a computer algorithm follows. Protein antigen sequence(s) are identified using Genbank or another sequence database. All possible 17-20mers derived from the sequence(s) are identified. All possible 11 or 12mers are derived in the same manner. 17-20mers that cover the most 11 or 12mers are selected, the 11 or 12mers that are now covered by the selected 17-20mers are marked. If not all of the 11 or 12mers are marked, a second round of selection of 17-20mers that cover the most non-marked 11 or 12mers is performed in the same manner. This is iterated until all 11 or 12mers are covered by 17-20mers. Using such an exemplary method can reduce the number of peptides to screen by, e.g., about 5- to 10-fold. In some embodiments, the library can be further modified or supplemented by deamidating one or more glutamate residues of peptide(s) in the library. The one or more glutamate residues of peptide(s) in the library may be generated by tissue transglutaminase (tTG) deamidation activity upon one or more glutamine residues of the peptide(s). This deamidation of glutamine to glutamate may cause the generation of peptides that can bind to HLA-DQ2 or -DQ8 molecules with high affinity. This reaction may occur in vitro by contacting the peptide with tTG outside of the subject or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, in some embodiments, a deamidation motif defined for transglutaminase-2 (QX₁PX₃, or QX₁X₂[F,Y,W,I,L,V], where X₁ and X₃ are not proline) may be used to identify glutamine residues for deamidation within peptide(s) in the library. Any one of the methods provided herein may also be used to identify rare antigen-specific T cells, where the antigen is a self-antigen (e.g., autoantigen) or a foreign antigen. Accordingly, antigen peptides can be designed for diseases or conditions that would be expected to be amenable to identification of disease- or condition-causing T-cell epitopes, such as in autoantigens or foreign antigens. Exemplary diseases and conditions that the methods are applicable to include, but are not limited to, autoimmune diseases, allergies, infectious diseases and conditions, and adverse immune conditions caused by administration of an isolated, recombinant or synthetic protein or peptide, such as therapeutic, to a subject (e.g., any undesired immune response against an isolated, recombinant or synthetic protein or peptide, such as therapeutic, to a subject).

In some embodiments of any one of the methods or kits provided, the antigen peptide or library of antigen peptides (e.g., a library of candidate antigen peptides) is an autoantigen peptide or library of autoantigen peptides. The autoantigen peptide may be, e.g., a peptide or library of peptides designed based on known targets for immune responses (e.g., antibody responses) associated with an autoimmune disease. Exemplary autoimmune diseases include, but are not limited to, rheumatoid arthritis, multiple sclerosis, immune-mediated or Type I diabetes mellitus, inflammatory bowel disease (e.g., Crohn's disease or ulcerative colitis), systemic lupus erythematosus, psoriasis, scleroderma, autoimmune thyroid disease, alopecia areata, Grave's disease, Guillain-Barré syndrome, celiac disease, Sjögren's syndrome, rheumatic fever, gastritis, autoimmune atrophic gastritis, autoimmune hepatitis, insulitis, oophoritis, orchitis, uveitis, phacogenic uveitis, myasthenia gravis, primary myxoedema, pernicious anemia, autoimmune haemolytic anemia, Addison's disease, scleroderma, Goodpasture's syndrome, nephritis, for example, glomerulonephritis, psoriasis, pemphigus vulgaris, pemphigoid, sympathetic opthalmia, idiopathic thrombocylopenic purpura, idiopathic feucopenia, Wegener's granulomatosis and poly/dermatomyositis. In some embodiments, the autoimmune disease is not Celiac disease. In some embodiments, the autoimmune disease is co-morbid with Celiac disease.

In some embodiments of any one of the methods or kits provided, the antigen peptide or library of antigen peptides (e.g., a library of candidate antigen peptides) is a foreign antigen peptide or library of foreign antigen peptides. The foreign antigen peptide or library of foreign antigen peptides may be, e.g., a peptide or library designed based on a known foreign antigen. Exemplary foreign antigens include, but are not limited to, peptides and proteins derived from pathogens such as viruses, bacteria, fungi, or protozoa. Other exemplary foreign antigens include allergens and recombinant, synthetic, or isolated proteins (including antibodies and fragments thereof) or peptides, such as biologics. Exemplary viruses include, but are not limited to, those in the Adenoviridae, Herpesviridae, Papillomaviridae, Polyomaviridae, Poxviridae, Hepadnaviridae, Parvoviridae, Astroviridae, Caliciviridae, Picornaviridae, Coronaviridae, Flaviviridae, Togaviridae, Hepeviridae, Retroviridae, Orthomyxoviridae, Arenaviridae, Bunyaviridae, Filoviridae, Paramyxoviridae, Rhabdoviridae, or Reoviridae families. Exemplary bacteria include, but are not limited to, those in the Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, or Yersinia genuses. Exemplary fungi include, but are not limited to, Candida, Aspergillus, Cryptococcus, Histoplasma capsulatum, Pneumocystis, Stachybotrys, Exserohilum, Cladosporium, Blastomyces dermatitidis, Coccidioides, Trichophyton mentagrophytes, Fusarium, Mucoromycotina, Sporothrix schenckii, or Paracoccidioides brasiliensis. Exemplary protozoa include, but are not limited to, Plasmodium spp., Entamoeba, Giardia, Trypanosoma brucei, Toxoplasma gondii, Acanthamoeba, Leishmania, Babesia, Balamuthia mandrillaris, Naegleria fowleri, Cryptosporidium, Cyclospora, and Toxoplasma gondii.

Exemplary recombinant, synthetic, or isolated proteins (e.g., biologics) include, but are not limited to, abatacept, adalimumab, alefacept, erythropoietin, etanercept, infliximab, trastuzumab, ustekinumab, denileukin difitox, golimumab, human growth hormone, human insulin, follicle-stimulating hormone, Coagulation Factor VIII, Coagulation Factor IX, Coagulation Factor VIIa, filgrastim, pegfilgrastim, alpha-glactosidase A, laronidase, galsulfase, Dornase alfa, Alteplase, alglucerase, Interferon, Insulin-like growth factor 1, Thymoglobulin, Hepatitis B Immune Globulin, Antihemophilic Factor/von Willebrand Factor Complex, Antihemophilic Factor, Crotalidae Polyvalent Immune Fab, digoxin immune FAB, Alpha-1 Proteinase Inhibitor, Antihemophilic Factor, Immune Globulin Intravenous, Rho(D) Immune Globulin Intravenous, thrombin, Hepatitis B Immune Globulin Intravenous, Protein C Concentrate, C1 Esterase Inhibitor, von Willebrand Factor/Coagulation Factor VIII Complex, Antithrombin, Fibrinogen Concentrate, Immune Globulin Subcutaneous, Varicella Zoster Immune Globulin, Coagulation Factor XIII A Subunit, Prothrombin Complex Concentrate, Antihemophilic Factor (Recombinant), Fc Fusion protein, Fc Fusion Protein, tocilizumab, ofatumumab, bevacizumab, tositumomab, alemtuzumab, arcitumomab, certolizumab pegol, ramucirumab, vedolizumab, cetuximab, obinutuzumab, trastuzumab, canakinumab, ranibizumab, gemtuzumab ozogamicin, imciromab pentetate, pertuzumab, denosumab, capromab pendetide, golimumab, basiliximab, eculizumab, ustekinumab, siltuximab, natalizumab, panitumumab, nofetumomab, denosumab, omalizumab, ipilimumab, daclizumab, and ibritumomab tiuxetan.

Exemplary allergens include, but are not limited to, plant allergens (e.g., pollen, ragweed allergen), insect allergens, insect sting allergens (e.g., bee sting allergens), animal allergens (e.g., pet allergens, such as animal dander or cat Fel d 1 antigen), latex allergens, mold allergens, fungal allergens, cosmetic allergens, drug allergens, food allergens, dust, insect venom, viruses, bacteria, etc. Exemplary food allergens include, but are not limited to milk allergens, egg allergens, nut allergens (e.g., peanut or tree nut allergens, etc. (e.g., walnuts, cashews, etc.)), fish allergens, shellfish allergens, soy allergens, legume allergens, seed allergens and wheat allergens. Exemplary insect sting allergens include allergens that are or are associated with bee stings, wasp stings, hornet stings, yellow jacket stings, etc. Exemplary insect allergens also include house dust mite allergens (e.g., Der P1 antigen) and cockroach allergens. Exemplary drug allergens include allergens that are or are associated with antibiotics, NSAIDs, anaesthetics, etc. Exemplary pollen allergens include grass allergens, tree allergens, weed allergens, flower allergens, etc. In some embodiments of any one of the methods or kits provided, an antigen peptide (e.g., an autoantigen peptide) is a peptide that stimulates an elevated level of IP-10 in a sample comprising rare antigen-specific T cells (e.g., rare autoantigen-specific T cells) from a subject compared to a control level of IP-10 but does not stimulate an elevated level of IFN-γ in a sample comprising rare antigen-specific T cells (e.g., rare autoantigen-specific T cells) from the subject compared to a control level of IFN-γ. In some embodiments, the control level of IFN-γ or IP-10 is a level in a sample that has not been contacted with the antigen peptide (e.g., the autoantigen peptide). In some embodiments, the antigen peptide is not a gluten peptide, e.g., not a gluten peptide as described herein. In some embodiments, the antigen peptide is not an islet autoantigen peptide, e.g., not an islet autoantigen peptide as described herein. In some embodiments, the antigen peptide is not a gluten peptide or an islet autoantigen peptide.

In some embodiments of any one of the compositions, methods or kits provided, the length of the antigen peptide may vary. In some embodiments of any one of the compositions, methods or kits provided, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments of any one of the compositions, methods or kits provided, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments of any one of the compositions, methods or kits provided, peptides are, e.g., 4-1000, 4-500, 4-100, 4-50, 4-40, 4-30, or 4-20 amino acids in length. In some embodiments of any one of the compositions, methods or kits provided, peptides are 4-20, 5-20, 6-20, 7-20, 8-20, 9-20, 10-20, 11-20, 12-20, 13-20, 14-20, or 15-20 amino acids in length. In some embodiments of any one of the compositions, methods or kits provided, peptides are 4-16, 5-16, 6-16, 7-16, 8-16, 9-16, 10-16, 11-16, 12-16, 13-16, 14-16, or 15-16 amino acids in length. In some embodiments of any one of the compositions, methods or kits provided, peptides are e.g., 5-30, 10-30, 15-30 or 20-30 amino acids in length. In some embodiments of any one of the compositions, methods or kits provided, peptides are 4-50, 5-50, 6-50, 7-50, 8-50, 9-50, 10-50, 11-50, 12-50, 13-50, 14-50, or 15-50 amino acids in length. In some embodiments of any one of the compositions, methods or kits provided, peptides are 8-30 amino acids in length.

Modifications to an antigen peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, pyrolation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc.

The phrases “protecting group” and “blocking group” as used herein, refers to modifications to the antigen peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The antigen peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- and/or C-terminal. Furthermore, one or more of the peptides may be PEGylated, where the PEG (polyethyleneoxy group) provides for enhanced lifetime in the blood stream. One or more of the antigen peptides may also be combined as a fusion or chimeric protein with other proteins, or with specific binding agents that allow targeting to specific moieties on a target cell.

An antigen peptide may also be chemically modified at the level of amino acid side chains, of amino acid chirality, and/or of the peptide backbone.

In some embodiments of any one of the methods or kits provided, a composition comprising at least one antigen peptide(s) is contemplated. In some embodiments of any one of the methods provided, the method comprises contacting the composition or the antigen peptide with a sample from a subject (e.g., a sample comprising a rare antigen— specific T cell).

### Gluten Peptides and Compositions Comprising Gluten Peptides

As used herein the term “gluten peptide” includes any peptide comprising a sequence derived from, or encompassed within, one or more of gluten proteins alpha (α), beta (β), γ (γ) and omega (ω) gliadins, and low and high molecular weight (LMW and HMW) glutenins in wheat, B, C and D hordeins in barley, β, γ and omega secalins in rye, and optionally avenins in oats, including deamidated variants thereof containing one or more glutamine to glutamate substitutions. In some embodiments, the gluten peptide(s) stimulate a CD4+ T cell specific response.

A gluten peptide may include one or more sequences of epitopes known to be recognized by a CD4⁺ T cell in a subject with Celiac disease, e.g., PELP (SEQ ID NO: 496), PELPY (SEQ ID NO: 497), QPELPYP (SEQ ID NO: 498), PQPELPY (SEQ ID NO: 500), FPQPELP, (SEQ ID NO: 501), PELPYPQ (SEQ ID NO: 502), FPQPELPYP (SEQ ID NO: 503), PYPQPELPY (SEQ ID NO:504), PFPQPELPY (SEQ ID NO: 505), PQPELPYPQ (SEQ ID NO: 506), PFPQPEQPF (SEQ ID NO: 507), PQPEQPFPW (SEQ ID NO: 508), PIPEQPQPY (SEQ ID NO: 509), PQPELPYPQ (SEQ ID NO: 510), FRPEQPYPQ (SEQ ID NO: 511), PQQSFPEQQ (SEQ ID NO: 512), IQPEQPAQL (SEQ ID NO: 513), QQPEQPYPQ (SEQ ID NO: 514), SQPEQEFPQ (SEQ ID NO: 515), PQPEQEFPQ (SEQ ID NO: 516), QQPEQPFPQ (SEQ ID NO: 517), PQPEQPFCQ (SEQ ID NO: 518), QQPFPEQPQ (SEQ ID NO: 519), PFPQPEQPF (SEQ ID NO: 520), PQPEQPFPW (SEQ ID NO: 521), PFSEQEQPV (SEQ ID NO: 522), FSQQQESPF (SEQ ID NO: 523), PFPQPEQPF (SEQ ID NO: 524), PQPEQPFPQ (SEQ ID NO: 525), PIPEQPQPY (SEQ ID NO: 526), PFPQPEQPF (SEQ ID NO: 527), PQPEQPFPQ (SEQ ID NO: 528), PYPEQEEPF (SEQ ID NO: 529), PYPEQEQPF (SEQ ID NO: 530), PFSEQEQPV (SEQ ID NO: 531), EGSFQPSQE (SEQ ID NO: 532), EQPQQPFPQ (SEQ ID NO: 533), EQPQQPYPE (SEQ ID NO: 534), QQGYYPTSPQ (SEQ ID NO: 535), EGSFQPSQE (SEQ ID NO: 536), PQQSFPEQE (SEQ ID NO: 537), or QGYYPTSPQ (SEQ ID NO: 538) (see, e.g., Sollid L M, Qiao S W, Anderson R P, Gianfrani C, Koning F. Nomenclature and listing of celiac disease relevant gluten epitopes recognized by CD4⁺ T cells. Immunogenetics. 2012; 64:455-60; PCT Publication Nos.: WO/2001/025793, WO/2003/104273, WO/2005/105129, and WO/2010/060155), or an amidated version of any one of these sequences. Preferably, in some embodiments of any one of the kits or methods provided, the gluten peptides that comprise sequences of epitopes such as those set forth in SEQ ID NO: 496, 497, etc., also comprise additional amino acids flanking either or both sides of the epitope. Preferably, in some embodiments of any one of the kits or methods provided, the gluten peptides are at least 8 or 9 amino acids in length.

Exemplary gluten peptides and method for synthesizing or obtaining such peptides are known in the art (see, e.g., PCT Publication Nos.: WO/2001/025793, WO/2003/104273, WO/2005/105129, and WO/2010/060155, which are incorporated herein by reference in their entirety). A gluten peptide can be recombinantly and/or synthetically produced. In some embodiments, a gluten peptide is chemically synthesized, e.g., using a method known in the art. Non-limiting examples of peptide synthesis include liquid-phase synthesis and solid-phase synthesis. In some embodiments, a gluten peptide is produced by enzymatic digestion, e.g., by enzymatic digestion of a larger polypeptide into short peptides.

In some embodiments, one or more glutamate residues of a gluten peptide may be generated by tissue transglutaminase (tTG) deamidation activity upon one or more glutamine residues of the gluten peptide. This deamidation of glutamine to glutamate can cause the generation of gluten peptides that can bind to HLA-DQ2 or -DQ8 molecules with high affinity. This reaction may occur in vitro by contacting the gluten peptide composition with tTG outside of the subject or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, PFPQPQLPY (SEQ ID NO: 539) could become PFPQPELPY (SEQ ID NO: 505) after processing by tTG. Conservative substitution of E with D is also contemplated herein (e.g., PFPQPELPY (SEQ ID NO: 505) could become PFPQPDLPY (SEQ ID NO: 540). Exemplary peptides including an E to D substitution include peptide comprising or consisting of PFPQPDLPY (SEQ ID NO: 540), PQPDLPYPQ (SEQ ID NO: 541), PFPQPDQPF (SEQ ID NO: 542), PQPDQPFPW (SEQ ID NO: 543), PIPDQPQPY (SEQ ID NO: 544), LQPFPQPDLPYPQPQ (SEQ ID NO: 545), QPFPQPDQPFPWQP (SEQ ID NO: 546), or PQQPIPDQPQPYPQQ (SEQ ID NO: 547). Such substituted peptides can be the gluten peptides of any of the methods and compositions provided herein. Accordingly, gluten peptides that have not undergone deamidation are also contemplated herein (e.g., gluten peptides comprising or consisting of PQLP (SEQ ID NO: 548), PQLPY (SEQ ID NO: 549), QPQLPYP (SEQ ID NO: 550), PQPQLPY (SEQ ID NO: 551), FPQPQLP, (SEQ ID NO: 552), PQLPYPQ (SEQ ID NO: 553), FPQPQLPYP (SEQ ID NO: 554), PYPQPQLPY (SEQ ID NO: 555), PFPQPQLPY (SEQ ID NO: 556), PQPQLPYPQ (SEQ ID NO: 557), PFPQPQQPF (SEQ ID NO: 558), PQPQQPFPW (SEQ ID NO: 559), PIPQQPQPY (SEQ ID NO: 560), LQPFPQPQLPYPQPQ (SEQ ID NO: 561), QPFPQPQQPFPWQP (SEQ ID NO: 562), or PEQPIPQQPQPYPQQ (SEQ ID NO: 563), PQPQLPYPQ (SEQ ID NO: 564), FRPQQPYPQ (SEQ ID NO: 565), PQQSFPQQQ (SEQ ID NO: 566), IQPQQPAQL (SEQ ID NO: 567), QQPQQPYPQ (SEQ ID NO: 568), SQPQQQFPQ (SEQ ID NO: 569), PQPQQQFPQ (SEQ ID NO: 570), QQPQQPFPQ (SEQ ID NO: 571), PQPQQPFCQ (SEQ ID NO: 572), QQPFPQQPQ (SEQ ID NO: 573), PFPQPQQPF (SEQ ID NO: 574), PQPQQPFPW (SEQ ID NO: 575), PFSQQQQPV (SEQ ID NO: 576), FSQQQQSPF (SEQ ID NO: 577), PFPQPQQPF (SEQ ID NO: 578), PQPQQPFPQ (SEQ ID NO: 579), PIPQQPQPY (SEQ ID NO: 580), PFPQPQQPF (SEQ ID NO: 581), PQPQQPFPQ (SEQ ID NO: 582), PYPEQQEPF (SEQ ID NO: 583), PYPEQQQPF (SEQ ID NO: 584), PFSQQQQPV (SEQ ID NO: 585), QGSFQPSQQ (SEQ ID NO: 586), QQPQQPFPQ (SEQ ID NO: 587), QQPQQPYPQ (SEQ ID NO: 588), QQGYYPTSPQ (SEQ ID NO: 589), QGSFQPSQQ (SEQ ID NO: 590), PQQSFPQQQ (SEQ ID NO: 591), QGYYPTSPQ (SEQ ID NO: 592), LQPFPQPELPYPQPQ (SEQ ID NO: 593), QPFPQPQQPFPWQP (SEQ ID NO: 594), or PQQPIPQQPQPYPQQ (SEQ ID NO: 595)).

A gluten peptide may also be an analog of any of the peptides described herein. Preferably, in some embodiments the analog is recognized by a CD4⁺ T cell that recognizes one or more of the epitopes listed herein. Exemplary analogs comprise a peptide that has a sequence that is, e.g., 75%, 80%, 85%, 90%, 95%, 96%, 97%, 98%, or 99% homologous to the epitopes specifically recited herein. In some embodiments, the analogs comprise a peptide that is, e.g., 75%, 80%, 85%, 90%, 95%, 96%, 97%, 98%, or 99% homologous to the peptides specifically recited herein. Analogs may also be a variant of any of the peptides provided, such variants can include conservative amino acid substitution variants, e.g., E to D substitution.

In some embodiments, analogs may include one or more amino acid substitutions as shown in Table A (see, e.g., Anderson et al. Antagonists and non-toxic variants of the dominant wheat gliadin T cell epitope in coeliac disease. Gut. 2006 April; 55(4): 485-491; and PCT Publication WO2003104273, the contents of which are incorporated herein by reference). The gluten peptides provided herein include analogs of SEQ ID NO:91 comprising one or more of the listed amino acid substitutions. In some embodiments, the analog is an analog of SEQ ID NO: 501 comprising one of the amino acid substitutions provided in Table A below.

TABLE A
Exemplary substitutions in the epitope FPQPELPYP (SEQ ID NO: 501)

| | Amino acid in epitope | | | | | | | | |
|---|---|---|---|---|---|---|---|---|---|
| | F | P | Q | P | E | L | P | Y | P |
| Exemplary | A, G, H, I, | A, F, I, M, | A, F, G, H, | — | D | M | S | I, S, | S, T, |
| Substitutions | L, M P, S, | S, T, V, | I, L, M, S, | | | | | V, W | Y |
| | T, W, Y | W, Y | T, V | | | | | | |

The length of the peptide may vary. In some embodiments of any one of the compositions, methods or kits provided, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments of any one of the compositions, methods or kits provided, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments of any one of the compositions, methods or kits provided, peptides are, e.g., 4-1000, 4-500, 4-100, 4-50, 4-40, 4-30, or 4-20 amino acids in length. In some embodiments of any one of the compositions, methods or kits provided, peptides are 4-20, 5-20, 6-20, 7-20, 8-20, 9-20, 10-20, 11-20, 12-20, 13-20, 14-20, or 15-20 amino acids in length. In some embodiments of any one of the compositions, methods or kits provided, peptides are e.g., 5-30, 10-30, 15-30 or 20-30 amino acids in length. In some embodiments of any one of the compositions, methods or kits provided, peptides are 4-50, 5-50, 6-50, 7-50, 8-50, 9-50, 10-50, 11-50, 12-50, 13-50, 14-50, or 15-50 amino acids in length. In some embodiments of any one of the compositions, methods or kits provided, peptides are 8-30 amino acids in length.

In some embodiments of any one of the methods or kits provided, a composition comprising at least one or one or more gluten peptide(s) is contemplated. In some embodiments of any one of the methods provided, the methods described herein comprise administering the composition to a subject (e.g., a subject having or suspected of having Celiac disease and T1D). In some embodiments of any one of the method provided, the methods described herein comprise contacting the composition with a sample from a subject (e.g., a sample comprising T cells).

In some embodiments of any one of the methods or kits provided, the composition comprises at least one of: (i) a first peptide comprising the amino acid sequence PFPQPELPY (SEQ ID NO: 505) and PQPELPYPQ (SEQ ID NO: 506), (ii) a second peptide comprising the amino acid sequence PFPQPEQPF (SEQ ID NO: 507) and PQPEQPFPW (SEQ ID NO: 508), and (iii) a third peptide comprising the amino acid sequence PIPEQPQPY (SEQ ID NO: 509). “First”, “second”, and “third” are not meant to imply an order of use or importance, unless specifically stated otherwise. In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises the amino acid sequence LQPFPQPELPYPQPQ (SEQ ID NO: 593); the second peptide comprises the amino acid sequence QPFPQPEQPFPWQP (SEQ ID NO: 594); and/or the third peptide comprises the amino acid sequence PEQPIPEQPQPYPQQ (SEQ ID NO: 595).

In some embodiments, it may be desirable to utilize the non-deamidated forms of such peptides, e.g., if the peptides are contained within a composition for administration to a subject where tissue transglutaminase will act in situ (see, e.g., Øyvind Molberg, Stephen McAdam, Knut E. A. Lundin, Christel Kristiansen, Helene Arentz-Hansen, Kjell Kett and Ludvig M. Sollid. T cells from celiac disease lesions recognize gliadin epitopes deamidated in situ by endogenous tissue transglutaminase. Eur. J. Immunol. 2001. 31: 1317-1323). Accordingly, in some embodiments, the composition comprises at least one of: (i) a first peptide comprising the amino acid sequence PFPQPQLPY (SEQ ID NO: 539) and PQPQLPYPQ (SEQ ID NO: 557), (ii) a second peptide comprising the amino acid sequence PFPQPQQPF (SEQ ID NO: 558) and PQPQQPFPW (SEQ ID NO: 559), or (iii) a third peptide comprising the amino acid sequence PIPQQPQPY (SEQ ID NO: 560). In some embodiments, the first peptide comprises LQPFPQPQLPYPQPQ (SEQ ID NO: 561); the second peptide comprises QPFPQPQQPFPWQP (SEQ ID NO: 562); and/or the third peptide comprises PEQPIPQQPQPYPQQ (SEQ ID NO: 563). In some embodiments, the peptides are 8-30 amino acids in length.

Modifications to a gluten peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, pyrolation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc.

The phrases “protecting group” and “blocking group” as used herein, refers to modifications to the peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- and/or C-terminal. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell.

A gluten peptide may also be chemically modified at the level of amino acid side chains, of amino acid chirality, and/or of the peptide backbone.

In some embodiments, the composition comprises at least one of a wheat gluten, a barley hordein, and a rye secalin. In some embodiments, the composition comprises at least two of a wheat gluten, a barley hordein, or a rye secalin. In some embodiments, the composition comprises a wheat gluten, a barley hordein, and a rye secalin. In some embodiments, the composition comprises a consistently known amount of a wheat gluten, a barley hordein, and/or a rye secalin. For example, the amount of wheat gluten, barley hordein, and/or rye secalin may be standardized such that each composition for each subject contains the same amount of wheat gluten, barley hordein, and/or rye secalin. In some embodiments, the wheat gluten, barley hordein, and/or rye secalin are present in an amount of at least 500 mg, e.g., 500 mg to 10 grams.

Administration of the composition comprising a gluten peptide may be self-administration by the subject or administration by a qualified individual, e.g., a medical practitioner such as a doctor or nurse. Such administration may be through any method known in the art. Compositions suitable for each administration route are well known in the art (see, e.g., Remington: The Science and Practice of Pharmacy, 22nd Ed., Pharmaceutical Press, 2012). In some embodiments, administration of the composition comprising a gluten peptide is oral administration.

Suitable forms of oral administration include foodstuffs (e.g., baked goods such as breads, cookies, muffins, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions and such compositions may contain one or more agents such as sweetening agents, flavoring agents, coloring agents and preserving agents in order to provide pharmaceutically elegant and palatable preparations.

### Peptide Production

The peptides described herein (e.g., antigen peptides, islet autoantigen peptides or gluten peptides as described herein) can be prepared in any suitable manner. For example, the peptides can be recombinantly and/or synthetically produced.

The peptides may be synthesised by standard chemistry techniques, including synthesis by an automated procedure using a commercially available peptide synthesiser. In general, peptides may be prepared by solid-phase peptide synthesis methodologies which may involve coupling each protected amino acid residue to a resin support, preferably a 4-methylbenzhydrylamine resin, by activation with dicyclohexylcarbodiimide to yield a peptide with a C-terminal amide. Alternatively, a chloromethyl resin (Merrifield resin) may be used to yield a peptide with a free carboxylic acid at the C-terminal. After the last residue has been attached, the protected peptide-resin is treated with hydrogen fluoride to cleave the peptide from the resin, as well as deprotect the side chain functional groups. Crude product can be further purified by gel filtration, high pressure liquid chromatography (HPLC), partition chromatography, or ion-exchange chromatography.

If desired, and as outlined above, various groups may be introduced into the peptide of the composition during synthesis or during expression, which allow for linking to other molecules or to a surface. For example, cysteines can be used to make thioethers, histidines for linking to a metal ion complex, carboxyl groups for forming amides or esters, amino groups for forming amides, and the like.

The peptides may also be produced using cell-free translation systems. Standard translation systems, such as reticulocyte lysates and wheat germ extracts, use RNA as a template; whereas “coupled” and “linked” systems start with DNA templates, which are transcribed into RNA then translated.

Alternatively, the peptides may be produced by transfecting host cells with expression vectors that comprise a polynucleotide(s) that encodes one or more peptides.

For recombinant production, a recombinant construct comprising a sequence which encodes one or more of the peptides is introduced into host cells by conventional methods such as calcium phosphate transfection, DEAE-dextran mediated transfection, microinjection, cationic lipid-mediated transfection, electroporation, transduction, scrape lading, ballistic introduction or infection.

One or more of the peptides may be expressed in suitable host cells, such as, for example, mammalian cells (for example, COS, CHO, BHK, 293 HEK, VERO, HeLa, HepG2, MDCK, W138, or NIH 3T3 cells), yeast (for example, Saccharomyces or Pichia), bacteria (for example, E. coli, P. pastoris, or B. subtilis), insect cells (for example, baculovirus in Sf9 cells) or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the peptide or variant thereof.

Suitable expression vectors include, for example, chromosomal, non-chromosomal and synthetic polynucleotides, for example, derivatives of SV40, bacterial plasmids, phage DNAs, yeast plasmids, vectors derived from combinations of plasmids and phage DNAs, viral DNA such as vaccinia viruses, adenovirus, adeno-associated virus, lentivirus, canary pox virus, fowl pox virus, pseudorabies, baculovirus, herpes virus and retrovirus. The polynucleotide may be introduced into the expression vector by conventional procedures known in the art.

The polynucleotide which encodes one or more peptides may be operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator. The expression vectors may also include an origin of replication and a selectable marker, such as the ampicillin resistance gene of E. coli to permit selection of transformed cells, i.e., cells that are expressing the heterologous polynucleotide. The nucleic acid molecule encoding one or more of the peptides may be incorporated into the vector in frame with translation initiation and termination sequences.

One or more of the peptides can be recovered and purified from recombinant cell cultures (i.e., from the cells or culture medium) by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and HPLC. Well known techniques for refolding proteins may be employed to regenerate active conformation when the peptide is denatured during isolation and or purification.

To produce a glycosylated peptide, it is preferred that recombinant techniques be used. To produce a glycosylated peptide, it is preferred that mammalian cells such as, COS-7 and Hep-G2 cells be employed in the recombinant techniques.

The peptides can also be prepared by cleavage of longer peptides, especially from food extracts.

Pharmaceutically acceptable salts of the peptides can be synthesised from the peptides which contain a basic or acid moiety by conventional chemical methods. Generally, the salts are prepared by reacting the free base or acid with stoichiometric amounts or with an excess of the desired salt-forming inorganic or organic acid or base in a suitable solvent.

IL-2 or an Agent that Stimulates IL-2 Expression

Aspects of the disclosure relate to use of IL-2 or an agent that stimulates IL-2 expression in any one of the methods or kits described herein. In some embodiments of any one of the methods provided, IL-2 or an agent that stimulates IL-2 expression is administered to a subject, e.g., a subject having or suspected of having an autoimmune disease. IL-2 is a cytokine, which is involved in proliferation of B and T lymphocytes.

The Genbank number for the human IL2 gene is 3558. Exemplary Genbank mRNA transcript IDs and protein IDs for IL-2 are NM_000586.3 and NP_000577.2, respectively. An exemplary protein sequence of IL-2 is provided below.

IL-2
(SEQ ID NO: 596)
LSCIALSLALVTNSAPTSSSTKKTQLQLEHLLLDLQMILNGINNYKNPKL
TRMLTFKFYMPKKATELKHLQCLEEELKPLEEVLNLAQSKNFHLRPRDLI
SNINVIVLELKGSETTFMCEYADETATIVEFLNRWITFCQSIIS

IL-2 protein may be recombinantly produced (e.g., in E. coli or a mammalian cell line) or isolated from biological material (e.g., from cells or tissues that secrete IL-2). Methods of producing and/or isolating proteins are known in the art.

IL-2 can be directly administered to patients and has been shown previously to have biological effects (see, e.g., Ahmadzadeh M. IL-2 administration increases CD4+CD25hiFoxp3+ regulatory T cells in cancer patients. Blood 107_2409 2006; and White MV Effects of in vivo administration of interleukin-2 (IL-2) and IL-4, alone and in combination, on ex vivo human basophil histamine release. Blood 79_1491 1992).

An agent that stimulates IL-2 expression is an agent that causes an increase in IL-2 expression (e.g., an increase in a level of IL-2 protein) in a subject after administration of the agent to the subject. An increase in IL-2 expression includes a level of IL-2 expression that is, for example, 1%, 5%, 10%, 20%, 30%, 40%, 50%, 60%, 70%, 80%, 90%, 100%, 150%, 200%, 300%, 400%, 500% or more above a control level of IL-2. A control level of IL-2 may be e.g., a level of IL-2 expression in the subject prior to administration of the agent or in a subject that has not been administered the agent. Exemplary agents that stimulate IL-2 expression include, but are not limited to, ingestion of gluten or gluten peptides in subjects with celiac disease, or administration of other antigens to sensitized subjects, or administration of agents that non-specifically activate T cells as demonstrated by an associated cytokine release syndrome, for example anti-CD3 (see, e.g., Chatenoud L. IN VIVO CELL ACTIVATION_FOLLOWING_OKT3 Administration. Transplantation 49_697 1990; and Baumgart D C. Et al. Transient cytokine-induced liver injury following administration of the humanized anti-CD3 antibody visilizumab (HuM291) in Crohn's disease. AmJGast 2009).

### T Cell Responses and Measurement Thereof

Aspects of the disclosure relate to a determination or measurement of a T cell response, e.g., a rare antigen-specific T cell response, in a sample from a subject, such as a subject having or suspected of having Celiac disease or T1D or Celiac disease and T1D or a subject having or suspected of having an autoimmune disease (optionally comorbid with Celiac disease). In some embodiments of any one of the methods provided, a first composition comprising a gluten peptide or gluten as described herein is administered to a subject and is capable of activating a CD4⁺ T cell in a subject, e.g., a subject with Celiac disease or T1D or Celiac disease and T1D. The term “activate” or “activating” or “activation” in relation to a CD4⁺ T cell refers to the presentation by an MHC molecule of an epitope on one cell to an appropriate T cell receptor on a CD4⁺ T cell, together with binding of a co-stimulatory molecule by the CD4⁺ T cell, thereby eliciting a “T cell response”, in this example, a CD4⁺ T cell response. Such a T cell response can be measured ex vivo, e.g., by measuring a T cell response in a sample, e.g., a sample comprising T cells from the subject. In some embodiments of any one of the methods provided, IL-2 or an agent that stimulates IL-2 expression is administered to a subject as described herein, e.g., a subject having or suspected of having an autoimmune disease, an allergy, an infectious disease or condition, or an adverse immune condition caused by administration of an isolated, recombinant or synthetic protein or peptide (optionally comorbid with Celiac disease). Without wishing to be bound by theory, administration of such agents or IL-2 is thought to cause T cell proliferation, which can amplify rare CD4⁺ T cells through “bystander” activation in a subject that recognizes autoantigen peptides. For example, oral gluten challenge has been shown to be followed by expansion of both gluten-reactive CD4+ T cells, and also CD8+ T cells circulating in blood 6-days later (see, e.g., Han. Dietary gluten triggers concomitant activation of CD4+ and CD8+αβ T cells and γδ T cells in celiac disease. PNAS 2013).

In some embodiments of any one of the methods provided, measuring a T cell response in a sample comprises contacting the sample with at least one islet autoantigen peptide or antigen peptide (e.g., autoantigen peptide) as described herein. For example, whole blood or PBMCs obtained from a subject, such as a subject who has been exposed to a gluten peptide (e.g., by administration of a first composition comprising a gluten peptide) or IL-2 or an agent that stimulates IL-2 expression, etc. may be contacted with the second composition in order to stimulate T cells in the sample. In some embodiments of any one of the methods provided, the sample is contacted with the at least one islet autoantigen peptide or antigen peptide as described herein for 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 36 or more, or 48 or more hours. In some embodiments of any one of the methods provided, the sample is contacted with the at least one islet autoantigen peptide or antigen peptide as described herein for 18 to 36, 20 to 30, or 22 to 26 hours. In some embodiments of any one of the methods provided, the sample is contacted with the at least one islet autoantigen peptide or antigen peptide as described herein for 24 hours.

Measuring a T cell response can be accomplished using any assay known in the art (see, e.g., Molecular Cloning: A Laboratory Manual, J. Sambrook, et al., eds., Fourth Edition, Cold Spring Harbor Laboratory Press, Cold Spring Harbor, N.Y., 2012, Current Protocols in Molecular Biology, F. M. Ausubel, et al., eds., John Wiley & Sons, Inc., New York).

In some embodiments of any one of the methods provided, measuring a T cell response in a sample comprises measuring a level of at least one cytokine in the sample. In some embodiments, measuring a rare antigen-specific T cell response in a sample comprises measuring a level of IP-10 in a sample comprising a rare antigen-specific T cell. In some embodiments, measuring a T cell response in a sample comprising T cells from a subject comprises contacting the sample with at least one antigen peptide, such as an islet autoantigen peptide, as described herein and measuring a level of at least one cytokine in the sample. In some embodiments, the at least one cytokine IFN-γ or IP-10. In some embodiments, the at least one cytokine is IP-10 and IFN-γ or IL-2 or IP-10 and IFN-γ and IL-2.

Interferon-γ (IFN-γ, also called IFNG, IFG, and IFI) is a dimerized soluble cytokine of the type II class of interferons. IFN-γ typically binds to a heterodimeric receptor consisting of Interferon γ receptor 1 (IFNGR1) and Interferon γ receptor 2 (IFNGR2). IFN-γ can also bind to the glycosaminoglycan heparan sulfate (HS). IFN-γ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops in a subject. In humans, the IFN-γ protein is encoded by the IFNG gene. The Genbank number for the human IFNG gene is 3458. Exemplary Genbank mRNA transcript IDs and protein IDs for IFN-γ are NM_000619.2 and NP_000610.2, respectively. An exemplary protein sequence of IFN-γ is provided below.

IFN- γ
(SEQ ID NO: 597)
QDPYVKEAENLKKYFNAGHSDVADNGTLFLGILKNWKEESDRKIMQSQIV
SFYFKLFKNFKDDQSIQKSVETIKEDMNVKFFNSNKKKRDDFEKLTNYSV
TDLNVQRKAIHELIQVMAELSPAAKTGKRKRSQMLFRGRRASQ

IFN-γ inducible protein-10 (IP-10, also referred to as C—X—C motif chemokine 10, CXCL10, small-inducible cytokine B10, SCYB10, C7, IFI10, crg-2, gIP-10, or mob-1) is a protein that in humans is encoded by the CXCL10 gene. IP-10 is a small cytokine belonging to the CXC chemokine family and binds to the chemokine receptor CXCR3. The Genbank ID number for the human CXCL10 gene is 3627. Exemplary Genbank mRNA transcript IDs and protein IDs for IP-10 are NM_001565.3 and NP_001556.2, respectively. An exemplary protein sequence of IP-10 is provided below.

IP-10
(SEQ ID NO: 598)
VPLSRTVRCTCISISNQPVNPRSLEKLEIIPASQFCPRVEIIATMKKKGE
KRCLNPESKAIKNLLKAVSKERSKRSP

Interleukin-2 (IL-2) is a protein that in humans is encoded by the IL2 gene. IL-2 is a secreted cytokine and binds, e.g., to the heterotrimeric protein receptor interleukin-2 receptor (IL-2R). The Genbank ID number for the human IL2 gene is 3558. Exemplary mRNA sequences and protein sequences for IL-2 are shown below.

>gi|125661059|ref|NM_000586.3| Homo sapiens inter-
leukin 2 (IL2), mRNA
(SEQ ID NO: 599)
AGTTCCCTATCACTCTCTTTAATCACTACTCACAGTAACCTCAACTCCTG
CCACAATGTACAGGATGCAACTCCTGTCTTGCATTGCACTAAGTCTTGCA
CTTGTCACAAACAGTGCACCTACTTCAAGTTCTACAAAGAAAACACAGCT
ACAACTGGAGCATTTACTGCTGGATTTACAGATGATTTTGAATGGAATTA
ATAATTACAAGAATCCCAAACTCACCAGGATGCTCACATTTAAGTTTTAC
ATGCCCAAGAAGGCCACAGAACTGAAACATCTTCAGTGTCTAGAAGAAGA
ACTCAAACCTCTGGAGGAAGTGCTAAATTTAGCTCAAAGCAAAAACTTTC
ACTTAAGACCCAGGGACTTAATCAGCAATATCAACGTAATAGTTCTGGAA
CTAAAGGGATCTGAAACAACATTCATGTGTGAATATGCTGATGAGACAGC
AACCATTGTAGAATTTCTGAACAGATGGATTACCTTTTGTCAAAGCATCA
TCTCAACACTGACTTGATAATTAAGTGCTTCCCACTTAAAACATATCAGG
CCTTCTATTTATTTAAATATTTAAATTTTATATTTATTGTTGAATGTATG
GTTTGCTACCTATTGTAACTATTATTCTTAATCTTAAAACTATAAATATG
GATCTTTTATGATTCTTTTTGTAAGCCCTAGGGGCTCTAAAATGGTTTCA
CTTATTTATCCCAAAATATTTATTATTATGTTGAATGTTAAATATAGTAT
CTATGTAGATTGGTTAGTAAAACTATTTAATAAATTTGATAAATAT
>gi|28178861|ref|NP_000577.2| interleukin-2 pre-
cursor [Homo sapiens]
(SEQ ID NO: 600)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT
>gi|28178861|ref|NP_000577.2| interleukin-2 mature
protein [Homo sapiens]
(SEQ ID NO: 601)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

In some embodiments, measuring a T cell response comprises measuring a level of at least one cytokine. Levels of at least one cytokine, e.g., IP-10, include levels of cytokine RNA, e.g., mRNA, and/or levels of cytokine protein. In a preferred embodiment, levels of the at least one cytokine, e.g., IP-10, are protein levels.

Assays for detecting cytokine RNA include, but are not limited to, Northern blot analysis, RT-PCR, sequencing technology, RNA in situ hybridization (using e.g., DNA or RNA probes to hybridize RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer with addressable location, such as Affymetrix microarray (Affymetrix®, Santa Clara, Calif.)). Designing nucleic acid binding partners, such as probes, is well known in the art. In some embodiments, the nucleic acid binding partners bind to a part of or an entire nucleic acid sequence of at least one cytokine, e.g., IFN-γ, IL-2 or IP-10, the sequence(s) being identifiable using the Genbank IDs described herein, the sequences provided herein, or as otherwise known in the art.

Assays for detecting protein levels include, but are not limited to, immunoassays (also referred to herein as immune-based or immuno-based assays, e.g., Western blot, ELISA, and ELISpot assays), Mass spectrometry, and multiplex bead-based assays. Binding partners for protein detection can be designed using methods known in the art and as described herein. In some embodiments, the protein binding partners, e.g., antibodies, bind to a part of or an entire amino acid sequence of at least one cytokine, e.g., IFN-γ, IL-2 or IP-10, the sequence(s) being identifiable using the Genbank IDs described herein, the sequences provided herein, or as otherwise known in the art. Other examples of protein detection and quantitation methods include multiplexed immunoassays as described for example in U.S. Pat. Nos. 6,939,720 and 8,148,171, and published U.S. Patent Application No. 2008/0255766, and protein microarrays as described for example in published U.S. Patent Application No. 2009/0088329.

In some embodiments, measuring a level of at least one cytokine comprises an enzyme-linked immunosorbent assay (ELISA) or enzyme-linked immunosorbent spot (ELISpot) assay. ELISA and ELISpot assays are well known in the art (see, e.g., U.S. Pat. Nos. 5,939,281, 6,410,252, and 7,575,870; Czerkinsky C, Nilsson L, Nygren H, Ouchterlony O, Tarkowski A (1983) “A solid-phase enzyme-linked immunospot (ELISPOT) assay for enumeration of specific antibody-secreting cells”. J Immunol Methods 65 (1-2): 109-121 and Lequin R (2005). “Enzyme immunoassay (EIA)/enzyme-linked immunosorbent assay (ELISA)”. Clin. Chem. 51(12): 2415-8).

An exemplary ELISA involves at least one binding partner, e.g., an antibody, with specificity for the at least one cytokine, e.g., IFN-γ, IL-2 or IP-10. The sample with an unknown amount of the at least one cytokine can be immobilized on a solid support (e.g., a polystyrene microtiter plate) either non-specifically (via adsorption to the surface) or specifically (via capture by another binding partner specific to the same at least one cytokine, as in a “sandwich” ELISA). After the antigen is immobilized, the binding partner for the at least one cytokine is added, forming a complex with the immobilized at least one cytokine. The binding partner can be attached to a detectable label as described herein (e.g., a fluorophor or an enzyme), or can itself be detected by an agent that recognizes the at least one cytokine binding partner that is attached to a detectable label as described herein (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

An exemplary ELISpot assay involves a binding agent for the at least one cytokine (e.g., an anti-IFN-γ) that is coated aseptically onto a PVDF (polyvinylidene fluoride)-backed microplate. Cells of interest (e.g., peripheral blood mononuclear cells) are plated out at varying densities, along with antigen (e.g., a gluten peptide as described herein), and allowed to incubate for a period of time (e.g., about 24 hours). The at least one cytokine secreted by activated cells is captured locally by the binding partner for the at least one cytokine on the high surface area PVDF membrane. After the at least one cytokine is immobilized, a second binding partner for the at least one cytokine is added, forming a complex with the immobilized at least one cytokine. The binding partner can be linked to a detectable label (e.g., a fluorophor or an enzyme), or can itself be detected by an agent that recognizes the binding partner for the at least one cytokine (e.g., a secondary antibody) that is linked to a detectable label (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

In some embodiments, a level of at least one cytokine is measured using a multiplex bead-based assay (see, e.g., MAGPix® from Luminex® Corp). An exemplary multiplex bead-based assay involves use of magnetic beads that are internally dyed with fluorescent dyes to produce a specific spectral address. Binding partners (e.g., antibodies) are conjugated to the surface of beads to capture the at least one cytokine, e.g., IP-10. The sample is loaded into a 96-well plate containing the beads and the sample is incubated to allow binding of the at least one cytokine, e.g., IP-10, to the beads. A second biotinylated binding partner for the at least one cytokine, e.g., IP-10, is added after the at least one cytokine, e.g., IP-10, binds to the beads. A streptavidin-conjugated detectable label is then bound to the biotin. Light emitting diodes are used to illuminate the samples, causing the fluorescent dyes in the beads to fluoresce, as well as the detectable label to fluoresce. The concentration of the at least one cytokine, e.g., IP-10, is then determined based on the level of fluorescence. An exemplary system for running a multiplex bead-based assay is the MAGPIX® system available from Luminex® Corporation (see, e.g., U.S. Pat. No. 8,031,918, U.S. Pat. No. 8,296,088, U.S. Pat. No. 8,274,656, U.S. Pat. No. 8,532,351, U.S. Pat. No. 8,542,897, U.S. Pat. No. 6,514,295, U.S. Pat. No. 6,599,331, U.S. Pat. No. 6,632,526, U.S. Pat. No. 6,929,859, U.S. Pat. No. 7,445,844, U.S. Pat. No. 7,718,262, U.S. Pat. No. 8,283,037, and U.S. Pat. No. 8,568,881, all of which are incorporated by reference herein, and in particular the systems provided herein).

In some embodiments of any one of the methods provided, a control T cell response, e.g., a control level of IP-10, is contemplated. In some embodiments, the control T cell response is a negative control T cell response, e.g., a negative control level of IP-10. Exemplary negative controls include, but are not limited to, a T cell response or level of IP-10 in a sample that has been contacted with a non-T cell-activating peptide (e.g., a peptide not recognized by T cells present in a sample from a subject), such as a non-CD4⁺-T cell-activating peptide, or a T cell response or level of IP-10 in sample that has not been contacted with a T cell-activating peptide (e.g., contacting the sample with a saline solution containing no peptides), such as a CD4⁺ T cell-activating peptide. Another exemplary control T cell response, e.g., a control level of IP-10, can be obtained using a sample from the subject before administration of a composition comprising a gluten peptide or IL-2 or an agent that stimulates IL-2 expression, such that a baseline T cell response, e.g., a baseline level of IP-10, can be established. Another exemplary control T cell response can be obtained using a sample from a subject that has been administered a placebo as described herein. Another exemplary control T cell response can be obtained using a sample that has been contacted with a composition comprising phosphate buffered saline or phosphate buffered saline and dimethyl sulfoxide.

In some embodiments of any one of the methods provided, the control T cell response is a positive control T cell response. In some embodiments of any one of the methods provided, a positive control T cell response is a T cell response in a sample that has been contacted with a T cell-activating peptide (e.g., a gluten peptide described herein) or a pathogen-derived recall antigen peptide mixture (e.g., CEF; a pool of 23 peptides consisting of MHC class I-restricted T-cell epitopes from human cytomegalovirus, Epstein Barr virus and influenza virus available from Mabtech (#3615-1; Nacka Strand, Sweden) or CEFT, a pool of 27 peptides consisting of MHC class I- and II-restricted T-cell epitopes from Clostridium tetani, Epstein-Barr virus, Human cytomegalovirus, Influenza A, available from Creative Peptides (#PPO-H107)).

Any control T cell responses can be measured using any one of the methods above or any other appropriate methods.

Any one or more T cell responses described herein may be performed as part of one assay (e.g., in multiple wells of a single plate) or as part of multiple assays (e.g., some or all of the T cell responses are measured using separate assays performed separately time and/or space).

An elevated T cell response, e.g., an elevated level of IP-10, IFN-gamma, and/or IL-2, includes a response that is, for example, 1%, 5%, 10%, 20%, 30%, 40%, 50%, 60%, 70%, 80%, 90%, 100%, 150%, 200%, 300%, 400%, 500% or more above a control T cell response, e.g., a control level of IP-10, IFN-gamma, and/or IL-2, respectively. In some embodiments, an elevated level of IP-10 is a level that is at least two-fold greater than a control level of IP-10. In some embodiments, an elevated level of IL-2 is a level that is at least two-fold greater than a control level of IL-2. In some embodiments, an elevated level of IFN-gamma is a level that is at least two-fold greater than a control level of IFN-gamma. A reduced T cell response, e.g., a reduced level of IP-10, IFN-gamma, and/or IL-2, includes a response that is, for example, 1%, 5%, 10%, 20%, 30%, 40%, 50%, 60%, 70%, 80%, 90%, 100%, 150%, 200%, 300%, 400%, 500% or more below a control T cell response, e.g., a control level of IP-10, IFN-gamma, and/or IL-2, respectively.

### Samples

Samples, as used herein, refer to biological samples taken or derived from a subject, e.g., a subject having or suspected of having Celiac disease, T1D or both or a subject having or suspected of having an autoimmune disease, an allergy, an infectious disease or condition, or an adverse immune condition caused by administration of an isolated, recombinant or synthetic protein or peptide, such as a therapeutic (optionally comorbid with Celiac disease). Examples of samples include tissue samples or fluid samples. Examples of fluid samples are whole blood, plasma, serum, and other bodily fluids that comprise T cells, e.g., rare antigen-specific T cells such as rare autoantigen-specific T cells. In some embodiments of any one of the methods provided, the sample comprises T cells. In some embodiments of any one of the methods provided, the sample comprises a rare antigen-specific T cell such as a rare autoantigen-specific T cell. In some embodiments, the sample comprises T cells, e.g., rare antigen-specific T cells, and monocytes and/or granulocytes. In some embodiments of any one of the methods provided, the sample comprises whole blood or peripheral blood mononuclear cells (PBMCs). The T cell, e.g., rare antigen-specific T cell, may be a CD4⁺ T cell. In some embodiments of any one of the methods provided, the methods described herein comprise obtaining or providing the sample. In some embodiments of any one of the methods provided, a first and second sample are contemplated. In some embodiments of any one of the methods provided, the first sample is obtained from a subject after administration of a composition comprising a gluten peptide as described herein. In some embodiments of any one of the methods provided, the first sample is obtained from a subject after administration of IL-2 or an agent that stimulates IL-2 expression as described herein. In some embodiments of any one of the methods provided, the second sample is obtained before administration of the composition, IL-2 or agent that stimulates IL-2 expression. In some embodiments of any one of the methods provided, the first sample is obtained from the subject six days after the administration of the composition or IL-2 or agent that stimulates IL-2 expression and the second sample is obtained before administration of the composition or IL-2 or agent that stimulates IL-2 expression. In some embodiments of any one of the methods provided, the second sample may be a sample obtained from a subject that has been administered a placebo as described herein. Additional samples, e.g., third, fourth, fifth, etc., are also contemplated if additional measurements of a T cell response are desired. Such additional samples may be obtained from a subject at any time, e.g., before or after administration of a composition comprising a gluten peptide and/or a placebo or before or after administration of IL-2 or an agent that stimulates IL-2 expression. In some embodiments of any one of the methods provided, a sample is obtained from the subject 1, 2, 3, 4, 5, 6, 7, 8, 9, 10, or more days after administration of a composition comprising a gluten peptide or IL-2 or an agent that stimulates IL-2 expression. In some embodiments of any one of the methods provided, a sample is obtained from the subject six days after administration of the composition comprising a gluten peptide or IL-2 or an agent that stimulates IL-2 expression. In some embodiments of any one of the methods provided, a sample is obtained from the subject 1, 2, 3, 4, 5, 6, 7, 8, 9, 10, or more days after administration of a placebo. In some embodiments of any one of the methods provided, a sample is obtained from the subject six days after administration of the placebo.

### Subjects

In some embodiments of any one of the methods provided, a subject may include any subject that is suspected of having Celiac disease, T1D or both. In some embodiments of any one of the methods provided, the subject may include any subject that has or is suspected of having Celiac disease, T1D or both. Preferably, the subject is a human. In some embodiments of any one of the methods provided, the subject has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). In some embodiments of any one of the methods provided, the subject is HLA-DQ2.5 positive (i.e., has both susceptibility alleles DQA1*05 and DQB1*02). In some embodiments of any one of the methods provided, a subject may have a family member that has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). The presence of susceptibility alleles can be detected by any nucleic acid detection method known in the art, e.g., by polymerase chain reaction (PCR) amplification of DNA extracted from the patient followed by hybridization with sequence-specific oligonucleotide probes. A subject may be identified as having or suspected of having T1D, Celiac disease or both using diagnostic methods known in the art and described herein (e.g., diagnostic assays for T1D and diagnostic assays for Celiac disease).

In some embodiments of any one of the methods provided, the subject has one or more symptoms of T1D. Exemplary symptoms of T1D include, but are not limited to, polyuria (frequent urination), polydipsia (increased thirst), polyphagia (increased hunger), or weight loss. In some embodiments of any one of the methods provided, the subject may have diabetic ketoacidosis. Symptoms of diabetic ketoacidosis include xeroderma (dry skin), rapid deep breathing, drowsiness, abdominal pain, and vomiting. Other symptoms of T1D are known in the art and within the knowledge of the skilled practitioner.

In some embodiments of any one of the methods provided, the subject is on a gluten-free diet.

In some embodiments of any one of the methods provided, a subject may include any subject that is suspected of having an autoimmune disease, an allergy, an infectious disease or condition, or an adverse immune condition caused by administration of an isolated, recombinant or synthetic protein or peptide. In some embodiments of any one of the methods provided, a subject may include any subject that is suspected of having or has been exposed to a foreign antigen as described herein.

In some embodiments of any one of the methods provided, the subject may include any subject that has or is suspected of having an autoimmune disease. Exemplary autoimmune diseases include, but are not limited to, rheumatoid arthritis, multiple sclerosis, immune-mediated or Type I diabetes mellitus, inflammatory bowel disease (e.g., Crohn's disease or ulcerative colitis), systemic lupus erythematosus, psoriasis, scleroderma, autoimmune thyroid disease, alopecia areata, Grave's disease, Guillain-Barré syndrome, celiac disease, Sjögren's syndrome, rheumatic fever, gastritis, autoimmune atrophic gastritis, autoimmune hepatitis, insulitis, oophoritis, orchitis, uveitis, phacogenic uveitis, myasthenia gravis, primary myxoedema, pernicious anemia, autoimmune haemolytic anemia, Addison's disease, scleroderma, Goodpasture's syndrome, nephritis, for example, glomerulonephritis, psoriasis, pemphigus vulgaris, pemphigoid, sympathetic opthalmia, idiopathic thrombocylopenic purpura, idiopathic feucopenia, Wegener's granulomatosis and poly/dermatomyositis. In some embodiments, the autoimmune disease is not Celiac disease.

In some embodiments of any one of the methods provided, a subject may include any subject that is suspected of having an autoimmune disease and Celiac disease. In some embodiments of any one of the methods provided, the subject may include any subject that has or is suspected of having an autoimmune disease and Celiac disease. Exemplary autoimmune diseases that are co-morbid with Celiac disease include, but are not limited to, autoimmune thyroiditis (including Grave's disease and Hashimoto's thyroiditis), Type-1 diabetes, latent autoimmune disease of adults (LADA), autoimmune adrenal insufficiency (Addison's disease), primary biliary cirrhosis, primary sclerosing cholangitis, chronic autoimmune hepatitis, systemic lupus erythematosus, rheumatoid arthritis, Sjögren's syndrome and scleroderma.

In some embodiments of any one of the methods provided, the subject has one or more symptoms of an autoimmune disease, an allergy, an infectious disease or condition, or an adverse immune condition caused by administration of an isolated, recombinant or synthetic protein or peptide. Symptoms of such diseases and conditions are known in the art and within the knowledge of the skilled practitioner.

### Controls and Control Subjects

In some embodiments of any one of the methods provided, the method comprises measuring a T cell response in a sample obtained from a subject after administration of a composition comprising a gluten peptide and comparing the T cell response to one or more control T cell responses. In some embodiments of any one of the methods provided, the method comprises measuring a T cell response in a sample obtained from a subject after administration of IL-2 or an agent that stimulates IL-2 expression and comparing the T cell response to one or more control T cell responses. In some embodiments of any one of the methods provided, the method comprises measuring a T cell response in a sample obtained from a subject without administration of a composition beforehand. In some embodiments of any one of the methods provided, the control T cell response is a T cell response in a sample obtained from the same subject prior to administration of the composition or the IL-2 or the agent that stimulates IL-2 expression. In some embodiments of any one of the methods provided, the control T cell response is a T cell response in a sample obtained from a different subject after administration of a placebo. In some embodiments of any one of the methods provided, the control T cell response is a T cell response in a sample contacted with a composition comprising phosphate buffered saline. In some embodiments of any one of the methods provided, the control T cell response is a T cell response in a sample contacted with a composition comprising phosphate buffered saline and dimethyl sulfoxide.

However, other or further controls are also contemplated. For example, a control T cell response may be a T cell response in a sample from a control subject (or subjects). In some embodiments of any one of the methods provided, a control subject is a subject having or suspected of having Celiac disease but does not have T1D or another autoimmune disease. In some embodiments of any one of the methods provided, a control subject has one or more HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein but does not have Celiac disease or T1D. In some embodiments of any one of the methods provided, a control subject does not have any of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein. In some embodiments of any one of the methods provided, a control subject is a healthy individual not having or suspected of having Celiac disease, T1D, an autoimmune disease other than T1D, an allergy, an infectious disease or condition, or an adverse immune condition caused by administration of an isolated, recombinant or synthetic protein or peptide. In some embodiments of any one of the methods provided, a control T cell response is a pre-determined value from a control subject or subjects, such that the control T cell response need not be measured every time the methods described herein are performed.

### Placebo

Aspects of the disclosure relate to administration of a placebo to a subject, such as a subject having or suspected of having Celiac disease, T1D or both. Any appropriate placebo is contemplated. A preferred placebo is nearly or entirely indistinguishable from the composition causing the desired effect. The desired effect herein, in preferred embodiments, is activation and/or mobilization of CD4⁺ T cells in a subject who has Celiac disease and T1D after administration of a composition comprising a gluten peptide. Without wishing to be bound by theory, it is believed that the gluten peptide serves as the active component causing the activation and/or mobilization of CD4⁺ T cells in a subject who has Celiac disease. The activation and/or mobilization of CD4⁺ T cells can be measured in a sample from the subject as described herein. Accordingly, in some embodiments, the placebo does not contain a gluten peptide (or is not in amount that causes what would be considered a positive T cell response) or is “gluten-free”. A placebo can be determined to be gluten-free using standard definitions (see, e.g., Codex Alimentarius as measured by accepted gluten food tests such as R5-ELISA <20 ppm or no detectable gluten TGA Australia). Depending on the route of administration, the degree of alteration and disguise of the placebo may vary. In some embodiments, the placebo contains the same components as the composition but does not contain a gluten peptide (or an amount that causes what would be considered a positive T cell response).

In some embodiments of any one of the methods provided, the gluten peptide composition and the placebo are administered orally (e.g., as foodstuffs). In such embodiments, the placebo and the composition should have a similar taste, texture and appearance such that a subject cannot distinguish between the two while consuming either foodstuff. For example, the composition comprising a gluten peptide may be a foodstuff (such a cookie, muffin, or bread) containing wheat gluten, barley hordein, and/or rye secalin. The corresponding placebo foodstuff in some embodiments does not contain any of wheat gluten, barley hordein, and rye secalin. It is expected that, generally, omission of wheat, barley, and rye will alter the taste, texture and/or appearance of the placebo foodstuff. To bring the taste, texture and/or appearance of the placebo closer to that of the foodstuff comprising a gluten peptide, the placebo may comprise additional components or comprise alterations or omissions of components found in the gluten-peptide-containing foodstuff. Such additional components include, e.g., fillers, sweetening agents, flavoring agents, coloring agents, thickening agents (e.g., xantam gum, arrowroot, or guar gum), and preserving agents. Such additional components should ideally not comprise a gluten peptide. Exemplary fillers include, but are not limited to, flours that have no gluten peptides such as Almond flour, Amaranth flour, Buckwheat flour, Chestnut flour, Coconut flour, Corn flour, Millet flour, Montina® flour, Quinoa flour, Rice flour, Sorghum flour, Teff flour, Garbanzo Bean flour, Soy flour, Potato flour, Tapioca flour, and combinations thereof. Accordingly, in some embodiments, the placebo does not contain a gluten peptide; and comprises at least one additional component and/or excludes or alters at least one component of the composition comprising a gluten peptide.

For example, the administration of the placebo may occur more than once, e.g., two or more times daily, daily, bi-weekly, or weekly. In some embodiments, placebo administration is daily for 1, 2, 3, 4, 5, 6, 7, 8, 9, 10, 11, 12, 13, 14 or more days, or 1, 2, 3, 4, 5, 6, 7, 8 or more weeks. In some embodiments, placebo administration is daily for 3 days. In some embodiments, placebo administration is at least once daily (i.e., 1, 2, 3, 4, 5 or more times daily) for 1, 2, 3, 4, 5, 6, 7, 8, 9, 10, 11, 12, 13, 14 or more days, or 1, 2, 3, 4, 5, 6, 7, 8 or more weeks. In some embodiments, administration is at least once daily (i.e., 1, 2, 3, 4, 5 or more times daily) for 3 days. In some embodiments, the placebo is administered to the subject three times a day for three days.

Administration of the placebo may be self-administration by the subject or administration by a qualified individual, e.g., a medical practitioner such as a doctor or nurse. Such administration may be through any method known in the art. Compositions suitable for each administration route are well known in the art (see, e.g., Remington: The Science and Practice of Pharmacy, 22nd Ed., Pharmaceutical Press, 2012). In some embodiments, administration of the placebo is oral administration.

Suitable forms of oral administration include foodstuffs (e.g., baked goods such as breads, cookies, muffins, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions and such compositions may contain one or more agents such as sweetening agents, flavoring agents, coloring agents and preserving agents in order to provide pharmaceutically elegant and palatable preparations.

### Other or Further Testing

In some embodiments of any one of the methods provided, methods described herein further comprise other or further testing of a subject (e.g., before or after a method described herein or as a result of a T cell response measurement). As used herein, “other testing” and “further testing” describe use of at least one diagnostic method, such as for T1D or Celiac disease. In some embodiments of any one of the methods provided, the method comprises performing further testing for T1D if a subject is identified as in need of further testing for T1D (e.g., as a result of a T cell response measurement). Such other testing may be performed as part of the methods described herein or after the methods described herein (e.g., as a companion diagnostic), or before use of the methods described herein (e.g., as a first-pass screen to eliminate certain subjects before use of the methods described herein, e.g., eliminating those that do not have one or more HLA-DQA and HLA-DQB susceptibility alleles).

Any diagnostic method or combinations thereof for T1D and/or Celiac disease is contemplated for other or further testing.

Exemplary other/further testing for T1D includes, but is not limited to autoantibody assays, a glycated hemoglobin test, a glucose tolerance test, a fasting blood sugar test, C-peptide levels, and/or urinary glucose and ketones. In some embodiments, a glycated hemoglobin level at or above 6.5, plasma glucose at or above 11.1 mmol/L (200 mg/dL), a fasting plasma glucose level at or above 7.0 mmol/L, the presence of autoantibodies, or a combination thereof, indicates the subject has T1D.

Autoantibodies may be detected using any method known in the art, e.g., by ELISA, histology, cytology, immunofluorescence or western blotting. In some embodiments, autoantibodies are detecting using an immunoassay. In some embodiments, autoantibodies comprise one or more of islet cell autoantibodies, insulin autoantibodies, 65-kDa isoform of glutamic acid decarboxylase (GAD65) autoantibodies, islet antigen-2 (IA-2) autoantibodies, and zinc transporter (ZnT8) autoantibodies. Islet cell autoantibodies may be detected by indirect immunofluorescence. GAD65, IA-2, and/or ZnT8 autoantibodies may be detected using radioimmunoassay or ELISA.

Glycated hemoglobin may be detected using high-performance liquid chromatography (HPLC), immunoassay, enzymatic assay, capillary electrophoresis, or boronate affinity chromatography.

A glucose tolerance test may comprise administering glucose to a subject and obtaining blood from the subject after the glucose administration to determine how quickly the glucose is cleared from the blood. In some embodiments, an oral glucose tolerance test (OGTT) is used and a standard dose of glucose is ingested by mouth and blood levels are measured from a sample collected two hours later. Glucose may be measured using any method known in the art.

A fasting blood sugar test may comprise measuring blood glucose levels after a subject has not eaten for at least 8 hours. Glucose may be measured using any method known in the art.

Elevated urinary glucose and ketones support the diagnosis of Type-1 diabetes, elevated urinary glucose without ketonuria is consistent with Type-2 diabetes.

Fasting serum levels of the C-peptide fragment of proinsulin reflect the pancreatic beta-cell secretion of insulin, and aid in the diagnosis of autoimmune diabetes by demonstrating reduced C-peptide levels compared to Type-2 diabetics.

Exemplary other/further testing for Celiac disease includes, but is not limited to, intestinal biopsy, serology (measuring the levels of one or more antibodies present in the serum), and genotyping (see, e.g., Walker-Smith J A, et al. Arch Dis Child 1990).

Detection of serum antibodies (serology) is contemplated. The presence of such serum antibodies can be detected using methods known to those of skill in the art, e.g., by ELISA, histology, cytology, immunofluorescence or western blotting. Such antibodies include, but are not limited to: IgA ant-endomysial antibody (IgA EMA), IgA anti-tissue transglutaminase antibody (IgA tTG), IgA anti-deamidated gliadin peptide antibody (IgA DGP), and IgG anti-deamidated gliadin peptide antibody (IgG DGP).

IgA EMA: IgA endomysial antibodies bind to endomysium, the connective tissue around smooth muscle, producing a characteristic staining pattern that is visualized by indirect immunofluorescence. The target antigen has been identified as tissue transglutaminase (tTG or transglutaminase 2). IgA endomysial antibody testing is thought to be moderately sensitive and highly specific for untreated (active) Celiac disease.

IgA tTG: The antigen is tTG. Anti-tTG antibodies are thought to be highly sensitive and specific for the diagnosis of Celiac disease. Enzyme-linked immunosorbent assay (ELISA) tests for IgA anti-tTG antibodies are now widely available and are easier to perform, less observer-dependent, and less costly than the immunofluorescence assay used to detect IgA endomysial antibodies. The diagnostic accuracy of IgA anti-tTG immunoassays has been improved further by the use of human tTG in place of the nonhuman tTG preparations used in earlier immunoassay kits. Kits for IgA tTG are commercially available (INV 708760, 704525, and 704520, INOVA Diagnostics, San Diego, Calif.).

Deamidated gliadin peptide-IgA (DGP-IgA) and deamidated gliadin peptide-IgG (DGP-IgG) are also contemplated herein and can be evaluated with commercial kits (INV 708760, 704525, and 704520, INOVA Diagnostics, San Diego, Calif.).

Genetic testing (genotyping) is also contemplated. Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRB5 & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

### Kits

Another aspect of the disclosure relates to kits. In some embodiments, the kit comprises (a) a means for detecting a T cell response; and (b) at least one antigen peptide, such as an islet autoantigen peptide, as described herein.

In some embodiments of any one of the kits provided, means for detecting a T cell response is a binding partner, e.g., an antibody that binds to a cytokine. In some embodiments, the binding partner that binds to a cytokine is a binding partner or plurality of binding partners that binds to IL-2, IFN-γ and/or IP-10. In some embodiments, the binding partner is a first binding partner that binds to IP-10 and a second binding partner that binds to IFN-γ or IL-2. In some embodiments, the binding partner is a first binding partner that binds to IP-10, a second binding partner that binds to IFN-γ, and a third binding partner that binds to IL-2.

In some embodiments of any one of the kits provided, the kit comprises (a) a means for detecting a level of IP-10; and (b) at least one antigen peptide (e.g., at least one autoantigen peptide) as described herein. In some embodiments of any one of the kits provided, the means for detecting a level of IP-10 is an antibody that binds to IP-10. In some embodiments of any one of the kits provided, the kit further comprises means for detecting a level of IFN-γ. In some embodiments of any one of the kits provided, the means for detecting a level of IFN-γ is an antibody that binds to IFN-γ. In some embodiments of any one of the kits provided, the kit further comprises means for detecting a level of IL-2. In some embodiments of any one of the kits provided, the means for detecting a level of IL-2 is an antibody that binds to IL-2. In some embodiments of any one of the kits provided, the kit further comprises means for detecting a level of IL-2 and IFN-γ. In some embodiments of any one of the kits provided, the means for detecting a level of IL-2 and IFN-γ is an antibody that binds to IL-2 and IFN-γ, respectively.

In some embodiments of any one of the kits provided, the kit further comprises a composition comprising a gluten peptide as described herein. In some embodiments of any one of the kits provided, the composition comprises at least one of a wheat gluten, a barley hordein, and a rye secalin. In some embodiments of any one of the kits provided, the kit further comprises a placebo. In some embodiments of any one of the kits provided, the composition and the placebo are foodstuffs.

In some embodiments of any one of the kits provided, the kit further comprises IL-2 or an agent that stimulates IL-2 expression.

In some embodiments of any one of the kits provided, kit comprises a container, such as a vial or tube, for whole blood. In some embodiments of any one of the kits provided, the at least one islet autoantigen peptide or antigen peptide is dried on the wall of the container for whole blood. In some embodiments of any one of the kits provided, the islet autoantigen peptide or antigen peptide is contained within a solution separate from the container, such that the islet autoantigen peptide or antigen peptide may be added to the container after blood collection. In some embodiments of any one of the kits provided, the islet autoantigen peptide or antigen peptide is in lyophilized form in a separate container, such that the islet autoantigen peptide may be reconstituted and added to the container after blood collection. In some embodiments of any one of the kits provided, the container further contains an anti-coagulant, such as heparin. In some embodiments of any one of the kits provided, the container is structured to hold a defined volume of blood, e.g., 1 mL or 5 mL. In some embodiments of any one of the kits provided, the container is present in the kit in duplicate or triplicate.

In some embodiments of any one of the kits provided, the kit further comprises a negative control container and/or a positive control container. The negative control container may be, for example, an empty container or a container containing a non-T cell-activating peptide (e.g., dried onto the wall of the container), such as a non-CD4+-T cell-activating peptide. The positive control container may contain, for example, a mitogen such as PHA-L (e.g., 10 units PHA-L). In some embodiments of any one of the kits provided, the negative control container and/or positive control container are structured to hold a defined volume of blood, e.g., 1 mL or 5 mL. In some embodiments of any one of the kits provided, the negative control container and/or positive control container are present in the kit in duplicate or triplicate. In some embodiments of any one of the kits provided, the kit comprises any combination of the components mentioned above.

Any suitable binding partner is contemplated. In some embodiments of any one of the kits provided, the binding partner is any molecule that binds specifically to a cytokine, e.g., IL-2, IFN-γ or IP-10. As described herein, “binds specifically” means that the molecule is more likely to bind to a portion of or the entirety of a protein to be measured than to a portion of or the entirety of another protein. In some embodiments of any one of the kits provided, the binding partner is an antibody, which includes antigen-binding fragments thereof, such as Fab, F(ab)2, Fv, single chain antibodies, Fab and sFab fragments, F(ab′)2, Fd fragments, scFv, or dAb fragments. Methods for making antibodies and antigen-binding fragments are well known in the art (see, e.g. Molecular Cloning: A Laboratory Manual, supra; Lewin's Genes XI, Jones & Bartlett Learning, 11th ed., 2012; Roitt's Essential Immunology, Wiley-Blackwell, 12th Ed., 2011; Current Protocols in Immunology, Wiley Online Library, 2014; WO2006/040153; WO2006/122786; and WO2003/002609). Binding partners also include other peptide molecules and aptamers that bind specifically. Methods for producing peptide molecules and aptamers are well known in the art (see, e.g., published US Patent Application No. 2009/0075834, U.S. Pat. Nos. 7,435,542, 7,807,351, and 7,239,742). In some embodiments, the binding partner is any molecule that binds specifically to an IL-2, IFN-γ or IP-10 mRNA. As described herein, “binds specifically to an mRNA” means that the molecule is more likely to bind to a portion of or the entirety of the mRNA to be measured (e.g., by complementary base-pairing) than to a portion of or the entirety of another mRNA or other nucleic acid. In some embodiments of any one of the kits provided, the binding partner that binds specifically to an mRNA is a nucleic acid, e.g., a probe.

In some embodiments of any one of the kits provided, the kit further comprises a first and second binding partner for a cytokine. In some embodiments of any one of the kits provided, the first and second binding partners are antibodies. In some embodiments of any one of the kits provided, the second binding partner is bound to a surface. The second binding partner may be bound to the surface covalently or non-covalently. The second binding partner may be bound directly to the surface, or may be bound indirectly, e.g., through a linker. Examples of linkers, include, but are not limited to, carbon-containing chains, polyethylene glycol (PEG), nucleic acids, monosaccharide units, and peptides. The surface can be made of any material, e.g., metal, plastic, paper, or any other polymer, or any combination thereof. In some embodiments, the first binding partner is washed over the cytokine bound to the second binding partner (e.g., as in a sandwich ELISA). The first binding partner may comprise a detectable label, or an agent that recognizes the first binding partner (e.g., a secondary antibody) may comprise a detectable label.

Any suitable agent that recognizes a binding partner is contemplated. In some embodiments, the binding partner is any molecule that binds specifically to the binding partner. In some embodiments of any one of the kits provided, the agent is an antibody (e.g., a secondary antibody). Agents also include other peptide molecules and aptamers that bind specifically to a binding partner. In some embodiments of any one of the kits provided, the binding partner comprises a biotin moiety and the agent is a composition that binds to the biotin moiety (e.g., an avidin or streptavidin).

In some embodiments of any one of the kits provided, the binding partner and/or the agent comprise a detectable label. Any suitable detectable label is contemplated. Detectable labels include any composition detectable by spectroscopic, photochemical, biochemical, immunochemical, chemical, or other physical means, e.g., an enzyme, a radioactive label, a fluorophore, an electron dense reagent, biotin, digoxigenin, or a hapten. Such detectable labels are well-known in the art and can be detectable through use of, e.g., an enzyme assay, a chromogenic assay, a luminometric assay, a fluorogenic assay, or a radioimmune assay. The reaction conditions to perform detection of the detectable label depend upon the detection method selected.

In some embodiments of any one of the kits provided, the kit further comprises instructions for performing a method herein and/or for detecting a T cell response (e.g., detecting a cytokine indicative of the T cell response, e.g., a rare antigen-specific T cell response) in a sample from a subject having or suspected of having Celiac disease, T1D or both or a subject otherwise described herein, such as a subject having or suspected of having an autoimmune disease, an allergy, an infectious disease or condition, or an adverse immune condition caused by administration of an isolated, recombinant or synthetic protein or peptide (optionally comorbid with Celiac disease). Instructions can be in any suitable form, e.g., as a printed insert or a label.

### GENERAL TECHNIQUES AND DEFINITIONS

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, J. Sambrook et al., Molecular Cloning: A Laboratory Manual, Cold Spring Harbour Laboratory Press (2012); T. A. Brown (editor), Essential Molecular Biology: A Practical Approach, Volumes 1 and 2, IRL Press (2000 and 2002); D. M. Glover and B. D. Hames (editors), Current Protocols in Molecular Biology, Greene Pub. Associates and Wiley-Interscience (1988, including all updates until present); Edward A. Greenfield (editor) Antibodies: A Laboratory Manual, Cold Spring Harbour Laboratory, (2013); and J. E. Coligan et al. (editors), Current Protocols in Immunology, John Wiley & Sons (including all updates until present).

In any one aspect or embodiment provided herein “comprising” may be replaced with “consisting essentially of” or “consisting of”.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Example 1

### A Randomized, Double-Masked, Placebo-Controlled, Gluten Challenge Study to Evaluate Induction of Islet Autoantigen-Specific T Cells in Patients with Type-1 Diabetes and Celiac Disease After Oral Gluten Challenge

The present study is intended to advance the understanding of how the immune response to gluten in celiac disease influences the islet-specific autoimmune T cell response associated with T1D. Previously, it has been shown in patients with celiac disease on gluten-free diet that reactivating the immune response associated with celiac disease by eating gluten-containing food for three days leads to the appearance of gluten-reactive T cells in blood six days later¹⁸. It is thought that the immune stimulation provided by gluten in patients with celiac disease who also have T1D causes not only gluten-reactive T cells but also islet-autoantigen-specific T cells to appear in the peripheral blood. This “bystander” stimulation of islet-specific T cells can occur if T cells specific for gluten are activated and secrete T-cell growth factors such as interleukin-2 that stimulate proliferation of not just gluten-specific T cells but also islet autoantigen-specific T cells in the same local draining lymph nodes.

Islet autoantigen-specific T cells are measured in blood along with gluten-reactive and recall antigen-specific T cells before and on the sixth day after commencing gluten food challenge, when gluten-reactive T cells are most plentiful in the peripheral blood. The first part of the Testing portion of the study seeks to optimize detection of rare islet autoantigen-specific T cells using fresh blood in a novel, highly sensitive laboratory test. In the second part of the study (Placebo-controlled) the optimized T cell detection assay will be used to test blood from an expanded group of subjects with both T1D and Celiac Disease, and a negative control group who have Celiac Disease but not T1D or islet autoimmunity as measured by islet-specific-autoantibodies. Blood drawn after gluten challenge will also be used to localize which parts of three islet autoantigens implicated in T1D (proinsulin, glutamic acid decarboxylase-65 [GAD65], and insulinoma-associated antigen-2 [IA-2]) are recognized by circulating T cells.

Showing functional linkage between gluten immunity and islet autoimmunity supports the concept that restoring or “strengthening” immune tolerance to gluten might also promote linked-tolerance to islet autoantigens in T1D¹⁹. And furthermore, mobilizing islet autoantigen-specific T cells in blood allowing them to be characterized without prolonged manipulation in the laboratory could overcome a significant obstacle to designing “antigen-specific” diagnostics and therapeutics for T1D.

### SUMMARY

Dietary gluten can play a role in causing or enhancing islet autoimmunity, but the mechanism is not understood. In patient-based studies over the last 14-years, short-term “gluten challenge” has provided a detailed understanding of the immune response underlying celiac disease, and allowed a therapeutic vaccine to be designed. In the present study, gluten challenge is being used for the first time to study patients affected by celiac disease as well as T1D to test islet autoimmunity as the effect of reactivation of gluten immunity.

### Rationale

### Gluten Challenge

Subjects eat approximately 9 grams of gluten daily for three days in addition to their usual gluten-free diet. Immune stimulation provided by oral gluten challenge in patients with comorbid T1D and celiac disease following a gluten free diet is expected to mobilize both gluten-specific and islet autoantigen-specific T cells in blood.

### Patient Selection

Subjects are aged 18 to 50 years. All subjects enrolled in the Pilot portion have both T1D and celiac disease. In the Placebo-controlled portion, ⅔ have both T1D and celiac disease, and % have celiac disease but not comorbid T1D. Subjects with both T1D and celiac disease who participate in Pilot portion would be eligible to re-enroll in the Placebo-controlled portion provided the time between gluten challenges is greater than 60 days. Subjects with celiac disease but not T1D who receive the gluten challenge, serve as a positive control for mobilization of gluten-reactive T cells, and all those without T1D serve as a negative control for induction of islet autoantigen-reactive T cells.

Only patients following strict gluten-free diet are enrolled because gluten-reactive T cells have not been identified in blood from patients with active celiac disease who are regularly exposed to gluten. Gluten-reactive T cells are mobilized in patients with celiac disease who have either of the two common genetic variants of celiac disease—either HLA-DQ2 or HLA-DQ8^{18,27}. For the present study subjects with HLA-DQ2 are selected for participation. Over 90% of patients with celiac disease have HLA-DQ2, and the gluten-reactive peptides recognized by T cells are better characterized for HLA-DQ2-associated celiac disease than those for HLA-DQ8-associated disease^{2,28}. A recent study indicated that amongst patients with T1D and celiac disease 77% (10/13) possessed HLA-DQ2.5 compared to 91% (70/77) with celiac disease alone²⁹.

Because up to 350 ml of blood would be collected during a study portion a normal hemoglobin level is required for entry into the study.

### Potential Risks

### Gluten Challenge

Ever since gluten was first incriminated in celiac disease by Wilhelm Dicke in 1950^{30,31}, reintroduction of gluten into the diet of patients with celiac disease following gluten free diet has been used in research and, in doubtful cases, to resolve whether patients truly have celiac disease. By definition, all patients with confirmed celiac disease eventually show deterioration of small bowel histology, but typically this occurs after 1 to 8 weeks while consuming 10 g gluten daily⁶. During extended gluten challenge, patients most commonly report tiredness, unwellness, and diarrhea, but a significant number report no symptoms⁶. Symptoms reported by 181 volunteers with celiac disease during un-blinded 3-day oral gluten challenge with wheat, barley or rye are shown below in the Table 1. In a recent study from Boston, Leffler et al. showed un-blinded gluten challenge with either 3 or 7.5 g/day gluten produced no significant histological deterioration after 3-days, but 75% showed deterioration by two-weeks²². In this study, a gastrointestinal symptom score index showed deterioration after three days into the 14-day gluten challenge, but symptom scores had normalized at follow-up two weeks after completing the two-week gluten challenge.

In the present study, volunteers consume approximately 9 g/day of gluten for three days. Over 400 English, Australian, Norwegian, Italian and Finnish volunteers with celiac disease have undergone un-blinded three-day gluten challenge of this type for the purpose of studying gluten-specific T cells^{18,23-26}. There have been no significant adverse events reported, all symptoms have resolved upon return to gluten-free diet, and well over 90% of subjects have completed the full three-day challenge. In un-blinded gluten challenges, about a quarter to a half of subjects are asymptomatic or report only tiredness, and the remainder report digestive symptoms that resolve with return to gluten-free diet. Symptoms affecting the gastrointestinal tract can range from mouth ulceration, nausea, bloating, abdominal pain, diarrhea, and occasionally vomiting. Upper gastrointestinal tract symptoms tend to predominate early and can have an onset within 2-6 hours after commencing gluten challenge. Vomiting when present resolves within hours even if gluten consumption continues. Two studies examining small bowel histology have now indicated gluten challenge for three days does not cause intestinal injury^{21,22}.

TABLE 1
Symptoms during 3-day ~10 g/day gluten challenge in adults
with celiac disease usually following strict gluten free diet (%)*

| Challenge | Wheat | Rye | Barley | All |
|---|---|---|---|---|
| Total (N) | 103 | 37 | 41 | 181 |
| Asymptomatic % | 16 | 22 | 15 | 17 |
| Nausea % | 32 | 22 | 17 | 27 |
| Bloating % | 35 | 19 | 42 | 33 |
| Vomiting % | 23 | 11 | 5 | 17 |
| Diarrhea % | 28 | 38 | 27 | 30 |
| Abdo. Pain % | 16 | 30 | 15 | 18 |
| Lethargy % | 29 | 24 | 22 | 27 |
| Constipation % | 10 | 14 | 17 | 12 |

*Tye-Din J A. Gluten peptides recognized by T cells in coeliac disease. Doctoral Thesis. University of Melbourne 2008. And: Tye-Din, J. A., et al. Comprehensive, quantitative mapping of T cell epitopes in gluten in celiac disease. Science translational medicine 2, 41ra51 (2010).

### Glycemic Control and Insulin Treatment

Digestive upsets and inability to eat regularly may affect glycemia. Insulin adjustments may be needed.

### Blood Collection

Up to 350 mL of blood is collected in the Pilot or Placebo-controlled portions of the study, no more than 200 mL is collected at any one time. This total volume is less than that collected during blood donation (450-500 mL) and is not considered harmful. Fainting and light-headedness may occur during blood collection, but subjects are resting seated or lying during blood collection.

### Study Objectives:

### Primary Objective:

1. To quantify and compare T cell responses to pancreatic islet autoantigens, immunodominant gluten peptides, and pathogen-derived recall antigens before and after oral gluten challenge in patients with both T1D and celiac disease and in patients with celiac disease alone.

### Secondary Objectives:

1. To establish the safety of oral gluten challenge in patients with comorbid celiac disease and T1D.

2. To define islet autoantigen-derived peptides recognized by T cells mobilized after oral gluten challenge.

### Primary Outcome Measures

### Laboratory Assessment of Blood for T Cell Responses

The presence of T cells specific for proinsulin, GAD65 and IA-2 autoantigens, immunodominant gluten peptides, or recall antigens is assessed using two complimentary cytokine release assays and either peripheral blood mononuclear cells (PBMC) separated from fresh blood or fresh whole blood incubated directly with antigens. Coded heparinized blood (without identifiable PHI) from study subjects is transported to the laboratory within two hours of collection using ambient temperature-controlled transporters. To enumerate antigen-specific T cells secreting interferon-γ, ELISpot assays using PBMC is performed. After overnight incubation, plates are developed, dried, and spot forming units determined for each well using an automated Zeiss counter (Zellnet Consulting). Experimental conditions and wells are arranged in order to optimize later analysis using a widely used on-line ELISpot analysis tool (http://www.scharp.org/zoe/runDFR/). Plasma from whole blood incubated for 24 h with antigens is used to assess antigen-stimulated interferon-γ (from T cells) and interferon-γ inducible protein-10 (IP-10, from monocytes and granulocytes stimulated by interferon-γ). A magnetic bead-based assay (MAGPix®) is used to measure interferon-γ and IP-10. Interim data analysis of antigen dose response studies in cytokine release assays in the Pilot portion is also used to select a single optimal antigen concentration for each of antigens assessed in Placebo-controlled portion. Data from MAGPix® assays in Pilot portion may also allow this assay to be used in place of interferon-γ ELISpot assays in Placebo-controlled portion to identify epitopes in islet autoantigens recognized by T cells in blood on Day−6 after gluten challenge. Mean of triplicate antigen-stimulated responses greater than 2× mean of six replicate wells with medium only are considered positive.

GO CRITERIA FOR PLACEBO-CONTROLLED PORTION: If one subject in the Pilot shows a positive response to any one of the islet auto antigen peptide pools then this will prompt initiation of enrollment in the Placebo-controlled Study.

### Primary Outcome Measure:

Interferon-γ T cell response on Day 6 measured by IFN-γ ELISpot assay, where a positive is defined by Distribution Free Resampling (DFR) of spot forming units in triplicate wells with any one of the six autoantigen peptide pools compared to replicate wells with medium only.

### Secondary Outcome Measures

1. Safety of Gluten Challenge in Patients with Celiac Disease and T1D:
Rates of adverse events after gluten challenge are determined.

### 2. Islet Autoantigen-Derived Peptides Recognized by T Cells

To define islet autoantigen-derived peptides recognized by T cells mobilized after oral gluten challenge whole blood secretion of IFNγ or IP-10 measured by MAGPix@ assay as described for the primary objective except that each peptide are tested in a single assay. Positive IFNγ responses to peptides are defined as being greater than 2× response to medium only, and confirmed by positive IP-10 response in the same plasma sample (IP-10 response≧twice mean negative control IP-10 response), or by repeat positive assessment of IFNγ by MAGPix@ using frozen plasma from the same peptide-whole blood incubation.

### Study Design

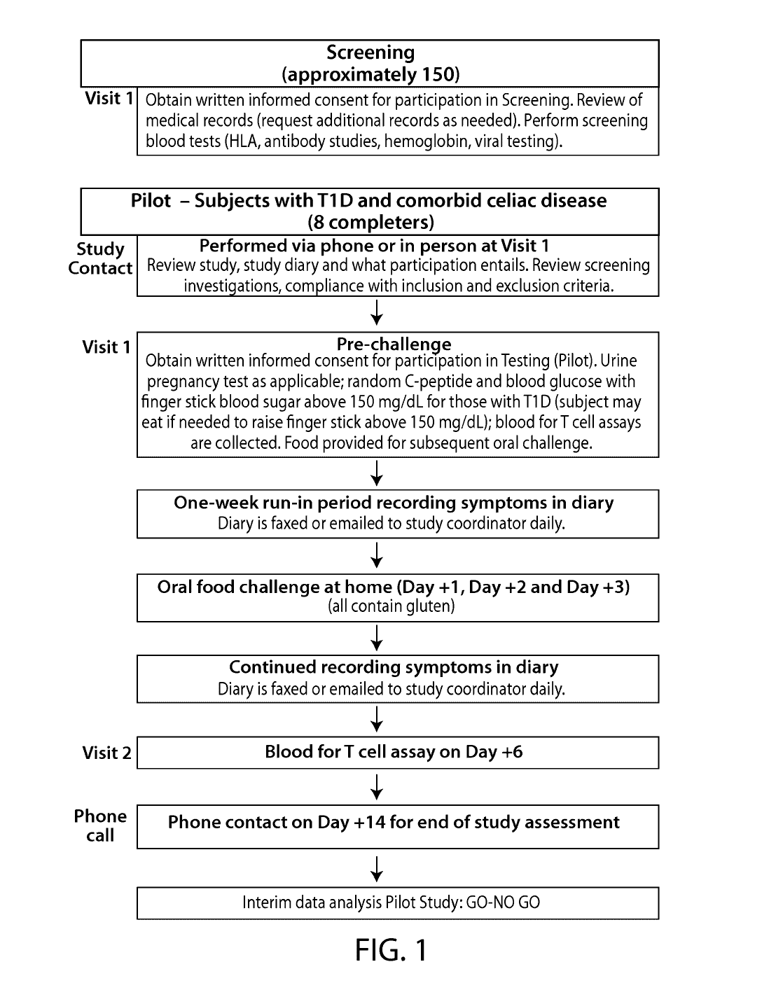

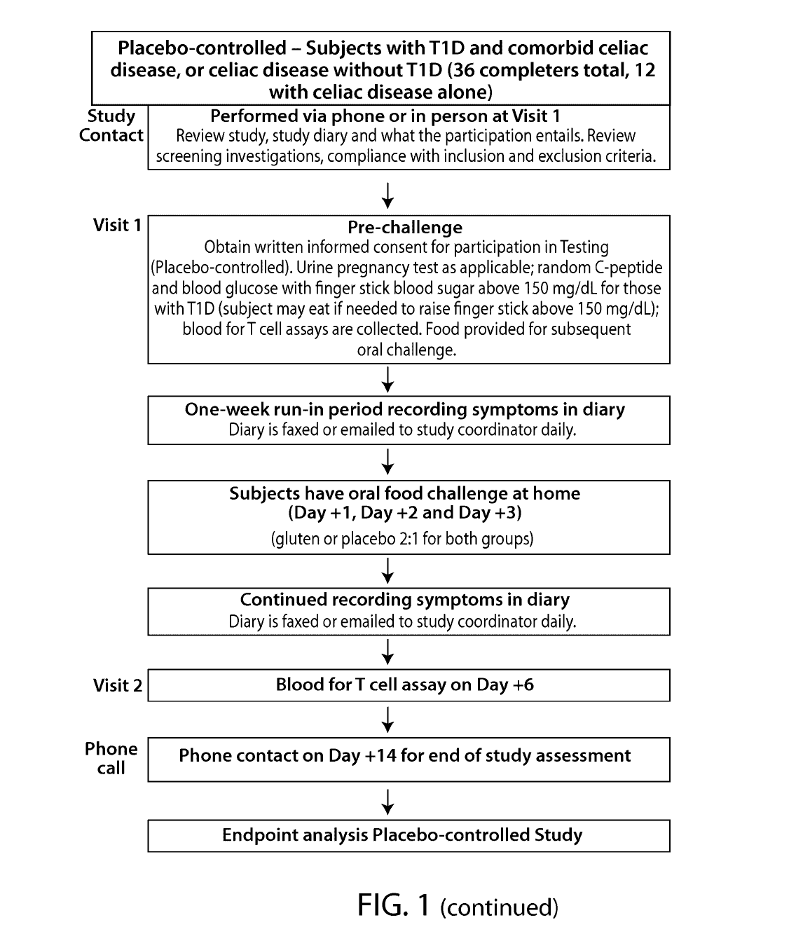

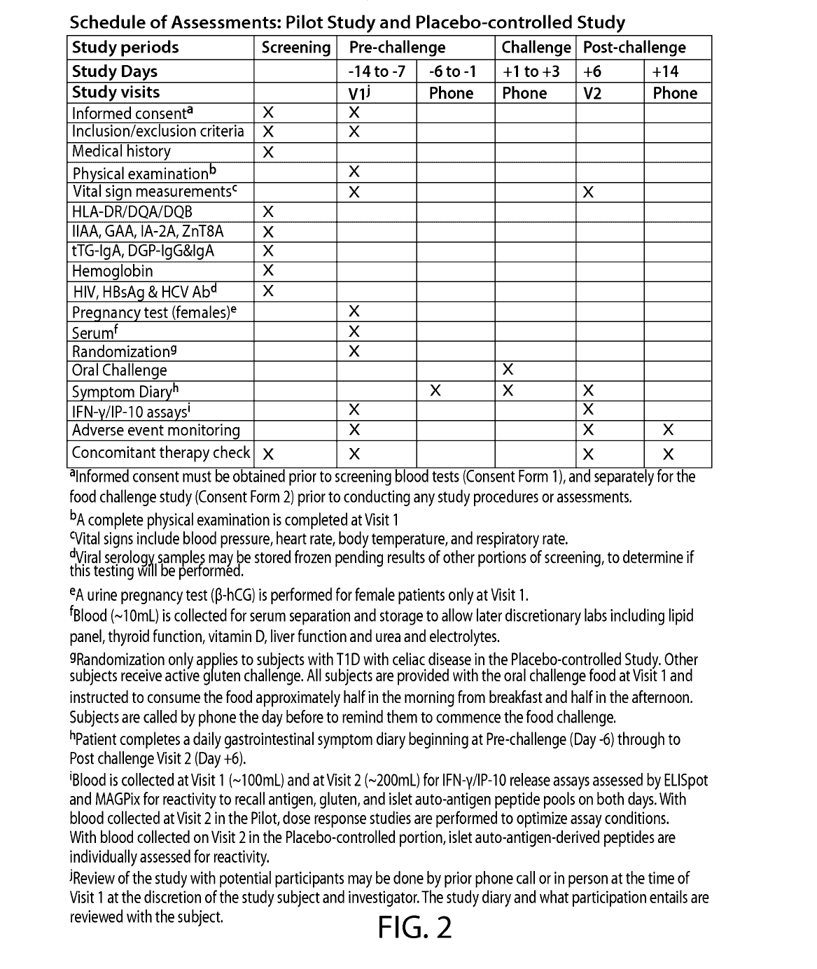

A schematic of the study design is shown in FIG. 1.

Design: A Randomized, Double-Masked, Placebo-Controlled intervention study preceded by an un-blinded pilot to optimize special laboratory assays.
Intervention: Non-pharmaceutical, clinical food challenge
Study Population: Otherwise healthy men and women aged between 18 and 50 years diagnosed with celiac disease, some with comorbid T1D all following strict gluten-free diet.

### Single Study Site

Two study phases: The Pilot portion includes 8 subjects with T1D and comorbid celiac disease who undergo an open gluten food challenge for three days. The Placebo-controlled portion includes 24 subjects with T1D and comorbid celiac disease who undergo a double-blinded placebo-controlled gluten food challenge for three days. The latter portion of the study also includes 12 subjects with celiac disease who do not have comorbid T1D. For any participant who does not complete a phase of the study a replacement will be sought so total number of subjects may be greater than those described above as completers. A 20% dropout rate with an estimated total enrollment about 52 subjects to achieve the above group sizes is anticipated.

The intervention in Placebo-controlled portion is essentially the same as the Pilot Study except that the three-day gluten challenge is now double-blinded and placebo-controlled. In the Pilot and Placebo-controlled portions, blood for assays of T cells will be collected before and six days after commencing 3-day gluten/placebo challenge. Although the Pilot portion is exploratory, and not designed for statistical significance, it is anticipated that it would support that islet autoreactive T cells are mobilized in blood after gluten challenge in at least some individuals with T1D and celiac disease. The primary hypothesis is formally tested in Placebo-controlled portion.

The expected duration of subject participation: 21 days. Participants in the Pilot portion may also enroll in the Placebo-controlled portion as long as at least 60 days elapses between gluten challenges. Such participation would entail a separate consent and an additional 21 days of participation.
Hypothesis: The immune stimulation provided by oral gluten challenge in patients with both T1D and celiac disease following a gluten free diet mobilizes both gluten-specific and islet autoantigen-specific T cells in blood.

### Primary Objective:

To quantify and compare T cell responses to pancreatic islet autoantigens, immunodominant gluten peptides, and pathogen-derived recall antigens before and after oral gluten challenge in patients with both T1D and celiac disease and in patients with celiac disease alone.
Secondary objectives:
1. To establish the safety of oral gluten challenge in patients with comorbid Celiac Disease and T1D.
2. To define islet autoantigen-derived peptides recognized by T cells mobilized after oral gluten challenge.

### Visit Schedule:

### Screening

Informed consent is obtained prior to participation.
The screening blood tests include:
HLA-DRB1, HLA-DQA and HLA-DQB genotype
Islet autoantibody panel (insulin-IAA, GAD65-GAA, IA-2A, and zinc transporter-8-ZnT8A)
Celiac disease-specific serology panel (transglutaminase [tTG]-IgA, deamdiated gliadin peptide [DGP]-IgA and -IgG)
Hemoglobin
Human immuno-deficiency virus-1 and -2 (HIV1+2), hepatitis B virus (HBV) and hepatitis C virus (HCV) serology (sample may be frozen for later testing pending above results).
   Medical information collected includes:
Celiac disease history including prior celiac serology and small bowel histology
Age of diagnosis of T1D if applicable
Complications of celiac disease or T1D
Compliance with gluten free diet, including duration
Medication history including treatment with systemic biological agents or systemic immunomodulatory agents
Allergies, in particular nut or peanut allergies
History of angina
History of other autoimmune diseases

### Pilot Study

Prior to or at time of visit 1 the study, what participation entails including the study diary is reviewed with potential participants.

### Visit 1

### Pre-Challenge:

Study visit takes place one to two weeks before planned oral challenge. The visit is scheduled in the morning. The visit includes:
Addressing any of the subject's questions about the study.
The subject providing written informed consent.
Physical exam.
Urine pregnancy test for women of reproductive age.
Finger stick blood glucose level.
Blood for C-peptide and glucose. In persons with T1D if finger stick glucose level is <150 mg/dL then gluten free snack is provided and blood collection for glucose and C-peptide is performed later in the visit.
Blood collection for T cell assays (˜100 mL), and for glycosylated hemoglobin A1C, and serum (˜10 mL) is stored (frozen) for later discretionary testing such as liver function, urea and electrolytes, lipid profile, thyroid function, and Vitamin D depending upon results.
Subjects is provided the active gluten food for the three-day oral challenge (Day+1, Day+2 and Day+3) and instructed when it should be consumed (approximately half in the morning and half in the afternoon).
Subjects are reminded not to consume any other food containing gluten.
Subjects are provided with Daily Symptom Diaries and instructed to complete them each morning from Day−6 until Day+6. Daily Symptom Diaries are forwarded to study coordinator daily by fax or email.

### Phone Call: Day−7 Pre-Challenge

Subjects are phoned, texted, or emailed seven days before they commence oral food challenge and reminded to commence filling in Daily Symptom diaries from the following day (Day−6).

### Phone Call: Day−1 Pre-Challenge

Subjects are phoned, texted, or emailed the day before they commence oral food challenge for three days.

Visit 2 (Site Visit): Day+6 Post Challenge Subjects return to the study site on the morning of Day+6. Clinical and adverse event assessment are performed. Blood is collected for T cell assays (˜200 mL). The final Daily Symptom diary is collected.

### Phone Call: Day+14 Post Challenge—End of Study Assessment

Patients are contacted by phone call for adverse event assessment, and End-of-Study (EOS) assessment.

### Placebo-Controlled Study

Prior to or at time of visit 1 the study, what participation entails including the study diary is reviewed with potential participants.

### Visit 1

### Pre-Challenge:

Study visit takes place one to two weeks before planned oral challenge. The visit is scheduled in the morning. The visit includes:
Addressing any of the subject's questions about the study.
The subject providing written informed consent.
Physical exam.
Urine pregnancy test for women of reproductive age.
Finger stick blood glucose level.
Blood for C-peptide and glucose. In persons with T1D if finger stick glucose level is <150 mg/dL then gluten free snack is provided and blood collection for glucose and C-peptide is performed later in the visit.
Blood collection for T cell assays (˜100 mL), and for glycosylated hemoglobin A1C, and serum (˜10 mL) is stored (frozen) for later discretionary testing such as liver function, urea and electrolytes, lipid profile, thyroid function, and Vitamin D depending upon results.
Subjects are provided the food for the three-day oral challenge (Day+1, Day+2 and Day+3) and instructed when it should be consumed (approximately half in the morning and half in the afternoon). They are not told if the food contains gluten or not. Randomization to gluten versus placebo is 2:1.
Subjects are reminded not to consume any (other) food containing gluten.
Subjects are provided with Daily Symptom Diaries and instructed to complete them each morning from Day−6 until Day+6. Daily Symptom Diaries are forwarded to study coordinator daily by fax or email.

### Phone Call: Day−7 Pre-Challenge

Subjects are phoned, texted, or emailed seven days before they commence oral food challenge and reminded to commence filling in Daily Symptom diaries from the following day (Day−6).

### Phone Call: Day−1 Pre-Challenge

Subjects are phoned, texted, or emailed the day before they commence oral food challenge for three days.

### Visit 2 (Site Visit): Day+6 Post Challenge

Subjects returns to the study site on the morning of Day+6. Clinical and adverse event assessment is performed. Blood is collected for T cell assays (˜200 mL). The final Daily Symptom diary is collected.

### Phone Call: Day+14 Post Challenge—End of Study Assessment

Patients are contacted by phone call for adverse event assessment, and End-of-Study (EOS) assessment.

### Data Analysis

### Pilot

A descriptive analysis of T cell assay findings are performed using the data from the 8 subjects who complete 3-day active gluten challenge, and any other patients who commence but do not complete the three-day challenge. ELISpot responses to recall, gluten, and islet autoantigen pools is graded as positive or negative according to DFR analysis of six replicate medium only wells and six replicate test wells. An interferon-γ response measured by MAGPix@ assay is regarded as positive when mean islet autoantigen IFN-γ response is greater than or equal to twice mean negative control IFN-γ response. An IP-10 response on measured by MAGPix@ assay will be regarded as positive when mean islet autoantigen IP-10 response is greater than or equal to twice mean negative control IP-10 response.

One subject with a positive Day+6 islet autoantigen-specific T-cell ELISpot response supported by a positive MAGAPix@ IFN-γ response, and acceptable safety of the gluten challenge prompts initiation of the Placebo-controlled portion of the study.

### Placebo-Controlled

In the Placebo-controlled portion, IFN-γ ELISpot, and MAGPix@ IFN-γ and IP-10 responses are analyzed as they were in the Pilot.

In addition to describing the frequency of positive responses to individual peptide pools, the change in response between Day 0 and Day+6 is determined for each of the patient groups (1. Celiac disease with comorbid T1D having active gluten challenge; 2. Celiac disease with comorbid T1D having placebo challenge; 3. Celiac disease without T1D having active gluten challenge; and 4. Celiac disease without T1D having placebo challenge). Change in the mean test response minus negative control response between Day 0 and Day 6 is analyzed for each peptide pool and compared using a One-tailed Paired T-test, or if data are not Gaussian in distribution then they are compared by non-parametric Wilcoxon paired ranked sum test to determine whether gluten challenge induces antigen-specific responses. P value less than 0.05 is considered significant in either test.

In the Placebo-controlled portion, each individual peptide derived from islet autoantigens are incubated in a single well (of a 96-well plate) with PBMCs (ELISpot) OR whole blood collected (MAGPix@) on Day 6 after oral gluten challenge. Data for single peptide incubations are considered positive if the response in the IFN-γ ELISpot, and MAGPix@ IFN-γ is greater than 2× higher than the mean negative control. Positive responses in the MAGPix@ IP-10 assay re considered positive if greater than 2× higher than the mean negative control and documented separately from the IFN-γ and will be considered supportive of positive IFN-γ ELISpot and MAGPix@ findings but not necessary to confirm positive responses in these assays. The frequency of “positive” IFN-γ ELISpot, IFN-γ MAGPix@, IP-10 MAGPix@ responses to individual peptides are described in tabular form.

Adverse events are tabulated for subjects with T1D and celiac disease undergoing double-blinded oral challenge (i.e., active gluten or placebo gluten). The severity and frequency of adverse events for subjects undergoing open challenge (from the Pilot) with gluten and those with celiac disease but not T1D are also be described

### Primary Endpoint:

Interferon-γ T cell response on Day 6 measured by IFN-γ ELISpot assay, where a positive is defined by Distribution free resampling (DFR) of spot forming units in triplicate wells with any one of the six autoantigen peptide pools compared to replicate wells with medium only.

### Secondary Endpoints:

1. Intervention-emergent Adverse Events (i.e. following initiation of the oral challenge)

2. Interferon-γ Response on Day 6 measured by MAGPix@ assay, where a positive is defined by mean islet autoantigen IFN-γ response ≧twice mean negative control IFN-γ response

3. IP-10 Response on Day 6 measured by MAGPix@ assay, where a positive is defined by mean islet autoantigen IP-10 response ≧twice mean negative control IP-10 response

NOTE: The primary and secondary endpoints for Placebo-controlled portion are the same as for Pilot. However, a data-driven modification of these endpoints may be introduced prior to initiation of the Placebo-controlled portion.

### Study Enrollment and Withdrawal

### Subject Inclusion Criteria

(1) Patient has signed and understood the informed consent form before initiation of any study-specific procedures.
(2) Patient is between 18 and 50 years old (inclusive).
(3) For those participants in the T1D group they must have a clinical diagnosis of T1D diabetes and at least one anti-islet antibody detected.
(4) Participant has a celiac disease diagnosis consistent with the criteria defined in the National Institutes of Health Consensus Statement 2004 (Department of Health and Human Services, 2004): small bowel histology showing at least villous atrophy, and serology showing elevated transglutaminase IgA or abnormal endomysial immunofluorescence while gluten is being regularly consumed.
(5) Patient has HLA-DQ2.5 genotype (both DQA1*05 and DQB1*02, homozygous or heterozygous)

### Subject Exclusion Criteria

Patients meeting any of the following criteria are excluded from the study:
(1) Individual has not been prescribed and/or has not followed a GFD for at least 12 months or has had known gluten exposure within two months prior to screening.
(2) Subject with elevation in transglutaminase [tTG]-IgA, deamdiated gliadin peptide [DGP]-IgA or IgG to a level >/=50% above upper limit of normal range for that assay
(3) Individual has uncontrolled complications of T1D which, in the opinion of the investigator, would impact the immune response or pose an increased risk to the patient.
(4) Individual has uncontrolled complications of celiac disease or unstable autoimmune disease which, in the opinion of the investigator, would impact the immune response or pose an increased risk to the patient.
(5) Individual has had treatment with systemic biological agents (e.g., adalimumab, etanercept, infliximab, certolizumab pegol) less than six months prior to screening.
(6) Individual has taken systemic immunomodulatory agents (eg, azathioprine, methotrexate) less than 30 days prior to screening.
(7) Human immuno-deficiency virus-1 and -2 (HIV1+2) infection or active, untreated hepatitis B virus (HBV) or hepatitis C virus (HCV) infection
(8) Individual has any nut (including peanut) allergy
(9) Hemoglobin level is below sex specific normal range for the lab
(10) Individuals with celiac disease without known comorbid T1D who possess one or more abnormal serology test for Islet autoantibody panel (insulin-IAA, GAD65-GAA, IA-2A, and zinc transporter-8A-ZnT8A).
(11) Medical history of angina
(12) Individual is lactating or pregnant
(13) If a woman of childbearing potential, unwilling to abstain from becoming pregnant during the period of participation. Acceptable methods of birth control include transdermal patch, intrauterine devices/systems, oral, implantable, or injectable contraceptives, sexual abstinence, double-barrier method, and vasectomized partner.
(14) Individual is unable and/or unwilling to comply with study requirements.

### Treatment Assignment Procedures

All subjects in Pilot undergo active gluten challenge.

During the Placebo-controlled portion, subjects are randomized 2:1 to either active gluten challenge or placebo challenge. The celiac disease with comorbid T1D and celiac disease alone groups are randomized separately to ensure the desired 2:1 gluten to placebo ratio in each group.

### Randomization Procedures

All patients are assigned a unique patient number during the Screening Period. Once a patient number is assigned, it is not reused, even if the patient withdraws from the study before receiving food challenge.

During the Pilot portion, the Study Coordinator provides the active gluten challenge food to all subjects. This portion is not randomized or masked.

During the Placebo-controlled portion randomization occurs at the study site by pulling the numbers of pre-masked food packets out of a hat. The celiac disease with comorbid T1D and celiac disease alone groups are randomized separately to ensure the desired 2:1 gluten to placebo ratio in each group.

### Masking Procedures

The Sponsor provides the Study Site with plastic-vacuum sealed food packets to be consumed for the challenge, which are labeled with the randomization code but have no marks by which it can be identified as gluten containing or placebo. The Study Site randomly assigns a food packet to a given participant, recording the distribution. The Sponsor does not know which participants received which food packets and the Study Site does not know which food packets contain gluten maintaining the masking during data acquisition. In case of emergency requiring unmasking, the Study Site can unseal the corresponding envelope to the food challenge given to the particular individual participant without otherwise compromising the masking of other participants.

During the Placebo-controlled portion, the Study Coordinator provides the food challenge to the subjects with T1D and celiac disease in a masked manner as described. The gluten-containing food and the food placebo containing no gluten are identical in terms of physical appearance, consistency and taste. The patient, investigator, and all other study center personnel remain blinded to the content of the food.

During the Pilot, active gluten challenge food to all subjects. This portion is not randomized or masked.

### Reasons for Withdrawal

A study subject is discontinued from participation in the study if: Any clinical adverse event (AE), laboratory abnormality, intercurrent illness, or other medical condition or situation occurs such that continued participation in the study would not be in the best interest of the subject.

Development of any exclusion criteria may be cause for discontinuation.

Subjects are free to withdraw from participating in the study at any time upon request.

Patient participation in the study may be stopped at any time at the discretion of the investigator or at the request of the Sponsor.

### Handling of Withdrawals

Subjects who withdraw during screening are replaced, and not be included in data analysis.

Subjects who withdraw after screening but before food challenge are replaced, and not included in data analysis. They are not encouraged to return for follow-up visits.

Subjects who withdraw after commencing food challenge are replaced, and are not included in regular data analysis, but they are encouraged to return for all subsequent visits.

Whenever possible, all patients who withdraw from the study prematurely undergo all End-of-Study assessments. For patients who fail to return for final assessments, reasonable efforts re used by the study center personnel to contact the patient in an attempt to have them comply with the protocol. Reasonable efforts include telephone calls, email and text messaging. It is important to obtain complete follow-up data on any patient withdrawn because of an AE or SAE. In every case, efforts must be made to undertake protocol-specified, safety, and all follow-up procedures.

In order to maximize available immunological response data in this study, it is important for patient to consume all of the oral challenge as dispensed. Subjects who do not complete the three-day oral challenge are replaced, and immunological data from those who do not compete all three days of the food challenge are analyzed separately from those who do complete the three day challenge.

### Study Intervention

### Food Challenge Description

The active food challenge includes approximately 9 grams of gluten. Gluten is delivered in the form of food bars containing 20% gluten, 12% sugar, and 12% glucose. The food challenge includes a total of 5.4 g sugar and 5.4 g glucose daily. The placebo food challenge is identical in taste, appearance and texture, but does not contain gluten. Both food bars contain nuts, including peanuts. The food bars are presented in sealed plastic wraps and marked with an expiry date.

### Modification of Study Intervention

All subjects are encouraged to complete the three-day food challenge. Some subjects may elect not consume the full dose or duration of the food challenge due to adverse events. Subjects are instructed to contact the Principal Investigator before omitting a component of the challenge. Subjects are instructed to consume approximately half the food challenge in the morning beginning at breakfast and half in the afternoon.

### Accountability Procedures for the Study

All food bars are labeled with a unique identifier. Food bars for oral challenges are stored at the Study site under the supervision of the Study Coordinator. At Visit 1, the Study Coordinator will distribute the food to the subject instructing the patient on how much to consume each day. Unused food is returned to the site at Visit 2.

### Concomitant Medications/Treatments

Patients are not eligible for enrollment if they have been treated with systemic biological agents (eg, adalimumab, etanercept, infliximab, certolizumab pegol) less than six months prior to screening, or systemic immunomodulatory agents (eg, azathioprine, methotrexate) less than 30 days prior to screening.

### Rescue Treatments

During the 21-day enrollment period, nausea or heartburn can be treated by the patient's usual medications or by the addition of antacids; abdominal pain can be treated by acetaminophen (up to two 500 mg tablets six-hourly); diarrhea can be treated with up to two lomotil tablets four times daily (diphenoxylate hydrochloride 2.5 mg atropine sulfate 0.025 mg).

Self-administered insulin dose may need to be adjusted according to blood sugar levels and whether food is being consumed regularly. The Principal Investigator is available at any time to advise subjects regarding management of blood sugar levels.

### Clinical Evaluations

Medical history is obtained by interview and from medical records to confirm celiac disease has been diagnosed by small bowel histology showing villous atrophy, crypt hyperplasia and intra-epithelial lymphocytosis while a gluten-containing diet was consumed. Compliance with gluten free diet is undertaken by medical history. Absence of a history of nut allergy is obtained. Evidence of adverse events is obtained by open-ended questioning of the subjects, for example, “Have you experienced any new or different symptoms during the study?”

Medications history to confirm that the patient has not had treatment with systemic biological agents (eg, adalimumab, etanercept, infliximab, certolizumab pegol) in the previous six months prior to screening, and that the patient has not taken systemic immunomodulatory agents (eg, azathioprine, methotrexate) less than 30 days prior to screening.

Physical examination with assessment of vital signs is undertaken on Visit 1, and if an adverse event occurs.

### Laboratory Evaluations

### Clinical Laboratory Evaluations

All tests are performed prior to oral food challenge

### (1) During Pre-Enrollment

HLA-DRB1, HLA-DQA and HLA-DQB genotype
Islet autoantibody panel (insulin-IAA, GAD65-GAA, IA-2A, and zinc transporter-8-ZnT8A)
Celiac disease-specific serology panel (transglutaminase [tTG]-IgA, deamdiated gliadin peptide [DGP]-IgA and -IgG)
Hemoglobin
Human immuno-deficiency virus-1 and -2 (HIV1+2), hepatitis B virus (HBV) and hepatitis C virus (HCV) serology
   (2) After enrollment, before oral food challenge
glycosylated hemoglobin A1C (in T1D patients)
Insulin C-peptide (in T1D patients)
Biochemistry: creatinine, total bilirubin, alanine aminotransferase (ALT), aspartate aminotransferase (AST) (discretionary)
Serum lipid profile: Triglycerides, total cholesterol, low density lipoprotein, and high density lipoprotein (discretionary)
Thyroid function: Thyroxine stimulating hormore (TSH) (discretionary)
Vitamin D (discretionary)
Finger prick blood glucose level (in T1D patients)
Urine pregnancy test (in women of reproductive age)

### Special Assays or Procedures

### Laboratory Assessment of Blood for T Cell Responses

Blood collected before and six days after oral food challenge are tested for the presence of T cells specific for proinsulin, GAD65 and IA-2 autoantigens, immunodominant gluten peptides, or recall antigens. Assessment is using two complimentary cytokine release assays and either peripheral blood mononuclear cells (PBMC) separated from fresh blood or fresh whole blood incubated directly with antigens. Heparinized blood from volunteers are transported to the laboratory within two hours of collection using ambient temperature-controlled transporters. To enumerate antigen-specific T cells secreting interferon-γ, ELISpot assays using PBMC are performed. After overnight incubation, plates are developed, dried, and spot forming units determined for each well using an automated Zeiss counter (performed by a blinded reader ay Zellnet Consulting). Experimental conditions and wells are arranged in order to optimize later analysis using a widely used on-line ELISpot analysis tool (www.scharp.org/zoe/runDFR/). Plasma from whole blood incubated for 24 h with antigens is used to assess antigen-stimulated interferon-γ (from T cells) and interferon-γ inducible protein-10 (IP-10, from monocytes and granulocytes stimulated by interferon-γ). A magnetic bead-based assay (MAGPix®) is used to measure interferon-γ and IP-10. Interim data analysis of antigen dose response studies in cytokine release assays in Pilot Study is also used to select a single optimal antigen concentration for each of antigens assessed in Placebo-controlled Study. Data from MAGPix® assays in Pilot Study may also allow this assay to be used in place of interferon-γ ELISpot assays in Placebo-controlled Study to identify epitopes in islet autoantigens recognized by T cells in blood on Day−6 after gluten challenge. Mean of triplicate antigen-stimulated responses greater than 2× mean of six replicate wells with medium only are considered positive.

### Specimen Preparation, Handling, and Shipping

### Instructions for Specimen Preparation, Handling, and Storage

Fresh blood for clinical laboratory tests is collected directly into vacutainers. Tubes are sent to the diagnostic laboratory as applicable (Hemoglobin, Hemoglobin A1c, glucose). Other tubes are processed by the study team and sent for additional testing or stored as described.

Blood for T cell assays is drawn via a canula directly into a blood collection bag with heparin pre-added to the collection bag. Blood for T cell assays is transported to the laboratory and maintained at 20-30° C. Peripheral blood cells that are not used for fresh T cell assays are cryopreserved for later use in T cell assays of recall, gluten, and islet autoantigen-specific T cells.

### Specification of Safety Parameters

Standard reporting of adverse events is a secondary outcome measure.

### Methods and Timing for Assessing, Recording, and Analyzing Safety Parameters Adverse Events

Adverse events (AEs) and concomitant medications are assessed at each visit. Safety is assessed by the incidence of AEs and severe adverse events (SAEs). Adverse events are assessed utilizing the “Toxicity Grading Scale for Healthy Adult and Adolescent Volunteers Enrolled in Preventive Vaccine Clinical Trials” (Department of Health and Human Services, 2009). Adverse events are summarized for both the Pilot and Placebo-controlled portions, presenting the numbers and percent of patients having an adverse event (AE) and having AEs in each system organ class and preferred term.
The investigator is responsible for reporting all AEs that are observed or reported during the study, regardless of their relationship to the food challenge or their clinical significance.

An AE is defined as any untoward medical occurrence in a patient enrolled into this study, regardless of its causal relationship to food challenge. Patients are instructed to contact the investigator at any time after signing the informed consent form if any symptoms develop.

An SAE is defined as any event that results in death, is immediately life threatening, requires inpatient hospitalization or prolongation of existing hospitalization, results in persistent or significant disability/incapacity, or is a congenital anomaly/birth defect. Important medical events that may not result in death, be life threatening, or require hospitalization may be considered an SAE when, based upon appropriate medical judgment, they may jeopardize the patient and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

### Planned Interim Analyses

Interim data analysis of antigen dose response studies in laboratory cytokine release assays in the Pilot Study are also used to select a single optimal antigen concentration for each of antigens assessed in the Placebo-controlled portion.

Data from MAGPix@ assays in Pilot are compared for equivalence or superiority to interferon-γ ELISpot assays. This may allow MAGPix@ assay to be used in place of the ELISpot, which requires more blood per condition tested, in the Placebo-controlled Study to screen individual peptides with blood collected on Day−6 after gluten challenge.

Interim analysis of T cell responses to islet autoantigen peptide pools are undertaken before proceeding to the Placebo-controlled study: If one subject in the Pilot shows a positive response to any one of the islet auto antigen peptide pools then this prompts initiation of enrollment in the Placebo-controlled Study.

### Final Analysis Plan

### Pilot

A descriptive analysis of T cell assay findings are performed using the data from the 8 subjects who complete 3-day active gluten challenge, and any other patients who commence but do not complete the three-day challenge. ELISpot responses to recall, gluten, islet autoantigen pools are graded as positive or negative according to DFR analysis of six replicate medium only wells and six replicate test wells. An interferon-γ response measured by MAGPix@ assay is regarded as positive when mean islet autoantigen IFN-γ response is greater than or equal to twice mean negative control IFN-γ response. An IP-10 response on measured by MAGPix@ assay is regarded as positive when mean islet autoantigen IP-10 response is greater than or equal to twice mean negative control IP-10 response.

The severity and frequency of adverse events for subjects undergoing open challenge with gluten and those with celiac disease but not Type-1 diabetes are described in table.

One subject with a positive Day+6 islet autoantigen-specific T-cell ELISpot response supported by a positive MAGAPix IFN-γ response, and acceptable safety of the gluten challenge prompts initiation of the Placebo-controlled Study.

### Placebo-Controlled

In the Placebo-controlled Study, IFN-γ ELISpot, and MAGPix@ IFN-γ and IP-10 responses are analysed as they were in the Pilot Study.

In addition to describing the frequency of positive responses to individual peptide pools, the change in response between Day 0 and Day+6 is determined for each of the study groups. Change in the mean test response minus negative control response between Day 0 and Day 6 is analyzed for each peptide pool and compared using a One-tailed Paired T-test, or if data are not Gaussian in distribution then they are compared by non-parametric Wilcoxon paired ranked sum test to determine whether gluten challenge induces antigen-specific responses. P value less than 0.05 is considered significant in either test.

In the Placebo-controlled Study, each individual peptide derived from islet autoantigens is incubated in a single well (of a 96-well plate) with PBMCs (ELISpot) or whole blood collected (MAGPix@) on Day 6 after oral gluten challenge. Data for single peptide incubations are considered positive if the response in the IFN-γ ELISpot, and MAGPix@ IFN-γ is greater than 2× higher than the mean negative control. Positive responses in the MAGPix@ IP-10 assay are considered positive if greater than 2× higher than the mean negative control and documented separately from the IFN-γ and are considered supportive of positive IFN-γ ELISpot and MAGPix@ findings but not necessary to confirm positive responses in these assays. The frequency of “positive” IFN-γ ELISpot, IFN-γ MAGPix@, IP-10 MAGPix@ responses to individual peptides are described in tabular form.

### Primary Endpoint:

Interferon-γ T cell response on Day 6 measured by IFN-γ ELISpot assay, where a positive is defined by Distribution free resampling (DFR) (http://www.scharp.org/zoe/runDFR/) of spot forming units in triplicate wells with any one of the six autoantigen peptide pools compared to six replicate wells with medium only.

### Secondary Endpoints:

1. Intervention-emergent Adverse Events (i.e. following initiation of the oral challenge)
2. Interferon-γ Response on Day 6 measured by MAGPix@ assay, where a positive is defined by mean islet autoantigen IFN-γ response≧twice mean negative control IFN-γ response
3. IP-10 Response on Day 6 measured by MAGPix@ assay, where a positive is defined by mean islet autoantigen IP-10 response≧twice mean negative control IP-10 response

NOTE: The primary and secondary endpoints for Placebo-controlled Study are the same as for Pilot Study. However, a data-driven modification of the ranking of these endpoints may be introduced prior to initiation of Placebo-controlled portion.

### LITERATURE REFERENCES

1. Green P H, Cellier C. Celiac disease. The New England journal of medicine 2007; 357:1731-43.
2. Karell K, Louka A S, Moodie S J, et al. HLA types in celiac disease patients not carrying the DQA1*05-DQB1*02 (DQ2) heterodimer: results from the European Genetics Cluster on Celiac Disease. Human immunology 2003; 64:469-77.
3. Fasano A, Berti I, Gerarduzzi T, et al. Prevalence of celiac disease in at-risk and not-at-risk groups in the United States: a large multicenter study. Archives of internal medicine 2003; 163:286-92.
4. Katz K D, Rashtak S, Lahr B D, et al. Screening for celiac disease in a North American population: sequential serology and gastrointestinal symptoms. The American journal of gastroenterology 2011; 106:1333-9.
5. Husby S, Koletzko S, Korponay-Szabo I R, et al. European Society for Pediatric Gastroenterology, Hepatology, and Nutrition guidelines for the diagnosis of coeliac disease. Journal of pediatric gastroenterology and nutrition 2012; 54:136-60.
6. Kumar P J, O'Donoghue D P, Stenson K, Dawson A M. Reintroduction of gluten in adults and children with treated coeliac disease. Gut 1979; 20:743-9.
7. Sollid L M, Khosla C. Novel therapies for coeliac disease. Journal of internal medicine 2011; 269:604-13.
8. Smyth D J, Plagnol V, Walker N M, et al. Shared and distinct genetic variants in type 1 diabetes and celiac disease. The New England journal of medicine 2008; 359:2767-77.
9. Atkinson M A. The pathogenesis and natural history of type 1 diabetes. Cold Spring Harbor perspectives in medicine 2012; 2.
10. Triolo T M, Armstrong T K, McFann K, et al. Additional autoimmune disease found in 33% of patients at type 1 diabetes onset. Diabetes care 2011; 34:1211-3.
11. Simell S, Hoppu S, Simell T, et al. Age at development of type 1 diabetes- and celiac disease-associated antibodies and clinical disease in genetically susceptible children observed from birth. Diabetes care 2010; 33:774-9.
12. Bakker S F, Tushuizen M E, Stokvis-Brantsma W H, et al. Frequent delay of coeliac disease diagnosis in symptomatic patients with type 1 diabetes mellitus: Clinical and genetic characteristics. European journal of internal medicine 2013.
13. Sildorf S M, Fredheim S, Svensson J, Buschard K. Remission without insulin therapy on gluten-free diet in a 6-year old boy with type 1 diabetes mellitus. BMJ case reports 2012; 2012.
14. Galipeau H J, Rulli N E, Jury J, et al. Sensitization to gliadin induces moderate enteropathy and insulitis in nonobese diabetic-DQ8 mice. Journal of immunology 2011; 187:4338-46.
15. Ejsing-Duun M, Josephsen J, Aasted B, Buschard K, Hansen A K. Dietary gluten reduces the number of intestinal regulatory T cells in mice. Scandinavian journal of immunology 2008; 67:553-9.
16. Klemetti P, Savilahti E, Ilonen J, Akerblom H K, Vaarala O. T-cell reactivity to wheat gluten in patients with insulin-dependent diabetes mellitus. Scandinavian journal of immunology 1998; 47:48-53.
17. Hummel S, Pfluger M, Hummel M, Bonifacio E, Ziegler A G. Primary dietary intervention study to reduce the risk of islet autoimmunity in children at increased risk for type 1 diabetes: the BABYDIET study. Diabetes care 2011; 34:1301-5.
18. Anderson R P, Degano P, Godkin A J, Jewell D P, Hill A V. In vivo antigen challenge in celiac disease identifies a single transglutaminase-modified peptide as the dominant A-gliadin T-cell epitope. Nature medicine 2000; 6:337-42.
19. Qin S, Cobbold S P, Pope H, et al. “Infectious” transplantation tolerance. Science 1993; 259:974-7.
20. Rostom A, Murray J A, Kagnoff M F. American Gastroenterological Association (AGA) Institute technical review on the diagnosis and management of celiac disease. Gastroenterology 2006; 131:1981-2002.
21. Brottveit M, Raki M, Bergseng E, et al. Assessing possible celiac disease by an HLA-DQ2-gliadin Tetramer Test. The American journal of gastroenterology 2011; 106:1318-24.
22. Leffler D, Schuppan D, Pallav K, et al. Kinetics of the histological, serological and symptomatic responses to gluten challenge in adults with coeliac disease. Gut 2012.
23. Anderson R P, van Heel D A, Tye-Din J A, et al. T cells in peripheral blood after gluten challenge in coeliac disease. Gut 2005; 54:1217-23.
24. Raki M, Fallang L E, Brottveit M, et al. Tetramer visualization of gut-homing gluten-specific T cells in the peripheral blood of celiac disease patients. Proceedings of the National Academy of Sciences of the United States of America 2007; 104:2831-6.
25. Camarca A, Radano G, Di Mase R, et al. Short wheat challenge is a reproducible in-vivo assay to detect immune response to gluten. Clinical and experimental immunology 2012; 169:129-36.
26. Tye-Din J A, Stewart J A, Dromey J A, et al. Comprehensive, quantitative mapping of T cell epitopes in gluten in celiac disease. Science translational medicine 2010; 2:41ra51.
27. Henderson K N, Tye-Din J A, Reid H H, et al. A structural and immunological basis for the role of human leukocyte antigen DQ8 in celiac disease. Immunity 2007; 27:23-34.
28. Sollid L M, Qiao S W, Anderson R P, Gianfrani C, Koning F. Nomenclature and listing of celiac disease relevant gluten T-cell epitopes restricted by HLA-DQ molecules. Immunogenetics 2012; 64:455-60.
29. Doolan A, Donaghue K, Fairchild J, Wong M, Williams A J. Use of HLA typing in diagnosing celiac disease in patients with type 1 diabetes. Diabetes care 2005; 28:806-9.
30. Dicke W K, Weijers H A, Van De Kamer J H. Coeliac disease. II. The presence in wheat of a factor having a deleterious effect in cases of coeliac disease. Acta paediatrica 1953; 42:34-42.
31. Van De Kamer J H, Weijers H A, Dicke W K. Coeliac disease. IV. An investigation into the injurious constituents of wheat in connection with their action on patients with coeliac disease. Acta paediatrica 1953; 42:223-31.

### Example 2

### Islet Autoantigen Peptide Library

Libraries of peptides were derived from protein sequences sourced from Genbank according to searches utilizing the following the following terms:

### Insulin/Proinsulin/Preproinsulin Peptide Library

34 17mer peptides encompassing all unique 11mer sequences in Genbank entries retrieved using the Genbank search with the terms:
(1) Insulin[All Fields] AND (“Homo sapiens”[Organism] OR (“Homo sapiens”[Organism] OR homo sapiens[All Fields])), or
(2) Proinsulin[All Fields] AND (“Homo sapiens”[Organism] OR homo sapiens[All Fields]), or
(3) Preproinsulin[All Fields] AND (“Homo sapiens” [Organism] OR homo sapiens[All Fields])
Glutamic decarboxylase 65 (GAD65) and 67 (GAD67) Peptide Library
150 17mer peptides encompassing all unique 11mer sequences in Genbank entries retrieved using the Genbank search with the terms:
(1) Glutamic[All Fields] AND decarboxylase[All Fields] AND 65[All Fields] AND (“Homo sapiens” [Organism] OR homo sapiens[All Fields])
(2) Glutamic[All Fields] AND decarboxylase[All Fields] AND 67[All Fields] AND (“Homo sapiens” [Organism] OR homo sapiens[All Fields]), or
(3) (Glutamic decarboxylase[All Fields]) AND (“Homo sapiens” [Organism] OR homo sapiens[All Fields]), or
(4) (“glutamate decarboxylase” [All Fields]) AND (“Homo sapiens” [Organism])

### Insulinoma Antigen-2 or Tyrosine Phosphatase Like Autoantigen or (IA-2; ICA512, PTPRN) Peptide Library

255 17mer peptides encompassing all unique 11mer sequences in Genbank entries retrieved using the Genbank search with the terms:
(Receptor-type tyrosine-protein phosphatase-like N[Protein Name] OR (Receptor-type[All Fields] AND tyrosine-protein[All Fields] AND phosphatase-like[All Fields] AND N[All Fields])) AND (“Homo sapiens” [Organism] OR homo sapiens[All Fields])
The amino-acid sequences of proteins sourced from Genbank were:

### Insulin/Proinsulin/Preproinsulin Peptide Library

34 17mer peptides encompassing all unique 11mer sequences in Genbank entries retrieved using the Genbank search:

>CAA23828.1
(SEQ ID NO: 1)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AFK93533.1
(SEQ ID NO: 2)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>NP_001172026.1
(SEQ ID NO: 3)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>NP_001172027.1
(SEQ ID NO: 4)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>NP_000198.1
(SEQ ID NO: 5)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AAA59172.1
(SEQ ID NO: 6)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>CAA49913.1
(SEQ ID NO: 7)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>P01308.1
(SEQ ID NO: 8)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>0601246A
(SEQ ID NO: 9)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AAW83741.1
(SEQ ID NO: 10)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AAA59172.1
(SEQ ID NO: 11)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AAN39451.1
(SEQ ID NO: 12)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AAA59173.1
(SEQ ID NO: 13)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AAH05255.1
(SEQ ID NO: 14)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AAP35454.1
(SEQ ID NO: 15)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
>AAA59179.1
(SEQ ID NO: 16)
MALWMRLLPLLALLALWGPDPAAAFVNQHLCGSHLVEALYLVCGERGFFY
TPKTRREAEVGQVELGGGPGAGSLQPLALEGSLQKRGIVEQCCTSICSLY
QLENYCN
>AEG19452.1
(SEQ ID NO: 17)
WGPDPAAAFVNQHLCGSHLVEALYLVCGERGFFYTPKTRREAEDLQVGQV
ELGGGPGAGSLQPLALEGSLQKRGIVEQCCTSICSLYQLENYCN
>CAA08766.1
(SEQ ID NO: 18)
MALWMRLLPLLALLALWGPDPAAAFVNQHLCGSHLVEALYLVCGERGFFY
TPKTRREAE
>ABI63346.1
(SEQ ID NO: 19)
MALWMRLLPLLALLALWGPDPAAAFVNQHLCGSHLVEALYLVCGERGFFY
TPKTRREAEDLQGSLQPLALEGSLQKRGIVEQCCTSICSLYQLENYCN
>AAP35454.1E
(SEQ ID NO: 20)
MALWMRLLPLLALLALWGPDPAAAFVNQHLCGSHLVEALYLVCGERGFFY
TPKTRREAEDLQVGEVELGGGPGAGSLQPLALEGSLQKRGIVEQCCTSIC
SLYQLENYCN
>AAA59179.1E
(SEQ ID NO: 21)
MALWMRLLPLLALLALWGPDPAAAFVNQHLCGSHLVEALYLVCGERGFFY
TPKTRREAEVGEVELGGGPGAGSLQPLALEGSLQKRGIVEQCCTSICSLY
QLENYCN
>AEG19452.1E
(SEQ ID NO: 22)
WGPDPAAAFVNQHLCGSHLVEALYLVCGERGFFYTPKTRREAEDLQVGEV
ELGGGPGAGSLQPLALEGSLQKRGIVEQCCTSICSLYQLENYCN
>CAA08766.1E
(SEQ ID NO: 23)
MALWMRLLPLLALLALWGPDPAAAFVNQHLCGSHLVEALYLVCGERGFFY
TPKTRREAE
>ABI63346.1E
(SEQ ID NO: 24)
MALWMRLLPLLALLALWGPDPAAAFVNQHLCGSHLVEALYLVCGERGFFY
TPKTRREAEDLEGSLQPLALEGSLQKRGIVEQCCTSICSLYQLENYCN

Glutamic decarboxylase 65 (GAD65) and 67 (GAD67) Peptide Library
150 17mer peptides encompassing all unique 11mer sequences in Genbank entries retrieved using the Genbank search:

>NP_001127838.1
(SEQ ID NO: 25)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>NP_001127838.1E
(SEQ ID NO: 26)
MASPGSGFWSFGSEDGSGDSENPGTARAWCQVAQKFTGGIGNKLCALLYGDAEKPAESG
GSQPPRAAARKAACACDEKPCSCSKVDVNYAFLHATDLLPACDGERPTLAFLQDVMNIL
LEYVVKSFDRSTKVIDFHYPNELLQEYNWELADQPQNLEEILMHCETTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKEKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>NP_000809.1
(SEQ ID NO: 27)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>Q05329.1
(SEQ ID NO: 28)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAA58491.1
(SEQ ID NO: 29)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>CAC09233.1
(SEQ ID NO: 30)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAA62367.1
(SEQ ID NO: 31)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>|EAW86103.1
(SEQ ID NO: 32)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>EAW86104.1
(SEQ ID NO: 33)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDGLMQNCNQMHASYLFQQDKHYDLSYDTGDKALQCGRHVDVFKLWLMWRAK
GTTGFEAHVDKCLELAEYLYNIIKNREGYEMVFDGKPQHTNVCFWYIPPSLRTLEDNEER
MSRLSKVAPVIKARMMEYGTTMVSYQPLGDKVNFFRMVISNPAATHQDIDFLIEEIERLG
QDL
>EAW86102.1
(SEQ ID NO: 34)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAI26330.1
(SEQ ID NO: 35)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAI26328.1
(SEQ ID NO: 36)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>AAP88040.1
(SEQ ID NO: 37)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>CAH73659.1
(SEQ ID NO: 38)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>Q5VZ30
(SEQ ID NO: 39)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGHPRY
FNQLSTGLDMVGLAADWLTSTANTNMFTYEIAPVFVLLEYVTLKKMREIIGWPGGSGDGI
FSPGGAISNMYAMMIARFKMFPEVKEKGMAALPRLIAFTSEHSHFSLKKGAAALGIGTDS
VILIKCDERGKMIPSDLERRILEAKQKGFVPFLVSATAGTTVYGAFDPLLAVADICKKYKI
WMHVDAAWGGGLLMSRKHKWKLSGVERANSVTWNPHKMMGVPLQCSALLVREEGL
MQNCNQMHASYLFQQDKHYDLSYDTGDKALQCGRHVDVFKLWLMWRAKGTTGFEAH
VDKCLELAEYLYNIIKNREGYEMVFDGKPQHTNVCFWYIPPSLRTLEDNEERMSRLSKVA
PVIKARMMEYGTTMVSYQPLGDKVNFFRMVISNPAATHQDIDFLIEEIERLGQDL
>CAB62572.1
(SEQ ID NO: 40)
LKYAIKTGHPRYFNQLSTGLDMVGLAADWLTSTANTNMFTYEIAPVFVLLEYVTLKKMR
EIIGWPGGSGDGIFSPGGAISNMYAMMIARFKMFPEVKEKGMAALPRLIAFTSEHSHFSLK
KGAAALGIGTDSVILIKCDERGKMIPSDLERRILEAKQKGFVPFLVSATAGTTVYGAFDPL
LAVADICKKYKIWMHVDAAWGGGLLMSRKHKWKLSGVERANSVTWNPHKMMGVPLQ
CSALLVREEGLMQNCNQMHASYLFQQDKHYDLSYDTGDKALQCGRHVDVFKLWLMW
RAKGTTGFEAHVDKCLELAEYLYNIIKNREGYEMVFDGKPQHTNVCFWYIPPSLRTLEDN
EERMSRLSKVAPVIKARMMEYGTTMVSYQPLGDKVNFFRMVISNPAATHQDIDFLIEEIE
RLGQDL
>CAB62572.1E
(SEQ ID NO: 41)
LKYAIKTGHPRYFNQLSTGLDMVGLAADWLTSTANTNMFTYEIAPVFVLLEYVTLKKMR
EIIGWPGGSGDGIFSPGGAISNMYAMMIARFKMFPEVKEKGMAALPRLIAFTSEHSHFSLK
KGAAALGIGTDSVILIKCDERGKMIPSDLERRILEAKEKGFVPFLVSATAGTTVYGAFDPL
LAVADICKKYKIWMHVDAAWGGGLLMSRKHKWKLSGVERANSVTWNPHKMMGVPLQ
CSALLVREEGLMQNCNQMHASYLFQQDKHYDLSYDTGDKALQCGRHVDVFKLWLMW
RAKGTTGFEAHVDKCLELAEYLYNIIKNREGYEMVFDGKPQHTNVCFWYIPPSLRTLEDN
EERMSRLSKVAPVIKARMMEYGTTMVSYQPLGDKVNFFRMVISNPAATHQDIDFLIEEIE
RLGQDL
>CAA49554.1
(SEQ ID NO: 42)
MSPIHHHHHHLVPRGSEASNSGFWSFGSEDGSGDSENPGTARAWCQVAQKFTGGIGNKL
CALLYGDAEKPAESGGSQPPRAAARKAACACDQKPCSCSKVDVNYAFLHATDLLPACD
GERPTLAFLQDVMNILLQYVVKSFDRSTKVIDFHYPNELLQEYNWELADQPQNLEEILMH
CQTTLKYAIKTGHPRYFNQLSTGLDMVGLAADWLTSTANTNMFTYEIAPVFVLLEYVTL
KKMREIIGWPGGSGDGIFSPGGAISNMYAMMIARFKMFPEVKEKGMAALPRLIAFTSEHS
HFSLKKGAAALGIGTDSVILIKCDERGKMIPSDLERRILEAKQKGFVPFLVSATAGTTVYG
AFDPLLAVADICKKYKIWMHVDAAWGGGLLMSRKHKWKLSGVERANSVTWNPHKMM
GVPLQCSALLVREEGLMQNCNQMHASYLFQQDKHYDLSYDTGDKALQCGRHVDVFKL
WLMWRAKGTTGFEAHVDKCLELAEYLYNIIKNREGYEMVFDGKPQHTNVCFWYIPPSLR
TLEDNEERMSRLSKVAPVIKARMMEYGTTMVSYQPLGDKVNFFRMVISNPAATHQDIDF
LIEEIERLGQDL
>CAA49554.1E
(SEQ ID NO: 43)
MSPIHHHHHHLVPRGSEASNSGFWSFGSEDGSGDSENPGTARAWCQVAQKFTGGIGNKL
CALLYGDAEKPAESGGSQPPRAAARKAACACDEKPCSCSKVDVNYAFLHATDLLPACDG
ERPTLAFLQDVMNILLEYVVKSFDRSTKVIDFHYPNELLQEYNWELADQPQNLEEILMHC
ETTLKYAIKTGHPRYFNQLSTGLDMVGLAADWLTSTANTNMFTYEIAPVFVLLEYVTLK
KMREIIGWPGGSGDGIFSPGGAISNMYAMMIARFKMFPEVKEKGMAALPRLIAFTSEHSH
FSLKKGAAALGIGTDSVILIKCDERGKMIPSDLERRILEAKEKGFVPFLVSATAGTTVYGAF
DPLLAVADICKKYKIVVMHVDAAWGGGLLMSRKHKWKLSGVERANSVTWNPHKMMGV
PLQCSALLVREEGLMQNCNQMHASYLFQEDKHYDLSYDTGDKALQCGRHVDVFKLWL
MWRAKGTTGFEAHVDKCLELAEYLYNIIKNREGYEMVFDGKPQHTNVCFWYIPPSLRTL
EDNEERMSRLSKVAPVIKARMMEYGTTMVSYQPLGDKVNFFRMVISNPAATHQDIDFLIE
EIERLGQDL
>AAB28987.1
(SEQ ID NO: 44)
ISNMYAMMIARFKMFPEVKEKGMAALPRLIAFTSEHSHFSLKKGAAALGIGTDSVILIKCD
ERGKMIPSDLERRILEAKQKGFVPFLVSATAGTTVYGAFDPLLAVADICKKYKIVVMHVDA
AWGGGLLMSRKHKWKLSGVERANSVTWNPHKMMGVPLQCSALLVREEGLMQNCNQM
HASYLFQQDKHYDLSYDTGDKALQCGRHVDVFKLWLMWRAKGTTGFEAHVDKCLELA
EYLYNIIKNREGYEMVFDGKPQHTNVCFWYIPPSLRTLEDNEERMSRLSKVAPVIKARMM
EYGTTMVSYQPLGDKVNFFRMVISNPAATHQDIDFLIEEIERLGQDL
>AAB28987.1E
(SEQ ID NO: 45)
ISNMYAMMIARFKMFPEVKEKGMAALPRLIAFTSEHSHFSLKKGAAALGIGTDSVILIKCD
ERGKMIPSDLERRILEAKEKGFVPFLVSATAGTTVYGAFDPLLAVADICKKYKIWMHVDA
AWGGGLLMSRKHKWKLSGVERANSVTWNPHKMMGVPLQCSALLVREEGLMQNCNQM
HASYLFQEDKHYDLSYDTGDKALQCGRHVDVFKLWLMWRAKGTTGFEAHVDKCLELA
EYLYNIIKNREGYEMVFDGKPQHTNVCFWYIPPSLRTLEDNEERMSRLSKVAPVIKARMM
EYGTTMVSYQPLGDKVNFFRMVISNPAATHQDIDFLIEEIERLGQDL
>EAW86101.1
(SEQ ID NO: 46)
MASPGSGFWSFGSEDGSGDSENPGTARAWCQVAQKFTGGIGNKLCDLLPACDGERPTLA
FLQDVMNILLQYVVKSFDR
>EAW86101.1E
(SEQ ID NO: 47)
MASPGSGFWSFGSEDGSGDSENPGTARAWCQVAQKFTGGIGNKLCDLLPACDGERPTLA
FLQDVMNILLEYVVKSFDR
>CAH73660.1
(SEQ ID NO: 48)
MNILLQYVVKSFDRSTKVIDFHYPNELLQEYNWELADQPQNLEEILMHCQTTLKYAIKTG
HPRYFNQLSTGLDMVGLAADWLTSTANTNMFTYEIAPVFVLLEYVTLKKMREIIGWPGG
SGDGIFSPGT
>CAH73660.1E
(SEQ ID NO: 49)
MNILLEYVVKSFDRSTKVIDFHYPNELLQEYNWELADQPQNLEEILMHCETTLKYAIKTG
HPRYFNQLSTGLDMVGLAADWLTSTANTNMFTYEIAPVFVLLEYVTLKKMREIIGWPGG
SGDGIFSPGT
>CAH73658.1
(SEQ ID NO: 50)
MASPGSGFWSFGSEDGSGDSENPGTARAWCQVAQKFTGGIGNKLCALLYGDAEKPAESG
GSQPPRAAARKAACACDQKPCSCSKVDVNYAFLHATDLLPACDGERPTLAFLQDVMNIL
LQYVVKSFDRSTKVIDFHYPNELLQEYNWELADQPQNLEEILMHCQTTLKYAIKTGIVSS
KIIKLFFRLQ
>CAH73658.1E
(SEQ ID NO: 51)
MASPGSGFWSFGSEDGSGDSENPGTARAWCQVAQKFTGGIGNKLCALLYGDAEKPAESG
GSQPPRAAARKAACACDEKPCSCSKVDVNYAFLHATDLLPACDGERPTLAFLQDVMNIL
LEYVVKSFDRSTKVIDFHYPNELLQEYNWELADQPQNLEEILMHCETTLKYAIKTGIVSSK
IIKLFFRLQ

Insulinoma antigen-2 or Tyrosine phosphatase like autoantigen or (IA-2; ICA512, PTPRN)
Peptide Library: 255 17mer peptides encompassing all unique 11mer sequences in Genbank entries retrieved using the Genbank search:

>Q16849.1
(SEQ ID NO: 52)
MRRPRRPGGLGGSGGLRLLLCLLLLSSRPGGCSAVSAHGCLFDRRLCSHLEVCIQDGLFG
QCQVGVGQARPLLQVTSPVLQRLQGVLRQLMSQGLSWHDDLTQYVISQEMERIPRLRPP
EPRPRDRSGLAPKRPGPAGELLLQDIPTGSAPAAQHRLPQPPVGKGGAGASSSLSPLQAEL
LPPLLEHLLLPPQPPHPSLSYEPALLQPYLFHQFGSRDGSRVSEGSPGMVSVGPLPKAEAPA
LFSRTASKGIFGDHPGHSYGDLPGPSPAQLFQDSGLLYLAQELPAPSRARVPRLPEQGSSSR
AEDSPEGYEKEGLGDRGEKPASPAVQPDAALQRLAAVLAGYGVELRQLTPEQLSTLLTLL
QLLPKGAGRNPGGVVNVGADIKKTMEGPVEGRDTAELPARTSPMPGHPTASPTSSEVQQ
VPSPVSSEPPKAARPPVTPVLLEKKSPLGQSQPTVAGQPSARPAAEEYGYIVTDQKPLSLA
AGVKLLEILAEHVHMSSGSFINISVVGPALTFRIRHNEQNLSLADVTQQAGLVKSELEAQT
GLQILQTGVGQREEAAAVLPQTAHSTSPMRSVLLTLVALAGVAGLLVALAVALCVRQHA
RQQDKERLAALGPEGAHGDTTFEYQDLCRQHMATKSLFNRAEGPPEPSRVSSVSSQFSDA
AQASPSSHSSTPSWCEEPAQANMDISTGHMILAYMEDHLRNRDRLAKEWQALCAYQAEP
NTCATAQGEGNIKKNRHPDFLPYDHARIKLKVESSPSRSDYINASPIIEHDPRMPAYIATQG
PLSHTIADFWQMVWESGCTVIVMLTPLVEDGVKQCDRYWPDEGASLYHVYEVNLVSEHI
WCEDFLVRSFYLKNVQTQETRTLTQFHFLSWPAEGTPASTRPLLDFRRKVNKCYRGRSCP
IIVHCSDGAGRTGTYILIDMVLNRMAKGVKEIDIAATLEHVRDQRPGLVRSKDQFEFALTA
VAEEVNAILKALPQ
>Q16849.1E
(SEQ ID NO: 53)
MRRPRRPGGLGGSGGLRLLLCLLLLSSRPGGCSAVSAHGCLFDRRLCSHLEVCIEDGLFGE
CEVGVGQARPLLQVTSPVLQRLEGVLRQLMSQGLSWHDDLTEYVISQEMERIPRLRPPEP
RPRDRSGLAPKRPGPAGELLLQDIPTGSAPAAEHRLPQPPVGKGGAGASSSLSPLQAELLP
PLLEHLLLPPQPPHPSLSYEPALLQPYLFHQFGSRDGSRVSEGSPGMVSVGPLPKAEAPALF
SRTASKGIFGDHPGHSYGDLPGPSPAQLFQDSGLLYLAQELPAPSRARVPRLPEQGSSSRA
EDSPEGYEKEGLGDRGEKPASPAVQPDAALQRLAAVLAGYGVELRQLTPEQLSTLLTLLQ
LLPKGAGRNPGGVVNVGADIKKTMEGPVEGRDTAELPARTSPMPGHPTASPTSSEVQEVP
SPVSSEPPKAARPPVTPVLLEKKSPLGQSQPTVAGQPSARPAAEEYGYIVTDEKPLSLAAG
VKLLEILAEHVHMSSGSFINISVVGPALTFRIRHNEQNLSLADVTQEAGLVKSELEAETGL
QILQTGVGQREEAAAVLPQTAHSTSPMRSVLLTLVALAGVAGLLVALAVALCVRQHARQ
QDKERLAALGPEGAHGDTTFEYQDLCRQHMATKSLFNRAEGPPEPSRVSSVSSQFSDAAQ
ASPSSHSSTPSWCEEPAQANMDISTGHMILAYMEDHLRNRDRLAKEWQALCAYQAEPNT
CATAQGEGNIKKNRHPDFLPYDHARIKLKVESSPSRSDYINASPIIEHDPRMPAYIATEGPL
SHTIADFWEMVWESGCTVIVMLTPLVEDGVKQCDRYWPDEGASLYHVYEVNLVSEHIW
CEDFLVRSFYLKNVQTQETRTLTEFHFLSWPAEGTPASTRPLLDFRRKVNKCYRGRSCPII
VHCSDGAGRTGTYILIDMVLNRMAKGVKEIDIAATLEHVRDERPGLVRSKDEFEFALTAV
AEEVNAILKALPQ
>NP_001186692.1
(SEQ ID NO: 54)
MRRPRRPGGLGGSGGLRLLLCLLLLSSRPGGCSAVSAHGCLFDRRLCSHLEVCIQDGLFG
QCQVGVGQARPLLQVTSPVLQRLQGVLRQLMSQGLSWHDDLTQYVISQEMERIPRLRPP
EPRPRDRSGLAPKRPGPAGELLLQDIPTGSAPAAQHRLPQPPVGKGGAGASSSLSPLQAEL
LPPLLEHLLLPPQPPHPSLSYEPALLQPYLFHQFGSRDGSRVSEGSPGMVSVGPLPKAEAPA
LFSRTASKGIFGDHPGHSYGDLPGPSPAQLFQDSGLLYLAQELPAPSRARVPRLPEQGSSSR
AEDSPEGYEKEGLGDRGEKPASPAVQPDAALQRLAAVLAGYGVELRQLTPEQLSTLLTLL
QLLPKGAGRNPGGVVNVGADIKKTMEGPVEGRDTAELPARTSPMPGHPTASPTSSEVQQ
VPSPVSSEPPKAARPPVTPVLLEKKSPLGQSQPTVAGQPSARPAAEEYGYIVTDQNVVGPA
LTFRIRHNEQNLSLADVTQQAGLVKSELEAQTGLQILQTGVGQREEAAAVLPQTAHSTSP
MRSVLLTLVALAGVAGLLVALAVALCVRQHARQQDKERLAALGPEGAHGDTTFEYQDL
CRQHMATKSLFNRAEGPPEPSRVSSVSSQFSDAAQASPSSHSSTPSWCEEPAQANMDISTG
HMILAYMEDHLRNRDRLAKEWQALCAYQAEPNTCATAQGEGNIKKNRHPDFLPYDHAR
IKLKVESSPSRSDYINASPIIEHDPRMPAYIATQGPLSHTIADFWQMVWESGCTVIVMLTPL
VEDGVKQCDRYWPDEGASLYHVYEVNLVSEHIWCEDFLVRSFYLKNVQTQETRTLTQFH
FLSWPAEGTPASTRPLLDFRRKVNKCYRGRSCPIIVHCSDGAGRTGTYILIDMVLNRMAK
GVKEIDIAATLEHVRDQRPGLVRSKDQFEFALTAVAEEVNAILKALPQ
>NP_001186693.1
(SEQ ID NO: 55)
MSQGLSWHDDLTQYVISQEMERIPRLRPPEPRPRDRSGLAPKRPGPAGELLLQDIPTGSAP
AAQHRLPQPPVGKGGAGASSSLSPLQAELLPPLLEHLLLPPQPPHPSLSYEPALLQPYLFHQ
FGSRDGSRVSEGSPGMVSVGPLPKAEAPALFSRTASKGIFGDHPGHSYGDLPGPSPAQLFQ
DSGLLYLAQELPAPSRARVPRLPEQGSSSRAEDSPEGYEKEGLGDRGEKPASPAVQPDAA
LQRLAAVLAGYGVELRQLTPEQLSTLLTLLQLLPKGAGRNPGGVVNVGADIKKTMEGPV
EGRDTAELPARTSPMPGHPTASPTSSEVQQVPSPVSSEPPKAARPPVTPVLLEKKSPLGQSQ
PTVAGQPSARPAAEEYGYIVTDQKPLSLAAGVKLLEILAEHVHMSSGSFINISVVGPALTF
RIRHNEQNLSLADVTQQAGLVKSELEAQTGLQILQTGVGQREEAAAVLPQTAHSTSPMRS
VLLTLVALAGVAGLLVALAVALCVRQHARQQDKERLAALGPEGAHGDTTFEYQDLCRQ
HMATKSLFNRAEGPPEPSRVSSVSSQFSDAAQASPSSHSSTPSWCEEPAQANMDISTGHMI
LAYMEDHLRNRDRLAKEWQALCAYQAEPNTCATAQGEGNIKKNRHPDFLPYDHARIKL
KVESSPSRSDYINASPIIEHDPRMPAYIATQGPLSHTIADFWQMVWESGCTVIVMLTPLVE
DGVKQCDRYWPDEGASLYHVYEVNLVSEHIWCEDFLVRSFYLKNVQTQETRTLTQFHFL
SWPAEGTPASTRPLLDFRRKVNKCYRGRSCPIIVHCSDGAGRTGTYILIDMVLNRMAKGV
KEIDIAATLEHVRDQRPGLVRSKDQFEFALTAVAEEVNAILKALPQ

The peptide library designed is shown in Table 2.

TABLE 2
Islet autoantigen peptide library

| | | SEQ ID |
|---|---|---|
| Islet Autoantigen | Sequence | NO: |
| IA-2 | H-VSSEPPKAARPPVTPVL-OH | 56 |
| IA-2 | H-HSSTPSWCEEPAQANMD-OH | 57 |
| IA-2 | H-KSLFNRAEGPPEPSRVS-OH | 58 |
| IA-2 | H-ALCVRQHARQQDKERLA-OH | 59 |
| IA-2 | H-TGHMILAYMEDHLRNRD-OH | 60 |
| IA-2 | H-LLPPLLEHLLLPPQPPH-OH | 61 |
| IA-2 | H-SPLQAELLPPLLEHLLL-OH | 62 |
| IA-2 | H-EHVRDQRPGLVRSKDQF-OH | 63 |
| IA-2 | H-ASPTSSEVQQVPSPVSS-OH | 64 |
| IA-2 | H-PGHPTASPTSSEVQQVP-OH | 65 |
| IA-2 | H-GVKLLEILAEHVHMSSG-OH | 66 |
| IA-2 | H-EGYEKEGLGDRGEKPAS-OH | 67 |
| IA-2 | H-TEFHFLSWPAEGTPAST-OH | 68 |
| IA-2 | H-LKNVQTQETRTLTQFHF-OH | 69 |
| IA-2 | H-AAEEYGYIVTDEKPLSL-OH | 70 |
| IA-2 | H-GRDTAELPARTSPMPGH-OH | 71 |
| IA-2 | H-QLLPKGAGRNPGGVVNV-OH | 72 |
| IA-2 | H-DFLVRSFYLKNVQTQET-OH | 73 |
| IA-2 | H-SSTPSWCEEPAQANMDI-OH | 74 |
| IA-2 | H-LAAVLAGYGVELRQLTP-OH | 75 |
| IA-2 | H-PIIEHDPRMPAYIATEG-OH | 76 |
| IA-2 | H-LLTLVALAGVAGLLVAL-OH | 77 |
| IA-2 | H-PASTRPLLDFRRKVNKC-OH | 78 |
| IA-2 | H-GYGVELRQLTPEQLSTL-OH | 79 |
| IA-2 | H-RQHARQQDKERLAALGP-OH | 80 |
| IA-2 | H-GCSAVSAHGCLFDRRLC-OH | 81 |
| IA-2 | H-QQDKERLAALGPEGAHG-OH | 82 |
| IA-2 | H-PGGLGGSGGLRLLLCLL-OH | 83 |
| IA-2 | H-AQGEGNIKKNRHPDFLP-OH | 84 |
| IA-2 | H-PGHSYGDLPGPSPAQLF-OH | 85 |
| IA-2 | H-YHVYEVNLVSEHIWCED-OH | 86 |
| IA-2 | H-LSWHDDLTQYVISQEME-OH | 87 |
| IA-2 | H-PVLLEKKSPLGQSQPTV-OH | 88 |
| IA-2 | H-GDHPGHSYGDLPGPSPA-OH | 89 |
| IA-2 | H-YWPDEGASLYHVYEVNL-OH | 90 |
| IA-2 | H-QGPLSHTIADFWQMVWE-OH | 91 |
| IA-2 | H-LVKSELEAQTGLQILQT-OH | 92 |
| IA-2 | H-GVKEIDIAATLEHVRDE-OH | 93 |
| IA-2 | H-VSEGSPGMVSVGPLPKA-OH | 94 |
| IA-2 | H-GRNPGGVVNVGADIKKT-OH | 95 |
| IA-2 | H-GLQILQTGVGQREEAAA-OH | 96 |
| IA-2 | H-HDDLTEYVISQEMERIP-OH | 97 |
| IA-2 | H-ALLQPYLFHQFGSRDGS-OH | 98 |
| IA-2 | H-YQAEPNTCATAQGEGNI-OH | 99 |
| IA-2 | H-LPKAEAPALFSRTASKG-OH | 100 |
| IA-2 | H-PKAARPPVTPVLLEKKS-OH | 101 |
| IA-2 | H-RPRDRSGLAPKRPGPAG-OH | 102 |
| IA-2 | H-ALTAVAEEVNAILKALP-OH | 103 |
| IA-2 | H-DFWQMVWESGCTVIVML-OH | 104 |
| IA-2 | H-DRLAKEWQALCAYQAEP-OH | 105 |
| IA-2 | H-RGRSCPIIVHCSDGAGR-OH | 106 |
| IA-2 | H-ARIKLKVESSPSRSDYI-OH | 107 |
| IA-2 | H-VSSVSSQFSDAAQASPS-OH | 108 |
| IA-2 | H-QFHFLSWPAEGTPASTR-OH | 109 |
| IA-2 | H-SDYINASPIIEHDPRMP-OH | 110 |
| IA-2 | H-PMPGHPTASPTSSEVQQ-OH | 111 |
| IA-2 | H-YILIDMVLNRMAKGVKE-OH | 112 |
| IA-2 | H-EQNLSLADVTQEAGLVK-OH | 113 |
| IA-2 | H-KGAGRNPGGVVNVGADI-OH | 114 |
| IA-2 | H-SGGLRLLLCLLLLSSRP-OH | 115 |
| IA-2 | H-AEEYGYIVTDQNVVGPA-OH | 116 |
| IA-2 | H-QAEPNTCATAQGEGNIK-OH | 117 |
| IA-2 | H-HGDTTFEYQDLCRQHMA-OH | 118 |
| IA-2 | H-TQYVISQEMERIPRLRP-OH | 119 |
| IA-2 | H-DIAATLEHVRDQRPGLV-OH | 120 |
| IA-2 | H-RIPRLRPPEPRPRDRSG-OH | 121 |
| IA-2 | H-KAEAPALFSRTASKGIF-OH | 122 |
| IA-2 | H-QEMERIPRLRPPEPRPR-OH | 123 |
| IA-2 | H-LCSHLEVCIQDGLFGQC-OH | 124 |
| IA-2 | H-WCEEPAQANMDISTGHM-OH | 125 |
| IA-2 | H-DLPGPSPAQLFQDSGLL-OH | 126 |
| IA-2 | H-REEAAAVLPQTAHSTSP-OH | 127 |
| IA-2 | H-GSFINISVVGPALTFRI-OH | 128 |
| IA-2 | H-PAQANMDISTGHMILAY-OH | 129 |
| IA-2 | H-PPQPPHPSLSYEPALLQ-OH | 130 |
| IA-2 | H-GLFGQCQVGVGQARPLL-OH | 131 |
| IA-2 | H-WQALCAYQAEPNTCATA-OH | 132 |
| IA-2 | H-AQHRLPQPPVGKGGAGA-OH | 133 |
| IA-2 | H-PPVTPVLLEKKSPLGQS-OH | 134 |
| IA-2 | H-SHTIADFWEMVWESGCT-OH | 135 |
| IA-2 | H-QDIPTGSAPAAEHRLPQ-OH | 136 |
| IA-2 | H-QLMSQGLSWHDDLTQYV-OH | 137 |
| IA-2 | H-GPALTFRIRHNEQNLSL-OH | 138 |
| IA-2 | H-EYGYIVTDQNVVGPALT-OH | 139 |
| IA-2 | H-RSKDEFEFALTAVAEEV-OH | 140 |
| IA-2 | H-SPMRSVLLTLVALAGVA-OH | 141 |
| IA-2 | H-TQQAGLVKSELEAQTGL-OH | 142 |
| IA-2 | H-LFDRRLCSHLEVCIQDG-OH | 143 |
| IA-2 | H-QSQPTVAGQPSARPAAE-OH | 144 |
| IA-2 | H-NEQNLSLADVTQQAGLV-OH | 145 |
| IA-2 | H-PAYIATQGPLSHTIADF-OH | 146 |
| IA-2 | H-IADFWEMVWESGCTVIV-OH | 147 |
| IA-2 | H-PSLSYEPALLQPYLFHQ-OH | 148 |
| IA-2 | H-EGNIKKNRHPDFLPYDH-OH | 149 |
| IA-2 | H-SHLEVCIEDGLFGECEV-OH | 150 |
| IA-2 | H-HCSDGAGRTGTYILIDM-OH | 151 |
| IA-2 | H-KNRHPDFLPYDHARIKL-OH | 152 |
| IA-2 | H-VLQRLQGVLRQLMSQGL-OH | 153 |
| IA-2 | H-QEVPSPVSSEPPKAARP-OH | 154 |
| IA-2 | H-AEGPPEPSRVSSVSSQF-OH | 155 |
| IA-2 | H-HPTASPTSSEVQEVPSP-OH | 156 |
| IA-2 | H-VQPDAALQRLAAVLAGY-OH | 157 |
| IA-2 | H-QTGLQILQTGVGQREEA-OH | 158 |
| IA-2 | H-CIEDGLFGECEVGVGQA-OH | 159 |
| IA-2 | H-ASSSLSPLQAELLPPLL-OH | 160 |
| IA-2 | H-ILAYMEDHLRNRDRLAK-OH | 161 |
| IA-2 | H-TPLVEDGVKQCDRYWPD-OH | 162 |
| IA-2 | H-IVMLTPLVEDGVKQCDR-OH | 163 |
| IA-2 | H-PQTAHSTSPMRSVLLTL-OH | 164 |
| IA-2 | H-TQETRTLTQFHFLSWPA-OH | 165 |
| IA-2 | H-QTGVGQREEAAAVLPQT-OH | 166 |
| IA-2 | H-ETRTLTQFHFLSWPAEG-OH | 167 |
| IA-2 | H-DLTEYVISQEMERIPRL-OH | 168 |
| IA-2 | H-LGPEGAHGDTTFEYQDL-OH | 169 |
| IA-2 | H-QFSDAAQASPSSHSSTP-OH | 170 |
| IA-2 | H-VESSPSRSDYINASPII-OH | 171 |
| IA-2 | H-LKNVQTQETRTLTEFHF-OH | 172 |
| IA-2 | H-RKVNKCYRGRSCPIIVH-OH | 173 |
| IA-2 | H-RPLLQVTSPVLQRLEGV-OH | 174 |
| IA-2 | H-FGSRDGSRVSEGSPGMV-OH | 175 |
| IA-2 | H-QRLAAVLAGYGVELRQL-OH | 176 |
| IA-2 | H-VCIQDGLFGQCQVGVGQ-OH | 177 |
| IA-2 | H-QDLCRQHMATKSLFNRA-OH | 178 |
| IA-2 | H-ADVTQEAGLVKSELEAE-OH | 179 |
| IA-2 | H-ANMDISTGHMILAYMED-OH | 180 |
| IA-2 | H-EDGVKQCDRYWPDEGAS-OH | 181 |
| IA-2 | H-QASPSSHSSTPSWCEEP-OH | 182 |
| IA-2 | H-SSEPPKAARPPVTPVLL-OH | 183 |
| IA-2 | H-LSSRPGGCSAVSAHGCL-OH | 184 |
| IA-2 | H-SSPSRSDYINASPIIEH-OH | 185 |
| IA-2 | H-KLLEILAEHVHMSSGSF-OH | 186 |
| IA-2 | H-PTSSEVQEVPSPVSSEP-OH | 187 |
| IA-2 | H-QRPGLVRSKDQFEFALT-OH | 188 |
| IA-2 | H-SAPAAEHRLPQPPVGKG-OH | 189 |
| IA-2 | H-EDHLRNRDRLAKEWQAL-OH | 190 |
| IA-2 | H-GLAPKRPGPAGELLLQD-OH | 191 |
| IA-2 | H-EHDPRMPAYIATQGPLS-OH | 192 |
| IA-2 | H-PRMPAYIATEGPLSHTI-OH | 193 |
| IA-2 | H-PPLLEHLLLPPQPPHPS-OH | 194 |
| IA-2 | H-VTDQKPLSLAAGVKLLE-OH | 195 |
| IA-2 | H-DEGASLYHVYEVNLVSE-OH | 196 |
| IA-2 | H-CATAQGEGNIKKNRHPD-OH | 197 |
| IA-2 | H-ALFSRTASKGIFGDHPG-OH | 198 |
| IA-2 | H-ELPAPSRARVPRLPEQG-OH | 199 |
| IA-2 | H-LVALAVALCVRQHARQQ-OH | 200 |
| IA-2 | H-LEHVRDERPGLVRSKDE-OH | 201 |
| IA-2 | H-VNLVSEHIWCEDFLVRS-OH | 202 |
| IA-2 | H-IAATLEHVRDERPGLVR-OH | 203 |
| IA-2 | H-GLFGECEVGVGQARPLL-OH | 204 |
| IA-2 | H-PYLFHQFGSRDGSRVSE-OH | 205 |
| IA-2 | H-AGASSSLSPLQAELLPP-OH | 206 |
| IA-2 | H-VRSKDQFEFALTAVAEE-OH | 207 |
| IA-2 | H-NRDRLAKEWQALCAYQA-OH | 208 |
| IA-2 | H-HLLLPPQPPHPSLSYEP-OH | 209 |
| IA-2 | H-GSPGMVSVGPLPKAEAP-OH | 210 |
| IA-2 | H-SFYLKNVQTQETRTLTQ-OH | 211 |
| IA-2 | H-KERLAALGPEGAHGDTT-OH | 212 |
| IA-2 | H-TLLTLLQLLPKGAGRNP-OH | 213 |
| IA-2 | H-AEEYGYIVTDQKPLSLA-OH | 214 |
| IA-2 | H-VALAGVAGLLVALAVAL-OH | 215 |
| IA-2 | H-GRTGTYILIDMVLNRMA-OH | 216 |
| IA-2 | H-KTMEGPVEGRDTAELPA-OH | 217 |
| IA-2 | H-HMSSGSFINISVVGPAL-OH | 218 |
| IA-2 | H-TSPVLQRLEGVLRQLMS-OH | 219 |
| IA-2 | H-EGLGDRGEKPASPAVQP-OH | 220 |
| IA-2 | H-LLQDIPTGSAPAAQHRL-OH | 221 |
| IA-2 | H-DVTQQAGLVKSELEAQT-OH | 222 |
| IA-2 | H-LNRMAKGVKEIDIAATL-OH | 223 |
| IA-2 | H-SELEAETGLQILQTGVG-OH | 224 |
| IA-2 | H-LAEHVHMSSGSFINISV-OH | 225 |
| IA-2 | H-IKKTMEGPVEGRDTAEL-OH | 226 |
| IA-2 | H-GDRGEKPASPAVQPDAA-OH | 227 |
| IA-2 | H-QETRTLTEFHFLSWPAE-OH | 228 |
| IA-2 | H-GPSPAQLFQDSGLLYLA-OH | 229 |
| IA-2 | H-NVGADIKKTMEGPVEGR-OH | 230 |
| IA-2 | H-SLADVTQQAGLVKSELE-OH | 231 |
| IA-2 | H-ERPGLVRSKDEFEFALT-OH | 232 |
| IA-2 | H-LTAVAEEVNAILKALPQ-OH | 233 |
| IA-2 | H-QPPVGKGGAGASSSLSP-OH | 234 |
| IA-2 | H-AHSTSPMRSVLLTLVAL-OH | 235 |
| IA-2 | H-HIWCEDFLVRSFYLKNV-OH | 236 |
| IA-2 | H-FEYQDLCRQHMATKSLF-OH | 237 |
| IA-2 | H-LPYDHARIKLKVESSPS-OH | 238 |
| IA-2 | H-EEYGYIVTDEKPLSLAA-OH | 239 |
| IA-2 | H-AGLVKSELEAETGLQIL-OH | 240 |
| IA-2 | H-AGVAGLLVALAVALCVR-OH | 241 |
| IA-2 | H-LFQDSGLLYLAQELPAP-OH | 242 |
| IA-2 | H-EKPLSLAAGVKLLEILA-OH | 243 |
| IA-2 | H-EQGSSSRAEDSPEGYEK-OH | 244 |
| IA-2 | H-PVEGRDTAELPARTSPM-OH | 245 |
| IA-2 | H-GVVNVGADIKKTMEGPV-OH | 246 |
| IA-2 | H-MVSVGPLPKAEAPALFS-OH | 247 |
| IA-2 | H-RQLTPEQLSTLLTLLQL-OH | 248 |
| IA-2 | H-LEAQTGLQILQTGVGQR-OH | 249 |
| IA-2 | H-PGPAGELLLQDIPTGSA-OH | 250 |
| IA-2 | H-PSRARVPRLPEQGSSSR-OH | 251 |
| IA-2 | H-GPAGELLLQDIPTGSAP-OH | 252 |
| IA-2 | H-PLLDFRRKVNKCYRGRS-OH | 253 |
| IA-2 | H-SQGLSWHDDLTEYVISQ-OH | 254 |
| IA-2 | H-DLTQYVISQEMERIPRL-OH | 255 |
| IA-2 | H-SARPAAEEYGYIVTDQK-OH | 256 |
| IA-2 | H-LQVTSPVLQRLQGVLRQ-OH | 257 |
| IA-2 | H-KQCDRYWPDEGASLYHV-OH | 258 |
| IA-2 | H-KKSPLGQSQPTVAGQPS-OH | 259 |
| IA-2 | H-QPTVAGQPSARPAAEEY-OH | 260 |
| IA-2 | H-SRVSEGSPGMVSVGPLP-OH | 261 |
| IA-2 | H-INASPIIEHDPRMPAYI-OH | 262 |
| IA-2 | H-VSAHGCLFDRRLCSHLE-OH | 263 |
| IA-2 | H-VGKGGAGASSSLSPLQA-OH | 264 |
| IA-2 | H-HMATKSLFNRAEGPPEP-OH | 265 |
| IA-2 | H-RRLCSHLEVCIEDGLFG-OH | 266 |
| IA-2 | H-RLEGVLRQLMSQGLSWH-OH | 267 |
| IA-2 | H-LEVCIQDGLFGQCQVGV-OH | 268 |
| IA-2 | H-CPIIVHCSDGAGRTGTY-OH | 269 |
| IA-2 | H-QGVLRQLMSQGLSWHDD-OH | 270 |
| IA-2 | H-PDAALQRLAAVLAGYGV-OH | 271 |
| IA-2 | H-FQDSGLLYLAQELPAPS-OH | 272 |
| IA-2 | H-ATEGPLSHTIADFWEMV-OH | 273 |
| IA-2 | H-ARPLLQVTSPVLQRLQG-OH | 274 |
| IA-2 | H-CTVIVMLTPLVEDGVKQ-OH | 275 |
| IA-2 | H-AAAVLPQTAHSTSPMRS-OH | 276 |
| IA-2 | H-RIRHNEQNLSLADVTQE-OH | 277 |
| IA-2 | H-TDQNVVGPALTFRIRHN-OH | 278 |
| IA-2 | H-PLSLAAGVKLLEILAEH-OH | 279 |
| IA-2 | H-LRPPEPRPRDRSGLAPK-OH | 280 |
| IA-2 | H-SLSYEPALLQPYLFHQF-OH | 281 |
| IA-2 | H-TLEHVRDERPGLVRSKD-OH | 282 |
| IA-2 | H-SSEVQQVPSPVSSEPPK-OH | 283 |
| IA-2 | H-WESGCTVIVMLTPLVED-OH | 284 |
| IA-2 | H-GQPSARPAAEEYGYIVT-OH | 285 |
| IA-2 | H-PASPAVQPDAALQRLAA-OH | 286 |
| IA-2 | H-RPRRPGGLGGSGGLRLL-OH | 287 |
| IA-2 | H-TGSAPAAQHRLPQPPVG-OH | 288 |
| IA-2 | H-SHTIADFWQMVWESGCT-OH | 289 |
| IA-2 | H-LYLAQELPAPSRARVPR-OH | 290 |
| IA-2 | H-YGYIVTDQKPLSLAAGV-OH | 291 |
| IA-2 | H-ISVVGPALTFRIRHNEQ-OH | 292 |
| IA-2 | H-QLSTLLTLLQLLPKGAG-OH | 293 |
| IA-2 | H-SLYHVYEVNLVSEHIWC-OH | 294 |
| IA-2 | H-PEPSRVSSVSSQFSDAA-OH | 295 |
| IA-2 | H-ASKGIFGDHPGHSYGDL-OH | 296 |
| IA-2 | H-DMVLNRMAKGVKEIDIA-OH | 297 |
| IA-2 | H-LLCLLLLSSRPGGCSAV-OH | 298 |
| IA-2 | H-QVGVGQARPLLQVTSPV-OH | 299 |
| IA-2 | H-YIATEGPLSHTIADFWE-OH | 300 |
| IA-2 | H-EFALTAVAEEVNAILKA-OH | 301 |
| IA-2 | H-VNKCYRGRSCPIIVHCS-OH | 302 |
| IA-2 | H-RAEDSPEGYEKEGLGDR-OH | 303 |
| IA-2 | H-MRRPRRPGGLGGSGGLR-OH | 304 |
| IA-2 | H-ECEVGVGQARPLLQVTS-OH | 305 |
| IA-2 | H-PRLPEQGSSSRAEDSPE-OH | 306 |
| IA-2 | H-GYIVTDEKPLSLAAGVK-OH | 307 |
| IA-2 | H-HPDFLPYDHARIKLKVE-OH | 308 |
| IA-2 | H-WPAEGTPASTRPLLDFR-OH | 309 |
| IA-2 | H-LPARTSPMPGHPTASPT-OH | 310 |
| GAD 65/67 | H-PIHHHHHHLVPRGSEAS-OH | 311 |
| GAD 65/67 | H-VILIKCDERGKMIPSDL-OH | 312 |
| GAD 65/67 | H-GDKVNFFRMVISNPAAT-OH | 313 |
| GAD 65/67 | H-MIPSDLERRILEAKEKG-OH | 314 |
| GAD 65/67 | H-MASPGSGFWSFGSEDGS-OH | 315 |
| GAD 65/67 | H-EMVFDGKPQHTNVCFWY-OH | 316 |
| GAD 65/67 | H-NQMHASYLFQQDKHYDL-OH | 317 |
| GAD 65/67 | H-TANTNMFTYEIAPVFVL-OH | 318 |
| GAD 65/67 | H-PQNLEEILMHCQTTLKY-OH | 319 |
| GAD 65/67 | H-GFWSFGSEDGSGDSENP-OH | 320 |
| GAD 65/67 | H-EYVTLKKMREIIGWPGG-OH | 321 |
| GAD 65/67 | H-YDTGDKALQCGRHVDVF-OH | 322 |
| GAD 65/67 | H-LCDLLPACDGERPTLAF-OH | 323 |
| GAD 65/67 | H-FTGGIGNKLCDLLPACD-OH | 324 |
| GAD 65/67 | H-LHATDLLPACDGERPTL-OH | 325 |
| GAD 65/67 | H-RLSKVAPVIKARMMEYG-OH | 326 |
| GAD 65/67 | H-QNCNQMHASYLFQEDKH-OH | 327 |
| GAD 65/67 | H-AFDPLLAVADICKKYKI-OH | 328 |
| GAD 65/67 | H-EAKQKGFVPFLVSATAG-OH | 329 |
| GAD 65/67 | H-CACDEKPCSCSKVDVNY-OH | 330 |
| GAD 65/67 | H-FDRSTKVIDFHYPNELL-OH | 331 |
| GAD 65/67 | H-VIDFHYPNELLQEYNWE-OH | 332 |
| GAD 65/67 | H-KTGIVSSKIIKLFFRLQ-OH | 333 |
| GAD 65/67 | H-PACDGERPTLAFLQDVM-OH | 334 |
| GAD 65/67 | H-SDLERRILEAKQKGFVP-OH | 335 |
| GAD 65/67 | H-IGTDSVILIKCDERGKM-OH | 336 |
| GAD 65/67 | H-VISNPAATHQDIDFLIE-OH | 337 |
| GAD 65/67 | H-AIKTGHPRYFNQLSTGL-OH | 338 |
| GAD 65/67 | H-VAQKFTGGIGNKLCALL-OH | 339 |
| GAD 65/67 | H-HVDAAWGGGLLMSRKHK-OH | 340 |
| GAD 65/67 | H-MQNCNQMHASYLFQQDK-OH | 341 |
| GAD 65/67 | H-YLFQQDKHYDLSYDTGD-OH | 342 |
| GAD 65/67 | H-DFHYPNELLQEYNWELA-OH | 343 |
| GAD 65/67 | H-ELLQEYNWELADQPQNL-OH | 344 |
| GAD 65/67 | H-AAARKAACACDEKPCSC-OH | 345 |
| GAD 65/67 | H-EKGMAALPRLIAFTSEH-OH | 346 |
| GAD 65/67 | H-YKIWMHVDGLMQNCNQM-OH | 347 |
| GAD 65/67 | H-VDVNYAFLHATDLLPAC-OH | 348 |
| GAD 65/67 | H-PRYFNQLSTGLDMVGLA-OH | 349 |
| GAD 65/67 | H-QDIDFLIEEIERLGQDL-OH | 350 |
| GAD 65/67 | H-ENPGTARAWCQVAQKFT-OH | 351 |
| GAD 65/67 | H-PQNLEEILMHCETTLKY-OH | 352 |
| GAD 65/67 | H-RPTLAFLQDVMNILLQY-OH | 353 |
| GAD 65/67 | H-AESGGSQPPRAAARKAA-OH | 354 |
| GAD 65/67 | H-RTLEDNEERMSRLSKVA-OH | 355 |
| GAD 65/67 | H-VPFLVSATAGTTVYGAF-OH | 356 |
| GAD 65/67 | H-DLSYDTGDKALQCGRHV-OH | 357 |
| GAD 65/67 | H-FNQLSTGLDMVGLAADW-OH | 358 |
| GAD 65/67 | H-YVVKSFDRSTKVIDFHY-OH | 359 |
| GAD 65/67 | H-NMFTYEIAPVFVLLEYV-OH | 360 |
| GAD 65/67 | H-GFEAHVDKCLELAEYLY-OH | 361 |
| GAD 65/67 | H-SEDGSGDSENPGTARAW-OH | 362 |
| GAD 65/67 | H-RAAARKAACACDQKPCS-OH | 363 |
| GAD 65/67 | H-CFWYIPPSLRTLEDNEE-OH | 364 |
| GAD 65/67 | H-TGLDMVGLAADWLTSTA-OH | 365 |
| GAD 65/67 | H-IKARMMEYGTTMVSYQP-OH | 366 |
| GAD 65/67 | H-THQDIDFLIEEIERLGQ-OH | 367 |
| GAD 65/67 | H-NMYAMMIARFKMFPEVK-OH | 368 |
| GAD 65/67 | H-MNILLEYVVKSFDRSTK-OH | 369 |
| GAD 65/67 | H-TARAWCQVAQKFTGGIG-OH | 370 |
| GAD 65/67 | H-QDVMNILLQYVVKSFDR-OH | 371 |
| GAD 65/67 | H-ASPGSGFWSFGSEDGSG-OH | 372 |
| GAD 65/67 | H-HFSLKKGAAALGIGTDS-OH | 373 |
| GAD 65/67 | H-YQPLGDKVNFFRMVISN-OH | 374 |
| GAD 65/67 | H-ILLQYVVKSFDRSTKVI-OH | 375 |
| GAD 65/67 | H-AACACDQKPCSCSKVDV-OH | 376 |
| GAD 65/67 | H-AATHQDIDFLIEEPEAN-OH | 377 |
| GAD 65/67 | H-HHLVPRGSEASNSGFWS-OH | 378 |
| GAD 65/67 | H-KLSGVERANSVTWNPHK-OH | 379 |
| GAD 65/67 | H-CSCSKVDVNYAFLHATD-OH | 380 |
| GAD 65/67 | H-DPLLAVADICKKYKIWM-OH | 381 |
| GAD 65/67 | H-AKEKGFVPFLVSATAGT-OH | 382 |
| GAD 65/67 | H-KGTTGFEAHVDKCLELA-OH | 383 |
| GAD 65/67 | H-GIFSPGGAISNMYAMMI-OH | 384 |
| GAD 65/67 | H-HASYLFQEDKHYDLSYD-OH | 385 |
| GAD 65/67 | H-GTTMVSYQPLGDKVNFF-OH | 386 |
| GAD 65/67 | H-RRILEAKQKGFVPFLVS-OH | 387 |
| GAD 65/67 | H-LADQPQNLEEILMHCET-OH | 388 |
| GAD 65/67 | H-LQDVMNILLQYVVKSFD-OH | 389 |
| GAD 65/67 | H-IARFKMFPEVKEKGMAA-OH | 390 |
| GAD 65/67 | H-QCSALLVREEGLMQNCN-OH | 391 |
| GAD 65/67 | H-FSLKKGAAALGIGTDSV-OH | 392 |
| GAD 65/67 | H-LCALLYGDAEKPAESGG-OH | 393 |
| GAD 65/67 | H-ELAEYLYNIIKNREGYE-OH | 394 |
| GAD 65/67 | H-CQVAQKFTGGIGNKLCD-OH | 395 |
| GAD 65/67 | H-QEDKHYDLSYDTGDKAL-OH | 396 |
| GAD 65/67 | H-GIGNKLCALLYGDAEKP-OH | 397 |
| GAD 65/67 | H-NYAFLHATDLLPACDGE-OH | 398 |
| GAD 65/67 | H-SEASNSGFWSFGSEDGS-OH | 399 |
| GAD 65/67 | H-GDAEKPAESGGSQPPRA-OH | 400 |
| GAD 65/67 | H-AAALGIGTDSVILIKCD-OH | 401 |
| GAD 65/67 | H-WNPHKMMGVPLQCSALL-OH | 402 |
| GAD 65/67 | H-MMEYGTTMVSYQPLGDK-OH | 403 |
| GAD 65/67 | H-IGNKLCDLLPACDGERP-OH | 404 |
| GAD 65/67 | H-KYKIWMHVDAAWGGGLL-OH | 405 |
| GAD 65/67 | H-CDQKPCSCSKVDVNYAF-OH | 406 |
| GAD 65/67 | H-ERRILEAKEKGFVPFLV-OH | 407 |
| GAD 65/67 | H-DGSGDSENPGTARAWCQ-OH | 408 |
| GAD 65/67 | H-ETTLKYAIKTGHPRYFN-OH | 409 |
| GAD 65/67 | H-RANSVTWNPHKMMGVPL-OH | 410 |
| GAD 65/67 | H-LEYVVKSFDRSTKVIDF-OH | 411 |
| GAD 65/67 | H-PGGSGDGIFSPGGAISN-OH | 412 |
| GAD 65/67 | H-AALPRLIAFTSEHSHFS-OH | 413 |
| GAD 65/67 | H-KHKWKLSGVERANSVTW-OH | 414 |
| GAD 65/67 | H-FTSEHSHFSLKKGAAAL-OH | 415 |
| GAD 65/67 | H-ADWLTSTANTNMFTYEI-OH | 416 |
| GAD 65/67 | H-REGYEMVFDGKPQHTNV-OH | 417 |
| GAD 65/67 | H-GTTVYGAFDPLLAVADI-OH | 418 |
| GAD 65/67 | H-ADICKKYKIWMHVDGLM-OH | 419 |
| GAD 65/67 | H-ICKKYKIWMHVDAAWGG-OH | 420 |
| GAD 65/67 | H-NWELADQPQNLEEILMH-OH | 421 |
| GAD 65/67 | H-EERMSRLSKVAPVIKAR-OH | 422 |
| GAD 65/67 | H-ILMHCQTTLKYAIKTGH-OH | 423 |
| GAD 65/67 | H-TYEIAPVFVLLEYVTLK-OH | 424 |
| GAD 65/67 | H-GAISNMYAMMIARFKMF-OH | 425 |
| GAD 65/67 | H-YIPPSLRTLEDNEERMS-OH | 426 |
| GAD 65/67 | H-LMHCETTLKYAIKTGIV-OH | 427 |
| GAD 65/67 | H-MSPIHHHHHHLVPRGSE-OH | 428 |
| GAD 65/67 | H-GDKALQCGRHVDVFKLW-OH | 429 |
| GAD 65/67 | H-GLAADWLTSTANTNMFT-OH | 430 |
| GAD 65/67 | H-GLLMSRKHKWKLSGVER-OH | 431 |
| GAD 65/67 | H-DVFKLWLMWRAKGTTGF-OH | 432 |
| GAD 65/67 | H-LMWRAKGTTGFEAHVDK-OH | 433 |
| GAD 65/67 | H-APVIKARMMEYGTTMVS-OH | 434 |
| GAD 65/67 | H-MGVPLQCSALLVREEGL-OH | 435 |
| GAD 65/67 | H-LKYAIKTGIVSSKIIKL-OH | 436 |
| GAD 65/67 | H-IWMHVDGLMQNCNQMHA-OH | 437 |
| GAD 65/67 | H-QPPRAAARKAACACDEK-OH | 438 |
| GAD 65/67 | H-QCGRHVDVFKLWLMWRA-OH | 439 |
| GAD 65/67 | H-FPEVKEKGMAALPRLIA-OH | 440 |
| GAD 65/67 | H-TGGIGNKLCALLYGDAE-OH | 441 |
| GAD 65/67 | H-RMVISNPAATHQDIDFL-OH | 442 |
| GAD 65/67 | H-GKPQHTNVCFWYIPPSL-OH | 443 |
| GAD 65/67 | H-IIGWPGGSGDGIFSPGT-OH | 444 |
| GAD 65/67 | H-WGGGLLMSRKHKWKLSG-OH | 445 |
| GAD 65/67 | H-VNFFRMVISNPAATHQD-OH | 446 |
| GAD 65/67 | H-VSATAGTTVYGAFDPLL-OH | 447 |
| GAD 65/67 | H-DERGKMIPSDLERRILE-OH | 448 |
| GAD 65/67 | H-NVCFWYIPPSLRTLEDN-OH | 449 |
| GAD 65/67 | H-PPRAAARKAACACDQKP-OH | 450 |
| GAD 65/67 | H-GSEASNSGFWSFGSEDG-OH | 451 |
| GAD 65/67 | H-ACACDEKPCSCSKVDVN-OH | 452 |
| GAD 65/67 | H-AFLQDVMNILLEYVVKS-OH | 453 |
| GAD 65/67 | H-VFVLLEYVTLKKMREII-OH | 454 |
| GAD 65/67 | H-NLEEILMHCETTLKYAI-OH | 455 |
| GAD 65/67 | H-LPRLIAFTSEHSHFSLK-OH | 456 |
| GAD 65/67 | H-YNIIKNREGYEMVFDGK-OH | 457 |
| GAD 65/67 | H-QTTLKYAIKTGIVSSKI-OH | 458 |
| GAD 65/67 | H-KMREIIGWPGGSGDGIF-OH | 459 |
| GAD 65/67 | H-VDKCLELAEYLYNIIKN-OH | 460 |
| GAD 65/67 | H-VREEGLMQNCNQMHASY-OH | 461 |
| Insulin/Proinsulin | FYTPKTRREAEDLQGSL | 462 |
| Insulin/Proinsulin | EDLQVGEVELGGGPGAG | 463 |
| Insulin/Proinsulin | RREAEDLEGSLQPLALE | 464 |
| Insulin/Proinsulin | CGSHLVEALYLVCGERG | 465 |
| Insulin/Proinsulin | REAEDLQVGQVELGGGP | 466 |
| Insulin/Proinsulin | ALLALWGPDPAAAFVNQ | 467 |
| Insulin/Proinsulin | YTPKTRREAEVGQVELG | 468 |
| Insulin/Proinsulin | GPGAGSLQPLALEGSLQ | 469 |
| Insulin/Proinsulin | QCCTSICSLYQLENYCN | 470 |
| Insulin/Proinsulin | VEALYLVCGERGFFYTP | 471 |
| Insulin/Proinsulin | NQHLCGSHLVEALYLVC | 472 |
| Insulin/Proinsulin | TPKTRREAEDLEGSLQP | 473 |
| Insulin/Proinsulin | SLQKRGIVEQCCTSICS | 474 |
| Insulin/Proinsulin | PKTRREAEVGEVELGGG | 475 |
| Insulin/Proinsulin | LQGSLQPLALEGSLQKR | 476 |
| Insulin/Proinsulin | YTPKTRREAEDLQVGQV | 477 |
| Insulin/Proinsulin | CGERGFFYTPKTRREAE | 478 |
| Insulin/Proinsulin | LYLVCGERGFFYTPKTR | 479 |
| Insulin/Proinsulin | EDLQVGQVELGGGPGAG | 480 |
| Insulin/Proinsulin | PKTRREAEVGQVELGGG | 481 |
| Insulin/Proinsulin | PAAAFVNQHLCGSHLVE | 482 |
| Insulin/Proinsulin | DLEGSLQPLALEGSLQK | 483 |
| Insulin/Proinsulin | VELGGGPGAGSLQPLAL | 484 |
| Insulin/Proinsulin | PLLALLALWGPDPAAAF | 485 |
| Insulin/Proinsulin | WGPDPAAAFVNQHLCGS | 486 |
| Insulin/Proinsulin | EAEVGQVELGGGPGAGS | 487 |
| Insulin/Proinsulin | MALWMRLLPLLALLALW | 488 |
| Insulin/Proinsulin | MRLLPLLALLALWGPDP | 489 |
| Insulin/Proinsulin | AEVGEVELGGGPGAGSL | 490 |
| Insulin/Proinsulin | RGFFYTPKTRREAEVGE | 491 |
| Insulin/Proinsulin | RREAEDLQGSLQPLALE | 492 |
| Insulin/Proinsulin | IVEQCCTSICSLYQLEN | 493 |
| Insulin/Proinsulin | PLALEGSLQKRGIVEQC | 494 |
| Insulin/Proinsulin | TRREAEDLQVGEVELGG | 495 |

This peptide library is for use in a study such as the one outlined in Example 1.

### Example 3

### Use of IP-10 to Measure Rare Antigen-Specific T Cells in T1D

The objective of the study was to quantify and compare T cell responses to pancreatic islet autoantigens, immunodominant gluten peptides, and pathogen-derived recall antigens before and after oral gluten challenge in patients with both Type-1 diabetes (T1D) and Celiac disease.

### Background

Dietary gluten may play a role in causing or enhancing islet autoimmunity, but the mechanism is not understood. In patient-based studies over the last 14-years, short-term “gluten challenge” has provided a detailed understanding of the immune response underlying Celiac disease. In the present study, gluten challenge was used for the first time to study patients affected by Celiac disease as well as T1D to detect CD4+ T cells specific for autoantigens implicated in T1D, and to test whether islet autoimmunity is affected by reactivation of gluten immunity.

In Celiac disease, the most prevalent genetic association is with the MHC-Class II alleles encoding HLA-DQ2.5, and gluten-derived epitopes recognized by Celiac disease-specific CD4+ T cells are preferentially presented by HLA-DQ2.5. Most of the 10% of patients with Celiac disease who are negative for HLA-DQA1*05 and DQB1*02, which encode HLA-DQ2.5, possess the HLA-DQA1*03 and DQB1*0302 alleles encoding HLA-DQ8, and in these patients distinctive gluten-specific epitopes are restricted by HLA-DQ8. The amino acid sequences of gluten-derived HLA-DQ2.5- and DQ8-restricted epitopes have been described (Sollid L M et al. Immunogenetics. 2012). Six-days after Celiac disease patients who usually exclude dietary gluten commence oral gluten challenge, certain gluten peptides stimulate interferon(IFN)-γ secretion when incubated with peripheral blood mononuclear cells (PBMC) or whole blood (Ontiveros N et al. Clin Exp Immunol 2014). IFN-γ secretion elicited by gluten peptides can be detected by ELISpot assay and is due to activated CD4+ T cells (Anderson R P et al., Nature Med 2000), which have been further characterized by flow cytometry using MHC-Class II multimers (Raki M et al., PNAS USA 2007). In HLA-DQ2.5+ Celiac disease patients who regularly consume gluten (untreated), the median frequency of circulating CD4+ T cells stained by HLA-DQ2.5 multimers specific for the two dominant wheat gluten epitopes is approximately 16 per million CD4+ T cells; and in HLA-DQ2.5+ Celiac disease patients who exclude dietary gluten the median frequency is 5 per million (Christophersen A. et al., UEGW J 2014). Oral gluten challenge transiently increases the frequencies of CD4+ T cells specific for dominant gluten epitopes by 10-100-fold (Anderson R P et al. Gut 2005; Raki M et al. PNAS USA 2007), allowing otherwise undetectable gluten epitope-specific T cells to be quantified and characterized by ex vivo IFN-γ secretion assays such as ELISpot using PBMC or ELISA using plasma from whole blood incubated with gluten peptides.

In recent studies using blood collected before and 6 days after oral gluten challenge in HLA-DQ2.5+ Celiac disease patients, IFN-γ inducible protein (IP)-10 and interleukin(IL)-2 were found to increase more consistently and to relatively higher concentrations in plasma from whole blood incubated with dominant gluten peptides. In the majority of patients, incubation of whole blood collected before oral gluten challenge with gluten peptides stimulated IP-10 and sometimes IL-2 levels that were significantly higher than whole blood incubated with culture medium alone. After oral gluten challenge, IP-10 and IL-2 as well as IFN-γ were elevated in whole blood incubated with gluten peptides.

This observation suggested that rare antigen-specific CD4+ T cells could be detected in cytokine release assays measuring IP-10, optionally in combination with IL-2 and/or IFN-γ, in plasma from whole blood incubated with candidate peptides encompassing cognate epitopes.

This novel approach to mapping epitopes for rare antigen-specific CD4+ T cells was tested in patients with Type-1 diabetes (T1D) who also had Celiac disease. T1D is also a T-cell mediated disease, but the epitopes recognized by disease-causing CD4+ T cells is unclear. The HLA-DR3-DQ2.5 and HLA-DR4-DQ8 haplotypes are strongly associated with T1D, and T-cell epitopes derived from autoantigens recognized by IgG in T1D have been reported. However, no single HLA-DR or DQ-restricted epitope has been consistently associated with T1D, and many regions of the most thoroughly studied islet autoantigens (preproinsulin, glutamic acid decarboxylase(GAD)-65, and insulinoma-associated antigen(IA)-2) are capable of being recognized by CD4+ T cells in MHC Class II transgenic mice, healthy human and/or T1D patients (Di Lorenzo T P et al., Clin Exp Immunol 2007).

In patients with T1D and Celiac disease, oral gluten challenge would allow CD4+ T cell responses to well-characterized gluten-derived epitopes to be used as a positive control for whole blood IFN-γ, IL-2 and IP-10 responses elicited by peptides derived from autoantigens implicated in T1D. Mapping epitopes relevant to T1D might then possible for the first time using fresh blood in overnight assays. In the current study, cytokine responses to pools and individual peptides encompassing all unique 12mers in the T1D autoantigens, preproinsulin, GAD65 and IA-2 were assessed before and after oral gluten challenge in T1D patients with Celiac disease following a gluten exclusion diet.

### Methods

Subjects were eligible if they were adults aged 18-55 yrs, had not received immunosuppressive medication within the previous 3-months, and adhered to a gluten-free diet. All subjects had biopsy-confirmed Celiac disease and were also insulin-treated Type-1 diabetics. Autoantibodies associated with T1D and Celiac disease were assessed (Barbara Davis Center, Denver Colo.; and Quest Diagnostics). Each of the four subjects showed elevated insulin autoantibodies, but autoantibodies specific for glutamic acid decarboxylase (GAD), insulinoma-associated antigen (IA)-2, or zinc-transporter-8 were not detected (Table 6). MHC Class-II alleles were determined (Barbara Davis Center, Denver Colo.) and showed each of the four subjects possessed alleles encoding HLA-DR3 and HLA-DQ2.5, and three also possessed HLA-DR4 and DQ8 (Table 7). All subjects strictly adhered to a gluten free diet until undergoing a 3-day oral food challenge. The food consumed during the challenge consisted of cookies prepared from wheat gluten, and barley and rye flour. Three cookies consumed daily were estimated to provide a total of 4.5 g wheat gluten, 3 g barley hordein, and 1.5 g rye secalin. Heparinized blood was collected before and 6 days after commencing the oral food challenge.

TABLE 6
Subject demographics and serology

| | | | Glutamic acid | IA-2 | Insulin | | Deamidated | |
|---|---|---|---|---|---|---|---|---|
| | | | decarboxylase | IgG | IgG | ZnT8-IgG | gliadin | tTG-IgA |
| | | | IgG (N < 20; | (N < 5,; | (N < 0.011, | (N < 0.021; | peptide IgA | (N < 0.050; |
| Subject | Age | Sex | hi > 25 | hi > 7) | hi > 0.013) | hi > 0.030) | IgA(N < 20) | hi > 0.100) |
| 1 | 28 | M | 0 | 0 | 0.259 | −0.004 | 21 | 0.033 |
| 7 | 23 | F | 12 | 2 | 0.168 | −0.005 | 6 | 0.094 |
| 6 | 44 | M | 0 | 0 | 1.206 | −0.007 | 22 | 0.079 |
| 2 | 22 | M | 0 | 0 | 0.089 | −0.003 | 28 | 0.006 |

TABLE 7
MHC Class II alleles of Subjects

| | T1D | | | | | | |
|---|---|---|---|---|---|---|---|
| | Susceptibility | | | | | | |
| Sub- | HLA-DR, | HLA- | HLA- | HLA- | HLA- | HLA- | HLA- |
| ject | and -DQ | DQA | DQA | DQB | DQB | DRB1 | DRB1 |
| 1 | DR3/4 DQ2.5/8 | 301 | 501 | 302 | 201 | 401 | 301 |
| 7 | DR3/4 DQ2.5/8 | 501 | 301 | 201 | 302 | 301 | 402 |
| 6 | DR3 DQ2.5 | 501 | 401 | 201 | 402 | 301 | 801 |
| 2 | DR3/4 DQ2.5/8 | 303 | 501 | 302 | 201 | 405 | 301 |

IFN-γ ELISpot and whole blood cytokine release assays were performed to assess T-cell responses to pools of 17mer peptides encompassing all unique 12mer amino-acid sequences derived from pancreatic islet antigens commonly recognized by autoantibodies in T1D: insulin, glutamic decarboxylase-65 (GAD65), and insulinoma-associated antigen(IA)-2 (purity 70% by HPLC, identity confirmed by LC-MS; synthesized by JPT Peptide Technologies GmbH, Germany) (Table 8). Libraries were designed according to Beissbarth et al (Bioninformatics T. et al. Suppl 1:i29-37) using protein sequences sourced from the public NCBI Genbank database (ncbi.nlm.nih.gov/protein). Table 8 summarizes the resulting peptide libraries.

TABLE 8
Islet autoantigen peptide libraries

| Library | Insulin | GAD65 | IA-2 |
|---|---|---|---|
| Antigen: | Preproinsulin | Glutamic | Insulinoma antigen-2 |
| | | decarboxylase 65 | (IA-2) or Tyrosine |
| | | (GAD65) | phosphatase like |
| | | | autoantigen (PTPRN) or |
| | | | (ICA512) |
| Search terms | “homo sapiens” | “homo sapiens” and | “Receptor-type tyrosine- |
| | and | “glutamate | protein phosphatase-like |
| | “preproinsulin”, or | decarboxylase”, or | N” |
| | “proinsulin”, or | “glutamic | |
| | “insulin” | decarboxylase”, or | |
| | | “glutamic” and | |
| | | “decarboxylase” and | |
| | | “65” | |
| Genbank entries | 19 | 20 | 3 |
| retrieved | | | |
| Unique 12mers | 154 | 692 | 1141 |
| Total 17mers in | 34 | 151 | 255 |
| library | | | |
| Pools per | 1 | 2 | 3 |
| library | | | |
| 17mers per pool | 34 | 75 or 76 | 85 |
| Deamidation - | 1 in AAP35454.1, | 4 in NP_001127838.1, | 15 in Q16849 |
| additional | AAA59179.1, | 1 in CAB62572.1, | |
| sequences | AEG19452.1, and | 4 in CAA49554.1, | |
| generated with | ABI63346.1 | 2 in AAB28987.1, | |
| glutamate | | 1 in EAW86101.1, | |
| substitutions at | | 2 in CAH73660.1, | |
| potential | | and 3 in CAH73658.1 | |
| deamidation | | | |
| sites¹ | | | |
| Genbank | 0601246A | EAW86103.1 | NP_001186692.1 |
| accession | AAA59172.1 | AAA58491.1 | NP_001186693.1 |
| numbers: | AAA59172.1 | AAA62367.1 | Q16849.1 |
| | AAA59173.1 | AAB28987.1 | |
| | AAA59179.1 | AAI26328.1 | |
| | AAH05255.1 | AAI26330.1 | |
| | AAN39451.1 | AAP88040.1 | |
| | AAP35454.1 | CAA49554.1 | |
| | AAW83741.1 | CAB62572.1 | |
| | ABI63346.1 | CAC09233.1 | |
| | AEG19452.1 | CAH73658.1 | |
| | AFK93533.1 | CAH73659.1 | |
| | CAA08766.1 | CAH73660.1 | |
| | CAA23828.1 | EAW86101.1 | |
| | CAA49913.1 | EAW86102.1 | |
| | NP_000198.1 | EAW86104.1 | |
| | NP_001172026.1 | NP_000809.1 | |
| | NP_001172027.1 | NP_001127838.1 | |
| | P01308.1 | Q05329.1 | |
| | | Q5VZ30 | |

¹If a glutamine residue in the primary sequence conformed to the deamidation motif defined for transglutaminase-2 (QX₁PX₃, or QX₁X₂[F, Y, W, I, L, V], where X₁ and X₃ are not proline) a second primary sequence was generated with glutamine replaced by glutamate. This deamidated primary sequences was included amongst those used to generate peptide libraries.

In addition, a pool of 3 gluten peptides consisting of immune-dominant HLA-DQ2.5-restricted T cell epitopes (95% purity by HPLC, identity confirmed by LC-MS; synthesized by CS Bio CA), a pool with 10 additional gluten peptides consisting of immuno-dominant HLA-DQ2.5, -DQ8, and DQ2.2 restricted T cell epitopes (90% purity by HPLC, identity confirmed by LC-MS; synthesized by Pepscan Netherlands), a pool of 14 gluten peptides consisting of 11 from the two smaller pools (90% purity by HPLC, identity confirmed by LC-MS; synthesized by Pepscan Netherlands), a pool of 71 gluten-derived peptides consisting of all the peptides in the three smaller pools as well as 55 additional peptides consisting of sequences implicated in HLA-DQ2.5-associated Celiac disease (purity 70% by HPLC, identity confirmed by LC-MS; JPT Peptide Technologies) (Tye-Din et al. Sci Transl Med 2010), and of 23 MHC class-I (CEF) (Product no. 3615-1, Mabtech AG, Sweden) and 14 MHC class-II restricted epitopes derived from recall viral antigens (PM-CEFT-MHC-II, JPT Peptide Technologies GmbH, Germany) were also assessed. Individual constituent peptides from the preproinsulin, GAD, IA-2 and gluten pools were assessed in whole blood cytokine release assays using blood collected 6 days after commencing oral gluten challenge.

In whole blood assays 225 μL volumes of fresh heparinized blood were incubated with 25 μL of peptide in phosphate buffered saline (PBS) with dimethylsulfoxide (DMSO) to a final concentration of 0.05% or 0.1% in 96-well round-bottom plates. Concentrations of constituent peptides in islet-autoantigen pools in whole blood assays were 0.4, 1 or 4 μg/mL; for CEF 0.1 μg/mL; CEFT 1 μg/mL; 3-gluten-peptide pool 10, 20 or 50 μg/mL, 13-gluten-peptide pool 5, 10, or 25 μg/mL; 14-gluten-peptide pool 5, 10, or 25 μg/mL; 71-gluten-peptide pool 1, 5 or 10 μg/mL. The final concentration of individual peptides incubated in whole blood with 10% PBS was 20 μg/mL with 0.0.5% DMSO. Whole blood assays were incubated for 24 hours at 37° C. in 5% CO₂. At the conclusion of the incubation period, approximately 120 μL plasma was separated from blood after centrifuging plates and then transferred to a corresponding well in a “mirror image” sterile 96-well plate. Plates containing plasma were sealed with adhesive plastic coverslips and frozen at −80° C. Multiplex bead-based cytokine assays (MAGPIX®) were performed on plasma thawed while being centrifuged at room temperature for 10 min. Plasma was pipetted directly into dedicated 96-well plates for magnetic bead-based assays to measure the concentrations of interferon(IFN)-γ-induced protein-10 (IP-10), interleukin(IL)-2 and IFN-γ. For peptide pools assessed before and after oral gluten challenge, triplicate plasma samples were assessed and the final concentration was expressed as the mean of triplicates after removal of outliers. The assay blank was determined by the mean levels of cytokines in plasma from blood incubated with 10% PBS and 0.1% DMSO. After oral gluten challenge, individual peptides in libraries were incubated in duplicate wells with whole blood. Plasma from duplicate wells was combined and assessed in triplicate wells for the multiplex cytokine assay. Each 96-well MAGPIX® plate was considered a separate assay that included 6 replicates with plasma from whole blood incubated with medium only. For each cytokine, a standard curve was determined using standards provided by the manufacturer from 3.2 to 10,000 pg/mL. If a cytokine concentration was reported as being <3.2 pg/mL a value of 3.2 pg/mL was recorded, and if >10,000 pg/mL a value of 10,000 pg/mL was recorded. Cytokine levels were considered elevated if the mean cytokine concentration in triplicate test wells was at least twice the mean level in 6 replicate wells in the same 96-well plate containing plasma from whole blood incubated with medium only.

In ELISpot assays, 0.4 million freshly isolated peripheral blood mononuclear cells (PBMC) re-suspended in 50 μL X-Vivo® serum-free medium were incubated with peptide pools dissolved in 40 μL X-Vivo® and 10 μL PBS. IFN-γ ELISpot assays were performed in pre-coated 96-well MAIP plates (Mabtech) that were incubated at 37° C. in 5% CO₂. After 18 h, PBMCs were discarded from ELISpot plates, and plates were washed and developed for later analysis by an automated ELISpot reader (Zellnet Inc., NJ). Spot forming counts were determined for each well.

### Results

### Gluten Peptide Pool Responses:

Cytokine release responses to pools and individual gluten-derived peptides were either unchanged or substantially increased after oral gluten challenge (Table 9 and Tables 10A-C). Amongst the whole blood cytokine release assays, responses to gluten peptides were least common and weakest in the whole blood IFN-γ release assay and most consistent and pronounced in the IP-10 assay after oral gluten challenge. However, IP-10 responses to the two larger gluten peptide pools were more than double those to medium alone in blood collected before as well as after oral gluten challenge in all four subjects. IL-2 responses to the highest concentrations (10-50 μg/mL) of gluten peptide pools were also more than double responses to medium alone in blood collected before oral gluten challenge in three subjects. Whole blood IFN-γ release and IFN-γ ELISpot responses to gluten peptide pools were not elevated before oral gluten challenge.

TABLE 9
IFNg ELISpot responses¹ to peptide pools²

| | Subject | | | | | | | |
|---|---|---|---|---|---|---|---|---|
| | 1 | | 7 | | 6 | | 2 | |
| | HLA-DR | | | | | | | |
| | 3, 4 | | 3, 4 | | 3 | | 3, 4 | |
| | HLA-DQ | | | | | | | |
| | 2.5, 8 | | 2.5, 8 | | 2.5 | | 2.5, 8 | |
| | Days since commencing oral challenge | | | | | | | |
| | 0 | 6 | 0 | 6 | 0 | 6 | 0 | 6 |
| Medium & PBMC 0.5% DMSO | 0 | 0 | 1 | 5 | 0 | 0 | 0 | 0 |
| Medium & PBMC 0.5% DMSO | 4 | 1 | 7 | 7 | 2 | 7 | 5 | 4 |
| Medium no PBMC 0.5% DMSO | 2 | 1 | 4 | 7 | 3 | 5 | 3 | 2 |
| PHA 10 ug/mL 0.5% DMSO | 2171 | 1730 | 1263 | 2109 | 1693 | 763 | 2487 | 2784 |
| Recall antigen pools | | | | | | | | |
| CEF each peptide 0.1 ug/mL | 249 | 75 | 12 | 45 | 42 | 74 | 11 | 14 |
| CEFT each peptide 1 ug/mL | 9 | 5 | 22 | 36 | 34 | 59 | 3 | 16 |
| Islet autoantigen-derived pools | | | | | | | | |
| Insulin - 34 peptides 17mers 20 ug/mL | 222 | 147 | 92 | 410 | 141 | 25 | 261 | 338 |
| GAD65 pool 1 - 75 17mers 20 ug/mL | 21 | 1 | 11 | 20 | 5 | 21 | 8 | 7 |
| GAD65 pool 2 - 76 17mers 20 ug/mL | 84 | 3 | 14 | 18 | 7 | 15 | 3 | 4 |
| IA2 pool 1 - 85 17mers 20 ug/mL | 6 | 3 | 8 | 17 | 19 | 32 | 2 | 7 |
| IA2 pool 2 - 85 17mers 20 ug/mL | 56 | 6 | 76 | 95 | 71 | 151 | 19 | 23 |
| IA2 pool 3 - 85 17mers 20 ug/mL | 23 | 1 | 9 | 22 | 24 | 40 | 3 | 7 |
| Gluten pools (epitope restrictions) | | | | | | | | |
| 71-peptides (DQ2.5/2.2/8) 50 uM | 2 | 4 | 8 | 53 | 8 | 354 | 2 | 11 |
| 14-peptides (DQ2.5/2.2/8) 25 uM | 2 | 4 | 8 | 53 | 8 | 354 | 2 | 11 |
| 13-peptides (DQ2.5/2.2/8) 25 uM | 2 | 2 | 4 | 65 | 2 | 281 | 3 | 9 |
| 3-peptides (DQ2.5) 50 ug/mL | 2 | 3 | 4 | 19 | 5 | 270 | 4 | 2 |

¹Spot forming units (sum of 3 wells, 0.4 million PBMC per well)
²Pools with individual peptides at concentrations indicated in a final concentration of 0.5% DMSO

TABLE 10A
Whole blood IFNg release to Gluten Peptide Pools

| Subject | | | | | | | |
|---|---|---|---|---|---|---|---|
| 1 | | 7 | | 6 | | 2 | |
| HLA-DR | | | | | | | |
| 3, 4 | | 3, 4 | | 3 | | 3, 4 | |
| HLA-DQ | | | | | | | |
| 2.5, 8 | | 2.5, 8 | | 2.5 | | 2.5, 8 | |
| Days since commencing oral challenge | | | | | | | |
| 0 | 6 | 0 | 6 | 0 | 6 | 0 | 6 |

Concentration pg/mL

| | | | | | | | | |
|---|---|---|---|---|---|---|---|---|
| Medium 0.05% | 55 | 66 | 239 | 150 | 97 | 98 | 3.8 | 4.9 |
| CEF 0.1 ug/mL | 44 | 53 | 458 | 172 | 96 | 85 | 4.0 | 4.4 |
| CEFT 1 ug/mL | 56 | 66 | 394 | 254 | 112 | 81 | 7.4 | 8.3 |
| 71-peptides (DQ2.5/2.2/8) 10 ug/mL | 76 | 75 | 391 | 247 | 93 | 277 | 6.2 | 25.1 |
| 71-peptides (DQ2.5/2.2/8) 5 ug/mL | 32 | 73 | 348 | 224 | 100 | 305 | 4.2 | 15.7 |
| 71-peptides (DQ2.5/2.2/8) 1 ug/mL | 87 | 71 | 201 | 216 | 83 | 185 | 3.5 | 13.3 |
| 14-peptides (DQ2.5/2.2/8) 25 uM | 54 | 78 | 450 | 238 | 83 | 105 | 6.5 | 4.5 |
| 14-peptides (DQ2.5/2.2/8) 10 uM | 52 | 63 | 154 | 201 | 74 | 109 | 3.7 | 3.9 |
| 14-peptides (DQ2.5/2.2/8) 5 uM | 43 | 65 | 133 | 213 | 91 | 136 | 3.8 | 6.8 |
| 13-peptides (DQ2.5/2.2/8) 25 uM | 71 | 69 | 454 | 220 | 90 | 198 | 4.0 | 8.1 |
| 13-peptides (DQ2.5/2.2/8) 10 uM | 60 | 76 | 424 | 409 | 74 | 163 | 3.2 | 9.8 |
| 13-peptides (DQ2.5/2.2/8) 5 uM | 51 | 76 | 169 | 292 | 93 | 134 | 3.2 | 12.7 |
| 3-peptides (DQ2.5) 50 ug/mL | 61 | 74 | 410 | 227 | 85 | 234 | 3.9 | 8.3 |
| 3-peptides (DQ2.5) 20 ug/mL | 58 | 58 | 78 | 266 | 98 | 245 | 3.2 | 5.8 |
| 3-peptides (DQ2.5) 10 ug/mL | 69 | 64 | 296 | 205 | 91 | 192 | 3.2 | 5.7 |
| (pE)PEQPIPEQPQPYPQQ-NH2 | 42 | 72 | 170 | 102 | 96 | 278 | 3.3 | 8.6 |
| (SEQ ID NO: 602) 10 uM | | | | | | | | |
| (pE)QPFPQPEQPFPWQP-NH2 | 60 | 62 | 158 | 150 | 94 | 258 | 4.5 | 6.4 |
| (SEQ ID NO: 603) 10 uM | | | | | | | | |
| (pE)LQPFPQPELPYPQPQ-NH2 | 39 | 55 | 407 | 194 | 102 | 200 | 4.1 | 13.9 |
| (SEQ ID NO: 604) 10 uM | | | | | | | | |

Fold-change over medium only (bold > 2)

| | | | | | | | | |
|---|---|---|---|---|---|---|---|---|
| Medium 0.05% | 1.00 | 1.00 | 1.00 | 1.00 | 1.00 | 1.00 | 1.00 | 1.00 |
| CEF 0.1 ug/mL | 0.80 | 0.81 | 1.92 | 1.14 | 0.99 | 0.86 | 1.03 | 0.90 |
| CEFT 1 ug/mL | 1.03 | 1.01 | 1.65 | 1.69 | 1.16 | 0.83 | 1.94 | 1.70 |
| 71-peptides (DQ2.5/2.2/8) 10 ug/mL | 1.39 | 1.14 | 1.64 | 1.64 | 0.97 | 2.83 | 1.62 | 5.15 |
| 71-peptides (DQ2.5/2.2/8) 5 ug/mL | 0.59 | 1.11 | 1.46 | 1.49 | 1.04 | 3.11 | 1.09 | 3.23 |
| 71-peptides (DQ2.5/2.2/8) 1 ug/mL | 1.59 | 1.08 | 0.84 | 1.44 | 0.86 | 1.89 | 0.90 | 2.74 |
| 14-peptides (DQ2.5/2.2/8) 25 uM | 0.99 | 1.19 | 1.88 | 1.58 | 0.85 | 1.07 | 1.71 | 0.93 |
| 14-peptides (DQ2.5/2.2/8) 10 uM | 0.96 | 0.95 | 0.65 | 1.34 | 0.76 | 1.11 | 0.96 | 0.80 |
| 14-peptides (DQ2.5/2.2/8) 5 uM | 0.79 | 0.98 | 0.56 | 1.41 | 0.94 | 1.39 | 0.98 | 1.40 |
| 13-peptides (DQ2.5/2.2/8) 25 uM | 1.30 | 1.04 | 1.90 | 1.46 | 0.94 | 2.02 | 1.03 | 1.66 |
| 13-peptides (DQ2.5/2.2/8) 10 uM | 1.10 | 1.16 | 1.77 | 2.72 | 0.76 | 1.66 | 0.84 | 2.01 |
| 13-peptides (DQ2.5/2.2/8) 5 uM | 0.93 | 1.15 | 0.71 | 1.94 | 0.96 | 1.36 | 0.84 | 2.61 |
| 3-peptides (DQ2.5) 50 ug/mL | 1.12 | 1.13 | 1.72 | 1.51 | 0.88 | 2.38 | 1.01 | 1.71 |
| 3-peptides (DQ2.5) 20 ug/mL | 1.06 | 0.88 | 0.33 | 1.77 | 1.01 | 2.50 | 0.84 | 1.18 |
| 3-peptides (DQ2.5) 10 ug/mL | 1.27 | 0.98 | 1.24 | 1.36 | 0.94 | 1.96 | 0.84 | 1.17 |
| (pE)PEQPIPEQPQPYPQQ-NH2 | 0.76 | 1.09 | 0.71 | 0.68 | 0.99 | 2.84 | 0.85 | 1.77 |
| (SEQ ID NO: 602) 10 uM | | | | | | | | |
| (pE)QPFPQPEQPFPWQP-NH2 | 1.09 | 0.95 | 0.66 | 1.00 | 0.98 | 2.63 | 1.19 | 1.32 |
| (SEQ ID NO: 603) 10 uM | | | | | | | | |
| (pE)LQPFPQPELPYPQPQ-NH2 | 0.71 | 0.84 | 1.71 | 1.29 | 1.05 | 2.03 | 1.06 | 2.85 |
| (SEQ ID NO: 604) 10 uM | | | | | | | | |

TABLE 10B
Whole blood IL-2 release to Gluten Peptide Pools

| Subject | | | | | | | |
|---|---|---|---|---|---|---|---|
| 1 | | 7 | | 6 | | 2 | |
| HLA-DR | | | | | | | |
| 3, 4 | | 3, 4 | | 3 | | 3, 4 | |
| HLA-DQ | | | | | | | |
| 2.5, 8 | | 2.5, 8 | | 2.5 | | 2.5, 8 | |
| Days since commencing oral challenge | | | | | | | |
| 0 | 6 | 0 | 6 | 0 | 6 | 0 | 6 |

Concentration pg/mL

| | | | | | | | | |
|---|---|---|---|---|---|---|---|---|
| Medium 0.05% | 3.2 | 3.2 | 62.7 | 42.1 | 5.0 | 4.2 | 3.2 | 3.2 |
| CEF 0.1 ug/mL | 3.2 | 3.2 | 195.5 | 53.9 | 4.5 | 3.7 | 3.2 | 3.2 |
| CEFT 1 ug/mL | 3.2 | 3.2 | 228.5 | 135.5 | 22.2 | 20.8 | 6.5 | 7.4 |
| 71-peptides (DQ2.5/2.2/8) 10 ug/mL | 15.0 | 13.1 | 169.9 | 162.6 | 30.5 | 110.2 | 3.2 | 50.4 |
| 71-peptides (DQ2.5/2.2/8) 5 ug/mL | 3.7 | 26.5 | 118.5 | 117.2 | 36.6 | 139.3 | 3.2 | 19.0 |
| 71-peptides (DQ2.5/2.2/8) 1 ug/mL | 3.4 | 4.4 | 61.8 | 127.6 | 16.9 | 73.6 | 3.2 | 13.1 |
| 14-peptides (DQ2.5/2.2/8) 25 uM | 5.8 | 25.9 | 165.7 | 191.2 | 3.2 | 6.0 | 3.2 | 3.2 |
| 14-peptides (DQ2.5/2.2/8) 10 uM | 4.1 | 7.6 | 32.7 | 117.0 | 8.5 | 14.8 | 3.2 | 3.2 |
| 14-peptides (DQ2.5/2.2/8) 5 uM | 3.2 | 11.7 | 37.5 | 125.7 | 4.9 | 23.5 | 3.2 | 3.2 |
| 13-peptides (DQ2.5/2.2/8) 25 uM | 3.2 | 9.6 | 162.1 | 167.2 | 11.7 | 92.2 | 3.2 | 3.2 |
| 13-peptides (DQ2.5/2.2/8) 10 uM | 3.2 | 5.8 | 110.4 | 128.2 | 12.5 | 89.7 | 3.2 | 3.2 |
| 13-peptides (DQ2.5/2.2/8) 5 uM | 3.2 | 14.2 | 56.2 | 186.2 | 7.4 | 60.0 | 3.2 | 29.6 |
| 3-peptides (DQ2.5) 50 ug/mL | 3.2 | 10.6 | 149.5 | 121.4 | 7.6 | 82.6 | 3.2 | 3.2 |
| 3-peptides (DQ2.5) 20 ug/mL | 3.2 | 3.2 | 20.8 | 131.4 | 22.7 | 110.3 | 3.2 | 3.2 |
| 3-peptides (DQ2.5) 10 ug/mL | 3.2 | 5.6 | 92.8 | 106.5 | 9.0 | 58.0 | 3.2 | 3.2 |
| (pE)PEQPIPEQPQPYPQQ-NH2 | 3.2 | 3.2 | 106.1 | 27.8 | 23.3 | 134.3 | 3.2 | 22.4 |
| (SEQ ID NO: 602) 10 uM | | | | | | | | |
| (pE)QPFPQPEQPFPWQP-NH2 | 3.2 | 3.2 | 41.4 | 55.1 | 17.9 | 115.9 | 3.2 | 5.2 |
| (SEQ ID NO: 603) 10 uM | | | | | | | | |
| (pE)LQPFPQPELPYPQPQ-NH2 | 3.2 | 3.2 | 144.4 | 71.2 | 12.5 | 88.5 | 3.2 | 12.2 |
| (SEQ ID NO: 604) 10 uM | | | | | | | | |

Fold-change over medium only (bold > 2)

| | | | | | | | | |
|---|---|---|---|---|---|---|---|---|
| Medium 0.05% | 1.00 | 1.00 | 1.00 | 1.00 | 1.00 | 1.00 | 1.00 | 1.00 |
| CEF 0.1 ug/mL | 1.00 | 1.00 | 3.12 | 1.28 | 0.91 | 0.87 | 1.00 | 1.00 |
| CEFT 1 ug/mL | 1.00 | 1.00 | 3.65 | 3.22 | 4.48 | 4.92 | 2.03 | 2.30 |
| 71-peptides (DQ2.5/2.2/8) 10 ug/mL | 4.70 | 4.11 | 2.71 | 3.86 | 6.17 | 25.99 | 1.00 | 15.75 |
| 71-peptides (DQ2.5/2.2/8) 5 ug/mL | 1.14 | 8.28 | 1.89 | 2.78 | 7.39 | 32.84 | 1.00 | 5.94 |
| 71-peptides (DQ2.5/2.2/8) 1 ug/mL | 1.06 | 1.38 | 0.99 | 3.03 | 3.42 | 17.36 | 1.00 | 4.10 |
| 14-peptides (DQ2.5/2.2/8) 25 uM | 1.82 | 8.08 | 2.64 | 4.54 | 0.65 | 1.41 | 1.00 | 1.00 |
| 14-peptides (DQ2.5/2.2/8) 10 uM | 1.28 | 2.38 | 0.52 | 2.78 | 1.72 | 3.48 | 1.00 | 1.00 |
| 14-peptides (DQ2.5/2.2/8) 5 uM | 1.00 | 3.65 | 0.60 | 2.99 | 0.99 | 5.55 | 1.00 | 1.00 |
| 13-peptides (DQ2.5/2.2/8) 25 uM | 1.00 | 2.99 | 2.59 | 3.97 | 2.36 | 21.74 | 1.00 | 1.00 |
| 13-peptides (DQ2.5/2.2/8) 10 uM | 1.00 | 1.82 | 1.76 | 3.04 | 2.52 | 21.16 | 1.00 | 1.00 |
| 13-peptides (DQ2.5/2.2/8) 5 uM | 1.00 | 4.44 | 0.90 | 4.42 | 1.49 | 14.16 | 1.00 | 9.25 |
| 3-peptides (DQ2.5) 50 ug/mL | 1.00 | 3.30 | 2.39 | 2.88 | 1.53 | 19.49 | 1.00 | 1.00 |
| 3-peptides (DQ2.5) 20 ug/mL | 1.00 | 1.00 | 0.33 | 3.12 | 4.58 | 26.02 | 1.00 | 1.00 |
| 3-peptides (DQ2.5) 10 ug/mL | 1.00 | 1.75 | 1.48 | 2.53 | 1.81 | 13.67 | 1.00 | 1.00 |
| (pE)PEQPIPEQPQPYPQQ-NH2 | 1.00 | 1.00 | 1.69 | 0.66 | 4.71 | 31.67 | 1.00 | 7.00 |
| (SEQ ID NO: 602) 10 uM | | | | | | | | |
| (pE)QPFPQPEQPFPWQP-NH2 | 1.00 | 1.00 | 0.66 | 1.31 | 3.62 | 27.33 | 1.00 | 1.61 |
| (SEQ ID NO: 603) 10 uM | | | | | | | | |
| (pE)LQPFPQPELPYPQPQ-NH2 | 1.00 | 1.00 | 2.30 | 1.69 | 2.52 | 20.88 | 1.00 | 3.82 |
| (SEQ ID NO: 604) 10 uM | | | | | | | | |

TABLE 10C
Whole blood IP-10 release to Gluten Peptide Pools

| Subject | | | | | | | |
|---|---|---|---|---|---|---|---|
| 1 | | 7 | | 6 | | 2 | |
| HLA-DR | | | | | | | |
| 3, 4 | | 3, 4 | | 3 | | 3, 4 | |
| HLA-DQ | | | | | | | |
| 2.5, 8 | | 2.5, 8 | | 2.5 | | 2.5, 8 | |
| Days since commencing oral challenge | | | | | | | |
| 0 | 6 | 0 | 6 | 0 | 6 | 0 | 6 |

Concentration pg/mL

| | | | | | | | | |
|---|---|---|---|---|---|---|---|---|
| Medium 0.05% | 266 | 335 | 743 | 892 | 277 | 201 | 266 | 312 |
| CEF 0.1 ug/mL | 225 | 346 | 1211 | 764 | 318 | 644 | 241 | 282 |
| CEFT 1 ug/mL | 586 | 604 | 10000 | 10000 | 5439 | 8789 | 937 | 1235 |
| 71-peptides (DQ2.5/2.2/8) 10 ug/mL | 688 | 1546 | 1805 | 10000 | 3479 | 10000 | 561 | 3696 |
| 71-peptides (DQ2.5/2.2/8) 5 ug/mL | 763 | 3100 | 1212 | 10000 | 3143 | 10000 | 464 | 2957 |
| 71-peptides (DQ2.5/2.2/8) 1 ug/mL | 582 | 854 | 2061 | 10000 | 2744 | 10000 | 578 | 1969 |
| 14-peptides (DQ2.5/2.2/8) 25 uM | 949 | 2132 | 2662 | 10000 | 219 | 1852 | 266 | 265 |
| 14-peptides (DQ2.5/2.2/8) 10 uM | 326 | 1554 | 1378 | 10000 | 902 | 3621 | 242 | 273 |
| 14-peptides (DQ2.5/2.2/8) 5 uM | 226 | 852 | 1448 | 10000 | 523 | 9960 | 271 | 540 |
| 13-peptides (DQ2.5/2.2/8) 25 uM | 328 | 945 | 1482 | 10000 | 886 | 10000 | 258 | 421 |
| 13-peptides (DQ2.5/2.2/8) 10 uM | 264 | 1010 | 907 | 10000 | 1238 | 10000 | 214 | 1330 |
| 13-peptides (DQ2.5/2.2/8) 5 uM | 234 | 1379 | 910 | 10000 | 448 | 10000 | 252 | 2724 |
| 3-peptides (DQ2.5) 50 ug/mL | 233 | 980 | 955 | 10000 | 320 | 10000 | 228 | 498 |
| 3-peptides (DQ2.5) 20 ug/mL | 249 | 419 | 536 | 10000 | 2479 | 10000 | 229 | 671 |
| 3-peptides (DQ2.5) 10 ug/mL | 316 | 643 | 843 | 10000 | 1329 | 10000 | 253 | 695 |
| (pE)PEQPIPEQPQPYPQQ-NH2 | 235 | 325 | 713 | 2549 | 3409 | 10000 | 336 | 1269 |
| (SEQ ID NO: 602) 10 uM | | | | | | | | |
| (pE)QPFPQPEQPFPWQP-NH2 | 218 | 501 | 900 | 10000 | 1934 | 10000 | 376 | 1140 |
| (SEQ ID NO: 603) 10 uM | | | | | | | | |
| (pE)LQPFPQPELPYPQPQ-NH2 | 201 | 384 | 977 | 3293 | 1616 | 10000 | 320 | 1844 |
| (SEQ ID NO: 604) 10 uM | | | | | | | | |

Fold-change over medium only (bold > 2)

| | | | | | | | | |
|---|---|---|---|---|---|---|---|---|
| Medium 0.05% | 1.00 | 1.00 | 1.00 | 1.00 | 1.00 | 1.00 | 1.00 | 1.00 |
| CEF 0.1 ug/mL | 0.85 | 1.03 | 1.63 | 0.86 | 1.15 | 3.21 | 0.91 | 0.90 |
| CEFT 1 ug/mL | 2.21 | 1.80 | 13.46 | 11.21 | 19.62 | 43.73 | 3.53 | 3.95 |
| 71-peptides (DQ2.5/2.2/8) 10 ug/mL | 2.59 | 4.61 | 2.43 | 11.21 | 12.55 | 49.76 | 2.11 | 11.83 |
| 71-peptides (DQ2.5/2.2/8) 5 ug/mL | 2.87 | 9.25 | 1.63 | 11.21 | 11.34 | 49.76 | 1.75 | 9.47 |
| 71-peptides (DQ2.5/2.2/8) 1 ug/mL | 2.19 | 2.55 | 2.77 | 11.21 | 9.90 | 49.76 | 2.18 | 6.30 |
| 14-peptides (DQ2.5/2.2/8) 25 uM | 3.58 | 6.36 | 3.58 | 11.21 | 0.79 | 9.21 | 1.00 | 0.85 |
| 14-peptides (DQ2.5/2.2/8) 10 uM | 1.23 | 4.64 | 1.85 | 11.21 | 3.25 | 18.02 | 0.91 | 0.87 |
| 14-peptides (DQ2.5/2.2/8) 5 uM | 0.85 | 2.54 | 1.95 | 11.21 | 1.89 | 49.56 | 1.02 | 1.73 |
| 13-peptides (DQ2.5/2.2/8) 25 uM | 1.24 | 2.82 | 1.99 | 11.21 | 3.19 | 49.76 | 0.97 | 1.35 |
| 13-peptides (DQ2.5/2.2/8) 10 uM | 1.00 | 3.01 | 1.22 | 11.21 | 4.47 | 49.76 | 0.81 | 4.26 |
| 13-peptides (DQ2.5/2.2/8) 5 uM | 0.88 | 4.11 | 1.23 | 11.21 | 1.62 | 49.76 | 0.95 | 8.72 |
| 3-peptides (DQ2.5) 50 ug/mL | 0.88 | 2.92 | 1.29 | 11.21 | 1.16 | 49.76 | 0.86 | 1.60 |
| 3-peptides (DQ2.5) 20 ug/mL | 0.94 | 1.25 | 0.72 | 11.21 | 8.94 | 49.76 | 0.86 | 2.15 |
| 3-peptides (DQ2.5) 10 ug/mL | 1.19 | 1.92 | 1.14 | 11.21 | 4.79 | 49.76 | 0.95 | 2.22 |
| (pE)PEQPIPEQPQPYPQQ-NH2 | 0.89 | 0.97 | 0.96 | 2.86 | 12.30 | 49.76 | 1.26 | 4.06 |
| (SEQ ID NO: 602) 10 uM | | | | | | | | |
| (pE)QPFPQPEQPFPWQP-NH2 | 0.82 | 1.49 | 1.21 | 11.21 | 6.98 | 49.76 | 1.42 | 3.65 |
| (SEQ ID NO: 603) 10 uM | | | | | | | | |
| (pE)LQPFPQPELPYPQPQ-NH2 | 0.76 | 1.15 | 1.32 | 3.69 | 5.83 | 49.76 | 1.20 | 5.90 |
| (SEQ ID NO: 604) 10 uM | | | | | | | | |

### T1D Autoantigen Peptide Pool Responses:

Responses to T1D autoantigen peptide pools in cytokine release assays, especially whole blood IP-10 release, were frequently elevated (Table 9 and Tables 11A-C). The highest concentration of peptide pools tested (4 μg/mL) almost always stimulated stronger responses than the lowest concentration (0.4 μg/mL). With the exception of pool 1 for GAD65 in one patient, each of the eight T1D autoantigen peptide pools elicited whole blood IP-10 responses that were more than double those to medium alone in all four subjects. The preproinsulin pool and IA-2 pool 2 frequently stimulated strongest cytokine release responses, especially in the IFN-γ ELISpot. Consistency and magnitude of cytokine release stimulated by T1D autoantigen peptide pools was not consistently changed after oral gluten challenge.

TABLE 11A
Whole blood IFNg release to Islet-autoantigen Peptide Pools

| Subject | | | | | | | |
|---|---|---|---|---|---|---|---|
| 1 | | 7 | | 6 | | 2 | |
| HLA-DR | | | | | | | |
| 3, 4 | | 3, 4 | | 3 | | 3, 4 | |
| HLA-DQ | | | | | | | |
| 2.5, 8 | | 2.5, 8 | | 2.5 | | 2.5, 8 | |
| Days since commencing oral challenge | | | | | | | |
| 0 | 6 | 0 | 6 | 0 | 6 | 0 | 6 |

Concentration pg/mL

| | | | | | | | | |
|---|---|---|---|---|---|---|---|---|
| Medium 0.05% | 91 | 117 | 473 | 78 | 88 | 105 | 4.5 | 6.4 |
| CEF 0.1 ug/mL | 64 | 123 | 496 | 114 | 74 | 107 | 4.0 | 5.7 |
| CEFT 1 ug/mL | 58 | 105 | 553 | 113 | 82 | 113 | 6.3 | 5.9 |
| Insulin - 34 peptides 17mers 4 ug/mL | 76 | 149 | 910 | 211 | 111 | 124 | 24.2 | 47.7 |
| Insulin - 34 peptides 17mers 1 ug/mL | 69 | 111 | 678 | 105 | 90 | 97 | 11.4 | 11.0 |
| Insulin - 34 peptides 17mers 0.4 ug/mL | 66 | 129 | 461 | 65 | 85 | 86 | 4.8 | 6.8 |
| GAD65 pool 1 - 75 17mers 4 ug/mL | 80 | 167 | 650 | 226 | 108 | 104 | 6.7 | 8.8 |
| GAD65 pool 1 - 75 17mers 1 ug/mL | 67 | 72 | 607 | 77 | 90 | 98 | 3.5 | 5.5 |
| GAD65 pool 1 - 75 17mers 0.4 ug/mL | 67 | 121 | 592 | 59 | 75 | 85 | 4.0 | 5.4 |
| GAD65 pool 2 - 76 17mers 4 ug/mL | 84 | 126 | 532 | 139 | 113 | 106 | 6.4 | 10.7 |
| GAD65 pool 2 - 76 17mers 1 ug/mL | 67 | 124 | 597 | 146 | 76 | 105 | 6.2 | 7.7 |
| GAD65 pool 2 - 76 17mers 0.4 ug/mL | 56 | 131 | 592 | 87 | 70 | 96 | 7.3 | 6.0 |
| IA2 pool 1 - 85 17mers 4 ug/mL | 142 | 118 | 563 | 142 | 108 | 98 | 8.0 | 9.1 |
| IA2 pool 1 - 85 17mers 1 ug/mL | 64 | 62 | 517 | 99 | 74 | 81 | 3.5 | 5.7 |
| IA2 pool 1 - 85 17mers 0.4 ug/mL | 71 | 127 | 496 | 105 | 66 | 93 | 4.8 | 4.1 |
| IA2 pool 2 - 85 17mers 4 ug/mL | 68 | 164 | 509 | 173 | 146 | 140 | 31.9 | 32.5 |
| IA2 pool 2 - 85 17mers 1 ug/mL | 103 | 131 | 318 | 71 | 94 | 126 | 9.3 | 8.8 |
| IA2 pool 2 - 85 17mers 0.4 ug/mL | 74 | 29 | 472 | 163 | 76 | 87 | 5.6 | 4.6 |
| IA2 pool 3 - 85 17mers 4 ug/mL | 71 | 82 | 198 | 75 | 95 | 91 | 13.0 | 8.7 |
| IA2 pool 3 - 85 17mers 1 ug/mL | 53 | 90 | 258 | 44 | 70 | 73 | 4.1 | 6.3 |
| IA2 pool 3 - 85 17mers 0.4 ug/mL | 64 | 111 | 453 | 92 | 71 | 70 | 4.2 | 5.0 |

Fold-change over medium only (bold > 2)

| | | | | | | | | |
|---|---|---|---|---|---|---|---|---|
| Medium 0.05% | 1.00 | 1.00 | 1.00 | 1.00 | 1.00 | 1.00 | 1.00 | 1.00 |
| CEF 0.1 ug/mL | 0.70 | 1.05 | 1.05 | 1.46 | 0.84 | 1.02 | 0.88 | 0.89 |
| CEFT 1 ug/mL | 0.63 | 0.90 | 1.17 | 1.44 | 0.93 | 1.07 | 1.40 | 0.93 |
| Insulin - 34 peptides 17mers 4 ug/mL | 0.83 | 1.27 | 1.92 | 2.69 | 1.26 | 1.18 | 5.41 | 7.49 |
| Insulin - 34 peptides 17mers 1 ug/mL | 0.75 | 0.94 | 1.43 | 1.35 | 1.01 | 0.92 | 2.54 | 1.73 |
| Insulin - 34 peptides 17mers 0.4 ug/mL | 0.72 | 1.10 | 0.97 | 0.83 | 0.96 | 0.82 | 1.08 | 1.06 |
| GAD65 pool 1 - 75 17mers 4 ug/mL | 0.87 | 1.43 | 1.37 | 2.89 | 1.22 | 0.99 | 1.50 | 1.37 |
| GAD65 pool 1 - 75 17mers 1 ug/mL | 0.73 | 0.61 | 1.28 | 0.98 | 1.01 | 0.94 | 0.77 | 0.86 |
| GAD65 pool 1 - 75 17mers 0.4 ug/mL | 0.74 | 1.03 | 1.25 | 0.76 | 0.85 | 0.81 | 0.88 | 0.84 |
| GAD65 pool 2 - 76 17mers 4 ug/mL | 0.92 | 1.08 | 1.12 | 1.78 | 1.28 | 1.01 | 1.43 | 1.68 |
| GAD65 pool 2 - 76 17mers 1 ug/mL | 0.74 | 1.06 | 1.26 | 1.87 | 0.86 | 1.01 | 1.38 | 1.21 |
| GAD65 pool 2 - 76 17mers 0.4 ug/mL | 0.61 | 1.12 | 1.25 | 1.12 | 0.80 | 0.91 | 1.63 | 0.94 |
| IA2 pool 1 - 85 17mers 4 ug/mL | 1.56 | 1.01 | 1.19 | 1.82 | 1.22 | 0.93 | 1.78 | 1.43 |
| IA2 pool 1 - 85 17mers 1 ug/mL | 0.71 | 0.53 | 1.09 | 1.27 | 0.84 | 0.78 | 0.77 | 0.89 |
| IA2 pool 1 - 85 17mers 0.4 ug/mL | 0.78 | 1.09 | 1.05 | 1.34 | 0.75 | 0.89 | 1.08 | 0.65 |
| IA2 pool 2 - 85 17mers 4 ug/mL | 0.74 | 1.40 | 1.07 | 2.21 | 1.65 | 1.33 | 7.11 | 5.10 |
| IA2 pool 2 - 85 17mers 1 ug/mL | 1.13 | 1.12 | 0.67 | 0.91 | 1.06 | 1.20 | 2.08 | 1.37 |
| IA2 pool 2 - 85 17mers 0.4 ug/mL | 0.81 | 0.24 | 1.00 | 2.08 | 0.86 | 0.83 | 1.26 | 0.71 |
| IA2 pool 3 - 85 17mers 4 ug/mL | 0.78 | 0.70 | 0.42 | 0.96 | 1.08 | 0.87 | 2.90 | 1.36 |
| IA2 pool 3 - 85 17mers 1 ug/mL | 0.58 | 0.77 | 0.55 | 0.56 | 0.79 | 0.70 | 0.90 | 0.98 |
| IA2 pool 3 - 85 17mers 0.4 ug/mL | 0.70 | 0.95 | 0.96 | 1.18 | 0.80 | 0.67 | 0.93 | 0.78 |

TABLE 11B
Whole blood IL-2 release to Islet-autoantigen Peptide Pools

| Subject | | | | | | | |
|---|---|---|---|---|---|---|---|
| 1 | | 7 | | 6 | | 2 | |
| HLA-DR | | | | | | | |
| 3, 4 | | 3, 4 | | 3 | | 3, 4 | |
| HLA-DQ | | | | | | | |
| 2.5, 8 | | 2.5, 8 | | 2.5 | | 2.5, 8 | |
| Days since commencing oral challenge | | | | | | | |
| 0 | 6 | 0 | 6 | 0 | 6 | 0 | 6 |

Concentration pg/mL

| | | | | | | | | |
|---|---|---|---|---|---|---|---|---|
| Medium 0.05% | 3.2 | 3.2 | 162 | 23 | 3.6 | 4.3 | 3.2 | 3.2 |
| CEF 0.1 ug/mL | 3.2 | 3.2 | 211 | 33 | 3.2 | 5.0 | 3.2 | 3.2 |
| CEFT 1 ug/mL | 3.2 | 3.2 | 236 | 47 | 17.1 | 24.4 | 8.4 | 3.2 |
| Insulin - 34 peptides 17mers 4 ug/mL | 5.2 | 6.3 | 311 | 94 | 15.8 | 16.7 | 21.7 | 34.0 |
| Insulin - 34 peptides 17mers 1 ug/mL | 5.1 | 3.2 | 205 | 34 | 5.8 | 5.0 | 6.4 | 3.2 |
| Insulin - 34 peptides 17mers 0.4 ug/mL | 3.2 | 3.2 | 136 | 19 | 3.5 | 3.2 | 3.2 | 3.2 |
| GAD65 pool 1 - 75 17mers 4 ug/mL | 14.0 | 3.2 | 236 | 95 | 10.4 | 7.2 | 3.2 | 3.2 |
| GAD65 pool 1 - 75 17mers 1 ug/mL | 3.2 | 3.2 | 216 | 23 | 3.6 | 4.8 | 3.2 | 3.2 |
| GAD65 pool 1 - 75 17mers 0.4 ug/mL | 3.2 | 3.2 | 181 | 18 | 3.2 | 3.2 | 3.2 | 3.2 |
| GAD65 pool 2 - 76 17mers 4 ug/mL | 6.0 | 3.2 | 190 | 55 | 14.2 | 9.7 | 3.2 | 3.2 |
| GAD65 pool 2 - 76 17mers 1 ug/mL | 4.2 | 3.2 | 193 | 28 | 7.1 | 5.6 | 6.4 | 3.2 |
| GAD65 pool 2 - 76 17mers 0.4 ug/mL | 3.2 | 3.2 | 213 | 28 | 5.2 | 3.9 | 3.2 | 3.2 |
| IA2 pool 1 - 85 17mers 4 ug/mL | 46.1 | 5.4 | 264 | 83 | 23.0 | 18.7 | 37.5 | 56.3 |
| IA2 pool 1 - 85 17mers 1 ug/mL | 3.2 | 3.2 | 172 | 32 | 4.8 | 7.1 | 20.5 | 12.1 |
| IA2 pool 1 - 85 17mers 0.4 ug/mL | 3.2 | 3.2 | 168 | 30 | 3.2 | 3.2 | 3.2 | 3.2 |
| IA2 pool 2 - 85 17mers 4 ug/mL | 3.8 | 21.6 | 170 | 85 | 44.9 | 27.6 | 40.2 | 39.4 |
| IA2 pool 2 - 85 17mers 1 ug/mL | 23.9 | 6.2 | 77 | 21 | 12.2 | 21.7 | 14.6 | 3.2 |
| IA2 pool 2 - 85 17mers 0.4 ug/mL | 6.6 | 3.2 | 203 | 55 | 3.8 | 4.2 | 3.2 | 3.2 |
| IA2 pool 3 - 85 17mers 4 ug/mL | 3.2 | 3.2 | 10 | 12 | 5.2 | 6.2 | 33.3 | 29.1 |
| IA2 pool 3 - 85 17mers 1 ug/mL | 3.2 | 3.2 | 67 | 10 | 3.2 | 3.2 | 14.6 | 14.8 |
| IA2 pool 3 - 85 17mers 0.4 ug/mL | 3.2 | 3.2 | 190 | 29 | 3.2 | 3.2 | 21.3 | 3.2 |

Fold-change over medium only (bold > 2)

| | | | | | | | | |
|---|---|---|---|---|---|---|---|---|
| Medium 0.05% | 1.00 | 1.00 | 1.00 | 1.00 | 1.00 | 1.00 | 1.00 | 1.00 |
| CEF 0.1 ug/mL | 1.00 | 1.00 | 1.30 | 1.41 | 0.88 | 1.15 | 1.00 | 1.00 |
| CEFT 1 ug/mL | 1.00 | 1.00 | 1.46 | 2.03 | 4.72 | 5.65 | 2.62 | 1.00 |
| Insulin - 34 peptides 17mers 4 ug/mL | 1.63 | 1.97 | 1.92 | 4.08 | 4.36 | 3.88 | 6.78 | 10.62 |
| Insulin - 34 peptides 17mers 1 ug/mL | 1.60 | 1.00 | 1.27 | 1.47 | 1.59 | 1.16 | 2.00 | 1.00 |
| Insulin - 34 peptides 17mers 0.4 ug/mL | 1.00 | 1.00 | 0.84 | 0.84 | 0.95 | 0.74 | 1.00 | 1.00 |
| GAD65 pool 1 - 75 17mers 4 ug/mL | 4.37 | 1.00 | 1.46 | 4.09 | 2.88 | 1.66 | 1.00 | 1.00 |
| GAD65 pool 1 - 75 17mers 1 ug/mL | 1.00 | 1.00 | 1.34 | 1.00 | 1.00 | 1.10 | 1.00 | 1.00 |
| GAD65 pool 1 - 75 17mers 0.4 ug/mL | 1.00 | 1.00 | 1.12 | 0.79 | 0.88 | 0.74 | 1.00 | 1.00 |
| GAD65 pool 2 - 76 17mers 4 ug/mL | 1.86 | 1.00 | 1.18 | 2.37 | 3.92 | 2.24 | 1.00 | 1.00 |
| GAD65 pool 2 - 76 17mers 1 ug/mL | 1.32 | 1.00 | 1.19 | 1.19 | 1.97 | 1.29 | 2.01 | 1.00 |
| GAD65 pool 2 - 76 17mers 0.4 ug/mL | 1.00 | 1.00 | 1.32 | 1.22 | 1.45 | 0.90 | 1.00 | 1.00 |
| IA2 pool 1 - 85 17mers 4 ug/mL | 14.41 | 1.70 | 1.63 | 3.61 | 6.35 | 4.34 | 11.71 | 17.59 |
| IA2 pool 1 - 85 17mers 1 ug/mL | 1.00 | 1.00 | 1.06 | 1.39 | 1.32 | 1.65 | 6.40 | 3.77 |
| IA2 pool 1 - 85 17mers 0.4 ug/mL | 1.00 | 1.00 | 1.04 | 1.28 | 0.88 | 0.75 | 1.00 | 1.00 |
| IA2 pool 2 - 85 17mers 4 ug/mL | 1.19 | 6.74 | 1.05 | 3.70 | 12.40 | 6.39 | 12.57 | 12.30 |
| IA2 pool 2 - 85 17mers 1 ug/mL | 7.48 | 1.93 | 0.48 | 0.90 | 3.36 | 5.03 | 4.56 | 1.00 |
| IA2 pool 2 - 85 17mers 0.4 ug/mL | 2.05 | 1.00 | 1.25 | 2.36 | 1.04 | 0.98 | 1.00 | 1.00 |
| IA2 pool 3 - 85 17mers 4 ug/mL | 1.00 | 1.00 | 0.06 | 0.51 | 1.45 | 1.43 | 10.40 | 9.09 |
| IA2 pool 3 - 85 17mers 1 ug/mL | 1.00 | 1.00 | 0.41 | 0.44 | 0.88 | 0.74 | 4.57 | 4.63 |
| IA2 pool 3 - 85 17mers 0.4 ug/mL | 1.00 | 1.00 | 1.18 | 1.24 | 0.88 | 0.74 | 6.67 | 1.00 |

TABLE 11C
Whole blood IP-10 release to Islet-autoantigen Peptide Pools

| Subject | | | | | | | |
|---|---|---|---|---|---|---|---|
| 1 | | 7 | | 6 | | 2 | |
| HLA-DR | | | | | | | |
| 3, 4 | | 3, 4 | | 3 | | 3, 4 | |
| HLA-DQ | | | | | | | |
| 2.5, 8 | | 2.5, 8 | | 2.5 | | 2.5, 8 | |
| Days since commencing oral challenge | | | | | | | |
| 0 | 6 | 0 | 6 | 0 | 6 | 0 | 6 |

Concentration pg/mL

| | | | | | | | | |
|---|---|---|---|---|---|---|---|---|
| Medium 0.05% | 10000 | 342 | 861 | 599 | 260 | 206 | 309 | 359 |
| CEF 0.1 ug/mL | 264 | 325 | 775 | 658 | 716 | 399 | 270 | 303 |
| CEFT 1 ug/mL | 275 | 355 | 7407 | 5957 | 5857 | 9218 | 815 | 887 |
| Insulin - 34 peptides 17mers 4 ug/mL | 3183 | 9830 | 10000 | 10000 | 10000 | 10000 | 7297 | 5697 |
| Insulin - 34 peptides 17mers 1 ug/mL | 4716 | 3055 | 10000 | 7466 | 2606 | 3322 | 4015 | 3093 |
| Insulin - 34 peptides 17mers 0.4 ug/mL | 579 | 986 | 2281 | 1361 | 365 | 343 | 308 | 772 |
| GAD65 pool 1 - 75 17mers 4 ug/mL | 1913 | 1568 | 2171 | 1157 | 5406 | 1264 | 580 | 565 |
| GAD65 pool 1 - 75 17mers 1 ug/mL | 663 | 906 | 1130 | 681 | 681 | 462 | 334 | 370 |
| GAD65 pool 1 - 75 17mers 0.4 ug/mL | 284 | 608 | 967 | 797 | 326 | 294 | 382 | 327 |
| GAD65 pool 2 - 76 17mers 4 ug/mL | 5459 | 793 | 10000 | 9488 | 1972 | 2608 | 465 | 902 |
| GAD65 pool 2 - 76 17mers 1 ug/mL | 2180 | 903 | 10000 | 3692 | 3112 | 1225 | 1535 | 752 |
| GAD65 pool 2 - 76 17mers 0.4 ug/mL | 541 | 657 | 5150 | 1618 | 1800 | 1061 | 263 | 619 |
| IA2 pool 1 - 85 17mers 4 ug/mL | 10000 | 2496 | 10000 | 9969 | 4531 | 2775 | 1528 | 1376 |
| IA2 pool 1 - 85 17mers 1 ug/mL | 3121 | 1146 | 3363 | 1259 | 1450 | 1884 | 543 | 586 |
| IA2 pool 1 - 85 17mers 0.4 ug/mL | 1347 | 838 | 1296 | 836 | 264 | 300 | 298 | 299 |
| IA2 pool 2 - 85 17mers 4 ug/mL | 1653 | 5006 | 10000 | 10000 | 10000 | 10000 | 4721 | 5850 |
| IA2 pool 2 - 85 17mers 1 ug/mL | 8939 | 3346 | 10000 | 4159 | 7063 | 10000 | 2118 | 1183 |
| IA2 pool 2 - 85 17mers 0.4 ug/mL | 1442 | 427 | 1799 | 3332 | 2153 | 2902 | 418 | 273 |
| IA2 pool 3 - 85 17mers 4 ug/mL | 255 | 801 | 1194 | 897 | 1130 | 646 | 1984 | 1351 |
| IA2 pool 3 - 85 17mers 1 ug/mL | 752 | 789 | 986 | 553 | 307 | 253 | 386 | 619 |
| IA2 pool 3 - 85 17mers 0.4 ug/mL | 502 | 386 | 921 | 2347 | 234 | 197 | 360 | 344 |

Fold-change over medium only (bold > 2)

| | | | | | | | | |
|---|---|---|---|---|---|---|---|---|
| Medium 0.05% | 37.89 | 1.00 | 1.00 | 1.00 | 1.00 | 1.00 | 1.00 | 1.00 |
| CEF 0.1 ug/mL | 1.00 | 0.95 | 0.90 | 1.10 | 2.76 | 1.94 | 0.87 | 0.84 |
| CEFT 1 ug/mL | 1.04 | 1.04 | 8.60 | 9.94 | 22.53 | 44.82 | 2.63 | 2.47 |
| Insulin - 34 peptides 17mers 4 ug/mL | 12.06 | 28.71 | 11.61 | 16.68 | 38.46 | 48.62 | 23.58 | 15.85 |
| Insulin - 34 peptides 17mers 1 ug/mL | 17.87 | 8.92 | 11.61 | 12.46 | 10.02 | 16.15 | 12.98 | 8.60 |
| Insulin - 34 peptides 17mers 0.4 ug/mL | 2.19 | 2.88 | 2.65 | 2.27 | 1.40 | 1.67 | 1.00 | 2.15 |
| GAD65 pool 1 - 75 17mers 4 ug/mL | 7.25 | 4.58 | 2.52 | 1.93 | 20.79 | 6.15 | 1.88 | 1.57 |
| GAD65 pool 1 - 75 17mers 1 ug/mL | 2.51 | 2.65 | 1.31 | 1.14 | 2.62 | 2.25 | 1.08 | 1.03 |
| GAD65 pool 1 - 75 17mers 0.4 ug/mL | 1.07 | 1.78 | 1.12 | 1.33 | 1.25 | 1.43 | 1.24 | 0.91 |
| GAD65 pool 2 - 76 17mers 4 ug/mL | 20.68 | 2.32 | 11.61 | 15.83 | 7.58 | 12.68 | 1.50 | 2.51 |
| GAD65 pool 2 - 76 17mers 1 ug/mL | 8.26 | 2.64 | 11.61 | 6.16 | 11.97 | 5.96 | 4.96 | 2.09 |
| GAD65 pool 2 - 76 17mers 0.4 ug/mL | 2.05 | 1.92 | 5.98 | 2.70 | 6.92 | 5.16 | 0.85 | 1.72 |
| IA2 pool 1 - 85 17mers 4 ug/mL | 37.89 | 7.29 | 11.61 | 16.63 | 17.43 | 13.49 | 4.94 | 3.83 |
| IA2 pool 1 - 85 17mers 1 ug/mL | 11.82 | 3.35 | 3.91 | 2.10 | 5.58 | 9.16 | 1.76 | 1.63 |
| IA2 pool 1 - 85 17mers 0.4 ug/mL | 5.10 | 2.45 | 1.50 | 1.39 | 1.01 | 1.46 | 0.96 | 0.83 |
| IA2 pool 2 - 85 17mers 4 ug/mL | 6.26 | 14.62 | 11.61 | 16.68 | 38.46 | 48.62 | 15.26 | 16.27 |
| IA2 pool 2 - 85 17mers 1 ug/mL | 33.87 | 9.77 | 11.61 | 6.94 | 27.17 | 48.62 | 6.84 | 3.29 |
| IA2 pool 2 - 85 17mers 0.4 ug/mL | 5.46 | 1.25 | 2.09 | 5.56 | 8.28 | 14.11 | 1.35 | 0.76 |
| IA2 pool 3 - 85 17mers 4 ug/mL | 0.97 | 2.34 | 1.39 | 1.50 | 4.35 | 3.14 | 6.41 | 3.76 |
| IA2 pool 3 - 85 17mers 1 ug/mL | 2.85 | 2.31 | 1.15 | 0.92 | 1.18 | 1.23 | 1.25 | 1.72 |
| IA2 pool 3 - 85 17mers 0.4 ug/mL | 1.90 | 1.13 | 1.07 | 3.92 | 0.90 | 0.96 | 1.16 | 0.96 |

### T1D Autoantigen Peptide-Specific Responses:

Each of the 440 individual T1D autoantigen-derived 17mer peptides (20 μg/mL) were incubated in a single well with whole blood collected 6-days after commencing oral gluten challenge. Amongst the 34 preproinsulin-derived peptides, one elicited IP-10 responses in all four subjects that were 2-fold greater than to medium alone. Three 17mers in the 155-member GAD65 peptide library elicited increased IP-10 release in all four subjects. One peptide amongst the 255 17mers in the IA-2 library consistently stimulated IP-10 and IL-2 release, and in two subjects also stimulated IFN-γ release, suggesting that it is the immuno-dominant peptide in IA-2. A second peptide, evoked IP-10 but not IL-2 or IFN-γ release in all four subjects.

### CONCLUSIONS

Oral gluten challenge does not enhance whole blood cytokine release in response to T1D autoantigen-derived peptides, as it does to gluten-derived peptides in Celiac disease. However, IP-10 and IL-2 both offer greater sensitivity than IFN-γ in whole blood cytokine release assays for gluten-reactive T cells in Celiac disease, and when applied to screening peptides derived from T1D autoantigens in patients with T1D and Celiac disease, identify amino acid sequences that are consistently immuno-dominant. One 17mer peptide from preproinsulin, three from GAD65 and two from IA-2 stimulated elevated whole blood IP-10 release in four of four T1D subjects. Several other sequences commonly but less consistently and less potently stimulated IP-10 release in the T1D subjects assessed in this study. Hence, application of multiplex cytokine and chemokine measurement to epitope mapping in T1D reveals a hierarchy of epitopes not previously evident in the classical T1D-associated autoantigens preproinsulin, GAD65 and IA-2.

### Example 4

### Use of IP-10 to Measure Rare Antigen-Specific T Cells

### Methods

HLA-DQ2.5-positive celiac disease subjects on gluten-free diet were used in this study. Blood was collected immediately before and 6 days after commencing 3-day oral gluten challenge. Whole blood or PBMCs were incubated with pools or single peptides derived from gluten or recall antigens. IFNγ and IP-10 levels were measured in plasma from the whole blood that was incubated in 96-well plates with peptides or peptide pools. Plasma cytokine/chemokine levels were measured by MAGPIX® multiplex bead assay (IFNγ and IP-10) or by ELISA (IFNγ and IP-10), and PBMC separated from the same blood sample were incubated in overnight IFNγ ELISpot assays.

### Results

It was observed that IP-10 was elevated in plasma from blood incubated with gluten peptide pools 6 days after commencing the oral gluten challenge. The elevation was more statistically significant with IP-10 (p<0.002) than with IFN-γ (not significant) (Table 4 and 5). Using a cut-off of stimulation index >1.25 and cytokine level >100 pg/mL to indicate a positive result, the IP-10 assay was positive in 7/10 subjects prior to gluten challenge and 10/10 subjects after gluten challenge, whereas the IFN-γ assay using gluten peptide pools, with a cut-off of stimulation index >1.25 and cytokine level >7.2 pg/mL to indicate a positive result, was positive in 1/10 subjects pre-gluten challenge and 7/10 subjects after gluten challenge. Thus, the IP-10 assay was very effective both before and after gluten challenge for detecting antigen-specific T cells.

TABLE 4
Whole blood secretion of IFNγ pre- and post-gluten challenge

| | | Day 0 | | Day 6 |
|---|---|---|---|---|
| | Day 0 | Gluten pep- | Day 6 | Gluten pep- |
| | Ratio | tides minus | Ratio | tides minus |
| IFNγ | Gluten pep- | Medium | Gluten pep- | Medium |
| MAGPIX | tides:Medium | (pg/mL) | tides:Medium | (pg/mL) |
| Minimum | 0.9969 | −0.1 | 0.72 | −259 |
| 25% | 1.086 | 1.205 | 0.9014 | −17.58 |
| Percentile | | | | |
| Median | 1.265 | 4.625 | 5.53 | 50.77 |
| 75% | 1.689 | 21.01 | 29.82 | 185.6 |
| Percentile | | | | |
| Maximum | 2.6 | 143 | 228.6 | 728.2 |

Day 0 = prior to oral gluten challenge, Day 6 = 6 days after commencing a 3-day gluten challenge.

TABLE 5
Whole blood secretion of IP10 pre- and post-gluten challenge

| | | Day 0 | | Day 6 |
|---|---|---|---|---|
| | Day 0 | Gluten pep- | Day 6 | Gluten pep- |
| | Ratio | tides minus | Ratio | tides minus |
| IP10 | Gluten pep- | Medium | Gluten pep- | Medium |
| MAGPIX | tides:Medium | (pg/mL) | tides:Medium | (pg/mL) |
| Minimum | 1.192 | 65.26 | 3.648 | 1230 |
| 25% | 1.235 | 100.7 | 5.861 | 2168 |
| Percentile | | | | |
| Median | 1.536 | 274.7 | 13.00 | 6998 |
| 75% | 1.876 | 608.8 | 17.99 | 9333 |
| Percentile | | | | |
| Maximum | 3.186 | 1453 | 26.15 | 9618 |

Day 0 = prior to oral gluten challenge, Day 6 = 6 days after commencing a 3-day gluten challenge.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of assaying an antigen-specific T cell response, the method comprising:
measuring a level of IP-10 in a sample comprising an antigen-specific T cell obtained from a subject.

2. The method of claim 1, wherein the antigen specific T cell response is a rare antigen-specific T cell response and wherein the antigen-specific T cell is a rare antigen-specific T cell.

3. The method of claim 1 or 2, wherein the subject is a subject that has previously been administered IL-2 or an agent that stimulates IL-2 expression.

4. The method of claim 1 or 2, wherein the method further comprises administering IL-2 or an agent that stimulates IL-2 expression to the subject prior to the measuring.

5. The method of any one of claims 1 to 4, wherein the subject has or is suspected of having autoimmune disease, an allergy, an infectious disease or condition, or an adverse immune condition caused by administration of an isolated, recombinant or synthetic protein or peptide.

6. The method of any one of claims 1 to 4, wherein the subject has or is suspected of having an autoimmune disease and the antigen-specific T cell is a autoantigen-specific T cell.

7. The method of claim 6, wherein the subject has or is suspected of having the autoimmune disease and Celiac disease.

8. The method of claim 7, wherein the subject is a subject that has previously been administered a composition comprising a gluten peptide.

9. The method of claim 7, wherein the method further comprises administering a composition comprising a gluten peptide to the subject prior to the measuring.

10. The method of claim 8 or 9, wherein the composition is or has previously been administered to the subject more than once.

11. The method of claim 10 wherein the composition is or has previously been administered to the subject at least once a day for three days.

12. The method of any one of claims 8 to 11, wherein the composition comprises at least one of a wheat gluten, a barley hordein, and a rye secalin.

13. The method of claim 12, wherein the composition comprises at least two of a wheat gluten, a barley hordein, and a rye secalin.

14. The method of claim 13, wherein the composition comprises a wheat gluten, a barley hordein, and a rye secalin.

15. The method of any one of claims 9 to 14, wherein the administration of the composition is oral administration.

16. The method of claim 15, wherein the composition is a foodstuff.

17. The method of any one of claims 8 to 16, wherein the sample is obtained from the subject six days after administration of the composition.

18. The method of any one of claims 1 to 17, wherein the sample comprises whole blood or peripheral blood mononuclear cells.

19. The method of any one of claims 1 to 5 or 7 to 18, wherein the measuring of the level of IP-10 in the sample comprises contacting the sample with an antigen peptide and measuring the level of IP-10 in the sample.

20. The method of claim 6, wherein the measuring of the level of IP-10 in the sample comprises contacting the sample with an autoantigen peptide and measuring the level of IP-10 in the sample.

21. The method of claim 19 or 20, wherein the level of IP-10 is measured with an enzyme-linked immunosorbent assay (ELISA).

22. The method of claim 19 or 20, wherein the level of IP-10 is measured with a multiplex bead-based assay.

23. The method of any one of claim 19, 21 or 22, wherein the method further comprises comparing the level of IP-10 with a control level of IP-10 to identify or aid in identifying the antigen peptide as being one that is recognized by the antigen-specific T cell.

24. The method of claim 23, wherein an elevated level of IP-10 compared to the control level indicates that the antigen peptide is recognized by the antigen-specific T cell and wherein a decreased or substantially the same level of IP-10 compared to the control level indicates that the antigen peptide is not recognized by the antigen-specific T cell.

25. The method of claim 23 or 24′, wherein a level of IP-10 that is at least two-folder greater than the control level indicates that the antigen peptide is recognized by the antigen-specific T cell.

26. The method of claim 20, wherein the method further comprises comparing the level of IP-10 with a control level of IP-10 to identify or aid in identifying the autoantigen peptide as being one that is recognized by the rare autoantigen-specific T cell.

27. The method of claim 26, wherein an elevated level of IP-10 compared to the control level indicates that the autoantigen peptide is recognized by the rare autoantigen-specific T cell and wherein a decreased or substantially the same level of IP-10 compared to the control level indicates that the autoantigen peptide is not recognized by the rare autoantigen-specific T cell.

28. The method of claim 26 or 27, wherein a level of IP-10 that is at least two-folder greater than the control level indicates that the autoantigen peptide is recognized by the rare autoantigen-specific T cell.

29. The method of any one of claims 1 to 28, wherein the method further comprises measuring a level of IFN-γ and/or IL-2 in the sample.

30. The method of claim 29, wherein a level of IFN-γ that is at least two-fold greater than a control level of IFN-γ and/or a level of IL-2 that is at least two-fold greater than a control level of IL-2 indicates that the antigen peptide is recognized by the antigen-specific T cell or that the autoantigen peptide is recognized by the rare autoantigen-specific T cell.

31. A kit, comprising:
(a) a means for detecting a level of IP-10; and
(b) at least one antigen peptide.

32. The kit of claim 31, wherein the at least one antigen peptide is at least one autoantigen peptide.

33. The kit of claim 31, wherein the at least one antigen peptide is at least one foreign antigen.

34. The kit of any one of claims 31 to 33, wherein the means for detecting a level of IP-10 is an antibody that binds to IP-10.

35. The kit any one of claims 31 to 34, wherein the kit further comprises a composition comprising a gluten peptide.

36. The kit any one of claims 31 to 34, wherein the kit further comprises IL-2 or an agent that stimulates IL-2 expression.

37. The kit of claim 35, wherein the composition comprises at least one of a wheat gluten, a barley hordein, and a rye secalin.

38. The kit of claim 37, wherein the composition comprises at least two of a wheat gluten, a barley hordein, and a rye secalin.

39. The kit of claim 38, wherein the composition comprises a wheat gluten, a barley hordein, and a rye secalin.

40. The kit of any one of claims 31 to 39, wherein the kit comprises a container, such as a vial or tube, for whole blood.

41. The kit of claim 40, wherein the at least antigen peptide is dried on the wall of the container for whole blood.

42. The kit of claim any one of claims 31 to 40, wherein the at least one antigen peptide is in a solution or lyophilized in a separate container.

43. The kit of any one of claims 40 to 42, further comprising an anticoagulant.

44. The kit of any one of claims 40 to 43, wherein the container for whole blood and/or other container are present in duplicate or triplicate.

45. The kit of any one of claims 40 to 44, wherein the kit further comprises a negative control container, such as a vial or tube.

46. The kit of any one of claims 40 to 45, wherein the kit further comprises a positive control container, such as a vial or tube.

47. The kit of any one of claims 31 to 46, wherein the kit further comprises means for detecting a level of IFN-γ and/or IL-2.

48. The kit of claim 47, wherein the means for detecting a level of IFN-γ is an antibody that binds to IFN-γ.

49. The kit of claim 47 or 48, wherein the means for detecting a level of IL-2 is an antibody that binds to IL-2.

50. A method of assaying a T cell response to an islet autoantigen peptide, the method comprising:
(a) administering a composition comprising a gluten peptide to a first subject having or suspected of having Type 1 Diabetes (T1D) and Celiac disease; and
(b) measuring a first T cell response to at least one islet autoantigen peptide in a first sample obtained from the first subject after the administration of the composition.

51. The method of claim 50, wherein the at least one islet autoantigen peptide is selected from a proinsulin peptide, a 65-kDa isoform of glutamic acid decarboxylase (GAD 65) peptide, or an islet antigen-2 (IA-2) peptide.

52. The method of claim 51, wherein the at least one autoantigen peptide is a peptide comprising a sequence as put forth in Table 3.

53. The method of any one of claims 50 to 52, wherein the first sample comprises whole blood or peripheral blood mononuclear cells.

54. The method of any one of claims 50 to 53, wherein the composition is administered to the first subject more than once.

55. The method of claim 54, wherein the composition is administered to the first subject at least once a day for three days.

56. The method of any one of claims 50 to 55, wherein the composition comprises at least one of a wheat gluten, a barley hordein, and a rye secalin.

57. The method of claim 56, wherein the composition comprises at least two of a wheat gluten, a barley hordein, and a rye secalin.

58. The method of claim 57, wherein the composition comprises a wheat gluten, a barley hordein, and a rye secalin.

59. The method of any one of claims 50 to 58, wherein the administration of the composition is oral administration.

60. The method of claim 59, wherein the composition is a foodstuff.

61. The method of any one of claims 50 to 60, wherein the measuring of the first T cell response in the first sample comprises contacting the first sample with the at least one islet autoantigen peptide and measuring a level of at least one cytokine in the first sample.

62. The method of claim 61, wherein the at least one cytokine is IL-2, IFN-γ or IP-10.

63. The method of claim 62, wherein the at least one cytokine is IP-10 and IL-2.

64. The method of claim 62, wherein the at least one cytokine is IP-10, IFN-γ and IL-2

65. The method of any one of claims 61 to 64, wherein the level of the at least one cytokine is measured with an enzyme-linked immunosorbent assay (ELISA).

66. The method of any one of claims 61 to 64, wherein the level of the at least one cytokine is measured with a multiplex bead-based assay.

67. The method of any one of claims 61 to 64, wherein the level of the at least one cytokine is measured with an enzyme-linked immunosorbent spot (ELISpot) assay.

68. The method of any one of claims 50 to 67, wherein the method further comprises comparing the first T cell response with a control T cell response to identify or aid in identifying the first subject as in need of further testing for T1D if the T cell response measured in the first sample is elevated compared to the control T cell response, or to identify or aid in identifying the first subject as not in need of further testing for T1D if the first T cell response is substantially the same or decreased compared to the control T cell response.

69. The method of claim 68, wherein the method further comprises performing further testing for T1D if the first subject is identified as in need of further testing for T1D.

70. The method of claim 69, wherein the further testing comprises a glycated hemoglobin test, a glucose tolerance test, a fasting blood sugar test, and/or an immunoassay for autoantibodies.

71. The method of claim 70, wherein autoantibodies comprises one or more of islet cell autoantibodies, insulin autoantibodies, 65-kDa isoform of glutamic acid decarboxylase (GAD65) autoantibodies, islet antigen-2 (IA-2) autoantibodies, and zinc transporter (ZnT8) autoantibodies.

72. The method of any one of claims 50 to 71, wherein the first sample is obtained from the first subject six days after administration of the composition.

73. The method of any one of claims 50 to 72, wherein the method further comprises:
(c) administering a placebo to a second subject having or suspected of having Type 1 Diabetes (T1D) and Celiac disease; and
(d) measuring a second T cell response to the at least one islet autoantigen peptide in a second sample obtained from the second subject after the administration of the placebo.

74. The method of claim 73, wherein the measuring of the first and second T cell response are performed together in one assay.

75. The method of claim 73 or 74, wherein the composition is administered to the first subject more than once and the placebo is administered to the second subject more than once.

76. The method of claim 75, wherein the composition is administered to the first subject at least once a day for three days and the placebo is administered to the second subject at least once a day for three days.

77. The method of any one of claims 73 to 76, wherein the administration of the composition and the placebo is oral administration.

78. The method of claim 77, wherein the composition and the placebo are foodstuffs.

79. The method of any one of claims 73 to 78, wherein the measuring of the first and second T cell response in the first and second sample comprises contacting the first and second samples with the at least one islet autoantigen peptide and measuring a level of at least one cytokine in the first and second samples.

80. The method of claim 79, wherein the at least one cytokine is IL-2, IFN-γ or IP-10.

81. The method of claim 80, wherein the at least one cytokine is IP-10 and IL-2.

82. The method of claim 80, wherein the at least one cytokine is IP-10, IFN-γ and IL-2

83. The method of any one of claims 79 to 82, wherein the level of the at least one cytokine is measured with an enzyme-linked immunosorbent assay (ELISA).

84. The method of any one of claims 79 to 82, wherein the level of the at least one cytokine is measured with an enzyme-linked immunosorbent spot (ELISpot) assay.

85. The method of claim any one of claims 79 to 82, wherein the level of the at least one cytokine is measured with a multiplex bead-based assay.

86. The method of any one of claims 73 to 85, wherein the method further comprises comparing the first T cell response with the second T cell response to identify or aid in identifying the first subject as in need of further testing for T1D if the T cell response measured in the first sample is elevated compared to the second T cell response, or to identify or aid in identifying the first subject as not in further testing for T1D if the first T cell response is substantially the same or decreased compared to the second T cell response.

87. The method of claim 86, wherein the method further comprises performing further testing for T1D if the first subject is identified as in need of further testing for T1D.

88. The method of claim 87, wherein the further testing comprises a glycated hemoglobin test, a glucose tolerance test, a fasting blood sugar test, and/or an immunoassay for autoantibodies.

89. The method of claim 88, wherein autoantibodies comprises one or more of islet cell autoantibodies, insulin autoantibodies, 65-kDa isoform of glutamic acid decarboxylase (GAD65) autoantibodies, islet antigen-2 (IA-2) autoantibodies, and zinc transporter (ZnT8) autoantibodies.

90. The method of any one of claims 73 to 89, wherein the second sample is obtained from the second subject six days after administration of the placebo.

91. The method of any one of claims 50 to 90, wherein the method further comprises performing another test on the first subject and/or second subject prior to or after the steps of the method, preferably, in some embodiments, performing a serology and/or genotyping assay.

92. The method of claim 91, wherein the performing a serology and/or genotyping assay occurs prior to all of the steps recited in the method.

93. The method of claim 91, wherein the performing a serology and/or genotyping assay occurs after all of the steps recited in the method.

94. The method of any one of claims 50 to 93, wherein the first subject and/or second subject is HLA-DQ2.5 positive.

95. A kit, comprising:
(a) a means for detecting a T cell response; and
(b) at least one islet autoantigen peptide.

96. The kit of claim 95, wherein the at least one islet autoantigen peptide is selected from a proinsulin peptide, a 65-kDa isoform of glutamic acid decarboxylase (GAD 65) peptide, or an islet antigen-2 (IA-2) peptide.

97. The kit of claim 96, wherein the at least one autoantigen peptide is a peptide comprising a sequence as put forth in Table 3.

98. The kit of any one of claims 95 to 97, wherein the means for detecting a T cell response is an antibody that binds to a cytokine.

99. The kit of claim 98, wherein the antibody that binds to a cytokine is an antibody that binds to IL-2, IFN-γ or IP-10.

100. The method of claim 99, wherein the antibody that binds to a cytokine is an antibody that binds IP-10 and an antibody that binds to IL-2.

101. The method of claim 99, wherein the antibody that binds to a cytokine is an antibody that binds IP-10, an antibody that binds to IFN-γ, and an antibody that binds to IL-2.

102. The kit of any of claims 95 to 101, wherein the kit further comprises a composition comprising a gluten peptide.

103. The kit of any of claims 95 to 102, wherein the kit further comprises a placebo.

104. The kit of claim 103, wherein the composition and the placebo are foodstuffs.

105. The kit of claim any one of claims 102-104, wherein the composition comprises at least one of a wheat gluten, a barley hordein, and a rye secalin.

106. The kit of claim 105, wherein the composition comprises at least two of a wheat gluten, a barley hordein, and a rye secalin.

107. The kit of claim 105, wherein the composition comprises a wheat gluten, a barley hordein, and a rye secalin.

108. The kit of any one of claims 95 to 107, wherein the kit comprises a container, such as a vial or tube, for whole blood.

109. The kit of claim 108, wherein the at least one islet autoantigen peptide is dried on the wall of the container for whole blood.

110. The kit of any one of claims 95 to 108, wherein the at least one islet autoantigen peptide is in a solution or lyophilized in a separate container.

111. The kit of any one of claims 108 to 110, further comprising an anticoagulant.

112. The kit of any one of claims 108 to 111, wherein the container for whole blood and/or other container are present in duplicate or triplicate.

113. The kit of any one of claims 95 to 112, wherein the kit further comprises a negative control container, such as a vial or tube.

114. The kit of any one of claims 95 to 113, wherein the kit further comprises a positive control container, such as a vial or tube.

115. A method of screening for peptides that activate antigen-specific T cells, the method comprising:
providing a plurality of antigen peptides comprising sequences derived from an antigen;
contacting a plurality of samples comprising antigen-specific T cells obtained from a subject with the plurality of antigen peptides; and
measuring a level of IP-10 in each of the samples within the plurality of samples.

116. The method of 115, where the plurality of antigen peptides is 10-10,000 peptides.

117. The method of claim 115 or 116, wherein each of the antigen peptides within the plurality of antigen peptides is 10 to 20 amino acids in length.

118. The method of any one of claims 115 to 117, wherein the plurality of antigen peptides comprise one or more peptides comprising one or more deamidated variants of the sequences derived from the antigen.

119. The method of any one of claims 115 to 118, wherein the subject has or is suspected of having an autoimmune disease, an allergy, an infectious disease or condition, or an adverse immune condition caused by administration of an isolated, recombinant or synthetic protein or peptide.

120. The method of any one of claims 115 to 119, wherein the antigen is an autoantigen or a foreign antigen.

121. The method of any one of claims 115 to 119, wherein the level of IP-10 is measured using an ELISA assay or a multiplex bead-based assay.

122. The method of any one of claims 115 to 121, wherein the method further comprises measuring a level of IL-2 and/or IFN-γ in each of the samples within the plurality of samples.

123. The method of claim 122, wherein the level of IL-2 and/or IFN-γ is measured using an ELISA assay or a multiplex bead-based assay.

124. The method of any one of claims 115 to 123, wherein the method further comprises:
identifying a peptide within the plurality of antigen peptides as a peptide that activates antigen-specific T cells if the level of IP-10 is elevated compared to a control level of IP-10.

125. The method of any one of claims 115 to 124, wherein the method further comprises:
identifying a peptide within the plurality of antigen peptides as a peptide that activates antigen-specific T cells if the level of IP-10 is at least two-fold greater than a control level of IP-10.

126. The method of claim 124 or 125, wherein the control level of IP-10 is a level of IP-10 in a sample that has been contacted with a composition comprising phosphate buffered saline.

127. The method of claim 122, wherein the method further comprises:
identifying a peptide within the plurality of antigen peptides as a peptide that activates antigen-specific T cells if the level of IP-10 is elevated compared to a control level of IP-10 and the level of IL-2 and/or IFN-γ and is elevated compared to a control level of IL-2 and/or IFN-γ.

128. The method of claim 122 or 127, wherein the method further comprises:
identifying a peptide within the plurality of antigen peptides as a peptide that activates antigen-specific T cells if the level of IP-10 is at least two-fold greater than a control level of IP-10 and the level of IL-2 and/or IFN-γ and is at least two-fold greater than a control level of IL-2 and/or IFN-γ.

129. The method of claim 127 or 128, wherein the control level of IP-10 and the control level of IL-2 and/or IFN-γ is a level of IP-10 and IL-2 and/or IFN-γ in a sample that has been contacted with a composition comprising phosphate buffered saline.

130. The method of any one of claims 115 to 129, wherein the antigen-specific T cells are rare antigen-specific T cells.


SEQUENCE LISTING (604 sequences)

SEQ ID NO: 1 - protein, 110 aa, Artificial Sequence
  Synthetic Polypeptide
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 110 aa, Artificial Sequence; identical to SEQ ID NO: 1
  Synthetic Polypeptide

SEQ ID NO: 3 - protein, 110 aa, Artificial Sequence; identical to SEQ ID NO: 1
  Synthetic Polypeptide

SEQ ID NO: 4 - protein, 110 aa, Artificial Sequence; identical to SEQ ID NO: 1
  Synthetic Polypeptide

SEQ ID NO: 5 - protein, 110 aa, Artificial Sequence; identical to SEQ ID NO: 1
  Synthetic Polypeptide

SEQ ID NO: 6 - protein, 110 aa, Artificial Sequence; identical to SEQ ID NO: 1
  Synthetic Polypeptide

SEQ ID NO: 7 - protein, 110 aa, Artificial Sequence; identical to SEQ ID NO: 1
  Synthetic Polypeptide

SEQ ID NO: 8 - protein, 110 aa, Artificial Sequence; identical to SEQ ID NO: 1
  Synthetic Polypeptide

SEQ ID NO: 9 - protein, 110 aa, Artificial Sequence; identical to SEQ ID NO: 1
  Synthetic Polypeptide

SEQ ID NO: 10 - protein, 110 aa, Artificial Sequence; identical to SEQ ID NO: 1
  Synthetic Polypeptide

SEQ ID NO: 11 - protein, 110 aa, Artificial Sequence; identical to SEQ ID NO: 1
  Synthetic Polypeptide

SEQ ID NO: 12 - protein, 110 aa, Artificial Sequence; identical to SEQ ID NO: 1
  Synthetic Polypeptide

SEQ ID NO: 13 - protein, 110 aa, Artificial Sequence; identical to SEQ ID NO: 1
  Synthetic Polypeptide

SEQ ID NO: 14 - protein, 110 aa, Artificial Sequence; identical to SEQ ID NO: 1
  Synthetic Polypeptide

SEQ ID NO: 15 - protein, 110 aa, Artificial Sequence; identical to SEQ ID NO: 1
  Synthetic Polypeptide

SEQ ID NO: 16 - protein, 107 aa, Artificial Sequence
  Synthetic Polypeptide
MALWMRLLPLLALLALWGPDPAAAFVNQHLCGSHLVEALYLVCGERGFFYTPKTRREAEVGQVELGGGPGAGSLQPLALEGSLQKRGIVEQCCTSICSLYQLENYCN

SEQ ID NO: 17 - protein, 94 aa, Artificial Sequence
  Synthetic Polypeptide
WGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 18 - protein, 59 aa, Artificial Sequence
  Synthetic Polypeptide
MALWMRLLPLLALLALWGPDPAAAFVNQHLCGSHLVEALYLVCGERGFFYTPKTRREAE

SEQ ID NO: 19 - protein, 98 aa, Artificial Sequence
  Synthetic Polypeptide
MALWMRLLPLLALLALWGPDPAAAFVNQHLCGSHLVEALYLVCGERGFFYTPKTRREAEDLQGSLQPLALEGSLQKRGIVEQCCTSICSLYQLENYCN

SEQ ID NO: 20 - protein, 110 aa, Artificial Sequence
  Synthetic Polypeptide
MALWMRLLPLLALLALWGPDPAAAFVNQHLCGSHLVEALYLVCGERGFFYTPKTRREAEDLQVGEVELGGGPGAGSLQPLALEGSLQKRGIVEQCCTSICSLYQLENYCN

SEQ ID NO: 21 - protein, 107 aa, Artificial Sequence
  Synthetic Polypeptide
MALWMRLLPLLALLALWGPDPAAAFVNQHLCGSHLVEALYLVCGERGFFYTPKTRREAEVGEVELGGGPGAGSLQPLALEGSLQKRGIVEQCCTSICSLYQLENYCN

SEQ ID NO: 22 - protein, 94 aa, Artificial Sequence
  Synthetic Polypeptide
WGPDPAAAFVNQHLCGSHLVEALYLVCGERGFFYTPKTRREAEDLQVGEVELGGGPGAGSLQPLALEGSLQKRGIVEQCCTSICSLYQLENYCN

SEQ ID NO: 23 - protein, 59 aa, Artificial Sequence; identical to SEQ ID NO: 18
  Synthetic Polypeptide

SEQ ID NO: 24 - protein, 98 aa, Artificial Sequence
  Synthetic Polypeptide
MALWMRLLPLLALLALWGPDPAAAFVNQHLCGSHLVEALYLVCGERGFFYTPKTRREAEDLEGSLQPLALEGSLQKRGIVEQCCTSICSLYQLENYCN

SEQ ID NO: 25 - protein, 585 aa, Artificial Sequence
  Synthetic Polypeptide
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 26 - protein, 585 aa, Artificial Sequence
  Synthetic Polypeptide
MASPGSGFWSFGSEDGSGDSENPGTARAWCQVAQKFTGGIGNKLCALLYGDAEKPAESGGSQPPRAAARKAACACDEKPCSCSKVDVNYAFLHATDLLPACDGERPTLAFLQDVMNILLEYVVKSFDRSTKVIDFHYPNELLQEYNWELADQPQNLEEILMHCETTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKEKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 27 - protein, 585 aa, Artificial Sequence; identical to SEQ ID NO: 25
  Synthetic Polypeptide

SEQ ID NO: 28 - protein, 585 aa, Artificial Sequence; identical to SEQ ID NO: 25
  Synthetic Polypeptide

SEQ ID NO: 29 - protein, 585 aa, Artificial Sequence; identical to SEQ ID NO: 25
  Synthetic Polypeptide

SEQ ID NO: 30 - protein, 585 aa, Artificial Sequence; identical to SEQ ID NO: 25
  Synthetic Polypeptide

SEQ ID NO: 31 - protein, 585 aa, Artificial Sequence; identical to SEQ ID NO: 25
  Synthetic Polypeptide

SEQ ID NO: 32 - protein, 585 aa, Artificial Sequence; identical to SEQ ID NO: 25
  Synthetic Polypeptide

SEQ ID NO: 33 - protein, 537 aa, Artificial Sequence
  Synthetic Polypeptide
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 34 - protein, 585 aa, Artificial Sequence; identical to SEQ ID NO: 25
  Synthetic Polypeptide

SEQ ID NO: 35 - protein, 585 aa, Artificial Sequence; identical to SEQ ID NO: 25
  Synthetic Polypeptide

SEQ ID NO: 36 - protein, 585 aa, Artificial Sequence; identical to SEQ ID NO: 25
  Synthetic Polypeptide

SEQ ID NO: 37 - protein, 585 aa, Artificial Sequence; identical to SEQ ID NO: 25
  Synthetic Polypeptide

SEQ ID NO: 38 - protein, 585 aa, Artificial Sequence; identical to SEQ ID NO: 25
  Synthetic Polypeptide

SEQ ID NO: 39 - protein, 585 aa, Artificial Sequence; identical to SEQ ID NO: 25
  Synthetic Polypeptide

SEQ ID NO: 40 - protein, 419 aa, Artificial Sequence
  Synthetic Polypeptide
LKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 41 - protein, 419 aa, Artificial Sequence
  Synthetic Polypeptide
LKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKEKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 42 - protein, 600 aa, Artificial Sequence [sequence omitted]

SEQ ID NO: 43 - protein, 600 aa, Artificial Sequence
  Synthetic Polypeptide
MSPIHHHHHHLVPRGSEASNSGFWSFGSEDGSGDSENPGTARAWCQVAQKFTGGIGNKLCALLYGDAEKPAESGGSQPPRAAARKAACACDEKPCSCSKVDVNYAFLHATDLLPACDGERPTLAFLQDVMNILLEYVVKSFDRSTKVIDFHYPNELLQEYNWELADQPQNLEEILMHCETTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKEKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQEDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 44 - protein, 340 aa, Artificial Sequence
  Synthetic Polypeptide
SNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 45 - protein, 341 aa, Artificial Sequence
  Synthetic Polypeptide
ISNMYAMMIARFKMFPEVKEKGMAALPRLIAFTSEHSHFSLKKGAAALGIGTDSVILIKCDERGKMIPSDLERRILEAKEKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQEDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 46 - protein, 78 aa, Artificial Sequence
  Synthetic Polypeptide
MASPGSGFWSFGSEDGSGDSENPGTARAWCQVAQKFTGGIGNKLCDLLPACDGERPTLAFLQDVMNILLQYVVKSFDR

SEQ ID NO: 47 - protein, 78 aa, Artificial Sequence
  Synthetic Polypeptide
MASPGSGFWSFGSEDGSGDSENPGTARAWCQVAQKFTGGIGNKLCDLLPACDGERPTLAFLQDVMNILLEYVVKSFDR

SEQ ID NO: 48 - protein, 129 aa, Artificial Sequence
  Synthetic Polypeptide
MNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGT

SEQ ID NO: 49 - protein, 129 aa, Artificial Sequence
  Synthetic Polypeptide
MNILLEYVVKSFDRSTKVIDFHYPNELLQEYNWELADQPQNLEEILMHCETTLKYAIKTGHPRYFNQLSTGLDMVGLAADWLTSTANTNMFTYEIAPVFVLLEYVTLKKMREIIGWPGGSGDGIFSPGT

SEQ ID NO: 50 - protein, 188 aa, Artificial Sequence
  Synthetic Polypeptide
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGIVSSKIIKLFFRLQ

SEQ ID NO: 51 - protein, 188 aa, Artificial Sequence
  Synthetic Polypeptide
MASPGSGFWSFGSEDGSGDSENPGTARAWCQVAQKFTGGIGNKLCALLYGDAEKPAESGGSQPPRAAARKAACACDEKPCSCSKVDVNYAFLHATDLLPACDGERPTLAFLQDVMNILLEYVVKSFDRSTKVIDFHYPNELLQEYNWELADQPQNLEEILMHCETTLKYAIKTGIVSSKIIKLFFRLQ

SEQ ID NO: 52 - protein, 979 aa, Artificial Sequence
  Synthetic Polypeptide
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 53 - protein, 979 aa, Artificial Sequence
  Synthetic Polypeptide
MRRPRRPGGLGGSGGLRLLLCLLLLSSRPGGCSAVSAHGCLFDRRLCSHLEVCIEDGLFGECEVGVGQARPLLQVTSPVLQRLEGVLRQLMSQGLSWHDDLTEYVISQEMERIPRLRPPEPRPRDRSGLAPKRPGPAGELLLQDIPTGSAPAAEHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQEVPSPVSSEPPKAARPPVTPVLLEKKSPLGQSQPTVAGQPSARPAAEEYGYIVTDEKPLSLAAGVKLLEILAEHVHMSSGSFINISVVGPALTFRIRHNEQNLSLADVTQEAGLVKSELEAETGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATEGPLSHTIADFWEMVWESGCTVIVMLTPLVEDGVKQCDRYWPDEGASLYHVYEVNLVSEHIWCEDFLVRSFYLKNVQTQETRTLTEFHFLSWPAEGTPASTRPLLDFRRKVNKCYRGRSCPIIVHCSDGAGRTGTYILIDMVLNRMAKGVKEIDIAATLEHVRDERPGLVRSKDEFEFALTAVAEEVNAILKALPQ

SEQ ID NO: 54 - protein, 950 aa, Artificial Sequence
  Synthetic Polypeptide
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQNVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 55 - protein, 889 aa, Artificial Sequence
  Synthetic Polypeptide
MSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 56 - protein, 17 aa, Artificial Sequence
  Synthetic Polypeptide
VSSEPPKAARPPVTPVL

SEQ ID NO: 57 - protein, 17 aa, Artificial Sequence
  Synthetic Polypeptide
HSSTPSWCEEPAQANMD

SEQ ID NO: 58 - protein, 17 aa, Artificial Sequence
  Synthetic Polypeptide
KSLFNRAEGPPEPSRVS

SEQ ID NO: 59 - protein, 17 aa, Artificial Sequence
  Synthetic Polypeptide
ALCVRQHARQQDKERLA

SEQ ID NO: 60 - protein, 17 aa, Artificial Sequence
  Synthetic Polypeptide
TGHMILAYMEDHLRNRD

SEQ ID NO: 61 - protein, 17 aa, Artificial Sequence
  Synthetic Polypeptide
LLPPLLEHLLLPPQPPH

SEQ ID NO: 62 - protein, 17 aa, Artificial Sequence
  Synthetic Polypeptide
SPLQAELLPPLLEHLLL

SEQ ID NO: 63 - protein, 17 aa, Artificial Sequence
  Synthetic Polypeptide
EHVRDQRPGLVRSKDQF

SEQ ID NO: 64 - protein, 17 aa, Artificial Sequence
  Synthetic Polypeptide
ASPTSSEVQQVPSPVSS

SEQ ID NO: 65 - protein, 17 aa, Artificial Sequence
  Synthetic Polypeptide
PGHPTASPTSSEVQQVP

SEQ ID NO: 66 - protein, 17 aa, Artificial Sequence
  Synthetic Polypeptide
GVKLLEILAEHVHMSSG

SEQ ID NO: 67 - protein, 17 aa, Artificial Sequence
  Synthetic Polypeptide
EGYEKEGLGDRGEKPAS

SEQ ID NO: 68 - protein, 17 aa, Artificial Sequence
  Synthetic Polypeptide
TEFHFLSWPAEGTPAST

SEQ ID NO: 69 - protein, 17 aa, Artificial Sequence
  Synthetic Polypeptide
LKNVQTQETRTLTQFHF

SEQ ID NO: 70 - protein, 17 aa, Artificial Sequence
  Synthetic Polypeptide
AAEEYGYIVTDEKPLSL

SEQ ID NO: 71 - protein, 17 aa, Artificial Sequence
  Synthetic Polypeptide
GRDTAELPARTSPMPGH

SEQ ID NO: 72 - protein, 17 aa, Artificial Sequence
  Synthetic Polypeptide
QLLPKGAGRNPGGVVNV

SEQ ID NO: 73 - protein, 17 aa, Artificial Sequence
  Synthetic Polypeptide
DFLVRSFYLKNVQTQET

SEQ ID NO: 74 - protein, 17 aa, Artificial Sequence
  Synthetic Polypeptide
SSTPSWCEEPAQANMDI

SEQ ID NO: 75 - protein, 17 aa, Artificial Sequence
  Synthetic Polypeptide
LAAVLAGYGVELRQLTP

SEQ ID NO: 76 - protein, 17 aa, Artificial Sequence
  Synthetic Polypeptide
PIIEHDPRMPAYIATEG

SEQ ID NO: 77 - protein, 17 aa, Artificial Sequence
  Synthetic Polypeptide
LLTLVALAGVAGLLVAL

SEQ ID NO: 78 - protein, 17 aa, Artificial Sequence
  Synthetic Polypeptide
PASTRPLLDFRRKVNKC

SEQ ID NO: 79 - protein, 17 aa, Artificial Sequence
  Synthetic Polypeptide
GYGVELRQLTPEQLSTL

SEQ ID NO: 80 - protein, 17 aa, Artificial Sequence
  Synthetic Polypeptide
RQHARQQDKERLAALGP

SEQ ID NO: 81 - protein, 17 aa, Artificial Sequence
  Synthetic Polypeptide
GCSAVSAHGCLFDRRLC

SEQ ID NO: 82 - protein, 17 aa, Artificial Sequence
  Synthetic Polypeptide
QQDKERLAALGPEGAHG

SEQ ID NO: 83 - protein, 17 aa, Artificial Sequence
  Synthetic Polypeptide
PGGLGGSGGLRLLLCLL

SEQ ID NO: 84 - protein, 17 aa, Artificial Sequence
  Synthetic Polypeptide
AQGEGNIKKNRHPDFLP

SEQ ID NO: 85 - protein, 17 aa, Artificial Sequence
  Synthetic Polypeptide
PGHSYGDLPGPSPAQLF

SEQ ID NO: 86 - protein, 17 aa, Artificial Sequence
  Synthetic Polypeptide
YHVYEVNLVSEHIWCED

SEQ ID NO: 87 - protein, 17 aa, Artificial Sequence
  Synthetic Polypeptide
LSWHDDLTQYVISQEME

SEQ ID NO: 88 - protein, 17 aa, Artificial Sequence
  Synthetic Polypeptide
PVLLEKKSPLGQSQPTV

SEQ ID NO: 89 - protein, 17 aa, Artificial Sequence
  Synthetic Polypeptide
GDHPGHSYGDLPGPSPA

SEQ ID NO: 90 - protein, 17 aa, Artificial Sequence
  Synthetic Polypeptide
YWPDEGASLYHVYEVNL

SEQ ID NO: 91 - protein, 17 aa, Artificial Sequence
  Synthetic Polypeptide
QGPLSHTIADFWQMVWE

SEQ ID NO: 92 - protein, 17 aa, Artificial Sequence
  Synthetic Polypeptide
LVKSELEAQTGLQILQT

SEQ ID NO: 93 - protein, 17 aa, Artificial Sequence
  Synthetic Polypeptide
GVKEIDIAATLEHVRDE

SEQ ID NO: 94 - protein, 17 aa, Artificial Sequence
  Synthetic Polypeptide
VSEGSPGMVSVGPLPKA

SEQ ID NO: 95 - protein, 17 aa, Artificial Sequence
  Synthetic Polypeptide
GRNPGGVVNVGADIKKT

SEQ ID NO: 96 - protein, 17 aa, Artificial Sequence
  Synthetic Polypeptide
GLQILQTGVGQREEAAA

SEQ ID NO: 97 - protein, 17 aa, Artificial Sequence
  Synthetic Polypeptide
HDDLTEYVISQEMERIP

SEQ ID NO: 98 - protein, 17 aa, Artificial Sequence
  Synthetic Polypeptide
ALLQPYLFHQFGSRDGS

SEQ ID NO: 99 - protein, 17 aa, Artificial Sequence
  Synthetic Polypeptide
YQAEPNTCATAQGEGNI

SEQ ID NO: 100 - protein, 17 aa, Artificial Sequence
  Synthetic Polypeptide
LPKAEAPALFSRTASKG

SEQ ID NO: 101 - protein, 17 aa, Artificial Sequence
  Synthetic Polypeptide
PKAARPPVTPVLLEKKS

SEQ ID NO: 102 - protein, 17 aa, Artificial Sequence
  Synthetic Polypeptide
RPRDRSGLAPKRPGPAG

SEQ ID NO: 103 - protein, 17 aa, Artificial Sequence
  Synthetic Polypeptide
ALTAVAEEVNAILKALP

SEQ ID NO: 104 - protein, 17 aa, Artificial Sequence
  Synthetic Polypeptide
DFWQMVWESGCTVIVML

SEQ ID NO: 105 - protein, 17 aa, Artificial Sequence
  Synthetic Polypeptide
DRLAKEWQALCAYQAEP

SEQ ID NO: 106 - protein, 17 aa, Artificial Sequence
  Synthetic Polypeptide
RGRSCPIIVHCSDGAGR

SEQ ID NO: 107 - protein, 17 aa, Artificial Sequence
  Synthetic Polypeptide
ARIKLKVESSPSRSDYI

SEQ ID NO: 108 - protein, 17 aa, Artificial Sequence
  Synthetic Polypeptide
VSSVSSQFSDAAQASPS

SEQ ID NO: 109 - protein, 17 aa, Artificial Sequence
  Synthetic Polypeptide
QFHFLSWPAEGTPASTR

SEQ ID NO: 110 - protein, 17 aa, Artificial Sequence
  Synthetic Polypeptide
SDYINASPIIEHDPRMP

SEQ ID NO: 111 - protein, 17 aa, Artificial Sequence
  Synthetic Polypeptide
PMPGHPTASPTSSEVQQ

SEQ ID NO: 112 - protein, 17 aa, Artificial Sequence
  Synthetic Polypeptide
YILIDMVLNRMAKGVKE

SEQ ID NO: 113 - protein, 17 aa, Artificial Sequence
  Synthetic Polypeptide
EQNLSLADVTQEAGLVK

SEQ ID NO: 114 - protein, 17 aa, Artificial Sequence
  Synthetic Polypeptide
KGAGRNPGGVVNVGADI

SEQ ID NO: 115 - protein, 17 aa, Artificial Sequence
  Synthetic Polypeptide
SGGLRLLLCLLLLSSRP

SEQ ID NO: 116 - protein, 17 aa, Artificial Sequence
  Synthetic Polypeptide
AEEYGYIVTDQNVVGPA

SEQ ID NO: 117 - protein, 17 aa, Artificial Sequence
  Synthetic Polypeptide
QAEPNTCATAQGEGNIK

SEQ ID NO: 118 - protein, 17 aa, Artificial Sequence
  Synthetic Polypeptide
HGDTTFEYQDLCRQHMA

SEQ ID NO: 119 - protein, 17 aa, Artificial Sequence
  Synthetic Polypeptide
TQYVISQEMERIPRLRP

SEQ ID NO: 120 - protein, 17 aa, Artificial Sequence
  Synthetic Polypeptide
DIAATLEHVRDQRPGLV

SEQ ID NO: 121 - protein, 17 aa, Artificial Sequence
  Synthetic Polypeptide
RIPRLRPPEPRPRDRSG

SEQ ID NO: 122 - protein, 17 aa, Artificial Sequence
  Synthetic Polypeptide
KAEAPALFSRTASKGIF

SEQ ID NO: 123 - protein, 17 aa, Artificial Sequence
  Synthetic Polypeptide
QEMERIPRLRPPEPRPR

SEQ ID NO: 124 - protein, 17 aa, Artificial Sequence
  Synthetic Polypeptide
LCSHLEVCIQDGLFGQC

SEQ ID NO: 125 - protein, 17 aa, Artificial Sequence
  Synthetic Polypeptide
WCEEPAQANMDISTGHM

SEQ ID NO: 126 - protein, 17 aa, Artificial Sequence
  Synthetic Polypeptide
DLPGPSPAQLFQDSGLL

SEQ ID NO: 127 - protein, 17 aa, Artificial Sequence
  Synthetic Polypeptide
REEAAAVLPQTAHSTSP

SEQ ID NO: 128 - protein, 17 aa, Artificial Sequence
  Synthetic Polypeptide
GSFINISVVGPALTFRI

SEQ ID NO: 129 - protein, 17 aa, Artificial Sequence
  Synthetic Polypeptide
PAQANMDISTGHMILAY

SEQ ID NO: 130 - protein, 17 aa, Artificial Sequence
  Synthetic Polypeptide
PPQPPHPSLSYEPALLQ

SEQ ID NO: 131 - protein, 17 aa, Artificial Sequence
  Synthetic Polypeptide
GLFGQCQVGVGQARPLL

SEQ ID NO: 132 - protein, 17 aa, Artificial Sequence
  Synthetic Polypeptide
WQALCAYQAEPNTCATA

SEQ ID NO: 133 - protein, 17 aa, Artificial Sequence
  Synthetic Polypeptide
AQHRLPQPPVGKGGAGA

SEQ ID NO: 134 - protein, 17 aa, Artificial Sequence
  Synthetic Polypeptide
PPVTPVLLEKKSPLGQS

SEQ ID NO: 135 - protein, 17 aa, Artificial Sequence
  Synthetic Polypeptide
SHTIADFWEMVWESGCT

SEQ ID NO: 136 - protein, 17 aa, Artificial Sequence
  Synthetic Polypeptide
QDIPTGSAPAAEHRLPQ

SEQ ID NO: 137 - protein, 17 aa, Artificial Sequence
  Synthetic Polypeptide
QLMSQGLSWHDDLTQYV

SEQ ID NO: 138 - protein, 17 aa, Artificial Sequence
  Synthetic Polypeptide
GPALTFRIRHNEQNLSL

SEQ ID NO: 139 - protein, 17 aa, Artificial Sequence
  Synthetic Polypeptide
EYGYIVTDQNVVGPALT

SEQ ID NO: 140 - protein, 17 aa, Artificial Sequence
  Synthetic Polypeptide
RSKDEFEFALTAVAEEV

SEQ ID NO: 141 - protein, 17 aa, Artificial Sequence
  Synthetic Polypeptide
SPMRSVLLTLVALAGVA

SEQ ID NO: 142 - protein, 17 aa, Artificial Sequence
  Synthetic Polypeptide
TQQAGLVKSELEAQTGL

SEQ ID NO: 143 - protein, 17 aa, Artificial Sequence
  Synthetic Polypeptide
LFDRRLCSHLEVCIQDG

SEQ ID NO: 144 - protein, 17 aa, Artificial Sequence
  Synthetic Polypeptide
QSQPTVAGQPSARPAAE

SEQ ID NO: 145 - protein, 17 aa, Artificial Sequence
  Synthetic Polypeptide
NEQNLSLADVTQQAGLV

SEQ ID NO: 146 - protein, 17 aa, Artificial Sequence
  Synthetic Polypeptide
PAYIATQGPLSHTIADF

SEQ ID NO: 147 - protein, 17 aa, Artificial Sequence
  Synthetic Polypeptide
IADFWEMVWESGCTVIV

SEQ ID NO: 148 - protein, 17 aa, Artificial Sequence
  Synthetic Polypeptide
PSLSYEPALLQPYLFHQ

SEQ ID NO: 149 - protein, 17 aa, Artificial Sequence
  Synthetic Polypeptide
EGNIKKNRHPDFLPYDH

SEQ ID NO: 150 - protein, 17 aa, Artificial Sequence
  Synthetic Polypeptide
SHLEVCIEDGLFGECEV

SEQ ID NO: 151 - protein, 17 aa, Artificial Sequence
  Synthetic Polypeptide
HCSDGAGRTGTYILIDM

SEQ ID NO: 152 - protein, 17 aa, Artificial Sequence
  Synthetic Polypeptide
KNRHPDFLPYDHARIKL

SEQ ID NO: 153 - protein, 17 aa, Artificial Sequence
  Synthetic Polypeptide
VLQRLQGVLRQLMSQGL

SEQ ID NO: 154 - protein, 17 aa, Artificial Sequence
  Synthetic Polypeptide
QEVPSPVSSEPPKAARP

SEQ ID NO: 155 - protein, 17 aa, Artificial Sequence
  Synthetic Polypeptide
AEGPPEPSRVSSVSSQF

SEQ ID NO: 156 - protein, 17 aa, Artificial Sequence
  Synthetic Polypeptide
HPTASPTSSEVQEVPSP

SEQ ID NO: 157 - protein, 17 aa, Artificial Sequence
  Synthetic Polypeptide
VQPDAALQRLAAVLAGY

SEQ ID NO: 158 - protein, 17 aa, Artificial Sequence
  Synthetic Polypeptide
QTGLQILQTGVGQREEA

SEQ ID NO: 159 - protein, 17 aa, Artificial Sequence
  Synthetic Polypeptide
CIEDGLFGECEVGVGQA

SEQ ID NO: 160 - protein, 17 aa, Artificial Sequence
  Synthetic Polypeptide
ASSSLSPLQAELLPPLL

SEQ ID NO: 161 - protein, 17 aa, Artificial Sequence
  Synthetic Polypeptide
ILAYMEDHLRNRDRLAK

SEQ ID NO: 162 - protein, 17 aa, Artificial Sequence
  Synthetic Polypeptide
TPLVEDGVKQCDRYWPD

SEQ ID NO: 163 - protein, 17 aa, Artificial Sequence
  Synthetic Polypeptide
IVMLTPLVEDGVKQCDR

SEQ ID NO: 164 - protein, 17 aa, Artificial Sequence
  Synthetic Polypeptide
PQTAHSTSPMRSVLLTL

SEQ ID NO: 165 - protein, 17 aa, Artificial Sequence
  Synthetic Polypeptide
TQETRTLTQFHFLSWPA

SEQ ID NO: 166 - protein, 17 aa, Artificial Sequence
  Synthetic Polypeptide
QTGVGQREEAAAVLPQT

SEQ ID NO: 167 - protein, 17 aa, Artificial Sequence
  Synthetic Polypeptide
ETRTLTQFHFLSWPAEG

SEQ ID NO: 168 - protein, 17 aa, Artificial Sequence
  Synthetic Polypeptide
DLTEYVISQEMERIPRL

SEQ ID NO: 169 - protein, 17 aa, Artificial Sequence
  Synthetic Polypeptide
LGPEGAHGDTTFEYQDL

SEQ ID NO: 170 - protein, 17 aa, Artificial Sequence
  Synthetic Polypeptide
QFSDAAQASPSSHSSTP

SEQ ID NO: 171 - protein, 17 aa, Artificial Sequence
  Synthetic Polypeptide
VESSPSRSDYINASPII

SEQ ID NO: 172 - protein, 17 aa, Artificial Sequence
  Synthetic Polypeptide
LKNVQTQETRTLTEFHF

SEQ ID NO: 173 - protein, 17 aa, Artificial Sequence
  Synthetic Polypeptide
RKVNKCYRGRSCPIIVH

SEQ ID NO: 174 - protein, 17 aa, Artificial Sequence
  Synthetic Polypeptide
RPLLQVTSPVLQRLEGV

SEQ ID NO: 175 - protein, 17 aa, Artificial Sequence
  Synthetic Polypeptide
FGSRDGSRVSEGSPGMV

SEQ ID NO: 176 - protein, 17 aa, Artificial Sequence
  Synthetic Polypeptide
QRLAAVLAGYGVELRQL

SEQ ID NO: 177 - protein, 17 aa, Artificial Sequence
  Synthetic Polypeptide
VCIQDGLFGQCQVGVGQ

SEQ ID NO: 178 - protein, 17 aa, Artificial Sequence
  Synthetic Polypeptide
QDLCRQHMATKSLFNRA

SEQ ID NO: 179 - protein, 17 aa, Artificial Sequence
  Synthetic Polypeptide
ADVTQEAGLVKSELEAE

SEQ ID NO: 180 - protein, 17 aa, Artificial Sequence
  Synthetic Polypeptide
ANMDISTGHMILAYMED

SEQ ID NO: 181 - protein, 17 aa, Artificial Sequence
  Synthetic Polypeptide
EDGVKQCDRYWPDEGAS

SEQ ID NO: 182 - protein, 17 aa, Artificial Sequence
  Synthetic Polypeptide
QASPSSHSSTPSWCEEP

SEQ ID NO: 183 - protein, 17 aa, Artificial Sequence
  Synthetic Polypeptide
SSEPPKAARPPVTPVLL

SEQ ID NO: 184 - protein, 17 aa, Artificial Sequence
  Synthetic Polypeptide
LSSRPGGCSAVSAHGCL

SEQ ID NO: 185 - protein, 17 aa, Artificial Sequence
  Synthetic Polypeptide
SSPSRSDYINASPIIEH

SEQ ID NO: 186 - protein, 17 aa, Artificial Sequence
  Synthetic Polypeptide
KLLEILAEHVHMSSGSF

SEQ ID NO: 187 - protein, 17 aa, Artificial Sequence
  Synthetic Polypeptide
PTSSEVQEVPSPVSSEP

SEQ ID NO: 188 - protein, 17 aa, Artificial Sequence
  Synthetic Polypeptide
QRPGLVRSKDQFEFALT

SEQ ID NO: 189 - protein, 17 aa, Artificial Sequence
  Synthetic Polypeptide
SAPAAEHRLPQPPVGKG

SEQ ID NO: 190 - protein, 17 aa, Artificial Sequence
  Synthetic Polypeptide
EDHLRNRDRLAKEWQAL

SEQ ID NO: 191 - protein, 17 aa, Artificial Sequence
  Synthetic Polypeptide
GLAPKRPGPAGELLLQD

SEQ ID NO: 192 - protein, 17 aa, Artificial Sequence
  Synthetic Polypeptide
EHDPRMPAYIATQGPLS

SEQ ID NO: 193 - protein, 17 aa, Artificial Sequence
  Synthetic Polypeptide
PRMPAYIATEGPLSHTI

SEQ ID NO: 194 - protein, 17 aa, Artificial Sequence
  Synthetic Polypeptide
PPLLEHLLLPPQPPHPS

SEQ ID NO: 195 - protein, 17 aa, Artificial Sequence
  Synthetic Polypeptide
VTDQKPLSLAAGVKLLE

SEQ ID NO: 196 - protein, 17 aa, Artificial Sequence
  Synthetic Polypeptide
DEGASLYHVYEVNLVSE

SEQ ID NO: 197 - protein, 17 aa, Artificial Sequence
  Synthetic Polypeptide
CATAQGEGNIKKNRHPD

SEQ ID NO: 198 - protein, 17 aa, Artificial Sequence
  Synthetic Polypeptide
ALFSRTASKGIFGDHPG

SEQ ID NO: 199 - protein, 17 aa, Artificial Sequence
  Synthetic Polypeptide
ELPAPSRARVPRLPEQG

SEQ ID NO: 200 - protein, 17 aa, Artificial Sequence
  Synthetic Polypeptide
LVALAVALCVRQHARQQ

SEQ ID NO: 201 - protein, 17 aa, Artificial Sequence
  Synthetic Polypeptide
LEHVRDERPGLVRSKDE

SEQ ID NO: 202 - protein, 17 aa, Artificial Sequence
  Synthetic Polypeptide
VNLVSEHIWCEDFLVRS

SEQ ID NO: 203 - protein, 17 aa, Artificial Sequence
  Synthetic Polypeptide
IAATLEHVRDERPGLVR

SEQ ID NO: 204 - protein, 17 aa, Artificial Sequence
  Synthetic Polypeptide
GLFGECEVGVGQARPLL

SEQ ID NO: 205 - protein, 17 aa, Artificial Sequence
  Synthetic Polypeptide
PYLFHQFGSRDGSRVSE

SEQ ID NO: 206 - protein, 17 aa, Artificial Sequence
  Synthetic Polypeptide
AGASSSLSPLQAELLPP

SEQ ID NO: 207 - protein, 17 aa, Artificial Sequence
  Synthetic Polypeptide
VRSKDQFEFALTAVAEE

SEQ ID NO: 208 - protein, 17 aa, Artificial Sequence
  Synthetic Polypeptide
NRDRLAKEWQALCAYQA

SEQ ID NO: 209 - protein, 17 aa, Artificial Sequence
  Synthetic Polypeptide
HLLLPPQPPHPSLSYEP

SEQ ID NO: 210 - protein, 17 aa, Artificial Sequence
  Synthetic Polypeptide
GSPGMVSVGPLPKAEAP

SEQ ID NO: 211 - protein, 17 aa, Artificial Sequence
  Synthetic Polypeptide
SFYLKNVQTQETRTLTQ

SEQ ID NO: 212 - protein, 17 aa, Artificial Sequence
  Synthetic Polypeptide
KERLAALGPEGAHGDTT

SEQ ID NO: 213 - protein, 17 aa, Artificial Sequence
  Synthetic Polypeptide
TLLTLLQLLPKGAGRNP

SEQ ID NO: 214 - protein, 17 aa, Artificial Sequence
  Synthetic Polypeptide
AEEYGYIVTDQKPLSLA

SEQ ID NO: 215 - protein, 17 aa, Artificial Sequence
  Synthetic Polypeptide
VALAGVAGLLVALAVAL

SEQ ID NO: 216 - protein, 17 aa, Artificial Sequence
  Synthetic Polypeptide
GRTGTYILIDMVLNRMA

SEQ ID NO: 217 - protein, 17 aa, Artificial Sequence
  Synthetic Polypeptide
KTMEGPVEGRDTAELPA

SEQ ID NO: 218 - protein, 17 aa, Artificial Sequence
  Synthetic Polypeptide
HMSSGSFINISVVGPAL

SEQ ID NO: 219 - protein, 17 aa, Artificial Sequence
  Synthetic Polypeptide
TSPVLQRLEGVLRQLMS

SEQ ID NO: 220 - protein, 17 aa, Artificial Sequence
  Synthetic Polypeptide
EGLGDRGEKPASPAVQP

SEQ ID NO: 221 - protein, 17 aa, Artificial Sequence
  Synthetic Polypeptide
LLQDIPTGSAPAAQHRL

SEQ ID NO: 222 - protein, 17 aa, Artificial Sequence
  Synthetic Polypeptide
DVTQQAGLVKSELEAQT

SEQ ID NO: 223 - protein, 17 aa, Artificial Sequence
  Synthetic Polypeptide
LNRMAKGVKEIDIAATL

SEQ ID NO: 224 - protein, 17 aa, Artificial Sequence
  Synthetic Polypeptide
SELEAETGLQILQTGVG

SEQ ID NO: 225 - protein, 17 aa, Artificial Sequence
  Synthetic Polypeptide
LAEHVHMSSGSFINISV

SEQ ID NO: 226 - protein, 17 aa, Artificial Sequence
  Synthetic Polypeptide
IKKTMEGPVEGRDTAEL

SEQ ID NO: 227 - protein, 17 aa, Artificial Sequence
  Synthetic Polypeptide
GDRGEKPASPAVQPDAA

SEQ ID NO: 228 - protein, 17 aa, Artificial Sequence
  Synthetic Polypeptide
QETRTLTEFHFLSWPAE

SEQ ID NO: 229 - protein, 17 aa, Artificial Sequence
  Synthetic Polypeptide
GPSPAQLFQDSGLLYLA

SEQ ID NO: 230 - protein, 17 aa, Artificial Sequence
  Synthetic Polypeptide
NVGADIKKTMEGPVEGR

SEQ ID NO: 231 - protein, 17 aa, Artificial Sequence
  Synthetic Polypeptide
SLADVTQQAGLVKSELE

SEQ ID NO: 232 - protein, 17 aa, Artificial Sequence
  Synthetic Polypeptide
ERPGLVRSKDEFEFALT

SEQ ID NO: 233 - protein, 17 aa, Artificial Sequence
  Synthetic Polypeptide
LTAVAEEVNAILKALPQ

SEQ ID NO: 234 - protein, 17 aa, Artificial Sequence
  Synthetic Polypeptide
QPPVGKGGAGASSSLSP

SEQ ID NO: 235 - protein, 17 aa, Artificial Sequence
  Synthetic Polypeptide
AHSTSPMRSVLLTLVAL

SEQ ID NO: 236 - protein, 17 aa, Artificial Sequence
  Synthetic Polypeptide
HIWCEDFLVRSFYLKNV

SEQ ID NO: 237 - protein, 17 aa, Artificial Sequence
  Synthetic Polypeptide
FEYQDLCRQHMATKSLF

SEQ ID NO: 238 - protein, 17 aa, Artificial Sequence
  Synthetic Polypeptide
LPYDHARIKLKVESSPS

SEQ ID NO: 239 - protein, 17 aa, Artificial Sequence
  Synthetic Polypeptide
EEYGYIVTDEKPLSLAA

SEQ ID NO: 240 - protein, 17 aa, Artificial Sequence
  Synthetic Polypeptide
AGLVKSELEAETGLQIL

SEQ ID NO: 241 - protein, 17 aa, Artificial Sequence
  Synthetic Polypeptide
AGVAGLLVALAVALCVR

SEQ ID NO: 242 - protein, 17 aa, Artificial Sequence
  Synthetic Polypeptide
LFQDSGLLYLAQELPAP

SEQ ID NO: 243 - protein, 17 aa, Artificial Sequence
  Synthetic Polypeptide
EKPLSLAAGVKLLEILA

SEQ ID NO: 244 - protein, 17 aa, Artificial Sequence
  Synthetic Polypeptide
EQGSSSRAEDSPEGYEK

SEQ ID NO: 245 - protein, 17 aa, Artificial Sequence
  Synthetic Polypeptide
PVEGRDTAELPARTSPM

SEQ ID NO: 246 - protein, 17 aa, Artificial Sequence
  Synthetic Polypeptide
GVVNVGADIKKTMEGPV

SEQ ID NO: 247 - protein, 17 aa, Artificial Sequence
  Synthetic Polypeptide
MVSVGPLPKAEAPALFS

SEQ ID NO: 248 - protein, 17 aa, Artificial Sequence
  Synthetic Polypeptide
RQLTPEQLSTLLTLLQL

SEQ ID NO: 249 - protein, 17 aa, Artificial Sequence
  Synthetic Polypeptide
LEAQTGLQILQTGVGQR

SEQ ID NO: 250 - protein, 17 aa, Artificial Sequence
  Synthetic Polypeptide
PGPAGELLLQDIPTGSA

SEQ ID NO: 251 - protein, 17 aa, Artificial Sequence
  Synthetic Polypeptide
PSRARVPRLPEQGSSSR

SEQ ID NO: 252 - protein, 17 aa, Artificial Sequence
  Synthetic Polypeptide
GPAGELLLQDIPTGSAP

SEQ ID NO: 253 - protein, 17 aa, Artificial Sequence
  Synthetic Polypeptide
PLLDFRRKVNKCYRGRS

SEQ ID NO: 254 - protein, 17 aa, Artificial Sequence
  Synthetic Polypeptide
SQGLSWHDDLTEYVISQ

SEQ ID NO: 255 - protein, 17 aa, Artificial Sequence
  Synthetic Polypeptide
DLTQYVISQEMERIPRL

SEQ ID NO: 256 - protein, 17 aa, Artificial Sequence
  Synthetic Polypeptide
SARPAAEEYGYIVTDQK

SEQ ID NO: 257 - protein, 17 aa, Artificial Sequence
  Synthetic Polypeptide
LQVTSPVLQRLQGVLRQ

SEQ ID NO: 258 - protein, 17 aa, Artificial Sequence
  Synthetic Polypeptide
KQCDRYWPDEGASLYHV

SEQ ID NO: 259 - protein, 17 aa, Artificial Sequence
  Synthetic Polypeptide
KKSPLGQSQPTVAGQPS

SEQ ID NO: 260 - protein, 17 aa, Artificial Sequence
  Synthetic Polypeptide
QPTVAGQPSARPAAEEY

SEQ ID NO: 261 - protein, 17 aa, Artificial Sequence
  Synthetic Polypeptide
SRVSEGSPGMVSVGPLP

SEQ ID NO: 262 - protein, 17 aa, Artificial Sequence
  Synthetic Polypeptide
INASPIIEHDPRMPAYI

SEQ ID NO: 263 - protein, 17 aa, Artificial Sequence
  Synthetic Polypeptide
VSAHGCLFDRRLCSHLE

SEQ ID NO: 264 - protein, 17 aa, Artificial Sequence
  Synthetic Polypeptide
VGKGGAGASSSLSPLQA

SEQ ID NO: 265 - protein, 17 aa, Artificial Sequence
  Synthetic Polypeptide
HMATKSLFNRAEGPPEP

SEQ ID NO: 266 - protein, 17 aa, Artificial Sequence
  Synthetic Polypeptide
RRLCSHLEVCIEDGLFG

SEQ ID NO: 267 - protein, 17 aa, Artificial Sequence
  Synthetic Polypeptide
RLEGVLRQLMSQGLSWH

SEQ ID NO: 268 - protein, 17 aa, Artificial Sequence
  Synthetic Polypeptide
LEVCIQDGLFGQCQVGV

SEQ ID NO: 269 - protein, 17 aa, Artificial Sequence
  Synthetic Polypeptide
CPIIVHCSDGAGRTGTY

SEQ ID NO: 270 - protein, 17 aa, Artificial Sequence
  Synthetic Polypeptide
QGVLRQLMSQGLSWHDD

SEQ ID NO: 271 - protein, 17 aa, Artificial Sequence
  Synthetic Polypeptide
PDAALQRLAAVLAGYGV

SEQ ID NO: 272 - protein, 17 aa, Artificial Sequence
  Synthetic Polypeptide
FQDSGLLYLAQELPAPS

SEQ ID NO: 273 - protein, 17 aa, Artificial Sequence
  Synthetic Polypeptide
ATEGPLSHTIADFWEMV

SEQ ID NO: 274 - protein, 17 aa, Artificial Sequence
  Synthetic Polypeptide
ARPLLQVTSPVLQRLQG

SEQ ID NO: 275 - protein, 17 aa, Artificial Sequence
  Synthetic Polypeptide
CTVIVMLTPLVEDGVKQ

SEQ ID NO: 276 - protein, 17 aa, Artificial Sequence
  Synthetic Polypeptide
AAAVLPQTAHSTSPMRS

SEQ ID NO: 277 - protein, 17 aa, Artificial Sequence
  Synthetic Polypeptide
RIRHNEQNLSLADVTQE

SEQ ID NO: 278 - protein, 17 aa, Artificial Sequence
  Synthetic Polypeptide
TDQNVVGPALTFRIRHN

SEQ ID NO: 279 - protein, 17 aa, Artificial Sequence
  Synthetic Polypeptide
PLSLAAGVKLLEILAEH

SEQ ID NO: 280 - protein, 17 aa, Artificial Sequence
  Synthetic Polypeptide
LRPPEPRPRDRSGLAPK

SEQ ID NO: 281 - protein, 17 aa, Artificial Sequence
  Synthetic Polypeptide
SLSYEPALLQPYLFHQF

SEQ ID NO: 282 - protein, 17 aa, Artificial Sequence
  Synthetic Polypeptide
TLEHVRDERPGLVRSKD

SEQ ID NO: 283 - protein, 17 aa, Artificial Sequence
  Synthetic Polypeptide
SSEVQQVPSPVSSEPPK

SEQ ID NO: 284 - protein, 17 aa, Artificial Sequence
  Synthetic Polypeptide
WESGCTVIVMLTPLVED

SEQ ID NO: 285 - protein, 17 aa, Artificial Sequence
  Synthetic Polypeptide
GQPSARPAAEEYGYIVT

SEQ ID NO: 286 - protein, 17 aa, Artificial Sequence
  Synthetic Polypeptide
PASPAVQPDAALQRLAA

SEQ ID NO: 287 - protein, 17 aa, Artificial Sequence
  Synthetic Polypeptide
RPRRPGGLGGSGGLRLL

SEQ ID NO: 288 - protein, 17 aa, Artificial Sequence
  Synthetic Polypeptide
TGSAPAAQHRLPQPPVG

SEQ ID NO: 289 - protein, 17 aa, Artificial Sequence
  Synthetic Polypeptide
SHTIADFWQMVWESGCT

SEQ ID NO: 290 - protein, 17 aa, Artificial Sequence
  Synthetic Polypeptide
LYLAQELPAPSRARVPR

SEQ ID NO: 291 - protein, 17 aa, Artificial Sequence
  Synthetic Polypeptide
YGYIVTDQKPLSLAAGV

SEQ ID NO: 292 - protein, 17 aa, Artificial Sequence
  Synthetic Polypeptide
ISVVGPALTFRIRHNEQ

SEQ ID NO: 293 - protein, 17 aa, Artificial Sequence
  Synthetic Polypeptide
QLSTLLTLLQLLPKGAG

SEQ ID NO: 294 - protein, 17 aa, Artificial Sequence
  Synthetic Polypeptide
SLYHVYEVNLVSEHIWC

SEQ ID NO: 295 - protein, 17 aa, Artificial Sequence
  Synthetic Polypeptide
PEPSRVSSVSSQFSDAA

SEQ ID NO: 296 - protein, 17 aa, Artificial Sequence
  Synthetic Polypeptide
ASKGIFGDHPGHSYGDL

SEQ ID NO: 297 - protein, 17 aa, Artificial Sequence
  Synthetic Polypeptide
DMVLNRMAKGVKEIDIA

SEQ ID NO: 298 - protein, 17 aa, Artificial Sequence
  Synthetic Polypeptide
LLCLLLLSSRPGGCSAV

SEQ ID NO: 299 - protein, 17 aa, Artificial Sequence
  Synthetic Polypeptide
QVGVGQARPLLQVTSPV

SEQ ID NO: 300 - protein, 17 aa, Artificial Sequence
  Synthetic Polypeptide
YIATEGPLSHTIADFWE

SEQ ID NO: 301 - protein, 17 aa, Artificial Sequence
  Synthetic Polypeptide
EFALTAVAEEVNAILKA

SEQ ID NO: 302 - protein, 17 aa, Artificial Sequence
  Synthetic Polypeptide
VNKCYRGRSCPIIVHCS

SEQ ID NO: 303 - protein, 17 aa, Artificial Sequence
  Synthetic Polypeptide
RAEDSPEGYEKEGLGDR

SEQ ID NO: 304 - protein, 17 aa, Artificial Sequence
  Synthetic Polypeptide
MRRPRRPGGLGGSGGLR

SEQ ID NO: 305 - protein, 17 aa, Artificial Sequence
  Synthetic Polypeptide
ECEVGVGQARPLLQVTS

SEQ ID NO: 306 - protein, 17 aa, Artificial Sequence
  Synthetic Polypeptide
PRLPEQGSSSRAEDSPE

SEQ ID NO: 307 - protein, 17 aa, Artificial Sequence
  Synthetic Polypeptide
GYIVTDEKPLSLAAGVK

SEQ ID NO: 308 - protein, 17 aa, Artificial Sequence
  Synthetic Polypeptide
HPDFLPYDHARIKLKVE

SEQ ID NO: 309 - protein, 17 aa, Artificial Sequence
  Synthetic Polypeptide
WPAEGTPASTRPLLDFR

SEQ ID NO: 310 - protein, 17 aa, Artificial Sequence
  Synthetic Polypeptide
LPARTSPMPGHPTASPT

SEQ ID NO: 311 - protein, 17 aa, Artificial Sequence
  Synthetic Polypeptide
PIHHHHHHLVPRGSEAS

SEQ ID NO: 312 - protein, 17 aa, Artificial Sequence
  Synthetic Polypeptide
VILIKCDERGKMIPSDL

SEQ ID NO: 313 - protein, 17 aa, Artificial Sequence
  Synthetic Polypeptide
GDKVNFFRMVISNPAAT

SEQ ID NO: 314 - protein, 17 aa, Artificial Sequence
  Synthetic Polypeptide
MIPSDLERRILEAKEKG

SEQ ID NO: 315 - protein, 17 aa, Artificial Sequence
  Synthetic Polypeptide
MASPGSGFWSFGSEDGS

SEQ ID NO: 316 - protein, 17 aa, Artificial Sequence
  Synthetic Polypeptide
EMVFDGKPQHTNVCFWY

SEQ ID NO: 317 - protein, 17 aa, Artificial Sequence
  Synthetic Polypeptide
NQMHASYLFQQDKHYDL

SEQ ID NO: 318 - protein, 17 aa, Artificial Sequence
  Synthetic Polypeptide
TANTNMFTYEIAPVFVL

SEQ ID NO: 319 - protein, 17 aa, Artificial Sequence
  Synthetic Polypeptide
PQNLEEILMHCQTTLKY

SEQ ID NO: 320 - protein, 17 aa, Artificial Sequence
  Synthetic Polypeptide
GFWSFGSEDGSGDSENP

SEQ ID NO: 321 - protein, 17 aa, Artificial Sequence
  Synthetic Polypeptide
EYVTLKKMREIIGWPGG

SEQ ID NO: 322 - protein, 17 aa, Artificial Sequence
  Synthetic Polypeptide
YDTGDKALQCGRHVDVF

SEQ ID NO: 323 - protein, 17 aa, Artificial Sequence
  Synthetic Polypeptide
LCDLLPACDGERPTLAF

SEQ ID NO: 324 - protein, 17 aa, Artificial Sequence
  Synthetic Polypeptide
FTGGIGNKLCDLLPACD

SEQ ID NO: 325 - protein, 17 aa, Artificial Sequence
  Synthetic Polypeptide
LHATDLLPACDGERPTL

SEQ ID NO: 326 - protein, 17 aa, Artificial Sequence
  Synthetic Polypeptide
RLSKVAPVIKARMMEYG

SEQ ID NO: 327 - protein, 17 aa, Artificial Sequence
  Synthetic Polypeptide
QNCNQMHASYLFQEDKH

SEQ ID NO: 328 - protein, 17 aa, Artificial Sequence
  Synthetic Polypeptide
AFDPLLAVADICKKYKI

SEQ ID NO: 329 - protein, 17 aa, Artificial Sequence
  Synthetic Polypeptide
EAKQKGFVPFLVSATAG

SEQ ID NO: 330 - protein, 17 aa, Artificial Sequence
  Synthetic Polypeptide
CACDEKPCSCSKVDVNY

SEQ ID NO: 331 - protein, 17 aa, Artificial Sequence
  Synthetic Polypeptide
FDRSTKVIDFHYPNELL

SEQ ID NO: 332 - protein, 17 aa, Artificial Sequence
  Synthetic Polypeptide
VIDFHYPNELLQEYNWE

SEQ ID NO: 333 - protein, 17 aa, Artificial Sequence
  Synthetic Polypeptide
KTGIVSSKIIKLFFRLQ

SEQ ID NO: 334 - protein, 17 aa, Artificial Sequence
  Synthetic Polypeptide
PACDGERPTLAFLQDVM

SEQ ID NO: 335 - protein, 17 aa, Artificial Sequence
  Synthetic Polypeptide
SDLERRILEAKQKGFVP

SEQ ID NO: 336 - protein, 17 aa, Artificial Sequence
  Synthetic Polypeptide
IGTDSVILIKCDERGKM

SEQ ID NO: 337 - protein, 17 aa, Artificial Sequence
  Synthetic Polypeptide
VISNPAATHQDIDFLIE

SEQ ID NO: 338 - protein, 17 aa, Artificial Sequence
  Synthetic Polypeptide
AIKTGHPRYFNQLSTGL

SEQ ID NO: 339 - protein, 17 aa, Artificial Sequence
  Synthetic Polypeptide
VAQKFTGGIGNKLCALL

SEQ ID NO: 340 - protein, 17 aa, Artificial Sequence
  Synthetic Polypeptide
HVDAAWGGGLLMSRKHK

SEQ ID NO: 341 - protein, 17 aa, Artificial Sequence
  Synthetic Polypeptide
MQNCNQMHASYLFQQDK

SEQ ID NO: 342 - protein, 17 aa, Artificial Sequence
  Synthetic Polypeptide
YLFQQDKHYDLSYDTGD

SEQ ID NO: 343 - protein, 17 aa, Artificial Sequence
  Synthetic Polypeptide
DFHYPNELLQEYNWELA

SEQ ID NO: 344 - protein, 17 aa, Artificial Sequence
  Synthetic Polypeptide
ELLQEYNWELADQPQNL

SEQ ID NO: 345 - protein, 17 aa, Artificial Sequence
  Synthetic Polypeptide
AAARKAACACDEKPCSC

SEQ ID NO: 346 - protein, 17 aa, Artificial Sequence
  Synthetic Polypeptide
EKGMAALPRLIAFTSEH

SEQ ID NO: 347 - protein, 17 aa, Artificial Sequence
  Synthetic Polypeptide
YKIWMHVDGLMQNCNQM

SEQ ID NO: 348 - protein, 17 aa, Artificial Sequence
  Synthetic Polypeptide
VDVNYAFLHATDLLPAC

SEQ ID NO: 349 - protein, 17 aa, Artificial Sequence
  Synthetic Polypeptide
PRYFNQLSTGLDMVGLA

SEQ ID NO: 350 - protein, 17 aa, Artificial Sequence
  Synthetic Polypeptide
QDIDFLIEEIERLGQDL

SEQ ID NO: 351 - protein, 17 aa, Artificial Sequence
  Synthetic Polypeptide
ENPGTARAWCQVAQKFT

SEQ ID NO: 352 - protein, 17 aa, Artificial Sequence
  Synthetic Polypeptide
PQNLEEILMHCETTLKY

SEQ ID NO: 353 - protein, 17 aa, Artificial Sequence
  Synthetic Polypeptide
RPTLAFLQDVMNILLQY

SEQ ID NO: 354 - protein, 17 aa, Artificial Sequence
  Synthetic Polypeptide
AESGGSQPPRAAARKAA

SEQ ID NO: 355 - protein, 17 aa, Artificial Sequence
  Synthetic Polypeptide
RTLEDNEERMSRLSKVA

SEQ ID NO: 356 - protein, 17 aa, Artificial Sequence
  Synthetic Polypeptide
VPFLVSATAGTTVYGAF

SEQ ID NO: 357 - protein, 17 aa, Artificial Sequence
  Synthetic Polypeptide
DLSYDTGDKALQCGRHV

SEQ ID NO: 358 - protein, 17 aa, Artificial Sequence
  Synthetic Polypeptide
FNQLSTGLDMVGLAADW

SEQ ID NO: 359 - protein, 17 aa, Artificial Sequence
  Synthetic Polypeptide
YVVKSFDRSTKVIDFHY

SEQ ID NO: 360 - protein, 17 aa, Artificial Sequence
  Synthetic Polypeptide
NMFTYEIAPVFVLLEYV

SEQ ID NO: 361 - protein, 17 aa, Artificial Sequence
  Synthetic Polypeptide
GFEAHVDKCLELAEYLY

SEQ ID NO: 362 - protein, 17 aa, Artificial Sequence
  Synthetic Polypeptide
SEDGSGDSENPGTARAW

SEQ ID NO: 363 - protein, 17 aa, Artificial Sequence
  Synthetic Polypeptide
RAAARKAACACDQKPCS

SEQ ID NO: 364 - protein, 17 aa, Artificial Sequence
  Synthetic Polypeptide
CFWYIPPSLRTLEDNEE

SEQ ID NO: 365 - protein, 17 aa, Artificial Sequence
  Synthetic Polypeptide
TGLDMVGLAADWLTSTA

SEQ ID NO: 366 - protein, 17 aa, Artificial Sequence
  Synthetic Polypeptide
IKARMMEYGTTMVSYQP

SEQ ID NO: 367 - protein, 17 aa, Artificial Sequence
  Synthetic Polypeptide
THQDIDFLIEEIERLGQ

SEQ ID NO: 368 - protein, 17 aa, Artificial Sequence
  Synthetic Polypeptide
NMYAMMIARFKMFPEVK

SEQ ID NO: 369 - protein, 17 aa, Artificial Sequence
  Synthetic Polypeptide
MNILLEYVVKSFDRSTK

SEQ ID NO: 370 - protein, 17 aa, Artificial Sequence
  Synthetic Polypeptide
TARAWCQVAQKFTGGIG

SEQ ID NO: 371 - protein, 17 aa, Artificial Sequence
  Synthetic Polypeptide
QDVMNILLQYVVKSFDR

SEQ ID NO: 372 - protein, 17 aa, Artificial Sequence
  Synthetic Polypeptide
ASPGSGFWSFGSEDGSG

SEQ ID NO: 373 - protein, 17 aa, Artificial Sequence
  Synthetic Polypeptide
HFSLKKGAAALGIGTDS

SEQ ID NO: 374 - protein, 17 aa, Artificial Sequence
  Synthetic Polypeptide
YQPLGDKVNFFRMVISN

SEQ ID NO: 375 - protein, 17 aa, Artificial Sequence
  Synthetic Polypeptide
ILLQYVVKSFDRSTKVI

SEQ ID NO: 376 - protein, 17 aa, Artificial Sequence
  Synthetic Polypeptide
AACACDQKPCSCSKVDV

SEQ ID NO: 377 - protein, 17 aa, Artificial Sequence
  Synthetic Polypeptide
AATHQDIDFLIEEPEAN

SEQ ID NO: 378 - protein, 17 aa, Artificial Sequence
  Synthetic Polypeptide
HHLVPRGSEASNSGFWS

SEQ ID NO: 379 - protein, 17 aa, Artificial Sequence
  Synthetic Polypeptide
KLSGVERANSVTWNPHK

SEQ ID NO: 380 - protein, 17 aa, Artificial Sequence
  Synthetic Polypeptide
CSCSKVDVNYAFLHATD

SEQ ID NO: 381 - protein, 17 aa, Artificial Sequence
  Synthetic Polypeptide
DPLLAVADICKKYKIWM

SEQ ID NO: 382 - protein, 17 aa, Artificial Sequence
  Synthetic Polypeptide
AKEKGFVPFLVSATAGT

SEQ ID NO: 383 - protein, 17 aa, Artificial Sequence
  Synthetic Polypeptide
KGTTGFEAHVDKCLELA

SEQ ID NO: 384 - protein, 17 aa, Artificial Sequence
  Synthetic Polypeptide
GIFSPGGAISNMYAMMI

SEQ ID NO: 385 - protein, 17 aa, Artificial Sequence
  Synthetic Polypeptide
HASYLFQEDKHYDLSYD

SEQ ID NO: 386 - protein, 17 aa, Artificial Sequence
  Synthetic Polypeptide
GTTMVSYQPLGDKVNFF

SEQ ID NO: 387 - protein, 17 aa, Artificial Sequence
  Synthetic Polypeptide
RRILEAKQKGFVPFLVS

SEQ ID NO: 388 - protein, 17 aa, Artificial Sequence
  Synthetic Polypeptide
LADQPQNLEEILMHCET

SEQ ID NO: 389 - protein, 17 aa, Artificial Sequence
  Synthetic Polypeptide
LQDVMNILLQYVVKSFD

SEQ ID NO: 390 - protein, 17 aa, Artificial Sequence
  Synthetic Polypeptide
IARFKMFPEVKEKGMAA

SEQ ID NO: 391 - protein, 17 aa, Artificial Sequence
  Synthetic Polypeptide
QCSALLVREEGLMQNCN

SEQ ID NO: 392 - protein, 17 aa, Artificial Sequence
  Synthetic Polypeptide
FSLKKGAAALGIGTDSV

SEQ ID NO: 393 - protein, 17 aa, Artificial Sequence
  Synthetic Polypeptide
LCALLYGDAEKPAESGG

SEQ ID NO: 394 - protein, 17 aa, Artificial Sequence
  Synthetic Polypeptide
ELAEYLYNIIKNREGYE

SEQ ID NO: 395 - protein, 17 aa, Artificial Sequence
  Synthetic Polypeptide
CQVAQKFTGGIGNKLCD

SEQ ID NO: 396 - protein, 17 aa, Artificial Sequence
  Synthetic Polypeptide
QEDKHYDLSYDTGDKAL

SEQ ID NO: 397 - protein, 17 aa, Artificial Sequence
  Synthetic Polypeptide
GIGNKLCALLYGDAEKP

SEQ ID NO: 398 - protein, 17 aa, Artificial Sequence
  Synthetic Polypeptide
NYAFLHATDLLPACDGE

SEQ ID NO: 399 - protein, 17 aa, Artificial Sequence
  Synthetic Polypeptide
SEASNSGFWSFGSEDGS

SEQ ID NO: 400 - protein, 17 aa, Artificial Sequence
  Synthetic Polypeptide
GDAEKPAESGGSQPPRA

SEQ ID NO: 401 - protein, 17 aa, Artificial Sequence
  Synthetic Polypeptide
AAALGIGTDSVILIKCD

SEQ ID NO: 402 - protein, 17 aa, Artificial Sequence
  Synthetic Polypeptide
WNPHKMMGVPLQCSALL

SEQ ID NO: 403 - protein, 17 aa, Artificial Sequence
  Synthetic Polypeptide
MMEYGTTMVSYQPLGDK

SEQ ID NO: 404 - protein, 17 aa, Artificial Sequence
  Synthetic Polypeptide
IGNKLCDLLPACDGERP

SEQ ID NO: 405 - protein, 17 aa, Artificial Sequence
  Synthetic Polypeptide
KYKIWMHVDAAWGGGLL

SEQ ID NO: 406 - protein, 17 aa, Artificial Sequence
  Synthetic Polypeptide
CDQKPCSCSKVDVNYAF

SEQ ID NO: 407 - protein, 17 aa, Artificial Sequence
  Synthetic Polypeptide
ERRILEAKEKGFVPFLV

SEQ ID NO: 408 - protein, 17 aa, Artificial Sequence
  Synthetic Polypeptide
DGSGDSENPGTARAWCQ

SEQ ID NO: 409 - protein, 17 aa, Artificial Sequence
  Synthetic Polypeptide
ETTLKYAIKTGHPRYFN

SEQ ID NO: 410 - protein, 17 aa, Artificial Sequence
  Synthetic Polypeptide
RANSVTWNPHKMMGVPL

SEQ ID NO: 411 - protein, 17 aa, Artificial Sequence
  Synthetic Polypeptide
LEYVVKSFDRSTKVIDF

SEQ ID NO: 412 - protein, 17 aa, Artificial Sequence
  Synthetic Polypeptide
PGGSGDGIFSPGGAISN

SEQ ID NO: 413 - protein, 17 aa, Artificial Sequence
  Synthetic Polypeptide
AALPRLIAFTSEHSHFS

SEQ ID NO: 414 - protein, 17 aa, Artificial Sequence
  Synthetic Polypeptide
KHKWKLSGVERANSVTW

SEQ ID NO: 415 - protein, 17 aa, Artificial Sequence
  Synthetic Polypeptide
FTSEHSHFSLKKGAAAL

SEQ ID NO: 416 - protein, 17 aa, Artificial Sequence
  Synthetic Polypeptide
ADWLTSTANTNMFTYEI

SEQ ID NO: 417 - protein, 17 aa, Artificial Sequence
  Synthetic Polypeptide
REGYEMVFDGKPQHTNV

SEQ ID NO: 418 - protein, 17 aa, Artificial Sequence
  Synthetic Polypeptide
GTTVYGAFDPLLAVADI

SEQ ID NO: 419 - protein, 17 aa, Artificial Sequence
  Synthetic Polypeptide
ADICKKYKIWMHVDGLM

SEQ ID NO: 420 - protein, 17 aa, Artificial Sequence
  Synthetic Polypeptide
ICKKYKIWMHVDAAWGG

SEQ ID NO: 421 - protein, 17 aa, Artificial Sequence
  Synthetic Polypeptide
NWELADQPQNLEEILMH

SEQ ID NO: 422 - protein, 17 aa, Artificial Sequence
  Synthetic Polypeptide
EERMSRLSKVAPVIKAR

SEQ ID NO: 423 - protein, 17 aa, Artificial Sequence
  Synthetic Polypeptide
ILMHCQTTLKYAIKTGH

SEQ ID NO: 424 - protein, 17 aa, Artificial Sequence
  Synthetic Polypeptide
TYEIAPVFVLLEYVTLK

SEQ ID NO: 425 - protein, 17 aa, Artificial Sequence
  Synthetic Polypeptide
GAISNMYAMMIARFKMF

SEQ ID NO: 426 - protein, 17 aa, Artificial Sequence
  Synthetic Polypeptide
YIPPSLRTLEDNEERMS

SEQ ID NO: 427 - protein, 17 aa, Artificial Sequence
  Synthetic Polypeptide
LMHCETTLKYAIKTGIV

SEQ ID NO: 428 - protein, 17 aa, Artificial Sequence
  Synthetic Polypeptide
MSPIHHHHHHLVPRGSE

SEQ ID NO: 429 - protein, 17 aa, Artificial Sequence
  Synthetic Polypeptide
GDKALQCGRHVDVFKLW

SEQ ID NO: 430 - protein, 17 aa, Artificial Sequence
  Synthetic Polypeptide
GLAADWLTSTANTNMFT

SEQ ID NO: 431 - protein, 17 aa, Artificial Sequence
  Synthetic Polypeptide
GLLMSRKHKWKLSGVER

SEQ ID NO: 432 - protein, 17 aa, Artificial Sequence
  Synthetic Polypeptide
DVFKLWLMWRAKGTTGF

SEQ ID NO: 433 - protein, 17 aa, Artificial Sequence
  Synthetic Polypeptide
LMWRAKGTTGFEAHVDK

SEQ ID NO: 434 - protein, 17 aa, Artificial Sequence
  Synthetic Polypeptide
APVIKARMMEYGTTMVS

SEQ ID NO: 435 - protein, 17 aa, Artificial Sequence
  Synthetic Polypeptide
MGVPLQCSALLVREEGL

SEQ ID NO: 436 - protein, 17 aa, Artificial Sequence
  Synthetic Polypeptide
LKYAIKTGIVSSKIIKL

SEQ ID NO: 437 - protein, 17 aa, Artificial Sequence
  Synthetic Polypeptide
IWMHVDGLMQNCNQMHA

SEQ ID NO: 438 - protein, 17 aa, Artificial Sequence
  Synthetic Polypeptide
QPPRAAARKAACACDEK

SEQ ID NO: 439 - protein, 17 aa, Artificial Sequence
  Synthetic Polypeptide
QCGRHVDVFKLWLMWRA

SEQ ID NO: 440 - protein, 17 aa, Artificial Sequence
  Synthetic Polypeptide
FPEVKEKGMAALPRLIA

SEQ ID NO: 441 - protein, 17 aa, Artificial Sequence
  Synthetic Polypeptide
TGGIGNKLCALLYGDAE

SEQ ID NO: 442 - protein, 17 aa, Artificial Sequence
  Synthetic Polypeptide
RMVISNPAATHQDIDFL

SEQ ID NO: 443 - protein, 17 aa, Artificial Sequence
  Synthetic Polypeptide
GKPQHTNVCFWYIPPSL

SEQ ID NO: 444 - protein, 17 aa, Artificial Sequence
  Synthetic Polypeptide
IIGWPGGSGDGIFSPGT

SEQ ID NO: 445 - protein, 17 aa, Artificial Sequence
  Synthetic Polypeptide
WGGGLLMSRKHKWKLSG

SEQ ID NO: 446 - protein, 17 aa, Artificial Sequence
  Synthetic Polypeptide
VNFFRMVISNPAATHQD

SEQ ID NO: 447 - protein, 17 aa, Artificial Sequence
  Synthetic Polypeptide
VSATAGTTVYGAFDPLL

SEQ ID NO: 448 - protein, 17 aa, Artificial Sequence
  Synthetic Polypeptide
DERGKMIPSDLERRILE

SEQ ID NO: 449 - protein, 17 aa, Artificial Sequence
  Synthetic Polypeptide
NVCFWYIPPSLRTLEDN

SEQ ID NO: 450 - protein, 17 aa, Artificial Sequence
  Synthetic Polypeptide
PPRAAARKAACACDQKP

SEQ ID NO: 451 - protein, 17 aa, Artificial Sequence
  Synthetic Polypeptide
GSEASNSGFWSFGSEDG

SEQ ID NO: 452 - protein, 17 aa, Artificial Sequence
  Synthetic Polypeptide
ACACDEKPCSCSKVDVN

SEQ ID NO: 453 - protein, 17 aa, Artificial Sequence
  Synthetic Polypeptide
AFLQDVMNILLEYVVKS

SEQ ID NO: 454 - protein, 17 aa, Artificial Sequence
  Synthetic Polypeptide
VFVLLEYVTLKKMREII

SEQ ID NO: 455 - protein, 17 aa, Artificial Sequence
  Synthetic Polypeptide
NLEEILMHCETTLKYAI

SEQ ID NO: 456 - protein, 17 aa, Artificial Sequence
  Synthetic Polypeptide
LPRLIAFTSEHSHFSLK

SEQ ID NO: 457 - protein, 17 aa, Artificial Sequence
  Synthetic Polypeptide
YNIIKNREGYEMVFDGK

SEQ ID NO: 458 - protein, 17 aa, Artificial Sequence
  Synthetic Polypeptide
QTTLKYAIKTGIVSSKI

SEQ ID NO: 459 - protein, 17 aa, Artificial Sequence
  Synthetic Polypeptide
KMREIIGWPGGSGDGIF

SEQ ID NO: 460 - protein, 17 aa, Artificial Sequence
  Synthetic Polypeptide
VDKCLELAEYLYNIIKN

SEQ ID NO: 461 - protein, 17 aa, Artificial Sequence
  Synthetic Polypeptide
VREEGLMQNCNQMHASY

SEQ ID NO: 462 - protein, 17 aa, Artificial Sequence
  Synthetic Polypeptide
FYTPKTRREAEDLQGSL

SEQ ID NO: 463 - protein, 17 aa, Artificial Sequence
  Synthetic Polypeptide
EDLQVGEVELGGGPGAG

SEQ ID NO: 464 - protein, 17 aa, Artificial Sequence
  Synthetic Polypeptide
RREAEDLEGSLQPLALE

SEQ ID NO: 465 - protein, 17 aa, Artificial Sequence
  Synthetic Polypeptide
CGSHLVEALYLVCGERG

SEQ ID NO: 466 - protein, 17 aa, Artificial Sequence
  Synthetic Polypeptide
REAEDLQVGQVELGGGP

SEQ ID NO: 467 - protein, 17 aa, Artificial Sequence
  Synthetic Polypeptide
ALLALWGPDPAAAFVNQ

SEQ ID NO: 468 - protein, 17 aa, Artificial Sequence
  Synthetic Polypeptide
YTPKTRREAEVGQVELG

SEQ ID NO: 469 - protein, 17 aa, Artificial Sequence
  Synthetic Polypeptide
GPGAGSLQPLALEGSLQ

SEQ ID NO: 470 - protein, 17 aa, Artificial Sequence
  Synthetic Polypeptide
QCCTSICSLYQLENYCN

SEQ ID NO: 471 - protein, 17 aa, Artificial Sequence
  Synthetic Polypeptide
VEALYLVCGERGFFYTP

SEQ ID NO: 472 - protein, 17 aa, Artificial Sequence
  Synthetic Polypeptide
NQHLCGSHLVEALYLVC

SEQ ID NO: 473 - protein, 17 aa, Artificial Sequence
  Synthetic Polypeptide
TPKTRREAEDLEGSLQP

SEQ ID NO: 474 - protein, 17 aa, Artificial Sequence
  Synthetic Polypeptide
SLQKRGIVEQCCTSICS

SEQ ID NO: 475 - protein, 17 aa, Artificial Sequence
  Synthetic Polypeptide
PKTRREAEVGEVELGGG

SEQ ID NO: 476 - protein, 17 aa, Artificial Sequence
  Synthetic Polypeptide
LQGSLQPLALEGSLQKR

SEQ ID NO: 477 - protein, 17 aa, Artificial Sequence
  Synthetic Polypeptide
YTPKTRREAEDLQVGQV

SEQ ID NO: 478 - protein, 17 aa, Artificial Sequence
  Synthetic Polypeptide
CGERGFFYTPKTRREAE

SEQ ID NO: 479 - protein, 17 aa, Artificial Sequence
  Synthetic Polypeptide
LYLVCGERGFFYTPKTR

SEQ ID NO: 480 - protein, 17 aa, Artificial Sequence
  Synthetic Polypeptide
EDLQVGQVELGGGPGAG

SEQ ID NO: 481 - protein, 17 aa, Artificial Sequence
  Synthetic Polypeptide
PKTRREAEVGQVELGGG

SEQ ID NO: 482 - protein, 17 aa, Artificial Sequence
  Synthetic Polypeptide
PAAAFVNQHLCGSHLVE

SEQ ID NO: 483 - protein, 17 aa, Artificial Sequence
  Synthetic Polypeptide
DLEGSLQPLALEGSLQK

SEQ ID NO: 484 - protein, 17 aa, Artificial Sequence
  Synthetic Polypeptide
VELGGGPGAGSLQPLAL

SEQ ID NO: 485 - protein, 17 aa, Artificial Sequence
  Synthetic Polypeptide
PLLALLALWGPDPAAAF

SEQ ID NO: 486 - protein, 17 aa, Artificial Sequence
  Synthetic Polypeptide
WGPDPAAAFVNQHLCGS

SEQ ID NO: 487 - protein, 17 aa, Artificial Sequence
  Synthetic Polypeptide
EAEVGQVELGGGPGAGS

SEQ ID NO: 488 - protein, 17 aa, Artificial Sequence
  Synthetic Polypeptide
MALWMRLLPLLALLALW

SEQ ID NO: 489 - protein, 17 aa, Artificial Sequence
  Synthetic Polypeptide
MRLLPLLALLALWGPDP

SEQ ID NO: 490 - protein, 17 aa, Artificial Sequence
  Synthetic Polypeptide
AEVGEVELGGGPGAGSL

SEQ ID NO: 491 - protein, 17 aa, Artificial Sequence
  Synthetic Polypeptide
RGFFYTPKTRREAEVGE

SEQ ID NO: 492 - protein, 17 aa, Artificial Sequence
  Synthetic Polypeptide
RREAEDLQGSLQPLALE

SEQ ID NO: 493 - protein, 17 aa, Artificial Sequence
  Synthetic Polypeptide
IVEQCCTSICSLYQLEN

SEQ ID NO: 494 - protein, 17 aa, Artificial Sequence
  Synthetic Polypeptide
PLALEGSLQKRGIVEQC

SEQ ID NO: 495 - protein, 17 aa, Artificial Sequence
  Synthetic Polypeptide
TRREAEDLQVGEVELGG

SEQ ID NO: 496 - protein, 4 aa, Artificial Sequence
  Synthetic Polypeptide
PELP

SEQ ID NO: 497 - protein, 5 aa, Artificial Sequence
  Synthetic Polypeptide
PELPY

SEQ ID NO: 498 - protein, 7 aa, Artificial Sequence
  Synthetic Polypeptide
QPELPYP

SEQ ID NO: 499 - protein, 7 aa, Artificial Sequence
  Synthetic Polypeptide
PQPELPY

SEQ ID NO: 500 - protein, 7 aa, Artificial Sequence
  Synthetic Polypeptide
PQPELPY

SEQ ID NO: 501 - protein, 7 aa, Artificial Sequence
  Synthetic Polypeptide
FPQPELP

SEQ ID NO: 502 - protein, 7 aa, Artificial Sequence
  Synthetic Polypeptide
PELPYPQ

SEQ ID NO: 503 - protein, 9 aa, Artificial Sequence
  Synthetic Polypeptide
FPQPELPYP

SEQ ID NO: 504 - protein, 9 aa, Artificial Sequence
  Synthetic Polypeptide
PYPQPELPY

SEQ ID NO: 505 - protein, 9 aa, Artificial Sequence
  Synthetic Polypeptide
PFPQPELPY

SEQ ID NO: 506 - protein, 9 aa, Artificial Sequence
  Synthetic Polypeptide
PQPELPYPQ

SEQ ID NO: 507 - protein, 9 aa, Artificial Sequence
  Synthetic Polypeptide
PFPQPEQPF

SEQ ID NO: 508 - protein, 9 aa, Artificial Sequence
  Synthetic Polypeptide
PQPEQPFPW

SEQ ID NO: 509 - protein, 9 aa, Artificial Sequence
  Synthetic Polypeptide
PIPEQPQPY

SEQ ID NO: 510 - protein, 9 aa, Artificial Sequence
  Synthetic Polypeptide
PQPELPYPQ

SEQ ID NO: 511 - protein, 9 aa, Artificial Sequence
  Synthetic Polypeptide
FRPEQPYPQ

SEQ ID NO: 512 - protein, 9 aa, Artificial Sequence
  Synthetic Polypeptide
PQQSFPEQQ

SEQ ID NO: 513 - protein, 9 aa, Artificial Sequence
  Synthetic Polypeptide
IQPEQPAQL

SEQ ID NO: 514 - protein, 9 aa, Artificial Sequence
  Synthetic Polypeptide
QQPEQPYPQ

SEQ ID NO: 515 - protein, 9 aa, Artificial Sequence
  Synthetic Polypeptide
SQPEQEFPQ

SEQ ID NO: 516 - protein, 9 aa, Artificial Sequence
  Synthetic Polypeptide
PQPEQEFPQ

SEQ ID NO: 517 - protein, 9 aa, Artificial Sequence
  Synthetic Polypeptide
QQPEQPFPQ

SEQ ID NO: 518 - protein, 9 aa, Artificial Sequence
  Synthetic Polypeptide
PQPEQPFCQ

SEQ ID NO: 519 - protein, 9 aa, Artificial Sequence
  Synthetic Polypeptide
QQPFPEQPQ

SEQ ID NO: 520 - protein, 9 aa, Artificial Sequence
  Synthetic Polypeptide
PFPQPEQPF

SEQ ID NO: 521 - protein, 9 aa, Artificial Sequence
  Synthetic Polypeptide
PQPEQPFPW

SEQ ID NO: 522 - protein, 9 aa, Artificial Sequence
  Synthetic Polypeptide
PFSEQEQPV

SEQ ID NO: 523 - protein, 9 aa, Artificial Sequence
  Synthetic Polypeptide
FSQQQESPF

SEQ ID NO: 524 - protein, 9 aa, Artificial Sequence
  Synthetic Polypeptide
PFPQPEQPF

SEQ ID NO: 525 - protein, 9 aa, Artificial Sequence
  Synthetic Polypeptide
PQPEQPFPQ

SEQ ID NO: 526 - protein, 9 aa, Artificial Sequence
  Synthetic Polypeptide
PIPEQPQPY

SEQ ID NO: 527 - protein, 9 aa, Artificial Sequence
  Synthetic Polypeptide
PFPQPEQPF

SEQ ID NO: 528 - protein, 9 aa, Artificial Sequence
  Synthetic Polypeptide
PQPEQPFPQ

SEQ ID NO: 529 - protein, 9 aa, Artificial Sequence
  Synthetic Polypeptide
PYPEQEEPF

SEQ ID NO: 530 - protein, 9 aa, Artificial Sequence
  Synthetic Polypeptide
PYPEQEQPF

SEQ ID NO: 531 - protein, 9 aa, Artificial Sequence
  Synthetic Polypeptide
PFSEQEQPV

SEQ ID NO: 532 - protein, 9 aa, Artificial Sequence
  Synthetic Polypeptide
EGSFQPSQE

SEQ ID NO: 533 - protein, 9 aa, Artificial Sequence
  Synthetic Polypeptide
EQPQQPFPQ

SEQ ID NO: 534 - protein, 9 aa, Artificial Sequence
  Synthetic Polypeptide
EQPQQPYPE

SEQ ID NO: 535 - protein, 10 aa, Artificial Sequence
  Synthetic Polypeptide
QQGYYPTSPQ

SEQ ID NO: 536 - protein, 9 aa, Artificial Sequence
  Synthetic Polypeptide
EGSFQPSQE

SEQ ID NO: 537 - protein, 9 aa, Artificial Sequence
  Synthetic Polypeptide
PQQSFPEQE

SEQ ID NO: 538 - protein, 9 aa, Artificial Sequence
  Synthetic Polypeptide
QGYYPTSPQ

SEQ ID NO: 539 - protein, 9 aa, Artificial Sequence
  Synthetic Polypeptide
PFPQPQLPY

SEQ ID NO: 540 - protein, 9 aa, Artificial Sequence
  Synthetic Polypeptide
PFPQPDLPY

SEQ ID NO: 541 - protein, 9 aa, Artificial Sequence
  Synthetic Polypeptide
PQPDLPYPQ

SEQ ID NO: 542 - protein, 9 aa, Artificial Sequence
  Synthetic Polypeptide
PFPQPDQPF

SEQ ID NO: 543 - protein, 9 aa, Artificial Sequence
  Synthetic Polypeptide
PQPDQPFPW

SEQ ID NO: 544 - protein, 9 aa, Artificial Sequence
  Synthetic Polypeptide
PIPDQPQPY

SEQ ID NO: 545 - protein, 15 aa, Artificial Sequence
  Synthetic Polypeptide
LQPFPQPDLPYPQPQ

SEQ ID NO: 546 - protein, 14 aa, Artificial Sequence
  Synthetic Polypeptide
QPFPQPDQPFPWQP

SEQ ID NO: 547 - protein, 15 aa, Artificial Sequence
  Synthetic Polypeptide
PQQPIPDQPQPYPQQ

SEQ ID NO: 548 - protein, 4 aa, Artificial Sequence
  Synthetic Polypeptide
PQLP

SEQ ID NO: 549 - protein, 5 aa, Artificial Sequence
  Synthetic Polypeptide
PQLPY

SEQ ID NO: 550 - protein, 7 aa, Artificial Sequence
  Synthetic Polypeptide
QPQLPYP

SEQ ID NO: 551 - protein, 7 aa, Artificial Sequence
  Synthetic Polypeptide
PQPQLPY

SEQ ID NO: 552 - protein, 7 aa, Artificial Sequence
  Synthetic Polypeptide
FPQPQLP

SEQ ID NO: 553 - protein, 7 aa, Artificial Sequence
  Synthetic Polypeptide
PQLPYPQ

SEQ ID NO: 554 - protein, 9 aa, Artificial Sequence
  Synthetic Polypeptide
FPQPQLPYP

SEQ ID NO: 555 - protein, 9 aa, Artificial Sequence
  Synthetic Polypeptide
PYPQPQLPY

SEQ ID NO: 556 - protein, 9 aa, Artificial Sequence
  Synthetic Polypeptide
PFPQPQLPY

SEQ ID NO: 557 - protein, 9 aa, Artificial Sequence
  Synthetic Polypeptide
PQPQLPYPQ

SEQ ID NO: 558 - protein, 9 aa, Artificial Sequence
  Synthetic Polypeptide
PFPQPQQPF

SEQ ID NO: 559 - protein, 9 aa, Artificial Sequence
  Synthetic Polypeptide
PQPQQPFPW

SEQ ID NO: 560 - protein, 9 aa, Artificial Sequence
  Synthetic Polypeptide
PIPQQPQPY

SEQ ID NO: 561 - protein, 15 aa, Artificial Sequence
  Synthetic Polypeptide
LQPFPQPQLPYPQPQ

SEQ ID NO: 562 - protein, 14 aa, Artificial Sequence
  Synthetic Polypeptide
QPFPQPQQPFPWQP

SEQ ID NO: 563 - protein, 15 aa, Artificial Sequence
  Synthetic Polypeptide
PEQPIPQQPQPYPQQ

SEQ ID NO: 564 - protein, 9 aa, Artificial Sequence
  Synthetic Polypeptide
PQPQLPYPQ

SEQ ID NO: 565 - protein, 9 aa, Artificial Sequence
  Synthetic Polypeptide
FRPQQPYPQ

SEQ ID NO: 566 - protein, 9 aa, Artificial Sequence
  Synthetic Polypeptide
PQQSFPQQQ

SEQ ID NO: 567 - protein, 9 aa, Artificial Sequence
  Synthetic Polypeptide
IQPQQPAQL

SEQ ID NO: 568 - protein, 9 aa, Artificial Sequence
  Synthetic Polypeptide
QQPQQPYPQ

SEQ ID NO: 569 - protein, 9 aa, Artificial Sequence
  Synthetic Polypeptide
SQPQQQFPQ

SEQ ID NO: 570 - protein, 9 aa, Artificial Sequence
  Synthetic Polypeptide
PQPQQQFPQ

SEQ ID NO: 571 - protein, 9 aa, Artificial Sequence
  Synthetic Polypeptide
QQPQQPFPQ

SEQ ID NO: 572 - protein, 9 aa, Artificial Sequence
  Synthetic Polypeptide
PQPQQPFCQ

SEQ ID NO: 573 - protein, 9 aa, Artificial Sequence
  Synthetic Polypeptide
QQPFPQQPQ

SEQ ID NO: 574 - protein, 9 aa, Artificial Sequence
  Synthetic Polypeptide
PFPQPQQPF

SEQ ID NO: 575 - protein, 9 aa, Artificial Sequence
  Synthetic Polypeptide
PQPQQPFPW

SEQ ID NO: 576 - protein, 9 aa, Artificial Sequence
  Synthetic Polypeptide
PFSQQQQPV

SEQ ID NO: 577 - protein, 9 aa, Artificial Sequence
  Synthetic Polypeptide
FSQQQQSPF

SEQ ID NO: 578 - protein, 9 aa, Artificial Sequence
  Synthetic Polypeptide
PFPQPQQPF

SEQ ID NO: 579 - protein, 9 aa, Artificial Sequence
  Synthetic Polypeptide
PQPQQPFPQ

SEQ ID NO: 580 - protein, 9 aa, Artificial Sequence
  Synthetic Polypeptide
PIPQQPQPY

SEQ ID NO: 581 - protein, 9 aa, Artificial Sequence
  Synthetic Polypeptide
PFPQPQQPF

SEQ ID NO: 582 - protein, 9 aa, Artificial Sequence
  Synthetic Polypeptide
PQPQQPFPQ

SEQ ID NO: 583 - protein, 9 aa, Artificial Sequence
  Synthetic Polypeptide
PYPEQQEPF

SEQ ID NO: 584 - protein, 9 aa, Artificial Sequence
  Synthetic Polypeptide
PYPEQQQPF

SEQ ID NO: 585 - protein, 9 aa, Artificial Sequence
  Synthetic Polypeptide
PFSQQQQPV

SEQ ID NO: 586 - protein, 9 aa, Artificial Sequence
  Synthetic Polypeptide
QGSFQPSQQ

SEQ ID NO: 587 - protein, 9 aa, Artificial Sequence
  Synthetic Polypeptide
QQPQQPFPQ

SEQ ID NO: 588 - protein, 9 aa, Artificial Sequence
  Synthetic Polypeptide
QQPQQPYPQ

SEQ ID NO: 589 - protein, 10 aa, Artificial Sequence
  Synthetic Polypeptide
QQGYYPTSPQ

SEQ ID NO: 590 - protein, 9 aa, Artificial Sequence
  Synthetic Polypeptide
QGSFQPSQQ

SEQ ID NO: 591 - protein, 9 aa, Artificial Sequence
  Synthetic Polypeptide
PQQSFPQQQ

SEQ ID NO: 592 - protein, 9 aa, Artificial Sequence
  Synthetic Polypeptide
QGYYPTSPQ

SEQ ID NO: 593 - protein, 15 aa, Artificial Sequence
  Synthetic Polypeptide
LQPFPQPELPYPQPQ

SEQ ID NO: 594 - protein, 14 aa, Artificial Sequence
  Synthetic Polypeptide
QPFPQPQQPFPWQP

SEQ ID NO: 595 - protein, 15 aa, Artificial Sequence
  Synthetic Polypeptide
PQQPIPQQPQPYPQQ

SEQ ID NO: 596 - protein, 144 aa, Artificial Sequence
  Synthetic Polypeptide
LSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIS

SEQ ID NO: 597 - protein, 143 aa, Artificial Sequence
  Synthetic Polypeptide
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 598 - protein, 77 aa, Artificial Sequence
  Synthetic Polypeptide
VPLSRTVRCTCISISNQPVNPRSLEKLEIIPASQFCPRVEIIATMKKKGEKRCLNPESKAIKNLLKAVSKERSKRSP

SEQ ID NO: 599 - DNA, 822 bp, Homo sapiens
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 600 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 601 - protein, 133 aa, Homo sapiens [sequence omitted]

SEQ ID NO: 602 - protein, 16 aa, Artificial Sequence
  Synthetic Polypeptide
XPEQPIPEQPQPYPQQ

SEQ ID NO: 603 - protein, 15 aa, Artificial Sequence
  Synthetic Polypeptide
XQPFPQPEQPFPWQP

SEQ ID NO: 604 - protein, 16 aa, Artificial Sequence
  Synthetic Polypeptide
XLQPFPQPELPYPQPQ